(19) United States
(12) Patent Application Publication
(10) Pub. No.: US 2024/0317723 A1
(43) Pub. Date: Sep. 26, 2024

(54) SUBSTITUTED AMINO AZA-HETEROARYL COMPOUNDS AS INHIBITORS OF THE HAEMATOPOIETIC PROGENITOR KINASE 1 (HPK1)

(71) Applicant: Ontario Institute for Cancer Research (OICR), Toronto (CA)

(72) Inventors: Rima Al-Awar, Toronto (CA); Methvin Isaac, Brampton (CA); Babu Joseph, Oakville (CA); Radek Laufer, Oakville (CA); Ganna Posternak, Toronto (CA); Michael Prakesch, Toronto (CA); David Uehling, Toronto (CA); Iain Watson, Toronto (CA); Brian Wilson, Mississauga (CA); Carlos Armando Zepeda-Velazquez, Mississauga (CA); Anh Chau, Toronto (CA); Tao Xin, Woodbridge (CA)

(73) Assignee: Ontario Institute for Cancer Research (OICR), Toronto, ON (CA)

(21) Appl. No.: 18/288,219

(22) PCT Filed: May 2, 2022
(86) PCT No.: PCT/CA2022/050676

§ 371 Date: Oct. 25, 2023

Related U.S. Application Data
(60) Provisional application No. 63/182,185, filed on Apr. 30, 2021.

(51) Int. Cl. C07D 405/14; A61K 31/497; A61K 31/517; A61K 31/5377; A61P 35/00; C07D 401/04; C07D 403/04; C07D 413/14

(52) U.S. Cl. CPC C07D 405/14; A61K 31/497; A61K 31/517; A61K 31/5377; A61P 35/00; C07D 401/04; C07D 403/04; C07D 413/14

(57) ABSTRACT

The present application relates to substituted amino aza-heteroaryl compounds of Formula (I) and substituted aza-heteroaryl compounds of Formula II or pharmaceutically acceptable salts, solvates and/or prodrugs thereof, to compositions comprising these compounds or pharmaceutically acceptable salts, solvates and/or prodrugs thereof, and various uses in the treatment of diseases, disorders or conditions that are treatable by inhibiting HPK1, such as cancer.

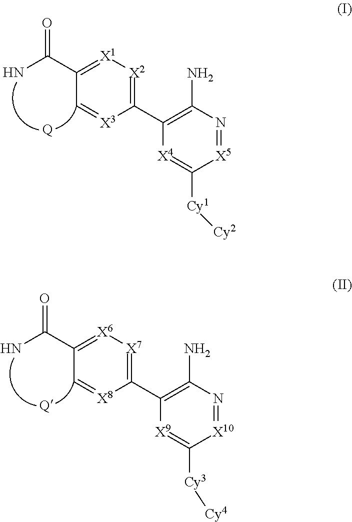

### RELATED APPLICATIONS

The present application claims the benefit of priority of co-pending U.S. provisional patent application No. 63/182,185 filed on Apr. 30, 2021, the contents of which are incorporated herein by reference in their entirety.

### FIELD

The present application relates to substituted amino aza-heteroaryl compounds, to processes for their preparation, to compositions comprising them, and to their use in therapy. More particularly, it relates to amino aza-heteroaryl compounds such as amino-pyrazine and amino-pyridazine derivatives useful in the treatment of diseases, disorders or conditions treatable by inhibiting HPK1.

### BACKGROUND

Tumors are genetically heterogeneous and have evolved mechanisms to hijack cellular growth and regulatory pathways, which makes it unlikely a single therapy will have a significant impact on patient survival. For this reason, immunotherapy has become an important paradigm in the treatment of some types of cancers. Immune effector cells such as T-cells and B-cells can suppress the proliferation of cancer cells by targeting abnormal, tumor-expressed antigens. For example, recent clinical testing of novel immunotherapy strategies (e.g. anti-PD1 and anti-PDL1) has demonstrated unprecedented and durable survival benefit even in advanced patients suffering from metastatic cancers. However, the overall excitement for this therapeutic approach is tempered by the observation that these responses to agents targeting the PD-1 axis are limited to a minority of cancer patients. Hence, in order to broaden the response rates in cancer patients, there is an urgent need to build on the tremendous promise of immunotherapy to more rapidly test rational combinations of small molecules with immuno-therapeutics. One such approach is the combination of a small molecule hematopoietic progenitor kinase 1 (HPK1) inhibitor with current anti-PD1/PDL1 immunotherapies. An HPK1 inhibitor should potentiate anti-tumor immune responses by stimulating T-cell proliferation and triggering tumor cell senescence and tumor clearance by T cells.

The hematopoietic progenitor kinase 1 (HPK1, MAP4K1), is a T-cell receptor (TCR)-proximal kinase involved in the regulation of proliferation and survival of primary T cells [Nat Immunol. 2007; 8(1):84-91.]. HPK1 is exclusively expressed in hematopoietic tissues and activates the c-Jun N-terminal kinase (JNK) and the NF-kB pathways [7]. Transient knockdown of HPK1 in T cells blocks activation of NF-kB [Crit Rev Oncol Hematol. 2008; 66(1):52-64]. Most strikingly, mice that received adoptive transfer of HPKI (−/−) T cells became resistant to lung tumor growth [Immunol Res. 2012; 54(1-3):262-5]. HPK1 has an N-terminal kinase domain and a C-terminal citron homology domain. Antigen receptor cross-linking leads to activation of HPK1 in T and B cells resulting in HPK1 relocation to the plasma membrane, autophosphorylation and transphosphorylation by protein kinase D1 (PKD1). Subsequent transphosphorylation by PKD1 and auto-phosphorylation within the kinase domain result in full activation of HPK1, which then regulates different cellular responses including apoptosis, activation-induced cell death and autoimmunity. HPK1 mediates negative regulation of the immune response via phosphorylation of SLP-76 (S376). Mutation of lysine-46 to methionine (designated HPK1-M46) in the ATP-binding site of the kinase domain abolishes catalytic activation of HPK1 resulting in a kinase-dead version of the full length kinase [Genes Dev. 1996; 10 (18):2251-64]. It has been reported that HPK1 inhibition in HPKI kinase-dead knock-in mice, when treated with anti-PD-1 or anti-PDL1 antibodies demonstrate enhanced efficacy in colon cancer models relative to anti-PD-1 or anti-PDL1 treatment alone (Cell Reports 2018, 25, 80-94, and PCT Patent Application Publication Nos. WO2016/205942 and WO2016/090300). Combining or sequencing immunotherapies that target distinct immune pathways is therefore a rational strategy to increase the magnitude of the antitumor immune response over that generated with a single agent.

HPK1 plays significant roles in regulating lymphocyte receptor signaling and function. Moreover, the restricted expression of HPK1 in hematopoietic cells and the roles of HPK1 in immune cells suggest that HPK1 would be an ideal drug target for enhancing antitumor immunity. Furthermore, data from preclinical studies suggest that gene-targeted disruption of HPK1 can promote the proliferation, survival, and function of various immune cells [e.g., T cells, NK cells, and dendritic cells (DC)], and synergistically inhibit tumor growth with anti-PD-1/PDL-1 mAb. Support for this strong rationale was evident by some reports in the literature that HPK1 kinase-dead knock-in mouse bearing colorectal tumors (MC38) showed significant growth arrest treated with an anti-PD1 or anti-PDL1 antibody (PCT Patent Application Publication No. WO2016/090300). Thus, combining a small molecule that inhibits HPK1 with another immunotherapy would appear to be a rational and more effective approach toward treating cancers.

Inhibiting kinases such as HPK1 therefore represents promising targets for immunooncology due to their role in limiting T-cell activation. At the same time, in the pursuit of these targets it is desirable that there be selectivity against other kinases that are involved in a robust T cell activation. An example of such a kinase includes, but is not limited to Lck (Sawasdikosol, et. al. Structure 27, 2019, 1-3).

### SUMMARY

Current cancer immunotherapy strategies seek to reverse immune tolerance either by modulating T cell co-receptor signals or boosting the recognition of tumor-associated antigens by using native biomolecules or mAbs. Selective HPK1 inhibitors, combined with other immuno-modulating agents should amplify the anti-tumor activity of immune cells. The present application discloses novel compounds that have such activity.

Accordingly, the application includes a compound of Formula (I), or a pharmaceutically acceptable salt, solvate and/or prodrug thereof,

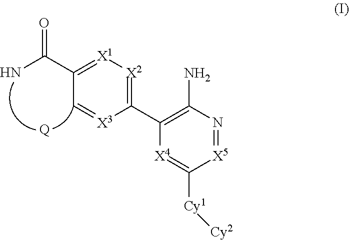

wherein:
X¹ is selected from N and CR¹;
X² and X³ are each independently selected from N and CR²;
X⁴ and X⁵ are each independently selected from N and CH, provided at least one of X⁴ and X⁵ is N;
Q is C₁₋₄alkylene optionally interrupted by a heteromoiety selected from O, S, S(O), SO₂ and NR³ and/or optionally substituted with one or more of R⁴ and/or optionally disubstituted on one carbon with R^{4a} and R^{4b}, provided that when Q comprises the heteromoiety the heteromoiety is separated from the ring amide NH by other than methylene; or
Q is C₂₋₄alkenylene optionally substituted with one or more of R^{4c}; or
Q is C═N or N═C optionally substituted with R^{4c};
R¹ is selected from H, halo, OR^{3a}, NR^{5a}R^{6a}, C₁₋₆alkyleneNR^{5a}R^{6a} and C₁₋₆alkyl;
R² is selected from H, halo and C₁₋₆alkyl;
R³ is selected from H and C₁₋₆alkyl;
each R⁴ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR⁵R⁶ and C₁₋₆alkyleneNR⁵R⁶;
R^{4a} and R^{4b} are joined to form, together with the carbon atom therebetween, a 3- to 6-membered, saturated or unsaturated ring optionally containing one heteromoiety selected from N, NH, NC₁₋₆alkyl, O, S, S(O), and SO₂ and optionally substituted with one or more of halo and C₁₋₆alkyl;
each R^{4c} is independently selected from halo, C₁₋₆alkyl, C₃₋₆cycloalkyl, C₃₋₆heterocycloalkyl, C₁₋₆alkyleneC₃₋₆cycloalkyl and C₁₋₆alkyleneC₃₋₆heterocycloalkyl, OH, OC₁₋₆alkyl, NR⁵R⁶, and C₁₋₆alkyleneNR⁵R⁶;
R⁵, R^{5a}, R⁶ and R^{6a} are each independently selected from H and C₁₋₆alkyl, or
R⁵ and R⁶ or R^{5a} and R^{6a} are joined to form, together with the nitrogen atom therebetween, a 3- to 7-membered, saturated or unsaturated ring optionally containing one additional heteromoiety selected from N, NH, NC₁₋₆alkyl, O, S, S(O), and SO₂ and optionally substituted with one or more of halo and C₁₋₆alkyl;
Cy¹ is C₆₋₁₀aryl or C₅₋₁₀heteroaryl, which is unsubstituted or substituted with one or more of R⁷;
each R⁷ is independently selected from halo, ═O, C₁₋₆alkyl, NR⁸R⁹, and C₁₋₆alkyleneNR⁸R⁹, C₃₋₇cycloalkyl, C₃₋₇heterocycloalkyl, C₁₋₆alkyleneC₃₋₇cycloalkyl and C₁₋₆alkyleneC₃₋₇heterocycloalkyl, the latter four groups being optionally substituted with one or more of R¹⁰;
R⁸ and R⁹ are each independently selected from H and C₁₋₆alkyl;
each R¹⁰ is independently selected from halo, C₁₋₆alkyl, CN and NR¹¹R^{11a};
R¹¹ and R^{11a} are each independently selected from H and C₁₋₆alkyl;
Cy² is a monocyclic C₃₋₇heterocycloalkyl which is substituted with one or more of R¹² or a bicyclic C₆₋₁₂heterocycloalkyl which is unsubstituted or substituted with one or more of R¹²;
each R¹² is independently selected from halo, CN, ═O, OH, C₁₋₆alkyl, C₂₋₆alkenyl, C₂₋₆alkynyl, C₃₋₁₀cycloalkyl, C₃₋₁₀heterocycloalkyl, C₁₋₆alkyleneC₃₋₁₀cycloalkyl, C₁₋₆alkyleneC₃₋₁₀heterocycloalkyl, C₁₋₆alkyleneOR¹³, C₁₋₆alkyleneNR¹³R¹⁴, OC₁₋₆alkyleneOR¹³, OC₁₋₆alkyleneNR¹³R¹⁴, SR¹³, C(O)R¹³, C(O)C₁₋₆alkyleneOR¹³, C(O)C₁₋₆alkyleneNR¹³R¹⁴, C(O)C₁₋₆alkyleneOC₁₋₆alkyleneNR¹³R¹⁴, C(O)NR¹³R¹⁴, CO₂R¹³, CO₂C₁₋₆alkyleneOR¹³, CO₂C₁₋₆alkyleneOC₁₋₆alkyleneNR¹³R¹⁴, NR¹³R¹⁴, NR¹⁵SO₂R¹³, S(O)R¹³, SO₂R¹³, SO₂NR¹³R¹⁴ and S(O)(NR¹⁵)R¹³;
R¹³ is selected from H, C₁₋₆alkyl, C₁₋₆alkyleneOR¹⁴, C₃₋₁₀cycloalkyl, C₃₋₁₀heterocycloalkyl, C₁₋₆alkyleneC₃₋₁₀cycloalkyl and C₁₋₆alkyleneC₃₋₁₀heterocycloalkyl,
R¹⁴ is selected from H and C₁₋₆alkyl; or
R¹³ and R¹⁴ are joined to form, together with the nitrogen atom therebetween, a 4- to 6-membered saturated or unsaturated ring, optionally containing one additional heteromoiety selected from N, NR¹⁶, O, S, S(O) and SO₂; and
R¹⁵ and R¹⁶ are independently selected from H and C₁₋₆alkyl;
wherein all available hydrogen atoms are optionally substituted with a fluorine atom, provided Cy² is not

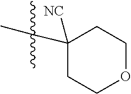 [SMILES: *C1(C#N)CCOCC1]

when Cy¹ is unsubstituted phenyl wherein

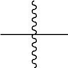

represents a point of covalent attachment to Cy¹.

The application also includes a compound of Formula (II), or a pharmaceutically acceptable salt, solvate and/or prodrug thereof,

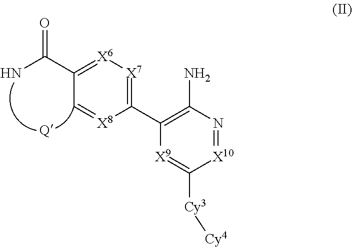

wherein
X⁶ is selected from N and CR¹⁷;
X⁷ and X⁸ are each independently selected from N and CR¹⁸;
X⁹ and X¹⁰ are each independently selected from N and CH, provided at least one of X⁹ and X¹⁰ is N;
Q′ is C₁₋₄alkylene optionally interrupted by a heteromoiety selected from O, S, S(O), SO₂ and NR¹⁹ and/or optionally substituted with one or more of R²⁰ and/or optionally disubstituted on one carbon with R²¹ and R^{21a}, provided that when Q comprises the heteromoiety the heteromoiety is separated from the ring amide NH by other than methylene; or
Q′ is C₂₋₄alkenylene optionally substituted with one or more of R²²; or
Q′ is C═N or N═C optionally substituted with R²²;
R¹⁷ is selected from H, halo, OR²³, NR²⁴R²⁵, C₁₋₆alkyleneNR²⁴R²⁵ and C₁₋₆alkyl;
R¹⁸ is selected from H, halo and C₁₋₆alkyl;
R¹⁹ is selected from H and C₁₋₆alkyl;
each R²⁰ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆ alkyl, NR²⁶R²⁷ and C₁₋₆alkyleneNR²⁶R²⁷;
R²¹ and R^{21a} are joined to form, together with the carbon atom therebetween, a 3- to 6-membered, saturated or unsaturated ring optionally containing one heteromoiety selected from N, NH, NC₁₋₆alkyl, O, S, S(O), and SO₂ and optionally substituted with one or more of halo and C₁₋₆alkyl;
each R²² is independently selected from halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR²⁶R²⁷ and C₁₋₆alkyleneNR²⁶R²⁷;
R²⁴, R²⁵, R²⁶ and R²⁷ are each independently selected from H and C₁₋₆alkyl, or
R²⁴ and R²⁵ or R²⁶ and R²⁷ are joined to form, together with the atom therebetween, a 3- to 7-membered saturated or unsaturated ring, optionally containing one additional heteromoiety selected from N, NH, NC₁₋₆alkyl, O, S, S(O), and SO₂ and optionally substituted with one or more of halo and C₁₋₆alkyl;
Cy³ is C₆₋₁₀aryl or C₅₋₁₀heteroaryl, which substituted with one or two of R²⁸, and optionally further substituted with one to three of R²⁹
each R²⁸ is independently selected from NR³⁰R³¹, C₁₋₆alkyleneNR³⁰R³¹, C₃₋₇heterocycloalkyl and C₁₋₆alkyleneC₃₋₇heterocycloalkyl, the latter two groups being optionally substituted with one or more of R³²
each R²⁹ is independently selected from halo, C₁₋₆alkyl, C₃₋₇cycloalkyl, and C₁₋₆alkyleneC₃₋₇cycloalkyl, the latter two groups being optionally substituted with one or more of R³²
R³⁰ and R³¹ are each independently selected from H and C₁₋₆alkyl;
each R³² is independently selected from halo, C₁₋₆alkyl, CN and NR³³R³⁴;
R³³ and R³⁴ are each independently selected from H and C₁₋₆alkyl;
Cy⁴ is C₃₋₁₄heterocycloalkyl, and Cy⁴ is unsubstituted or substituted with one or more of R³⁵
each R³⁵ is independently selected from halo, ═O, CN, OH, C₁₋₆alkyl, C₂₋₆alkenyl, C₂₋₆alkynyl, C₃₋₁₀cycloalkyl, C₃₋₁₀heterocycloalkyl, C₁₋₆alkyleneC₃₋₁₀cycloalkyl, C₁₋₆alkyleneC₃₋₁₀heterocycloalkyl, C₁₋₆alkyleneOR³⁶, C₁₋₆alkyleneNR³⁶R³⁷, OC₁₋₆alkyleneOR³⁶, OC₁₋₆alkyleneNR³⁶R³⁷, SR³⁶, C(O)R³⁶ C(O)C₁₋₆alkyleneOR³⁶, C(O)C₁₋₆alkyleneNR³⁶R³⁷, C(O)C₁₋₆alkyleneOC₁₋₆alkyleneNR³⁶R³⁷, C(O)NR³⁶R³⁷, CO₂R³⁶, CO₂C₁₋₆alkyleneOR³⁶, CO₂C₁₋₆alkyleneOC₁₋₆alkyeneNR³⁶R³⁷, NR³⁶R³⁷, NR³⁸SO₂R³⁶, S(O)R³⁶, SO₂R³⁶, SO₂NR³⁶R³⁷ and S(O)(NR³⁸)R³⁶;
R³⁶ is selected from H, C₁₋₆alkyl, C₁₋₆alkyleneOR¹⁴, C₃₋₁₀cycloalkyl, C₃₋₁₀heterocycloalkyl, C₁₋₆alkyleneC₃₋₁₀cycloalkyl and C₁₋₆alkyleneC₃₋₁₀heterocycloalkyl,
R³⁷ is selected from H and C₁₋₆alkyl; or
R³⁶ and R³⁷ are joined to form, together with the nitrogen atom therebetween, a 4- to 6-membered saturated or unsaturated ring, optionally containing one additional heteromoiety selected from N, NR³⁹, O, S, SO and SO₂; and
R³⁸ and R³⁹ are independently selected from H and C₁₋₆alkyl;
wherein all available hydrogen atoms are optionally substituted with a fluorine atom.

The present application also includes a composition comprising one or more compounds of the application and a carrier. In an embodiment, the composition is a pharmaceutical composition comprising one or more compounds of the application and a pharmaceutically acceptable carrier.

In an embodiment, the compounds of the application are used as medicaments. Accordingly, the application also includes a compound of the application for use as a medicament.

The compounds of the application have been shown to inhibit HPK1. Therefore the compounds of the application are useful for treating diseases, disorders or conditions that are treatable by inhibiting HPK1 activity. Accordingly, the present application also includes a method of treating a disease, disorder or condition that is treatable by inhibiting HPK1, comprising administering a therapeutically effective amount of one or more compounds of the application to a subject in need thereof.

The present application also includes a use of one or more compounds of the application for treatment of a disease, disorder or condition that is treatable by inhibiting HPK1, as well as a use of one or more compounds of the application for the preparation of a medicament for treatment of a disease, disorder or condition that is treatable by inhibiting HPK1. The application further includes one or more compounds of the application for use in treating a disease, disorder or condition that is treatable by inhibiting HPK1.

In a further embodiment, the disease, disorder or condition that is treatable by inhibiting HPK1 is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies. In another embodiment, the additional cancer treatment is selected from an antibody that binds to PD-1 or PDL-1.

The application additionally provides a process for the preparation of compounds of the application. General and specific processes are discussed in more detail and set forth in the Examples below.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component or effect, such as an additional or second compound, the second compound as used herein is different from the other compounds or first compound. A “third” compound is different from the other, first, and second compounds, and further enumerated or “additional” compounds are similarly different.

The term “and/or” as used herein means that the listed items are present, or used, individually or in combination. In effect, this term means that “at least one of” or “one or more” of the listed items is used or present. The term “and/or” with respect to enantiomers, prodrugs, salts and/or solvates thereof means that the compounds of the application exist as individual enantiomers, prodrugs, salts and hydrates, as well as a combination of, for example, a salt of a solvate of a compound of the application.

The term “compound of the application” or “compound of the present application” and the like as used herein refers to a compound of Formula I, I-A, I-B, I-C, I-D, I-E, II, II-A, II-B, II-C, II-D and II-E or salts, solvates and/or prodrugs thereof.

The term “composition of the application” or “composition of the present application” and the like as used herein refers to a composition comprising one or more compounds of the application.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

The present description refers to a number of chemical terms and abbreviations used by those skilled in the art. Nevertheless, definitions of selected terms are provided for clarity and consistency.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “cell” as used herein refers to a single cell or a plurality of cells and includes a cell either in a cell culture or in a subject.

The term “subject” as used herein includes all members of the animal kingdom including mammals, and suitably refers to humans. Thus the methods and uses of the present application are applicable to both human therapy and veterinary applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “prodrug” as used herein means a compound, or salt and/or solvate of a compound, that, after administration, is converted into an active drug.

The term “inert organic solvent” as used herein refers to a solvent that is generally considered as non-reactive with the functional groups that are present in the compounds to be combined together in any given reaction so that it does not interfere with or inhibit the desired synthetic transformation. Organic solvents are typically non-polar and dissolve compounds that are non soluble in aqueous solutions.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms. Ally alkyl groups are optionally fluoro-substituted.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms. All alkylene groups are optionally fluoro-substituted.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond. All alkenyl groups are optionally fluoro-substituted.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms. All alkynyl groups are optionally fluoro-substituted.

The term “cycloalkyl,” as used herein, whether it is used alone or as part of another group, means a saturated carbocyclic group containing a number of carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms. When a cycloalkyl group contains more than one ring, the rings may be fused, bridged, spirofused or linked by a bond. All cycloalkyl groups are optionally fluoro-substituted.

The term “aryl” as used herein, whether it is used alone or as part of another group, refers to carbocyclic groups containing at least one aromatic ring and contains 6 to 20 carbon atoms, such as phenyl, indanyl or naphthyl. All aryl groups are optionally fluoro-substituted.

The term “heterocycloalkyl” as used herein, whether it is used alone or as part of another group, refers to cyclic groups containing at least one non-aromatic ring containing from 3 to 20 atoms in which one or more of the atoms are a heteroatom selected from O, S and N and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. All heterocycloalkyl groups are optionally fluoro-substituted. The heteroatom in heterocycloalkyl groups is optionally substituted or oxidized where valency allows.

The term “heteroaryl” as used herein, whether it is used alone or as part of another group, refers to cyclic groups containing at least one heteroaromatic ring containing 5-10 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. All heteroaryl groups are optionally fluoro-substituted. The heteroatom in heteroaryl groups is optionally substituted or oxidized where valency allows.

The term “aza-heteroaryl” as used herein, whether it is used alone or as part of another group, refers to a heteroaryl group having two or more N atoms as the only heteroatom in the group.

All cyclic groups, including aryl and cyclo groups, and hetero versions thereof, contain one or more than one ring (i.e. are polycyclic). When a cyclic group contains more than one ring, the rings may be fused, bridged, and/or spirofused.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “fluoro-substituted” refers to the substitution of one or more, including all, available hydrogens in a referenced group with fluoro.

The terms “halo” or “halogen” as used herein, whether it is used alone or as part of another group, refers to a halogen atom and includes fluoro, chloro, bromo and iodo.

The term “available”, as in “available hydrogen atoms” or “available atoms” refers to atoms that would be known to a person skilled in the art to be capable of replacement by a substituent.

When a group is said to be substituted with multiple substituents, said substituents are independently selected therefore can be the same or different.

The term “cross-coupling” as used herein refers to chemical reactions in which two different starting materials, each of which is usually endowed with an activating group, are reacted together with the aid of a metal catalyst. The result is the loss of the two activating groups and the formation of a new covalent bond between the remaining fragments.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition treatable by inhibiting HPK1, or manifesting a symptom associated with a disease, disorder or condition treatable by inhibition of HPK1.

As used herein, the term “effective amount” or “therapeutically effective amount” means an amount of a compound, or one or more compounds, of the application that is effective, at dosages and for periods of time necessary to achieve the desired result.

The expression “inhibiting HPK1” as used herein refers to inhibiting, blocking and/or disrupting HPK1 enzymatic activity in a cell, in particular a T-cell or B-cell. The inhibiting, blocking and/or disrupting causes a therapeutic effect in the cell.

By “inhibiting, blocking and/or disrupting” it is meant any detectable inhibition, block and/or disruption in the presence of a compound compared to otherwise the same conditions, except for in the absence in the compound.

The term “disease, disorder or condition treatable by inhibiting HPK1” means that the disease, disorder or condition to be treated is affected by, modulated by and/or has some biological basis, either direct or indirect, that includes HPK1 activity, in particular, increased HPK1 activity. These diseases respond favourably when HPK1 activity associated with the disease, disorder or condition is inhibited by one or more of the compounds or compositions of the application.

The term “HPK1” as used herein refers to the hematopoetic progenitor kinase 1.

The term “administered” as used herein means administration of a therapeutically effective amount of a compound, or one or more compounds, or a composition of the application to a cell either in cell culture or in a subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder. Neoplasms can be benign (such as uterine fibroids and melanocytic nevi), potentially malignant (such as carcinoma in situ) or malignant (i.e. cancer).

The term “cancer” as used herein refers to cellular-proliferative disease states.

As used herein, the term “effective amount” means an amount effective, at dosages and for periods of time, necessary to achieve a desired result.

### II. Compounds and Compositions

### i) Compounds of Formula (I)

The present application describes a novel class of substituted heterocyclic HPK1 inhibitors.

Accordingly, the application includes a compound of Formula (I), or a pharmaceutically acceptable salt, solvate and/or prodrug thereof,

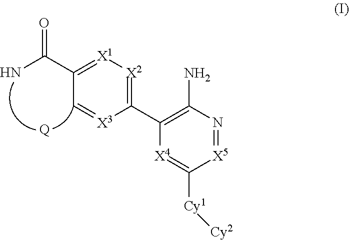

wherein:
X¹ is selected from N and CR¹;
X² and X³ are each independently selected from N and CR²;
X⁴ and X⁵ are each independently selected from N and CH, provided at least one of X⁴ and X⁵ is N;
Q is C₁₋₄alkylene optionally interrupted by a heteromoiety selected from O, S, S(O), SO₂ and NR³ and/or optionally substituted with one or more of R⁴ and/or optionally disubstituted on one carbon with R^{4a} and R^{4b}, provided that when Q comprises the heteromoiety the heteromoiety is separated from the ring amide NH by other than methylene; or
Q is C₂₋₄alkenylene optionally substituted with one or more of R^{4c}; or
Q is C═N or N═C optionally substituted with R^{4c};
R¹ is selected from H, halo, OR^{3a}, NR^{5a}R^{6a}, C₁₋₆alkyleneNR^{5a}R^{6a} and C₁₋₆alkyl;
R² is selected from H, halo and C₁₋₆alkyl;
R³ is selected from H and C₁₋₆alkyl;
each R⁴ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆ alkyl, NR⁵R⁶ and C₁₋₆alkyleneNR⁵R⁶;
R^{4a} and R^{4b} are joined to form, together with the carbon atom therebetween, a 3- to 6-membered, saturated or unsaturated ring optionally containing one heteromoiety selected from N, NH, NC₁₋₆alkyl, O, S, S(O), and SO₂ and optionally substituted with one or more of halo and C₁₋₆alkyl;
each R^{4c} is independently selected from halo, C₁₋₆alkyl, C₃₋₆cycloalkyl, C₃₋₆heterocycloalkyl, C₁₋₆alkyleneC₃₋₆cycloalkyl and C₁₋₆alkyleneC₃₋₆heterocycloalkyl, OH, OC₁₋₆alkyl, NR⁵R⁶, and C₁₋₆alkyleneNR⁵R⁶;
R⁵, R^{5a}, R⁶ and R^{6a} are each independently selected from H and C₁₋₆alkyl, or
R⁵ and R⁶ or R^{5a} and R^{6a} are joined to form, together with the nitrogen atom therebetween, a 3- to 7-membered, saturated or unsaturated ring optionally containing one additional heteromoiety selected from N, NH, NC₁₋₆alkyl, O, S, S(O), and SO₂ and optionally substituted with one or more of halo and C₁₋₆alkyl;
Cy¹ is C₆₋₁₀aryl or C₅₋₁₀heteroaryl, which is unsubstituted or substituted with one or more of R⁷;
each R⁷ is independently selected from halo, ═O, C₁₋₆alkyl, NR⁸R⁹, and C₁₋₆alkyleneNR⁸R⁹, C₃₋₇cycloalkyl, C₃₋₇heterocycloalkyl, C₁₋₆alkyleneC₃₋₇cycloalkyl and C₁₋₆alkyleneC₃₋₇heterocycloalkyl, the latter four groups being optionally substituted with one or more of R¹⁰;
R⁸ and R⁹ are each independently selected from H and C₁₋₆alkyl;
each R¹⁰ is independently selected from halo, C₁₋₆alkyl, CN and NR¹¹R^{11a};
R¹¹ and R^{11a} are each independently selected from H and C₁₋₆alkyl;
Cy² is a monocyclic C₃₋₇heterocycloalkyl which is substituted with one or more of R¹² or a bicyclic C₆₋₁₂heterocycloalkyl which is unsubstituted or substituted with one or more of R¹²;
each R¹² is independently selected from halo, CN, ═O, OH, C₁₋₆alkyl, C₂₋₆alkenyl, C₂₋₆alkynyl, C₃₋₁₀cycloalkyl, C₃₋₁₀heterocycloalkyl, C₁₋₆alkyleneC₃₋₁₀cycloalkyl, C₁₋₆alkyleneC₃₋₁₀heterocycloalkyl, C₁₋₆alkyleneOR¹³, C₁₋₆alkyleneNR¹³R¹⁴, OC₁₋₆alkyleneOR¹³, OC₁₋₆alkyleneNR¹³R¹⁴, SR¹³, C(O)R¹³, C(O)C₁₋₆alkyleneOR¹³, C(O)C₁₋₆alkyleneNR¹³R¹⁴, C(O)C₁₋₆alkyleneOC₁₋₆alkyleneNR¹³R¹⁴, C(O)NR¹³R¹⁴, CO₂R¹³, CO₂C₁₋₆alkyleneOR¹³, CO₂C₁₋₆alkyleneOC₁₋₆alkyleneNR¹³R¹⁴, NR¹³R¹⁴, NR¹⁵SO₂R¹³, S(O)R¹³, SO₂R¹³, SO₂NR¹³R¹⁴ and S(O)(NR¹⁵)R¹³;
R¹³ is selected from H, C₁₋₆alkyl, C₁₋₆alkyleneOR¹⁴, C₃₋₁₀cycloalkyl, C₃₋₁₀heterocycloalkyl, C₁₋₆alkyleneC₃₋₁₀cycloalkyl and C₁₋₆alkyleneC₃₋₁₀heterocycloalkyl,
R¹⁴ is selected from H and C₁₋₆alkyl; or
R¹³ and R¹⁴ are joined to form, together with the nitrogen atom therebetween, a 4- to 6-membered saturated or unsaturated ring, optionally containing one additional heteromoiety selected from N, NR¹⁶, O, S, S(O) and SO₂; and
R¹⁵ and R¹⁶ are independently selected from H and C₁₋₆alkyl;
wherein all available hydrogen atoms are optionally substituted with a fluorine atom, provided Cy² is not

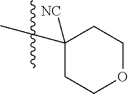 [SMILES: *C1(C#N)CCOCC1]

when Cy¹ is unsubstituted phenyl wherein

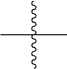

represents a point of covalent attachment to Cy¹.

In all embodiments below it is to be understood that all available hydrogen atoms are optionally substituted with a fluorine atom. This has not been repeated throughout. Thus in each embodiment where a group is listed that comprises available hydrogen atoms, it is to be understood that all such atoms are optionally replaced with fluorine atoms, for example each recitation of C₁₋₆alkyl is also a recitation of C₁₋₆ fluoroalkyl, unless stated otherwise.

In an embodiment, X² is N. In an embodiment, X² is CR¹.

In an embodiment, R¹ is selected from H, F, Cl, OR^{4a}, NR^{5a}R^{6a}, C₁₋₄alkyleneNR^{5a}R^{6a} and C₁₋₄alkyl.

In an embodiment, R¹ is selected from H, F, Cl and C₁₋₄alkyl. In an embodiment, R¹ is selected from H, F, Cl, CH₃, CH₂CH₃, CF₂H, CF₃, CFH₂, CH₂CF₂H and CH₂CF₃. In an embodiment, R¹ is selected from H, F, CF₃, CF₂H, CH₂CF₂H and CH₃. In an embodiment, R¹ is selected from H, F, CF₃, CF₂H and CH₂CF₂H. In an embodiment, R¹ is selected from H, F, CF₃ and CH₃. In an embodiment, R¹ is selected from H and F. In an embodiment, R¹ is F. In an embodiment, R¹ is H.

In an embodiment, R¹ is OR^{3a}. In an embodiment, R^{3a} is selected from H, CH₃, CH₂CH₃, CF₃, CFH₂, CF₂H, CH₂CF₂H, and CH₂CF₂H. In an embodiment, R^{3a} is selected from H and C₁₋₄alkyl. In an embodiment, R^{3a} is selected from H, CH₃ and CH₂CH₃. In an embodiment, R^{3a} is selected from H, CH₃, CH₂CH₃, CF₃, CFH₂, CH₂CF₂H, and CH₂CF₃. In an embodiment, R^{3a} is CF₂H. Accordingly, in an embodiment, R¹ is selected from OH, OCH₃, OCH₂CH₃, OCF₃, OCFH₂, OCHF₂, OCH₂CF₂H, and OCH₂CF₂H.

In an embodiment, R¹ is selected from NR^{5a}R^{6a} and C₁₋₄alkyleneNR^{5a}R^{6a}. In an embodiment, R¹ is selected from NR^{5a}R^{6a} and C₁₋₂alkyleneNR^{5a}R^{6a}. In an embodiment, R^{5a} and R^{6a} are each independently selected from H and C₁₋₄alkyl. In an embodiment, R^{5a} and R^{6a} are each independently selected from H, CH₃ and CF₃. In an embodiment, one of R^{5a} and R^{6a} is H and the other is CH₃. In an embodiment, R^{6a} and R^{5a} are both CH₃. In an embodiment, R^{5a} and R^{6a} are both H.

In an embodiment, R^{5a} and R^{6a} are joined to form, together with the nitrogen atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R^{5a} and R^{6a} are taken together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl, and optionally substituted with one or more of halo and C₁₋₆alkyl. In an embodiment, R^{5a} and R^{6a} are joined to form, together with the nitrogen atom therebetween, a 4- to 6-membered saturated ring, and optionally substituted with one or more of halo and C₁₋₆alkyl. In an embodiment R^{5a} and R^{6a} are joined to form, together with the nitrogen atom therebetween, aziridinyl, azetidinyl, pyrrolidinyl, or piperidinyl, and optionally substituted with one or more of halo and C₁₋₆ alkyl.

In an embodiment, one of X² and X³ is N and the other is CR². In an embodiment, X² is CR² and X³ is N. In an embodiment, both X² and X³ are independently CR².

In an embodiment, each R² is independently selected from H, halo and C₁₋₄alkyl. In an embodiment, each R² is independently selected from H, F, Cl and C₁₋₄alkyl. In an embodiment, each R² is independently selected from H, F, Cl, CH₃, CF₃, CH₂F and CHF₂. In an embodiment, R² is selected from CF₂H, CH₃ and CF₃. In an embodiment, each R² is independently selected from H, F, Cl, CH₃ and CF₃. In an embodiment, each R² is selected from H and F.

In an embodiment, one of X² and X³ is N and the other is CH. In an embodiment, X² is CH and X³ is N. In an embodiment, X² is CH and X³ is CF or CCl. In an embodiment, X² and X³ are both CF. In an embodiment, X² is selected from CH, CF, CCl, CCH₃ and CCF₃ and X³ is CH. In an embodiment, X² and X³ are both CH.

In an embodiment, X¹ is CR¹ and X² and X³ are both CR². In an embodiment, X¹ is N and an X² and X³ are independently CR². In an embodiment, X¹ is N and an X² and X³ are both CH. In an embodiment, X¹ is CR¹ and one of X² and X³ is N and the other is CR². In an embodiment, X¹ is CR¹ and one of X² and X³ is N and the other is CH.

In an embodiment, one of X⁴ and X⁵ is N and the other is CH. In an embodiment, X⁴ is N and X⁵ is CH. In embodiment, X⁴ is CH and X⁵ is N.

In an embodiment, Q is C₁₋₃alkylene optionally interrupted by a heteromoiety selected from O, S, S(O), SO₂, and NR³ and/or optionally substituted with one or more of R⁴.

In an embodiment, Q is C₁₋₃alkylene optionally interrupted by a heteromoiety selected from O, S, S(O), SO₂, and NR³. In an embodiment, Q is C₁₋₃ alkylene optionally interrupted by a heteromoiety selected from O, SO₂, and NR³. In an embodiment, Q is C₁₋₃alkylene optionally interrupted by O or NR³. In an embodiment, Q is C₂₋₃alkylene optionally interrupted by O or NR³ and/or optionally substituted with one or more of R⁴. In an embodiment, Q is C₂₋₃alkylene optionally interrupted by O and/or optionally substituted with one or two of R⁴. In an embodiment, Q is C₁₋₃alkylene optionally interrupted by a heteromoiety selected from O and SO₂. In an embodiment, Q is C₁₋₃alkylene optionally interrupted by O. In an embodiment, Q is C₁₋₂alkylene optionally interrupted by SO₂. In an embodiment, Q is C₁₋₂alkylene optionally interrupted SO₂. In an embodiment, Q is Q is CH₂SO₂.

In an embodiment, R³ is selected from H and C₁₋₄alkyl. In an embodiment, R³ is selected from H, CH₃ and CH₂CH₃. In an embodiment, R³ is selected from H, CH₃, CH₂CH₃, CF₂H, CF₃, CFH₂, CH₂CF₂H and CH₂CF₃. In an embodiment, R³ is selected from CF₂H, CH₃ and CF₃. In an embodiment, R³ is selected from H, CH₃, CH₂CH₃ and CF₃. In an embodiment, R³ is selected from CH₃ and CF₃. In an embodiment, R³ is H.

In an embodiment, Q is C₁₋₃alkylene and optionally substituted with one to three of R⁴. In an embodiment, Q is CH₂ or CH₂CH₂ and optionally substituted with one or two of R⁴. In an embodiment, Q is C₁alkylene and optionally substituted with one or two of R⁴. In an embodiment, Q is CH₂. In an embodiment, Q is CH₂CH₂.

In an embodiment, each R⁴ is independently selected from ═O, F, Cl, C₁₋₄alkyl C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR⁵R⁶, and C₁₋₆alkyleneNR⁵R⁶. In an embodiment, each R⁴ is independently selected from ═O, F, Cl, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₄alkyl, NR⁵R⁶. In an embodiment, one of R⁴ is ═O.

In an embodiment, each R⁴ is independently selected from F, Cl, OH, C₁₋₄alkyl OC₁₋₄alkyl and NR⁵R⁶. In an embodiment, each R⁴ is independently selected from F, Cl, OH, CH₃, CH₂CH₃, CF₂H, CF₃, CFH₂, CH₂CF₂H, OCH₃, OCH₂CH₃, OCF₃, OCF₂H, OCH(CH₃)₂ and NR⁵R⁶. In an embodiment, each R⁴ is independently selected from F, Cl, OH, CH₃, CF₂H, CF₃, CFH₂, OCH₃, OCF₃, OCF₂H and NR⁵R⁶. In an embodiment, one to three of R⁴ are independently selected from F, Cl, CH₃, CF₂H, CF₃, OCH₃, OCF₃, OCF₂H and NR⁵R⁶. In an embodiment, one to three of R⁴ are independently selected from F, Cl, CH₃, CF₂H, CF₃, OCH₃, OCF₃ and OCF₂H. In an embodiment, one to four of R⁴ are independently selected from F, CH₃, and OCH₃.

In an embodiment, each R⁴ is independently selected from F, C and C₁₋₄alkyl. In an embodiment, each R⁴ is independently selected from F, Cl, CH₃, CH₂CH₃, CF₂H, CF₃, CFH₂, CH₂CF₂H, and CH₂CH₂F. In an embodiment, each R⁴ is independently selected from F, Cl, CH₃, CF₂H, CF₃ and CH₂CF₂H. In an embodiment, R⁴ is selected from F, Cl, CH₃, and CF₃. In an embodiment, R⁴ is selected from F, CH₃, and CF₃. In an embodiment, each R⁴ is independently selected from F, CH₃, and CF₃. In an embodiment, each R⁴ is independently selected from F and CH₃. In an embodiment, at least one of R⁴ is F. In an embodiment, at one or two of R⁴ is F. In an embodiment, one or more, one to four, one to three, one or two, or one of R⁴ is CH₃.

In an embodiment, one or two R⁴ are is independently selected from C₃₋₆cycloalkyl, C₃₋₆heterocycloalkyl, C₁₋₄alkyleneC₃₋₆cycloalkyl and C₁₋₄alkyleneC₃₋₆heterocycloalkyl. In an embodiment, one or two of R⁴ are independently selected from C₃₋₆cycloalkyl, C₃₋₆heterocycloalkyl, C₁₋₂alkyleneC₃₋₆cycloalkyl and C₁₋₂alkyleneC₃₋₆heterocycloalkyl. In an embodiment, the cycloalkyl in R⁴ is selected from cyclopropyl, cyclobutyl, cyclopentyl and cyclohexyl. In an embodiment, the cycloalkyl in R⁴ is selected from cyclopropyl and cyclobutyl.

In an embodiment, the heterocycloalkyl of R⁴ is selected from aziridinyl, oxiranyl, thiiranyl, oxaxiridinyl, dioxiranyl, azetidinyl, oxetanyl, thieitanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g, piperazinyl), morpholinyl, thiomorpholinyl, dioxanyl, and dithianyl. In an embodiment, the heterocycloalkyl in R⁴ is selected from azetidinyl, oxetanyl, thietanyl, tetrahydrofuranyl, pyrrolidinyl, imidazolidiny and pyrazolidinyl.

In an embodiment, each R⁴ is independently selected from OH and OC₁₋₄alkyl. In an embodiment, each R⁴ is independently selected from OH and OC₁₋₄alkyl. In an embodiment, one or two R⁴ are independently selected from OH, OCH₃, OCH₂CH₃, OCF₃, OCF₂H and OCH(CH₃)₂. In an embodiment, one R⁴ is selected from OH, OCH₃, OCF₃, and OCF₂H.

In an embodiment, one of R⁴ is NR⁵R⁶ or C₁₋₄alkylene NR⁵R⁶. In an embodiment, one of R⁴ is NR⁵R⁶ or C₁₋₂alkyleneNR⁵R⁶. In an embodiment, one of R⁴ is NR⁵R⁶. In an embodiment, one R⁴ is C₁₋₂alkyleneNR⁵R⁶. In an embodiment, the R⁵ and R⁶ in the NR⁵R⁶ or C₁₋₂alkyleneNR⁵R⁶ of R⁴ are both CH₃ or both H. In an embodiment, one R⁴ is NR⁵R⁶ and R⁵ and R⁶ in R⁴ are both H. In an embodiment, R⁵ and R⁶ are each independently selected from H and C₁₋₄alkyl. In an embodiment, R⁵ and R⁶ are each independently selected from H, CH₃ and CF₃. In an embodiment, one of R⁵ and R⁶ is H and the other is CH₃. In an embodiment, R⁵ and R⁶ are both CH₃.

In an embodiment, R⁵ and R⁶ are joined to form, together with the nitrogen atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R⁵ and R⁶ are taken together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl, optionally substituted with one or more of halo and C₁₋₆alkyl. In an embodiment, R⁵ and R⁶ are joined to form, together with the nitrogen atom therebetween, a 4- to 6-membered heterocyclic ring, optionally substituted with one or more of halo and C₁₋₆alkyl. In an embodiment R⁵ and R⁶ are joined to form, together with the nitrogen atom therebetween, aziridinyl, azetidinyl, pyrrolidinyl or piperidinyl, optionally substituted with one or more of halo and C₁₋₆alkyl.

In an embodiment, Q is C₁₋₃alkylene and is unsubstituted. In an embodiment, Q is C₁₋₃alkylene and is substituted with one or two of R⁴. In an embodiment, Q is unsubstituted. In an embodiment, Q is substituted with one or two of R⁴. In an embodiment, Q is C₁₋₃alkylene and is substituted with one or two of R⁴, and one R⁴ is C₁₋₄alkyl. In an embodiment, Q is C₁₋₃alkylene and is substituted with one or two of R⁴, and one or R⁴ is selected from F, CH₃ and CF₃. In an embodiment, Q is C₁₋₃alkylene and is substituted with one or two of R⁴, and one of R⁴ is CH₃. In an embodiment, Q is unsubstituted. In an embodiment, Q is substituted with one or two of R⁴. In an embodiment, Q is substituted with one or two of R⁴, and at least one R⁴ is C₁₋₄alkyl. In an embodiment, Q is substituted with one or two of R⁴, and R⁴ is selected from F, CH₃ and CF₃. In an embodiment, Q is substituted with one or two of R⁴, and R⁴ is CH₃. In an embodiment, Q is C₁₋₃alkylene and is substituted with one to four of R⁴, and each R⁴ is independently selected from F, Cl, CH₃, CF₂H, CF₃, OCH₃, OCF₃ and OCF₂H. In an embodiment, Q is C₁₋₃alkylene and is substituted with one to four of R⁴, and each R⁴ is independently selected from F, CH₃ and OCH₃.

In an embodiment, Q is C₁₋₃alkylene and optionally disubstituted on one carbon atom with R^{4a} and R^{4b}. In an embodiment, Q is C₁alkylene or C₂alkylene, and optionally disubstituted on one carbon atom with R^{4a} and R^{4b}. In an embodiment, Q is CR^{4a}R^{4b}.

In an embodiment, R^{4a} and R^{4b} are joined to form, together with the carbon atom therebetween, a 3- to 6-membered saturated or unsaturated ring optionally containing one heteromoiety selected from N, NH, NC₁₋₆alkyl, O, S, S(O), and SO₂ and optionally substituted with one or more of halo and C₁₋₄alkyl.

In an embodiment, R^{4a} and R^{4b} are joined to form, together with the carbon atom therebetween, a 3- to 6-membered cycloalkyl ring, optionally substituted with one or more of halo and C₁₋₄alkyl. In an embodiment, R^{4a} and R^{4b} are joined to form, together with the carbon atom therebetween, to form a 3- to 6-membered cycloalkyl selected from cyclopropyl, cyclobutyl, cyclopentyl and cyclohexyl ring optionally substituted with one or more of halo and C₁₋₄alkyl. In an embodiment, R^{4a} and R^{4b} are joined to form, together with the carbon atom therebetween, a 3- to 6-membered cycloalkyl ring, optionally substituted with one or more of halo and C₁₋₄alkyl. In an embodiment, R^{4a} and R^{4b} are joined to form, together with the carbon atom therebetween, to form a 3- to 6-membered cycloalkyl selected from cyclopropyl, cyclobutyl, cyclopentyl and cyclohexyl. In an embodiment, R^{4a} and R^{4b} are joined to form, together with the carbon atom therebetween, a 3- to 5-membered cycloalkyl ring, and optionally substituted with one or more of halo and C₁₋₄alkyl. In an embodiment, R^{4a} and R^{4b} are joined to form, together with the carbon atom therebetween, a cyclopropyl, a cyclobutyl or a cyclopentyl ring optionally substituted with one or more of halo and C₁₋₄alkyl. In an embodiment, R^{4a} and R^{4b} are joined to form, together with the atom therebetween, a cyclopropyl or a cyclobutyl ring, optionally substituted with one or more of halo and C₁₋₄alkyl. In an embodiment, R^{4a} and R^{4b} are joined to form, together with the carbon atom therebetween, a cyclopropyl ring, optionally substituted with one or more of halo and C₁₋₄alkyl. In an embodiment, R^{4a} and R^{4b} are joined to form, together with the carbon atom therebetween, a cyclopropyl or a cyclobutyl ring. In an embodiment, R^{4a} and R^{4b} are joined to form, together with the atom therebetween, a cyclopropyl ring.

In an embodiment, R^{4a} and R^{4b} are joined to form, together with the carbon atom therebetween, a 3- to 6-membered heterocycloalkyl ring optionally substituted with one or more of halo and C₁₋₄alkyl. In an embodiment, R^{4a} and R^{4b} are joined to form, together with the carbon atom therebetween, a 3- to 6-membered heterocycloalkyl ring selected from aziridinyl, oxiranyl, thiiranyl, oxaxiridinyl, dioxiranyl, azetidinyl, oxetanyl, thietanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g, piperazinyl), morpholinyl, thiomorpholinyl, dioxanyl and dithianyl. In an embodiment, R^{4a} and R^{4b} are joined to form, together with the carbon atom therebetween, a 3- to 5-membered heterocycloalkyl ring. In an embodiment, R^{4a} and R^{4b} are joined to form, together with the carbon atom therebetween, a 3- to 5-membered heterocycloalkyl ring selected from oxiranyl, oxetanyl, azetidinyl, thietanyl, pyrrolidinyl, tetrahydrofuranyl, and tetrahydrothiophenyl. In an embodiment, R^{4a} and R^{4b} are joined to form, together with the atom therebetween, oxetanyl, or azetidinyl. In an embodiment, R^{4a} and R^{4b} are joined to form, together with the carbon atom therebetween, oxetanyl. In an embodiment, R^{4a} and R^{4b} are joined to form, together with the atom therebetween to form

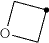

wherein “●” indicates a point of attachment to Q.

In an embodiment, Q is C₂₋₄alkenylene optionally substituted with one or two of R^{4c}. In an embodiment, Q is C═C optionally substituted with one or two of R^{4c}.

In an embodiment, Q is optionally selected from C═N and N═C and is optionally substituted with R^{4c}. In an embodiment, Q is C═N or N═C.

In an embodiment, each R^{4c} is independently selected from F, Cl, C₁₋₄alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR⁵R⁶, and C₁₋₆alkyleneNR⁵R⁶. In an embodiment, each R^{4c} is independently selected from F, Cl, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₄ alkyl, NR⁵R⁶, and C₁₋₄alkyleneNR⁵R⁶.

In an embodiment, each R^{4c} is independently selected from F, Cl, OH, C₁₋₄alkyl, OC₁₋₄alkyl, C₁₋₂alkyleneNR⁵R⁶ and NR⁵R⁶. In an embodiment, each R^{4c} is independently selected from F, Cl, OH, CH₃, CH₂CH₃, CF₂H, CF₃, CFH₂, CH₂CF₂H, OCH₃, OCH₂CH₃, OCF₃, OCF₂H, OCH(CH₃)₂, C₁₋₂alkyleneNR⁵R⁶ and NR⁵R⁶. In an embodiment, each R^{4c} is independently selected from F, Cl, OH, CH₃, CF₂H, CF₃, CFH₂, OCH₃, OCF₃, OCF₂H, C₁₋₂alkyleneNR⁵R⁶ and NR⁹R¹⁰. In an embodiment, one or two of R^{4c} are independently selected from F, Cl, CH₃, CF₂H, CF₃, OCH₃, OCF₃, OCF₂H, C₁₋₂alkyleneNR⁵R⁶ and NR⁵R⁶. In an embodiment, one or two of R^{4c} are independently selected from F, Cl, CH₃, CF₂H, CF₃, OCH₃, OCF₃, OCF₂H, C₁₋₂alkyleneNR⁵R⁶ and NR⁵R⁶.

In an embodiment, each R^{4c} is independently selected from F and C₁₋₄alkyl. In an embodiment, each R^{4c} is independently selected from F, Cl, CH₃, CH₂CH₃, CF₂H, CF₃, CFH₂, CH₂CF₂H, and CH₂CH₂F. In an embodiment, each R^{4c} is independently selected from F, Cl, CH₃, CF₂H, CF₃ and CH₂CF₂H. In an embodiment, each R^{4c} is independently selected from F, Cl, CH₃, and CF₃. In an embodiment, R^{4c} is selected from F, CH₃, and CF₃. In an embodiment, R^{4c} is selected from F and CH₃. In an embodiment, at least one of R^{4c} is F. In an embodiment, one or more, one to four, one to three, one or two, or one of R^{4c} is CH₃. In an embodiment, one of two R^{4c} is CH₃.

In an embodiment, each R^{4c} is independently selected from C₃₋₆cycloalkyl, C₃₋₆heterocycloalkyl, C₁₋₄alkyleneC₃₋₆cycloalkyl and C₁₋₄alkyleneC₃₋₆heterocycloalkyl. In an embodiment, one R^{4c} is selected from C₃₋₆cycloalkyl, C₃₋₆heterocycloalkyl, C₁₋₂alkyleneC₃₋₆cycloalkyl and C₁₋₂alkyleneC₃₋₆heterocycloalkyl. In an embodiment, the cycloalkyl in R^{4c} is selected from cyclopropyl, cyclobutyl, cyclopentyl and cyclohexyl. In an embodiment, the cycloalkyl in R^{4c} is selected from cyclopropyl and. In an embodiment, the heterocycloalkyl R^{4c} is selected from aziridinyl, oxiranyl, thiiranyl, oxaxiridinyl, dioxiranyl, azetidinyl, oxetanyl, thietanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g, piperazinyl), morpholinyl, thiomorpholinyl, and dioxanyl, dithianyl. In an embodiment, the heterocycloalkyl in R^{4c} is selected from azetidinyl, oxetanyl, thietanyl, tetrahydrofuranyl, pyrrolidinyl, imidazolidiny and pyrazolidinyl.

In an embodiment, each R^{4c} is independently selected from OH and OC₁₋₄alkyl. In an embodiment, each R^{4c} is selected from OH and OC₁₋₄alkyl. In an embodiment, each R^{4c} is selected from OH, OCH₃, OCF₃, OCF₂H, OCH₂CH₃ and OCH(CH₃)₂. In an embodiment, one R^{4c} is selected from OH, OCH₃, OCF₃, and OCF₂H.

In an embodiment, one or two of R^{4c} is selected from NR⁵R⁶ and C₁₋₄alkyleneNR⁵R⁶. In an embodiment, one or two of R^{4c} are independently C₁₋₄alkyleneNR⁵R⁶. In an embodiment, one of R^{4c} is C₁₋₂alkyleneNR⁵R⁶. In an embodiment, one of R^{4c} is NR⁵R⁶. In an embodiment, R⁵ and R⁶ in the NR⁵R⁶ or C₁₋₂alkyleneNR⁵R⁶ of R⁴ are both CH₃ or both H. In an embodiment, one R⁴ is NR⁵R⁶ or C₁₋₄alkyleneNR⁵R⁶ and R⁵ and R⁶ in R⁴ are both H

In an embodiment, R⁵ and R⁶ in R^{4c} are each independently selected from H and C₁₋₄alkyl. In an embodiment, R⁵ and R⁶ in R^{4c} are each independently selected from H, CH₃, and CF₃. In an embodiment, one of R⁵ and R⁶ in R^{4c} is H and the other is CH₃. In an embodiment, R⁵ and R⁶ in R^{4c} are both CH₃.

In an embodiment, R⁵ and R⁶ in R^{4c} are joined to form, together with the nitrogen atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R⁵ and R⁶ in R^{4c} are taken together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl, optionally substituted with one or more of halo and C₁₋₆alkyl. In an embodiment, R⁵ and R⁶ in R^{4c} are joined to form, together with the nitrogen atom therebetween, a 4 to 6-membered heterocyclic ring, optionally substituted with one or more of halo and C₁₋₆alkyl. In an embodiment R⁵ and R⁶ in R^{4c} are joined to form, together with the nitrogen atom therebetween, aziridinyl, azetidinyl, pyrrolidinyl, or piperidinyl, optionally substituted with one or more of halo and C₁₋₆alkyl.

In an embodiment, Q is C₁₋₃alkylene optionally interrupted by NR³. In an embodiment, R³ is selected from H and C₁₋₄alkyl. In an embodiment, R³ is selected from H, CH₃, CH₂CH₃, CF₂H, CF₃, CFH₂, CH₂CF₂H and CH₂CF₃. In an embodiment, R³ is selected from H, CH₃, CH₂CH₃, CF₂H, CF₃, CFH₂, CH₂CF₂H, and CH₂CF₃.

In an embodiment, Q is C₁alkylene or C₂alkylene, and disubstituted on one carbon atom with R^{4a} and R^{4b} and R^{4a} and R^{4b} are joined to form, together with the carbon atom therebetween, a 3- to 5-membered cycloalkyl ring, optionally substituted with one or more of halo and C₁₋₄alkyl. In an embodiment, Q is CR^{4a}R^{4b}. In R^{4a} and R^{4b} are joined to form, together with the carbon atom therebetween, a cyclopropyl or a cyclobutyl ring, optionally substituted with one or more of halo and C₁₋₄alkyl.

In an embodiment, Q is C₂₋₄alkenylene optionally substituted with one or two of R^{4c}, and each R^{4c} is independently selected from F, Cl, C₁₋₄alkyl and OC₁₋₄alkyl. In an embodiment, Q is C═C optionally substituted with one or two of R^{4c}, and each R^{4c} is independently selected from F, Cl, C₁₋₄alkyl and OC₁₋₄alkyl. In an embodiment, Q is C═C optionally substituted with one or two of R^{4c}, and each R^{4c} is independently selected from F, Cl, CH₃ and OCH₃ OCF₃ and OCF₂H. In an embodiment, Q is C═C substituted with one or two of R^{4c}, and each R^{4c} is selected from F and C₁₋₄alkyl. In an embodiment, R^{4c} is selected from F and CH₃. In an embodiment, Q is C═C substituted with F. In an embodiment, Q is C═C substituted with C. In an embodiment, Q is C═C substituted with OCH₃. In an embodiment, Q is C═C substituted with CH₃. In an embodiment, Q is C═C substituted with F and CH₃.

In an embodiment, Q is selected from C═N and N═C and is optionally substituted with one or two of R^{4c}, and each R^{4c} is independently selected from F, Cl, C₁₋₄alkyl, OC₁₋₄alkyl, NR⁵R⁶, cyclopropyl and cyclobutyl. In an embodiment, Q is selected from C═N and N═C and is optionally substituted with one or two of R^{4c}, and each R^{4c} is independently selected from F, Cl, CH₃, OCH₃, OCF₃ OCF₂H, NR⁵R⁶, cyclopropyl and cyclobutyl. In an embodiment, Q is selected from C═N and N═C and is optionally substituted with one or two R^{4c}, and each R^{4c} is independently selected from F, Cl, CH₃, OCH₃, OCF₃ and OCF₂H. In an embodiment, Q is selected from C═N and N═C is optionally substituted with R^{4c}, and R^{4c} is selected from F and CH₃. In an embodiment, Q is selected from C═N or N═C and is optionally substituted with R^{4c}, and R^{4c} is selected from cyclopropyl and cyclobutyl. In an embodiment, Q is C₁alkylene or C₂alkylene, and disubstituted on one carbon atom with R^{5a} and R^{5b}. In an embodiment, Q is selected from C═N or N═C and is optionally substituted with one or two R^{4c} and one R^{4c} is selected from C₁₋₂alkyleneNR⁵R⁶ and NR⁵R⁶.

In an embodiment, Q is unsubstituted. In an embodiment, Q is substituted with one or two of R⁴. In an embodiment, Q is substituted with one or two of R⁴, and R⁴ is C₁₋₄alkyl.

In an embodiment, Cy¹ is C₆₋₁₀aryl which is unsubstituted or substituted with one or more of R⁷. In an embodiment, Cy¹ is phenyl which is unsubstituted or substituted with one or more of R⁷. In an embodiment, Cy¹ is phenyl which is unsubstituted. In an embodiment, Cy¹ is phenyl which is substituted with one or two of R⁷.

In an embodiment, Cy¹ is C₅₋₁₀heteroaryl which is unsubstituted or substituted with one or more of R⁷. In an embodiment, Cy¹ is pyrrolyl, imidazolyl, oxazolyl, pyrazolyl or pyridinyl which is unsubstituted or substituted with one or more of R⁷. In an embodiment, Cy¹ is pyridine or pyrazolyl which is unsubstituted or substituted with one or more of R⁷.

In an embodiment, Cy¹ is unsubstituted. In an embodiment, Cy¹ is substituted with one to four of R⁷. In an embodiment, Cy¹ is substituted with one to three of R⁷. In an embodiment, Cy¹ is substituted with one or two of R⁷. In an embodiment, Cy¹ is substituted with one of R⁷.

In an embodiment, each R⁷ is independently selected from halo, C₁₋₄alkyl, NR⁸R⁹, C₁₋₄alkyleneNR⁸R⁹, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹⁰. In an embodiment, each R⁷ is independently selected from F, Cl, C₁₋₄alkyl, NR⁸R⁹, C₁₋₄alkyleneNR⁸R⁹, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹⁰.

In an embodiment, one to three R⁷ are independently selected from F, C₁ and C₁₋₄alkyl. In an embodiment, one to three R⁷ are independently selected from F, Cl, CH₃, CH₂CH₃, CH(CH₃)₂, CH₂CH₂CH₃, CH₂CH₂CH₂CH₃, CF₂H, CF₃, CFH₂, CH₂CH₂F, CH₂CF₂H, CH₂CF₃ CH₂CH₂F₂H, CH₂CH₂CH₂F₂H and CH(CH₃)₂. In an embodiment, one to three R⁷ are independently selected from F, Cl, CH₃, CH₂CH₃, CH(CH₃)₂, CF₂H, CF₃, CFH₂, CH₂CH₂F, CH₂CF₂H, CH₂CF₃, CH₂CH₂F₂H and CH₂CH₂CH₂F₂H. In an embodiment, each R⁷ is independently selected from F, Cl, CH₃, CH₂CH₃, CF₂H, CF₃, CFH₂, CH₂CF₂H and CH₂CF₂H. In an embodiment, each R⁷ is independently selected from F, Cl, CH₃, CH₂CH₃ and CF₃. In an embodiment, each R⁷ is independently F, CH₃ or CF₃. In an embodiment, R⁷ is CH₃ or CF₃. In an embodiment, one or more, one to four, one to three, one or two, or one of R⁷ is CF₃. In an embodiment, one or more of R⁷ is F. In an embodiment, one to three R⁷ is independently selected from F, Cl, CH₃, CH₂CH₃, CF₂H, CF₃, CFH₂, CH₂CF₂H and CH₂CF₃. In an embodiment, one to three R⁷ is independently selected from F, Cl, CH₃, CH₂CH₃ and CF₃. In an embodiment, one to three R⁷ is independently F, CH₃ or CF₃. In an embodiment, one to three R⁷ is independently CH₃ or CF₃. In an embodiment, one to three of R⁷ is F. In an embodiment, one to three, of R⁷ is CF₃.

In an embodiment, Cy¹ is phenyl which is substituted with one or two of R⁷, and one or more of R⁷ is F.

In an embodiment, one or two of R⁷ are independently selected from NR⁸R⁹ and C₁₋₄alkyleneNR⁸R⁹. In an embodiment, one R⁷ is selected from NR⁸R⁹ and C₁₋₃alkyleneNR⁸R⁹.

In an embodiment, one of R⁷ is NR⁸R⁹, and R⁸ and R⁹ are each independently selected from H and C₁₋₄alkyl. In an embodiment, R⁷ is NR⁸R⁹, and R⁸ and R⁹ are each independently selected from H, CH₃, CH₂CH₃, CF₂H, CF₃, CFH₂, CH₂CF₂H, CH₂CF₃. In an embodiment, one of R⁷ is NR⁸R⁹, and R⁸ and R⁹ are each independently selected from independently selected from H, CH₃, CH₂CH₃, CF₂H, CF₃, CFH₂, CH₂CF₂H and CH₂CF₃. In an embodiment, one of R⁷ is NR⁸R⁹ and R⁸ and R⁹ are independently selected from H, CF₃, CH₃ and CH₂CH₃. Accordingly, in an embodiment, one of R⁷ is selected from NH₂, N(CH₃)₂, NH(CH₃), N(CH₃)(CH₂CH₂), NH(CH₂CH₂) and N(CH₂CH₂). In an embodiment, one of R⁷ is selected from NH₂, N(CH₃)₂ and NH(CH₃).

In an embodiment, one of R⁷ is C₁₋₄alkyleneNR⁸R⁹ and R⁸ and R⁹ are each independently selected from H and C₁₋₄alkyl. In an embodiment, one of R⁷ is C₁₋₄alkyleneNR⁸R⁹, and R⁸ and R⁹ are each independently selected from H, CH₃, CH₂CH₃, CF₂H, CF₃, CFH₂, CH₂CF₂H, CH₂CF₃. In an embodiment, one of R⁷ is C₁₋₄alkyleneNR⁸R⁹ and R⁸ and R⁹ are each independently selected from H, CH₃, CH₂CH₃, CF₂H, CF₃, CFH₂, CH₂CF₂H and CH₂CF₃. In an embodiment, In an embodiment, one of R⁷ is C₁₋₄alkyleneNR⁸R⁹ and R⁸ and R⁹ are each independently selected from H, CF₃, CH₃ and CH₂CH₃. Accordingly, in an embodiment, one of R⁷ is selected from CH₂N(CH₂CH₃)₂, C(CH₃)₂NH₂, CH₂N(CH₃)₂, CH₂CH₂N(CH₃)₂ and CH₂N(CH₃)₂ and CH₂N(CH₃)₂. In an embodiment, one of R⁷ is CH₂N(CH₃)₂. In an embodiment, one of R⁷ is C₁₋₄alkyleneNR⁸R⁹ and R⁸ and R⁹ are each independently selected from H and CH₃. In an embodiment, one of R⁷ is C₁₋₃alkylene₄alkyleneNR⁸R⁹ and R⁸ and R⁹ are each are both H or are both CH₃.

In an embodiment, R⁸ and R⁹ are each independently selected from H and C₁₋₄alkyl. In an embodiment, R⁸ and R⁹ are independently selected from H, CH₃, CH₂CH₃, CF₂H, CF₃, CFH₂, CH₂CF₂H and CH₂CF₃. In an embodiment, R⁸ and Rare independently selected from H, CF₃ and CH₃. In an embodiment, R⁸ and R⁹ are independently selected from H and CH₃.

In an embodiment, Cy¹ is phenyl which is substituted with one or two of R⁷, and at least one of R⁷ is C₁₋₄alkyleneNR⁸R⁹. In an embodiment, Cy¹ is phenyl which is substituted with one or two of R⁷, and at least one of R⁷ is C₁₋₂alkyleneNR⁸R⁹. In an embodiment, Cy¹ is phenyl which is substituted with one or two of R⁷, and at least one of R⁷ is CH₂NR⁸R⁹.

In an embodiment there is only one R⁷ and R⁷ is NR⁸R⁹, and R⁸ and R⁹ are each independently selected from H and C₁₋₄alkyl. In an embodiment there is only one R⁷ and R⁷ is C₁₋₄alkyleneNR⁸R⁹ and R⁸ and R⁹ are each independently selected from H and C₁₋₄alkyl.

In an embodiment, one or two of R⁷ are independently selected from C₃₋₇cycloalkyl and C₃₋₇heterocycloalkyl, C₁₋₄alkyleneC₃₋₇cycloalkyl and C₁₋₄alkyleneC₃₋₇heterocycloalkyl, optionally substituted with one to three of R¹⁰. In an embodiment, one R⁷ is selected from C₁₋₃alkyleneC₃₋₇cycloalkyl and C₃₋₇cycloalkyl, optionally substituted with one or two of R¹⁰. In an embodiment, the C₃₋₇cycloalkyl in the C₁₋₃alkyleneC₃₋₇cycloalkyl and C₃₋₇cycloalkyl of R⁷ is selected from cyclopropyl, cyclobutyl, cyclopentyl, cyclohexyl, cycloheptyl, bicyclo[2.2.1]hexanyl and bicyclo[2.2.1]heptanyl, optionally substituted with one or two of R¹⁰.

In an embodiment, one or two of R⁷ are independently selected from C₃₋₇heterocycloalkyl and C₁₋₄alkyleneC₃₋₇heterocycloalkyl, optionally substituted with one to three of R¹⁰. In an embodiment, the C₃₋₇heterocycloalkyl in the C₃₋₇heterocycloalkyl and C₁₋₄alkyleneC₃₋₇heterocycloalkyl of R⁷ is selected from azetidinyl, oxetanyl, tetrahydrofuranyl, tetrahydrothiophenyl, pyrrolidinyl, pyrrolidin-2-onyl, azabicyclohexanyl, azabicycloheptanyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, dioxanyl, azepanyl, diazepanyl, oxepanyl, thiepanyl, azabicyclohexanyl, azabicycloheptanyl, oxabicyclohexanyl, and oxabicycloheptanyl optionally substituted with one to three of R¹⁰. In an embodiment, the C₃₋₇heterocycloalkyl in the C₃₋₇heterocycloalkyl and C₁₋₄alkyleneC₃₋₇heterocycloalkyl of R⁷ is diazepanyl. In an embodiment, one R⁷ is selected from C₄₋₆ heterocycloalkyl and C₁₋₄alkyleneC₄₋₆heterocycloalkyl, and the C₄₋₆heterocycloalkyl in the C₄₋₆heterocycloalkyl and C₁₋₄alkyleneC₄₋₆heterocycloalkyl of R⁷ is selected from azetidinyl, oxetanyl, tetrahydrofuranyl, tetrahydrothiophenyl, pyrrolidinyl, pyrrolidin-2-onyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, and dioxanyl, optionally substituted with one to three of R¹⁰. In an embodiment, the C₄₋₆heterocycloalkyl in the C₄₋₆heterocycloalkyl and C₁₋₄alkyleneC₄₋₆heterocycloalkyl of R⁷ is selected from tetrahydrofuranyl, tetrahydrothiophenyl, pyrrolidinyl, imidazolidinyl, pyrazolidinyl, pyrrolidin-2-onyl, thiazolidinyl and isothiazolidinyl, optionally substituted with one to three of R¹⁰. In an embodiment, the C₄₋₆heterocycloalkyl in the C₄₋₆heterocycloalkyl and C₁₋₄alkyleneC₄₋₆ heterocycloalkyl of R⁷ is selected from pyrrolidinyl, imidazolidinyl, pyrazolidinyl, thiazolidinyl, pyrrolidin-2-onyl, and isothiazolidinyl, optionally substituted with one to three of R¹⁰.

In an embodiment, one or two of R⁷ are independently selected from C₃₋₇heterocycloalkyl and C₁₋₄alkyleneC₃₋₇heterocycloalkyl, optionally substituted with one to three of R¹⁰. In an embodiment, the C₃₋₇heterocycloalkyl in the C₃₋₇heterocycloalkyl and C₁₋₄alkyleneC₃₋₇heterocycloalkyl of R⁷ is selected from azetidinyl, oxetanyl, tetrahydrofuranyl, tetrahydrothiophenyl, pyrrolidinyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, dioxanyl, azepanyl, diazepanyl, oxepanyl, thiepanyl, azabicyclohexanyl, azabicycloheptanyl, oxabicyclohexanyl, and oxabicycloheptanyl optionally substituted with one to three of R¹⁰. In an embodiment, one R⁷ is selected from C₄₋₆heterocycloalkyl and C₁₋₄alkyleneC₄₋₆heterocycloalkyl, and the C₄₋₆heterocycloalkyl in the C₄₋₆heterocycloalkyl and C₁₋₄alkyleneC₄₋₆heterocycloalkyl of R⁷ is selected from azetidinyl, oxetanyl, tetrahydrofuranyl, tetrahydrothiophenyl, pyrrolidinyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, and dioxanyl, optionally substituted with one to three of R¹⁰. In an embodiment, the C₄₋₆heterocycloalkyl in the C₄₋₆heterocycloalkyl and C₁₋₄alkyleneC₄₋₆heterocycloalkyl of R⁷ is selected from tetrahydrofuranyl, tetrahydrothiophenyl, pyrrolidinyl, imidazolidinyl, pyrazolidinyl, thiazolidinyl and isothiazolidinyl, optionally substituted with one to three of R¹⁰. In an embodiment, the C₄₋₆heterocycloalkyl in the C₄₋₆heterocycloalkyl and C₁₋₄alkyleneC₄₋₆heterocycloalkyl of R⁷ is selected from pyrrolidinyl, imidazolidinyl, pyrazolidinyl, thiazolidinyl and isothiazolidinyl, optionally substituted with one to three of R¹⁰. In an embodiment, the C₄₋₆heterocycloalkyl in the C₄₋₆heterocycloalkyl and C₁₋₄alkyleneC₄₋₆heterocycloalkyl of R⁷ is pyrrolidinyl, optionally substituted with one or two of R¹⁰.

In an embodiment, the C₃₋₇heterocycloalkyl in the C₃₋₇heterocycloalkyl and C₁₋₄alkyleneC₃₋₇heterocycloalkyl of R⁷ comprises at least one N atom. In an embodiment, the C₃₋₇heterocycloalkyl in the C₃₋₇heterocycloalkyl and C₁₋₄alkyleneC₃₋₇heterocycloalkyl of R⁷ is selected from azetidinyl, pyrrolidinyl, pyrrolidin-2-onyl, azabicyclohexanyl, azabicycloheptanyl, piperidinyl, piperazinyl and morpholinyl each of which optionally substituted with one or two of R¹⁰. In an embodiment, one R⁷ is C₄₋₆heterocycloalkyl, optionally substituted with one or two of R¹⁰, and the C₄₋₆heterocycloalkyl is pyrrolidinyl. In an embodiment, one R⁷ is C₄₋₆heterocycloalkyl, and the C₄₋₆heterocycloalkyl is pyrrolidine selected from

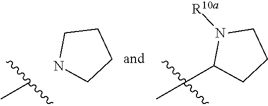

and optionally substituted with one or two of R¹⁰, wherein R^{10a} is selected from H and R¹⁰, and

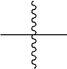

indicates a point of covalent attachment to Cy¹. In an embodiment, the pyrrolidinyl is selected from

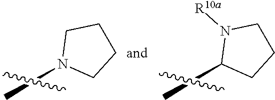

optionally substituted with one or two of R¹⁰, wherein R^{10a} is selected from H and R¹⁰, and

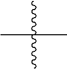

indicates a point of covalent attachment to Cy¹. In an embodiment, R^{10a} is H. In an embodiment, R^{10a} is R¹⁰.

In an embodiment, one of R⁷ is C₁₋₆alkyleneC₃₋₇heterocycloalkyl, optionally substituted with one to four of R¹⁰, and the C₃₋₇heterocycloalkyl in the C₁₋₆alkyleneC₃₋₇heterocycloalkyl is selected from azetidinyl, pyrrolidinyl, pyrrolidin-2-onyl, piperidinyl, piperazinyl and morpholinyl. In an embodiment, one of R⁷ is C₁₋₃alkyleneC₄₋₇ heterocycloalkyl, optionally substituted with one to four of R¹⁰, and the C₄₋₇ heterocycloalkyl in the C₁₋₆alkyleneC₃₋₇heterocycloalkyl is selected from azetidinyl, pyrrolidinyl, piperidinyl, piperazinyl and morpholinyl. Accordingly, in an embodiment, one of R⁷ is selected from C₁₋₃alkyleneazetidinyl, C₁₋₃alkylenepyrrolidinyl, C₁₋₃alkylenepiperidinyl, C₁₋₃alkylenepiperazinyl and C₁₋₃alkylenemorpholinyl optionally substituted with one to four of R¹⁰. In an embodiment, one of R⁷ is selected from CH₂azetidinyl, CH₂pyrrolidinyl, CH₂piperidinyl, CH₂piperazinyl and CH₂morpholinyl optionally substituted with one to four of R¹⁰. In an embodiment, one of R¹¹ is selected from

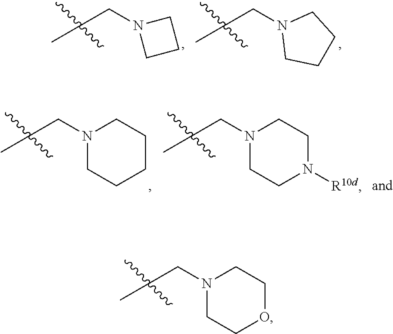

optionally substituted with one or two of R¹⁰, wherein R^{10d} is selected from H and R¹⁰, and

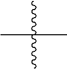

indicates a point of covalent attachment to Cy¹.

In an embodiment, one R⁷ is C₁₋₆alkyleneC₃₋₇heterocycloalkyl, optionally substituted with one or two of R¹⁰, and the C₃₋₇heterocycloalkyl in the C₁₋₆alkyleneC₃₋₇heterocycloalkyl is pyrrolidinyl. In an embodiment, one R⁷ is C₁₋₄alkylenepyrrolidinyl optionally substituted with one to three of R¹⁰. In an embodiment, one R⁷ is C₁₋₄alkyleneC₄₋₆heterocycloalkyl, and the C₄₋₆heterocycloalkyl in the C₁₋₄alkyleneC₄₋₆heterocycloalkyl is pyrrolidinyl optionally substituted with one to three of R¹⁰, wherein at least one R¹⁰ is F.

In an embodiment, each R¹⁰ is independently selected from F, Cl, CN, C₁₋₄alkyl and NR¹¹R^{11a}. In an embodiment, each R¹⁰ is independently selected from F, C₁₋₄alkyl and NR¹¹R^{11a}. In an embodiment, each R¹⁰ is independently selected from F and C₁₋₄alkyl. In an embodiment, each R¹⁰ is independently selected from F and CH₃, CH₂CH₃, CF₂H, CF₃, CFH₂, CH₂CF₂H and CH₂CF₃. In an embodiment, at least one of R¹⁰ is F. In an embodiment, one of R¹⁰ is NR¹¹R^{11a}.

In an embodiment, R¹¹ and R^{11a} are independently selected from H and C₁₋₄alkyl. In an embodiment, R¹¹ and R^{11a} are independently selected from H, CH₃, CH₂CH₃, CF₂H, CF₃, CFH₂, CH₂CF₂H and CH₂CF₃. In an embodiment, R¹¹ and R^{11a} are independently selected from H, CF₃ and CH₃. In an embodiment, R¹¹ and R^{11a} are independently selected from H and CH₃.

In an embodiment, one R⁷ is C₁₋₄alkylenepyrrolidinyl, optionally substituted with one to three of R¹⁰, wherein at least one R¹⁰ is F. In an embodiment, the C₄₋₆heterocycloalkyl in R⁷ is

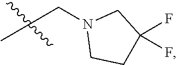 [SMILES: *CN1CCC(F)(F)C1]

wherein

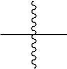

indicates a point of covalent attachment to Cy¹.

In an embodiment, one R⁷ is selected from C₃₋₇cycloalkyl, C₃-7heterocycloalkyl, C₁₋₄alkyleneC₃₋₇cycloalkyl and C₁₋₄alkyleneC₃₋₇heterocycloalkyl which are unsubstituted. In an embodiment, the C₃₋₇heterocycloalkyl in R⁷ is as defined above which is unsubstituted. In an embodiment, the C₃₋₇heterocycloalkyl in R⁷ is selected from pyrrolidinyl, imidazolidinyl, pyrazolidinyl, thiazolidinyl and isothiazolidinyl, which is unsubstituted. In an embodiment, the C₃₋₇heterocycloalkyl in R⁷ is pyrrolidine.

In an embodiment, one R⁷ is pyrrolidinyl which is unsubstituted. In an embodiment, one R⁷ is pyrrolidinyl selected from

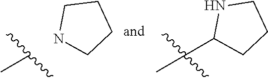

wherein

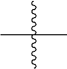

indicates a point of covalent attachment to Cy¹. In an embodiment, one R⁷ is selected from

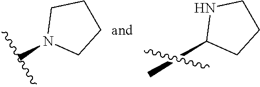

and wherein

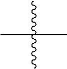

indicates a point of covalent attachment to Cy¹.

In an embodiment, Cy¹ is phenyl, pyrrole, or pyridinyl which is substituted with one to three of R⁷ and one of R⁷ is pyrrolidinyl optionally substituted with one or more of R¹⁰. In an embodiment, Cy¹ is phenyl which is substituted with one to three of R⁷ and one R⁷ is pyrrolidinyl, optionally substituted with one or more of R¹⁰.

In an embodiment, Cy¹ is phenyl, pyrrole, or pyridinyl which is substituted with one to three of R⁷ and one R⁷ is C₁₋₄alkylalkylenepyrrolidinyl optionally substituted with one or two of R¹⁰. In an embodiment, Cy¹ is phenyl which is substituted with one to three of R⁷ and one R⁷ is C₁₋₄alkylalkylenepyrrolidinyl optionally substituted with one or two of R¹⁰.

In an embodiment, Cy¹ is phenyl or pyridinyl which is substituted with one to three of R⁷ and one or two R⁷ are selected from NR⁸R⁹ and C₁₋₄alkyleneNR⁸R⁹, C₃₋₇heterocycloalkyl, and C₁₋₄alkyleneC₃₋₇heterocycloalkyl, the latter two groups being optionally substituted with one to four of R¹⁰. In an embodiment, Cy¹ is phenyl or pyridinyl which is substituted with one to three of R⁷, one or two R⁷ NR⁸R⁹. In an embodiment, Cy¹ is phenyl or pyridinyl which is substituted with one to three of R⁷, one or two R⁷ are selected from C₁₋₄alkyleneNR⁸R⁹. In an embodiment, Cy¹ is phenyl or pyridinyl which is substituted with one to three of R⁷, and one or two R⁷ are independently selected from CH₂N(CH₂CH₃)₂, C(CH₃)₂NH₂, CH₂N(CH₃)₂, CH₂CH₂N(CH₃)₂ and CH₂N(CH₃)₂ and CH₂N(CH₃)₂. In an embodiment, Cy¹ is phenyl or pyridinyl which is substituted with one to three of R⁷, and one R⁷ is CH₂N(CH₃)₂. In an embodiment, Cy¹ is phenyl or pyridinyl which is substituted with one to three of R⁷, and one or two R⁷ are selected from C₁₋₂alkyleneC₃₋₇heterocycloalkyl optionally substituted with one to four of R¹⁰.

In an embodiment, Cy² is a monocyclic C₃₋₇heterocycloalkyl which is substituted with one or more of R¹². In an embodiment, Cy² is azetidinyl, oxetanyl, tetrahydrofuranyl, tetrahydrothiophenyl, pyrrolidinyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, dihydropyranyl tetrahydropyranyl, thiomorpholinyl, morpholinyl, dioxanyl, azepanyl, diazepanyl, oxepanyl or thiepanyl, which is substituted with one to three of R¹². In an embodiment, Cy² is diaxepanyl, 5,6-dihydro-1,2,4-triazinyl, 3,4,5,6-tetrahydro-1,2,4-triazinyl, tetrahydrofuranyl, tetrahydropyranyl, pyrrolidinyl, piperidinyl, piperazinyl or morpholinyl, which is substituted with one to three of R¹². In an embodiment, Cy² is tetrahydrofuranyl, dihydropyranyl, tetrahydropyranyl, pyrrolidinyl, piperidinyl, piperazinyl or morpholinyl, which is substituted with one to three of R¹².

In an embodiment, Cy² is a monocyclic C₃₋₇heterocycloalkyl which is substituted with one or more of R¹². In an embodiment, Cy² is azetidinyl, oxetanyl, tetrahydrofuranyl, tetrahydrothiophenyl, pyrrolidinyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, dioxanyl, azepanyl, diazepanyl, oxepanyl or thiepanyl, which is substituted with one to three of R¹². In an embodiment, Cy² is diaxepanyl, 5,6-dihydro-1,2,4-triazinyl, 3,4,5,6-tetrahydro-1,2,4-triazinyl tetrahydrofuranyl, tetrahydropyranyl, pyrrolidinyl, piperidinyl, piperazinyl or morpholinyl, which is substituted with one to three of R¹². In an embodiment, Cy² is tetrahydrofuranyl, tetrahydropyranyl, pyrrolidinyl, piperidinyl, piperazinyl or morpholinyl, which is substituted with one to three of R¹². In an embodiment, Cy² is pyrrolidinyl, piperidinyl, piperazinyl or morpholinyl, which is substituted with one to three of R¹². In an embodiment, Cy² is selected from piperidinyl, piperazinyl, and morpholinyl, and Cy² is substituted with one or two of R¹². In an embodiment, Cy² is piperazinyl and Cy² is substituted with one or two of R¹². In an embodiment, Cy² is selected from tetrahydrofuranyl, dihydropyranyl, morpholinyl, and tetrahydropyranyl, which is substituted with one to three of R¹². In an embodiment, Cy² is tetrahydropyranyl, which is substituted with one to three of R¹².

In an embodiment, Cy² is a bicyclic heterocycle which is substituted with one to three of R¹². In an embodiment, Cy² is a bridged bicyclic heterocycle, fused bicyclic heterocycle or a spirofused bicyclic heterocycle which is substituted with one to three of R¹². In an embodiment, Cy² is a bridged bicyclic heterocycle which is substituted with one to three of R¹². In an embodiment, Cy² is a fused bicyclic heterocycle which is substituted with to three of R¹². In an embodiment, Cy² is a C₆-C₁₀ saturated bicyclic ring in which one or two of the ring carbon atoms is replaced with N, NH or NR^{12a}, depending on the valency requirements of the N wherein R^{12a} is selected from with H or R¹² and, and which is substituted with one to three of R¹². In an embodiment, Cy² is bridged azabicyclohexanyl, bridged diazabicycloheptanyl or bridged diazabicyclooctanyl which is substituted with one to three of R¹². In an embodiment, Cy² is selected from the following structures:

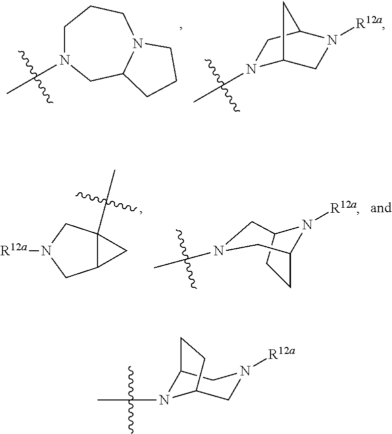

which is substituted with one to three of R¹², wherein

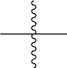

indicates a point of covalent attachment to Cy¹, and R^{12a} is selected from with H or R¹².

In an embodiment, Cy² is selected from the following structures

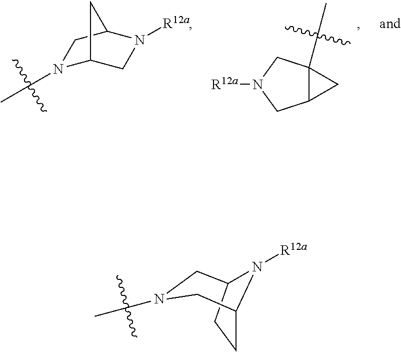

which is substituted with one to three of R¹², wherein

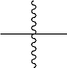

indicates a point of covalent attachment to Cy¹, and R^{12a} is selected from with H or R¹².

In an embodiment, Cy² is selected from tetrahydrofuropyrrolyl, hexapyrazinooxazinyl, hexahydropyrrolopyrazinyl and hexahydropyrrolodiazepiny and Cy² is substituted with one or more of R¹². In an embodiment, Cy² is selected from

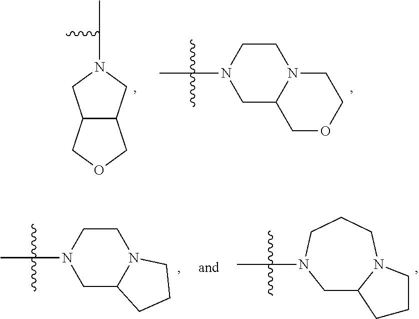

which is substituted with one or more of R¹²; and
wherein

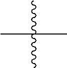

indicates a point of covalent attachment to Cy¹.

In an embodiment, Cy² is an unsubstituted bicyclic heterocycle.

In an embodiment, each R¹² is independently selected from halo, OH, ═O, C₁₋₄alkyl, C₃₋₁₀cycloalkyl, C₃₋₁₀heterocycloalkyl, C₁₋₄alkyleneC₃₋₁₀cycloalkyl, C₁₋₄alkyleneC₃₋₁₀heterocycloalkyl, C₁₋₄alkyleneOR¹³, C₁₋₄alkyleneNR¹³R¹⁴, OC₁₋₄alkyleneOR¹³, OC₁₋₄alkyleneNR¹³R¹⁴, C(O)R¹³, C(O)C₁₋₄alkyleneOR¹³, C(O)C₁₋₄alkyleneNR¹³R¹⁴, C(O)C₁₋₄alkyleneOC₁₋₄alkyleneNR¹³R¹⁴, C(O)NR¹³R¹⁴, CO₂R¹³, CO₂C₁₋₄alkyleneOR¹³, CO₂C₁₋₄alkyleneOC₁₋₄alkyleneNR¹³R¹⁴, NR¹³R¹⁴, NR¹⁵SO₂R¹³, SO₂R¹³ and SO₂NR¹³R¹⁴.

In an embodiment, R¹³ is selected from H, C₁₋₄alkyl, C₃₋₆cycloalkyl, C₁₋₄alkyleneC₃₋₆cycloalkyl, C₃₋₆heterocycloalkyl and C₁₋₄alkyleneC₃₋₆heterocycloalkyl. In an embodiment, R¹³ is selected from H, CH₃, CH₂CH₃, CF₂H, CF₃, CFH₂, CH₂CF₂H and CH₂CF₃, C₁₋₄alkyleneC₃₋₆cycloalkyl and C₁₋₄alkyleneC₃₋₆heterocycloalkyl.

In an embodiment, R¹⁴ is selected from H and C₁₋₄alkyl. In an embodiment, R¹⁴ is selected from H, CH₃ and CH₂CH₃, In an embodiment, R¹⁴ is selected from H, CH₃, CH₂CH₃, CF₂H, CF₃, CFH₂, CH₂CF₂H and CH₂CF₃. In an embodiment, R³ is selected from CF₂H, CH₃ and CF₃. In an embodiment, R¹⁴ is selected from H, CH₃, CH₂CH₃ and CF₃. In an embodiment, R¹⁴ is selected from CH₃ and CF₃. In an embodiment, R¹⁴ is H.

In an embodiment, R¹³ and R¹⁴ are joined to form, together with the nitrogen atom therebetween, a 4- to 6-membered saturated or unsaturated ring, optionally containing one additional heteromoiety selected from N, NR¹⁶, O, S, S(O) and SO₂. In an embodiment, R¹³ and R¹⁴ are joined to form, together the nitrogen atom therebetween to form 4- to 6-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl) and morpholinyl. In an embodiment, R¹³ and R¹⁴ are joined to form, together with the nitrogen atom therebetween, aziridinyl, azetidinyl, pyrrolidinyl, or piperidinyl.

In an embodiment, one R¹² is CO₂R¹³. In an embodiment, R¹³ is selected from H, C₁₋₄alkyl. Accordingly, in an embodiment, one R¹² is CO₂C₁₋₆alkyl. In an embodiment, R¹³ is selected from H, CH₃, CH₂CH₃, CH(CH₃)₂, CF₃, CFH₂, CH₂CF₂H and CH₂CF₃. Accordingly, in an embodiment, one R¹² is selected from CO₂CCH₃, CO₂CH₂CH₃, CO₂CF₂H, CO₂CF₃, CO₂CFH₂, CO₂CH₂CF₂H, CO₂CH₂CF₃, CO₂CH₂CH₂F₂H, CO₂CH₂CH₂CH₂F₂H, CO₂CH(CH₃)₂, and CO₂CH₂CH(CH₃)₂, an embodiment, one R¹² is CO₂CCH₃.

In an embodiment, one R¹² is C(O)R¹³. In an embodiment, R¹³ is selected from H, C₁₋₄alkyl. Accordingly, in an embodiment, one R¹² is C(O)C₁₋₆alkyl. In an embodiment, R¹³ is selected from H, CH₃, CH₂CH₃, CH(CH₃)₂, CF₃, CFH₂, CH₂CF₂H and CH₂CF₃. Accordingly In an embodiment, one R¹² is selected from COCCH₃, COCH₂CH₃, COCF₂H, COCF₃, COCFH₂, COCH₂CF₂H, COCH₂CF₃, COCH₂CH₂F₂H, COCH₂CH₂CH₂F₂H, COCH(CH₃)₂, and COCH₂CH(CH₃)₂. In an embodiment, one R¹² is COCH₃.

In an embodiment, each R¹² is independently selected from the substituents listed below:
OH, F, Cl, CF₃, CH₃, CH₂CH₃, CF₂H, CH₂CF₂H, CH₂CH₂CF₂H, CH₂CH₂CH₂F₂H

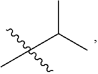 [SMILES: *C(C)C]

CH₂CH₂CH₂CF₃
OC₁₋₄alkyl, SO₂C₁₋₄alkyl, C₁₋₄alkyleneOH, C₁₋₄alkyleneOCH₃, C₁₋₄alkyleneC₃₋₁₀cycloalkyl
C₁₋₄alkyleneC₃₋₁₀hetereocycloalkyl, C₃₋₁₀cycloalkyl, C₃₋₁₀heterocycloalkyl,

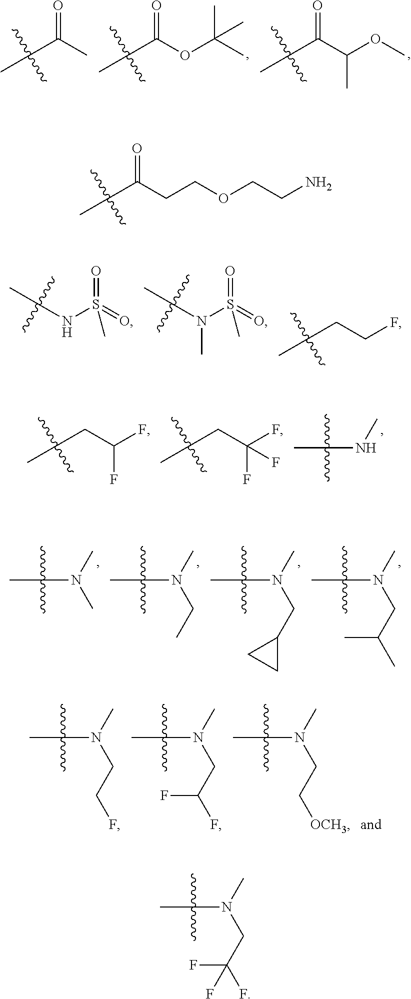

In an embodiment, each R¹² is independently selected from the substituents listed below:
OH, F, Cl, CF₃, CH₃, CH₂CH₃,

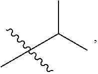 [SMILES: *C(C)C]

OC₁₋₄alkyl, SO₂C₁₋₄alkyl, C₁₋₄alkyleneOH, C₁₋₄alkyleneOCH₃, C₁₋₄alkyleneC₃₋₁₀cycloalkyl
C₁₋₄alkyleneC₃₋₁₀hetereocycloalkyl, C₃₋₁₀cycloalkyl, C₃₋₁₀heterocycloalkyl,

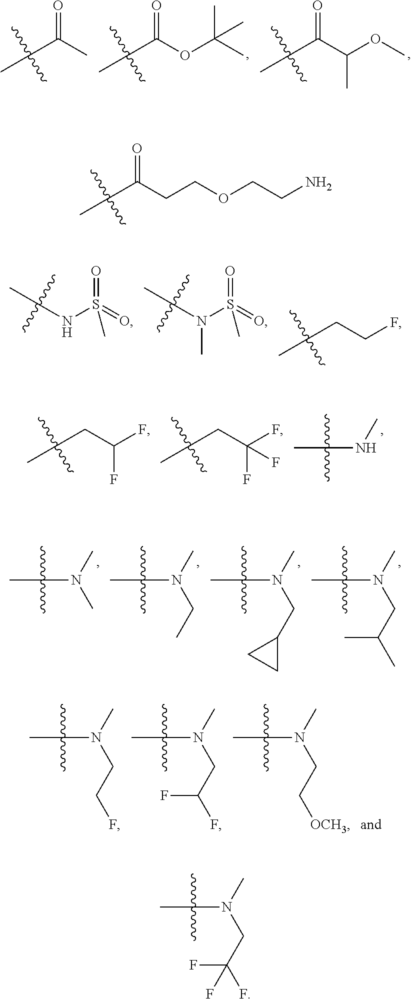

In an embodiment, one or two of R¹² are independently selected from C₃₋₆cycloalkyl, C₃₋₆heteroycloalkyl, C₁₋₄alkyleneC₃₋₆cycloalkyl and C₁₋₄alkyleneC₃₋₆heterocycloalkyl. In an embodiment, one R¹² is selected from C₃₋₅ycloalkyl, C₃-6heteroycloalkyl, C₁₋₄alkyleneC₃₋₅cycloalkyl and C₁₋₄alkyleneC₃₋₅heterocycloalkyl.

In an embodiment, one R¹² is selected from cyclopropyl, cyclobutyl and cyclopentyl. In an embodiment, one R¹² is independently selected from cyclopropyl and cyclobutyl.

In an embodiment, one R¹² is C₁₋₄alkyleneC₃₋₅cycloalkyl selected from C₁₋₄alkylenecyclopropyl, C₁₋₄alkylenecyclobutyl and C₁₋₄alkylenecyclopentyl. In an embodiment, one R¹² is selected from C₁₋₄alkylenecyclopropyl, C₁₋₄alkyleneC₃cyclobutyl and C₁₋₄alkyleneC₃cyclopentyl. In an embodiment, one R¹² is C₁₋₃alkylenecyclopropyl. In an embodiment, one R¹² is C₁₋₃alkylenecyclopropyl selected from

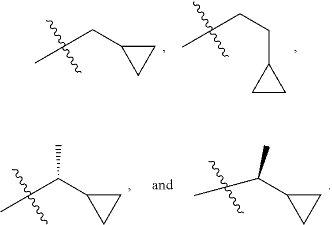

In an embodiment, one R¹² is selected from CH₃, CH₂CH₃, CF₂H, CF₃, CFH₂, CH₂CF₂H, CH₂CH₂CF₂H, CH₂CH₂CH₂CF₂H and CH₂CF₃. In an embodiment, one R¹² is selected from CH₃, CH₂CH₃, CF₂H, CF₃, CFH₂, CH₂CF₂H, and CH₂CF₃. In an embodiment, one R¹² is selected from CH₃, CH₂CH₃, CH(CH₃)₂. In an embodiment, one R²¹ is selected from CH₂CH₃ and CH(CH₃)₂. In an embodiment, one R¹² is selected from CH₂CH₃ and CH(CH₃)₂. In an embodiment, one R¹² is selected CF₂H, CH₂CF₂H, CH₂CH₂CF₂H, and CH₂CH₂CH₂CF₂H. In an embodiment, each R¹² is independently selected from CH₃, CH₂CH₃, CH(CH₃)₂, CF₂H, CF₃, CFH₂, CH₂CF₂H and CH₂CF₃. In an embodiment, each R¹² is independently selected from CH₂CH₃ and CH(CH₃)₂ which are optionally fluoro-substituted.

In an embodiment, one R¹² is selected from oxetanyl, tetrahydrofuranyl and tetrahydropyranyl. In an embodiment, one R¹² is selected from C₁₋₃alkyleneoxetanyl, C₁₋₃alkylenetetrahydrofuranyl and C₁₋₃alkylenetetrahydropyranyl.

In an embodiment, each R¹² is independently selected from the substituents listed below:
OH, F, Cl, CF₃, CH₃, CH₂CH₃,

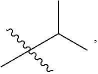 [SMILES: *C(C)C]

OC₁₋₄alkyl, SO₂C₁₋₄alkyl, C₁₋₄alkyleneOH, C₁₋₄alkyleneOCH₃,

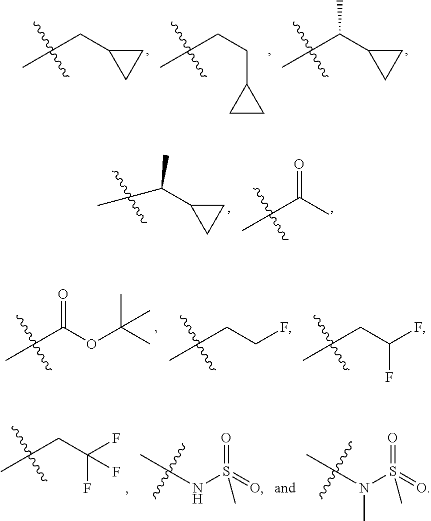

In an embodiment, Cy² is substituted with one to three of R¹². In an embodiment, Cy² is substituted with one or two of R¹². In an embodiment, Cy² is substituted with one R¹².

In an embodiment, Cy² is pyrrolidinyl, piperidinyl, piperazinyl or morpholinyl, and R¹² is CH₃, CH₂CH₃ or CH(CH₃)₂. In an embodiment, Cy² is pyrrolidinyl, piperidinyl, piperazinyl or morpholinyl, and R¹² is selected from CH₃, CH₂CH₃ or CH(CH₃)₂.

In an embodiment, Cy² is pyrrolidinyl, piperidinyl, piperazinyl or morpholinyl, and R¹² is C₁₋₃alkylenecyclopropyl.

In an embodiment, R¹⁵ and R¹⁶ are independently selected from H and C₁₋₄alkyl. In an embodiment, R¹⁵ and R¹⁶ are independently selected from H, CH₃, CH₂CH₃ and CH(CH₃).

In an embodiment, the compound of Formula (I) is a compound of Formula (I-A), or a pharmaceutically acceptable salt, solvate and/or prodrug thereof,

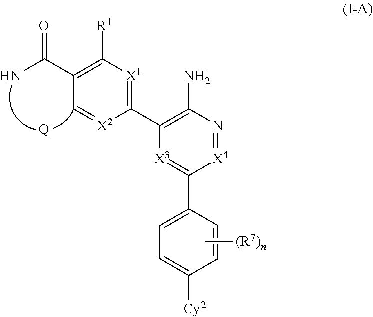

wherein Q, X¹, X², X³, X⁴, R¹, R⁷ and Cy² are as defined in Formula (I); and
n is an integer selected from 0 to 4.

In an embodiment, the compound of Formula (I) is a compound of Formula (I-B), or a pharmaceutically acceptable salt, solvate and/or prodrug thereof,

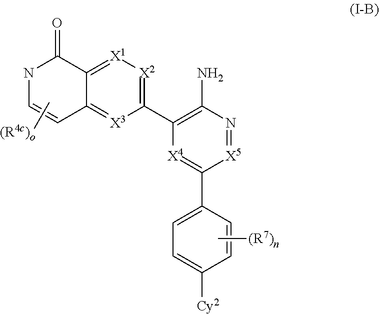

wherein X¹, X², X³, X⁴, X⁵, R^{4c}, R⁷ and Cy² are as defined in Formula (I);
n is an integer selected from 0 to 4; and
is an integer selected from 0 to 2.

In an embodiment, the compound of Formula (I) is a compound of Formula (I-C) or (I-D), or a pharmaceutically acceptable salt, solvate and/or prodrug thereof,

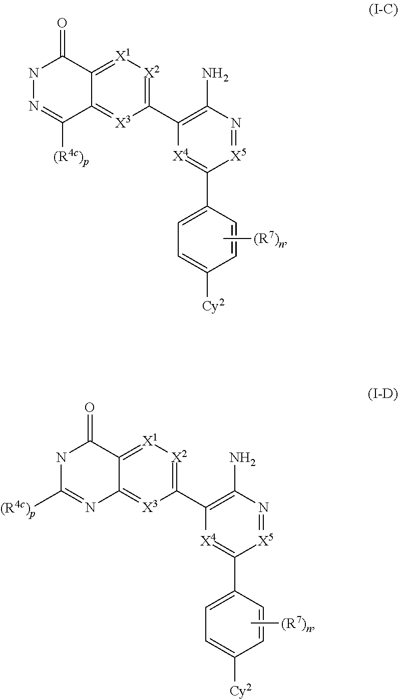

wherein X¹, X², X³, X⁴, X⁵, R^{4c}, R⁷ and Cy² are as defined in Formula (I);
p is an integer selected from 0 and 1; and
n is an integer selected from 0 to 4.

In an embodiment, each R⁷ in the compounds of Formula I-B is independently selected from F, Cl, CH₃, CH₂CH₃, CF₂H, CF₃, CFH₂, CH₂CF₂H and CH₂CF₂. In an embodiment, each R⁷ in the compounds of Formula I-B is selected independently from F, Cl, CH₃, CH₂CH₃ and CF₃. In an embodiment, each R⁷ in the compounds of Formula I-B is independently F, CH₃ or CF₃. In an embodiment, at least one R⁷ in the compounds of Formula I-B is CH₃ or CF₃. In an embodiment, R⁷ in the compounds of Formula I-B is F.

In an embodiment, one R⁷ in the compounds of Formula I-B is selected from C₃₋₇heterocycloalkyl and C₁₋₄alkyleneC₃₋₇heterocycloalkyl optionally substituted with one or two of R¹⁰. In an embodiment, the C₄₋₆heterocycloalkyl in the C₄₋₆heterocycloalkyl and C₁₋₄alkyleneC₄₋₆heterocycloalkyl of R⁷ is pyrrolidinyl, optionally substituted with one or two of R¹⁰. In an embodiment, one R¹⁰ is NR¹¹R^{11a}.

In an embodiment, n is an integer selected from 0 to 2.

In an embodiment, the compound of Formula (I) is a compound of Formula (I-E), or a pharmaceutically acceptable salt, solvate and/or prodrug thereof,

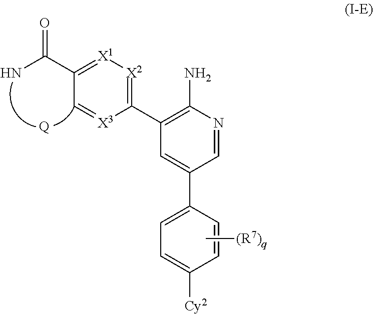

wherein Q, X¹, X², X³, and Cy² are as defined in Formula (I);
each R⁷ is independently selected from NR⁸R⁹, C₁₋₆alkyleneNR⁸R⁹, C₃₋₇heterocycloalkyl, and C₁₋₆alkyleneC₃₋₇heterocycloalkyl, the latter two groups being optionally substituted with one or more of R¹⁰;
q is an integer selected from 1 to 4;
wherein all available hydrogen atoms are optionally substituted with a fluorine atom.

In an embodiment, the compound of Formula I is selected from the compounds listed in Table 1:

TABLE 1

| Compound | | |
|---|---|---|
| I.D | Compound Name | Structure |
| I-1 | 6-(3-amino-6-(4-(4- methylpiperazin-1- yl)phenyl)pyrazin-2-yl)-3,4- dihydroisoquinolin-1(2H)-one | 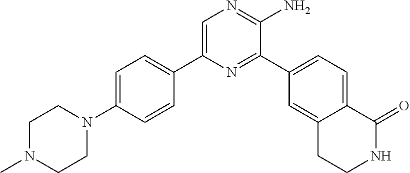 [SMILES: CN1CCN(c2ccc(-c3cnc(N)c(-c4ccc5c(c4)CCNC5=O)n3)cc2)CC1] |
| I-2 | 5-(3-amino-6-(4-(4- methylpiperazin-1- yl)phenyl)pyrazin-2-yl)-3,3- dimethylisoindolin-1-one | 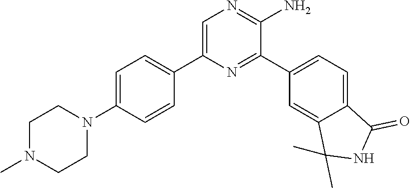 [SMILES: CN1CCN(c2ccc(-c3cnc(N)c(-c4ccc5c(c4)C(C)(C)NC5=O)n3)cc2)CC1] |
| I-3 | 6-(3-amino-6-(4-(4- isopropylpiperazin-1- yl)phenyl)pyrazin-2-yl)-3,4- dihydroisoquinolin-1(2H)-one | 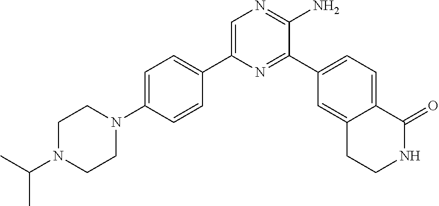 [SMILES: CC(C)N1CCN(c2ccc(-c3cnc(N)c(-c4ccc5c(c4)CCNC5=O)n3)cc2)CC1] |
| I-4 | 6-(3-amino-6-(3-fluoro-4-(4- methylpiperazin-1- yl)phenyl)pyrazin-2-yl)-3,4- dihydroisoquinolin-1(2H)-one | 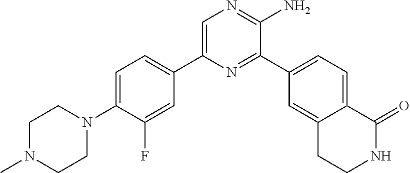 [SMILES: CN1CCN(c2ccc(-c3cnc(N)c(-c4ccc5c(c4)CCNC5=O)n3)cc2F)CC1] |
| I-5 | 6-(3-amino-6-(4-(4- methylpiperazin-1- yl)phenyl)pyrazin-2-yl)-8- fluoro-3,4-dihydroisoquinolin- 1(2H)-one | 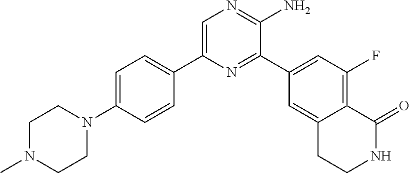 [SMILES: CN1CCN(c2ccc(-c3cnc(N)c(-c4cc(F)c5c(c4)CCNC5=O)n3)cc2)CC1] |
| I-6 | 6-(3-amino-6-(4-(4- hydroxypiperidin-1- yl)phenyl)pyrazin-2-yl)-3,4- dihydroisoquinolin-1(2H)-one | 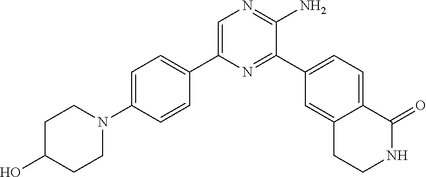 [SMILES: Nc1ncc(-c2ccc(N3CCC(O)CC3)cc2)nc1-c1ccc2c(c1)CCNC2=O] |
| I-7 | (R)-6-(3-amino-6-(4-(2- isopropylmorpholino)phenyl) pyrazin-2-yl)-3,4- dihydroisoquinolin-1(2H)-one | 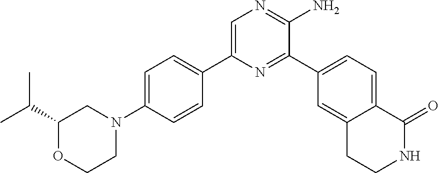 [SMILES: CC(C)[C@@H]1CN(c2ccc(-c3cnc(N)c(-c4ccc5c(c4)CCNC5=O)n3)cc2)CCO1] |
| I-8 | (S)-6-(3-amino-6-(4-(2- isopropylmorpholino)phenyl) pyrazin-2-yl)-3,4- dihydroisoquinolin-1(2H)-one | 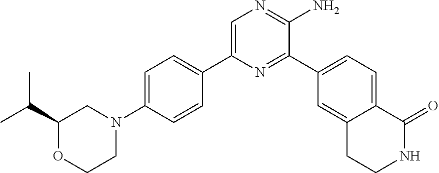 [SMILES: CC(C)[C@H]1CN(c2ccc(-c3cnc(N)c(-c4ccc5c(c4)CCNC5=O)n3)cc2)CCO1] |
| I-9 | (R)-N-(1-(4-(5-amino-6-(1- oxo-1,2,3,4- tetrahydroisoquinolin-6- yl)pyrazin-2- yl)phenyl)pyrrolidin-3-yl)-N- methylmethanesulfonamide | 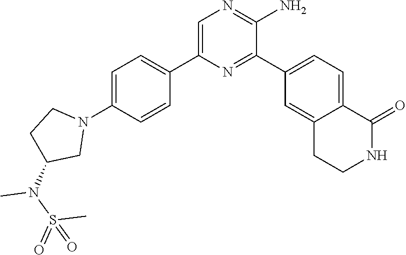 [SMILES: CN([C@@H]1CCN(c2ccc(-c3cnc(N)c(-c4ccc5c(c4)CCNC5=O)n3)cc2)C1)S(C)(=O)=O] |
| I-10 | S)-N-(1-(4-(5-amino-6-(1-oxo- 1,2,3,4-tetrahydroisoquinolin- 6-yl)pyrazin-2- yl)phenyl)pyrrolidin-3-yl)-N- methylmethanesulfonamide | 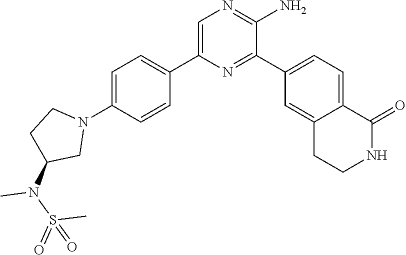 [SMILES: CN([C@H]1CCN(c2ccc(-c3cnc(N)c(-c4ccc5c(c4)CCNC5=O)n3)cc2)C1)S(C)(=O)=O] |
| I-11 | (R)-6-(3-amino-6-(4-(3- methylmorpholino)phenyl) pyrazin-2-yl)-3,4- dihydroisoquinolin-1(2H)-one | 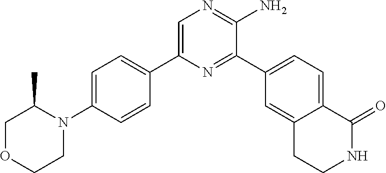 [SMILES: C[C@@H]1COCCN1c1ccc(-c2cnc(N)c(-c3ccc4c(c3)CCNC4=O)n2)cc1] |
| I-12 | 6-(3-amino-6-(4-(4- methylpiperazin-1- yl)phenyl)pyrazin-2-yl)-7- fluoro-3,4-dihydroisoquinolin- 1(2H)-one | 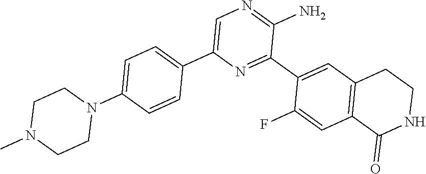 [SMILES: CN1CCN(c2ccc(-c3cnc(N)c(-c4cc5c(cc4F)C(=O)NCC5)n3)cc2)CC1] |
| I-13 | (R)-6-(3-amino-6-(2-fluoro-4- (2- isopropylmorpholino)phenyl) pyrazin-2-yl)-3,4- dihydroisoquinolin-1(2H)-one | 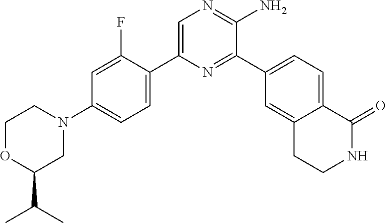 [SMILES: CC(C)[C@@H]1CN(c2ccc(-c3cnc(N)c(-c4ccc5c(c4)CCNC5=O)n3)c(F)c2)CCO1] |
| I-14 | (S)-6-(3-amino-6-(2-fluoro-4- (2- isopropylmorpholino)phenyl) pyrazin-2-yl)-3,4- dihydroisoquinolin-1(2H)-one | 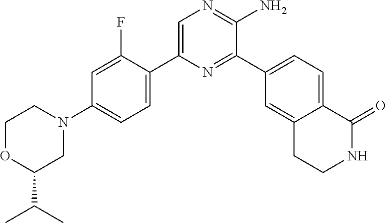 [SMILES: CC(C)[C@H]1CN(c2ccc(-c3cnc(N)c(-c4ccc5c(c4)CCNC5=O)n3)c(F)c2)CCO1] |
| I-15 | (R)-6-(3-amino-6-(2,3- difluoro-4-(2- isopropylmorpholino)phenyl) pyrazin-2-yl)-3,4- dihydroisoquinolin-1(2H)-one | 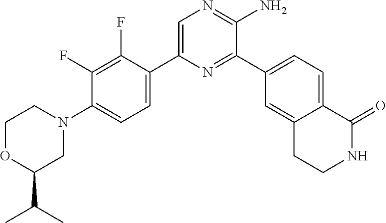 [SMILES: CC(C)[C@@H]1CN(c2ccc(-c3cnc(N)c(-c4ccc5c(c4)CCNC5=O)n3)c(F)c2F)CCO1] |
| I-16 | (S)-6-(3-amino-6-(2,3-difluoro- 4-(2- isopropylmorpholino)phenyl) pyrazin-2-yl)-3,4- dihydroisoquinolin-1(2H)-one | 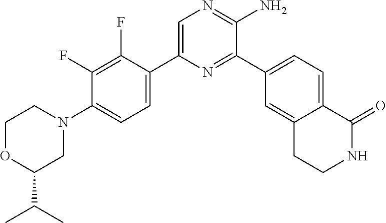 [SMILES: CC(C)[C@H]1CN(c2ccc(-c3cnc(N)c(-c4ccc5c(c4)CCNC5=O)n3)c(F)c2F)CCO1] |
| I-17 | (R)-6-(3-amino-6-(2-fluoro-4- (2-methylpiperidin-1- yl)phenyl)pyrazin-2-yl)-3,4- dihydroisoquinolin-1(2H)-one | 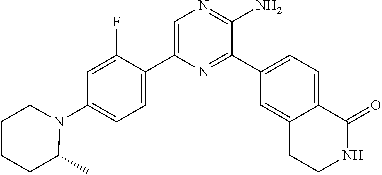 [SMILES: C[C@@H]1CCCCN1c1ccc(-c2cnc(N)c(-c3ccc4c(c3)CCNC4=O)n2)c(F)c1] |
| I-18 | (S)-6-(3-amino-6-(2-fluoro-4- (2-methylpiperidin-1- yl)phenyl)pyrazin-2-yl)-3,4- dihydroisoquinolin-1(2H)-one | 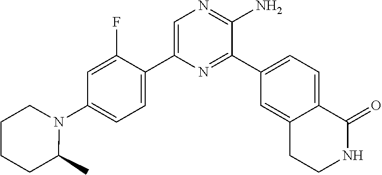 [SMILES: C[C@H]1CCCCN1c1ccc(-c2cnc(N)c(-c3ccc4c(c3)CCNC4=O)n2)c(F)c1] |
| I-19 | 6-(6-(4-(4-acetylpiperazin-1- yl)phenyl)-3-aminopyrazin-2- yl)-3,4-dihydroisoquinolin- 1(2H)-one | 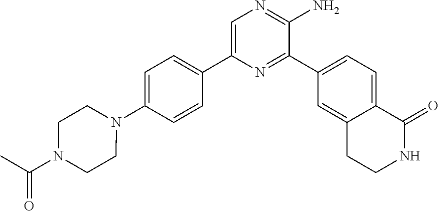 [SMILES: CC(=O)N1CCN(c2ccc(-c3cnc(N)c(-c4ccc5c(c4)CCNC5=O)n3)cc2)CC1] |
| I-20 | 6-(3-amino-6-(4-(3- (dimethylamino)pyrrolidin-1- yl)-2-fluorophenyl)pyrazin-2- yl)-3,4-dihydroisoquinolin- 1(2H)-one | 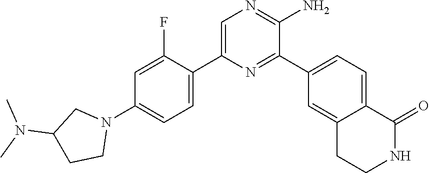 [SMILES: CN(C)C1CCN(c2ccc(-c3cnc(N)c(-c4ccc5c(c4)CCNC5=O)n3)c(F)c2)C1] |
| I-21 | 6-(3-amino-6-(4-(3- (dimethylamino)pyrrolidin-1- yl)-2,3-difluorophenyl)pyrazin- 2-yl)-3,4-dihydroisoquinolin- 1(2H)-one | 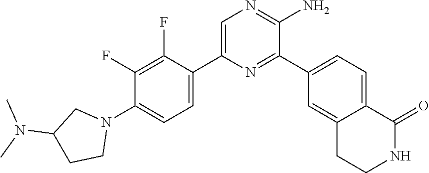 [SMILES: CN(C)C1CCN(c2ccc(-c3cnc(N)c(-c4ccc5c(c4)CCNC5=O)n3)c(F)c2F)C1] |
| I-22 | (R)-6-(3-amino-6-(2-fluoro-4- (3- methylmorpholino)phenyl) pyrazin-2-yl)-3,4- dihydroisoquinolin-1(2H)-one | 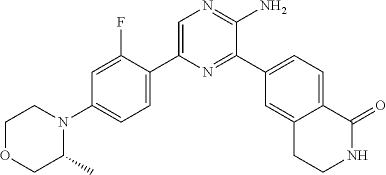 [SMILES: C[C@@H]1COCCN1c1ccc(-c2cnc(N)c(-c3ccc4c(c3)CCNC4=O)n2)c(F)c1] |
| I-23 | (S)-6-(3-amino-6-(2-fluoro-4- (3- methylmorpholino)phenyl) pyrazin-2-yl)-3,4- dihydroisoquinolin-1(2H)-one | 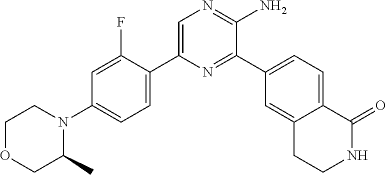 [SMILES: C[C@H]1COCCN1c1ccc(-c2cnc(N)c(-c3ccc4c(c3)CCNC4=O)n2)c(F)c1] |
| I-24 | 6-(3-amino-6-(4-(3- isopropylpiperidin-1- yl)phenyl)pyrazin-2-yl)-3,4- dihydroisoquinolin-1(2H)-one | 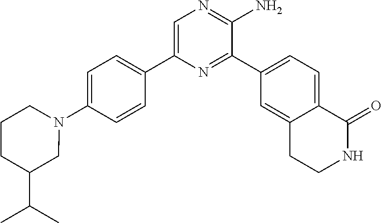 [SMILES: CC(C)C1CCCN(c2ccc(-c3cnc(N)c(-c4ccc5c(c4)CCNC5=O)n3)cc2)C1] |
| I-25 | 6-(3-amino-6-(2-fluoro-4-(3- isopropylpiperidin-1- yl)phenyl)pyrazin-2-yl)-3,4- dihydroisoquinolin-1(2H)-one | 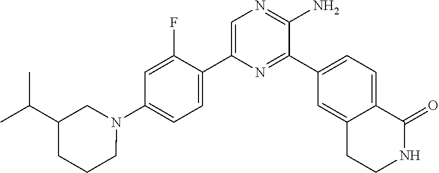 [SMILES: CC(C)C1CCCN(c2ccc(-c3cnc(N)c(-c4ccc5c(c4)CCNC5=O)n3)c(F)c2)C1] |
| I-26 | 6-(3-amino-6-(4-((1S,4S)-5- methyl-2,5- diazabicyclo[2.2.1]heptan-2- yl)phenyl)pyrazin-2-yl)-3,4- dihydroisoquinolin-1(2H)-one | 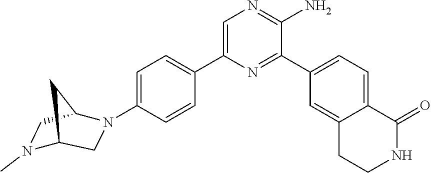 [SMILES: CN1C[C@@H]2C[C@H]1CN2c1ccc(-c2cnc(N)c(-c3ccc4c(c3)CCNC4=O)n2)cc1] |
| I-27 | (R)-6-(3-amino-6-(4-(2- isopropylmorpholino)phenyl) pyrazin-2-yl)-7-fluoro-3,4- dihydroisoquinolin-1(2H)-one | 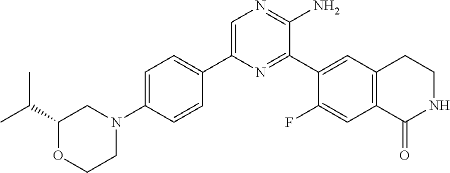 [SMILES: CC(C)[C@@H]1CN(c2ccc(-c3cnc(N)c(-c4cc5c(cc4F)C(=O)NCC5)n3)cc2)CCO1] |
| I-28 | (S)-6-(3-amino-6-(4-(2- isopropylmorpholino)phenyl) pyrazin-2-yl)-7-fluoro-3,4- dihydroisoquinolin-1(2H)-one | 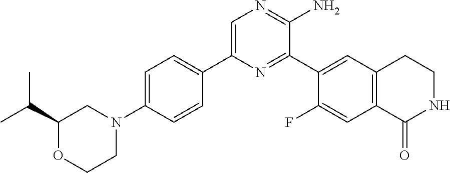 [SMILES: CC(C)[C@H]1CN(c2ccc(-c3cnc(N)c(-c4cc5c(cc4F)C(=O)NCC5)n3)cc2)CCO1] |
| I-29 | (R)-6-(3-amino-6-(2-fluoro-4- (2- isopropylmorpholino)phenyl) pyrazin-2-yl)-7-fluoro-3,4- dihydroisoquinolin-1(2H)-one | 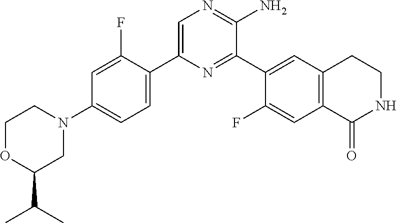 [SMILES: CC(C)[C@@H]1CN(c2ccc(-c3cnc(N)c(-c4cc5c(cc4F)C(=O)NCC5)n3)c(F)c2)CCO1] |
| I-30 | (S)-6-(3-amino-6-(2-fluoro-4- (2- isopropylmorpholino)phenyl) pyrazin-2-yl)-7-fluoro-3,4- dihydroisoquinolin-1(2H)-one | 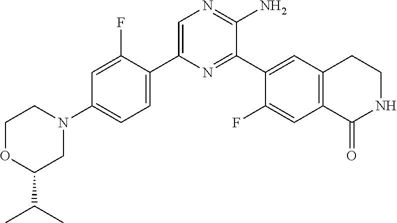 [SMILES: CC(C)[C@H]1CN(c2ccc(-c3cnc(N)c(-c4cc5c(cc4F)C(=O)NCC5)n3)c(F)c2)CCO1] |
| I-31 | (R)-6-(3-amino-6-(2,3- difluoro-4-(2- isopropylmorpholino)phenyl) pyrazin-2-yl)-7-fluoro-3,4- dihydroisoquinolin-1(2H)-one | 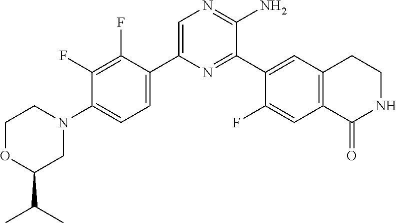 [SMILES: CC(C)[C@@H]1CN(c2ccc(-c3cnc(N)c(-c4cc5c(cc4F)C(=O)NCC5)n3)c(F)c2F)CCO1] |
| I-32 | (S)-6-(3-amino-6-(2,3-difluoro- 4-(2- isopropylmorpholino)phenyl) pyrazin-2-yl)-7-fluoro-3,4- dihydroisoquinolin-1(2H)-one | 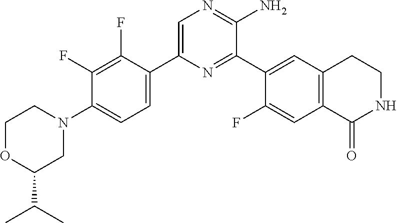 [SMILES: CC(C)[C@H]1CN(c2ccc(-c3cnc(N)c(-c4cc5c(cc4F)C(=O)NCC5)n3)c(F)c2F)CCO1] |
| I-33 | 6-(3-amino-6-(4-(1-methyl- 5,6-dihydro-1,2,4-triazin- 4(1H)-yl)phenyl)pyrazin-2-yl)- 3,4-dihydroisoquinolin-1(2H)- one | 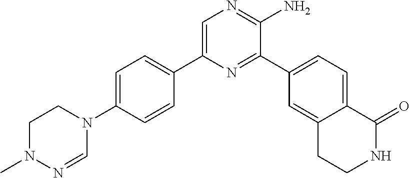 [SMILES: CN1CCN(c2ccc(-c3cnc(N)c(-c4ccc5c(c4)CCNC5=O)n3)cc2)C=N1] |
| I-34 | 6-(3-amino-6-(4-((1S,4S)-5- (2,2,2-trifluoroethyl)-2,5- diazabicyclo[2.2.1]heptan-2- yl)phenyl)pyrazin-2-yl)-3,4- dihydroisoquinolin-1(2H)-one | 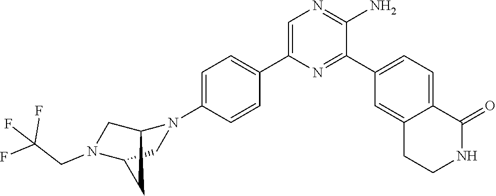 [SMILES: Nc1ncc(-c2ccc(N3C[C@@H]4C[C@H]3CN4CC(F)(F)F)cc2)nc1-c1ccc2c(c1)CCNC2=O] |
| I-35 | 6-(3-amino-6-(4-(4-(2,2,2- trifluoroethyl)piperazin-1- yl)phenyl)pyrazin-2-yl)-3,4- dihydroisoquinolin-1(2H)-one | 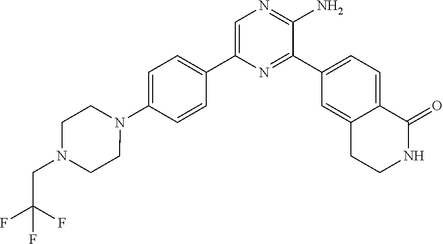 [SMILES: Nc1ncc(-c2ccc(N3CCN(CC(F)(F)F)CC3)cc2)nc1-c1ccc2c(c1)CCNC2=O] |
| I-36 | 6-(3-amino-6-(4-(1-(2,2,2- trifluoroethyl)piperidin-4- yl)phenyl)pyrazin-2-yl)-3,4- dihydroisoquinolin-1(2H)-one | 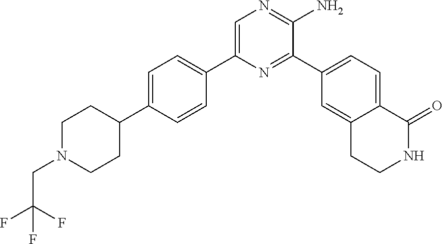 [SMILES: Nc1ncc(-c2ccc(C3CCN(CC(F)(F)F)CC3)cc2)nc1-c1ccc2c(c1)CCNC2=O] |
| I-37 | 6-(3-amino-6-(4-((1R,5S)-3- (2-fluoroethyl)-3- azabicyclo[3.1.0]hexan-1- yl)phenyl)pyrazin-2-yl)-3,4- dihydroisoquinolin-1(2H)-one | 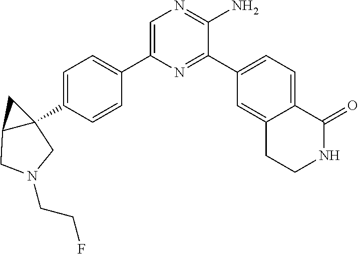 [SMILES: Nc1ncc(-c2ccc([C@@]34C[C@@H]3CN(CCF)C4)cc2)nc1-c1ccc2c(c1)CCNC2=O] |
| I-38 | (R)-6-(3-amino-6-(4-(3- isopropyl-4-methylpiperazin- 1-yl)phenyl)pyrazin-2-yl)-3,4- dihydroisoquinolin-1(2H)-one | 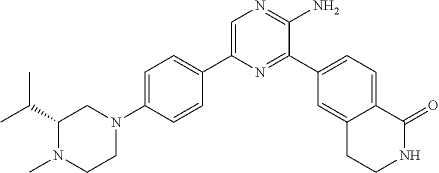 [SMILES: CC(C)[C@@H]1CN(c2ccc(-c3cnc(N)c(-c4ccc5c(c4)CCNC5=O)n3)cc2)CCN1C] |
| I-39 | (R)-6-(3-amino-6-(2-fluoro-4- (3-isopropyl-4- methylpiperazin-1- yl)phenyl)pyrazin-2-yl)-3,4- dihydroisoquinolin-1(2H)-one | 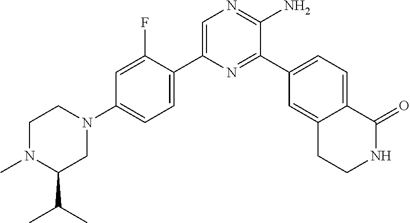 [SMILES: CC(C)[C@@H]1CN(c2ccc(-c3cnc(N)c(-c4ccc5c(c4)CCNC5=O)n3)c(F)c2)CCN1C] |
| I-40 | (S)-6-(3-amino-6-(4-(3- isopropyl-4-methylpiperazin- 1-yl)phenyl)pyrazin-2-yl)-3,4- dihydroisoquinolin-1(2H)-one | 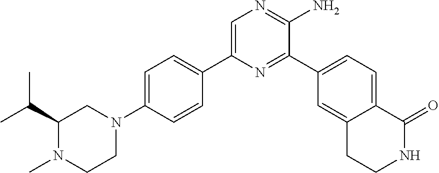 [SMILES: CC(C)[C@H]1CN(c2ccc(-c3cnc(N)c(-c4ccc5c(c4)CCNC5=O)n3)cc2)CCN1C] |
| I-41 | (S)-6-(3-amino-6-(2-fluoro-4- (3-isopropyl-4- methylpiperazin-1- yl)phenyl)pyrazin-2-yl)-3,4- dihydroisoquinolin-1(2H)-one | 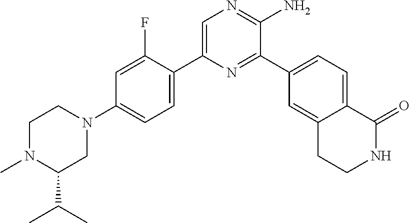 [SMILES: CC(C)[C@H]1CN(c2ccc(-c3cnc(N)c(-c4ccc5c(c4)CCNC5=O)n3)c(F)c2)CCN1C] |
| I-42 | (R)-6-(3-amino-6-(4-(2- methylpyrrolidin-1- yl)phenyl)pyrazin-2-yl)-3,4- dihydroisoquinolin-1(2H)-one | 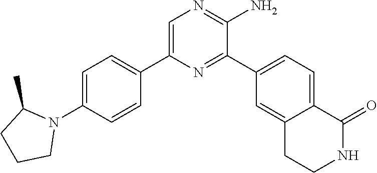 [SMILES: C[C@@H]1CCCN1c1ccc(-c2cnc(N)c(-c3ccc4c(c3)CCNC4=O)n2)cc1] |
| I-43 | (R)-6-(3-amino-6-(2-fluoro-4- (2-methylpyrrolidin-1- yl)phenyl)pyrazin-2-yl)-3,4- dihydroisoquinolin-1(2H)-one | 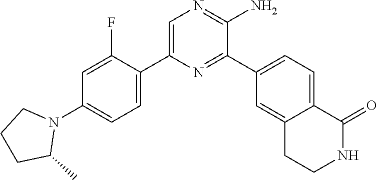 [SMILES: C[C@@H]1CCCN1c1ccc(-c2cnc(N)c(-c3ccc4c(c3)CCNC4=O)n2)c(F)c1] |
| I-44 | 6-(3-amino-6-(4-(4- isopropylpiperazin-1- yl)phenyl)pyrazin-2-yl)-7- fluoro-3,4-dihydroisoquinolin- 1(2H)-one | 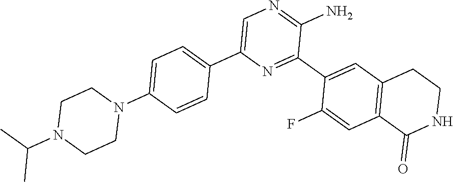 [SMILES: CC(C)N1CCN(c2ccc(-c3cnc(N)c(-c4cc5c(cc4F)C(=O)NCC5)n3)cc2)CC1] |
| I-45 | 6-(3-amino-6-(4-((1R,5S)-3- methyl-3- azabicyclo[3.1.0]hexan-1- yl)phenyl)pyrazin-2-yl)-7- fluoro-3,4-dihydroisoquinolin- 1(2H)-one | 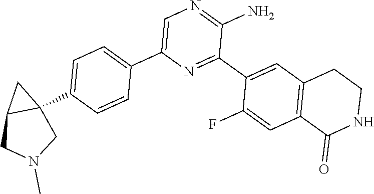 [SMILES: CN1C[C@H]2C[C@@]2(c2ccc(-c3cnc(N)c(-c4cc5c(cc4F)C(=O)NCC5)n3)cc2)C1] |
| I-46 | 6-(3-amino-6-(4-((1R,5S)-3- isopropyl-3- azabicyclo[3.1.0]hexan-1- yl)phenyl)pyrazin-2-yl)-7- fluoro-3,4-dihydroisoquinolin- 1(2H)-one | 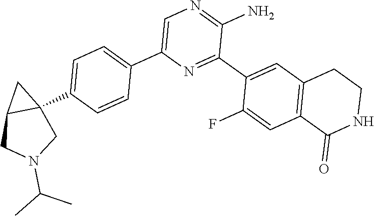 [SMILES: CC(C)N1C[C@H]2C[C@@]2(c2ccc(-c3cnc(N)c(-c4cc5c(cc4F)C(=O)NCC5)n3)cc2)C1] |
| I-47 | 6-(3-amino-6-(4-((1S,5R)-3- methyl-3- azabicyclo[3.1.0]hexan-1- yl)phenyl)pyrazin-2-yl)-7- fluoro-3,4-dihydroisoquinolin- 1(2H)-one | 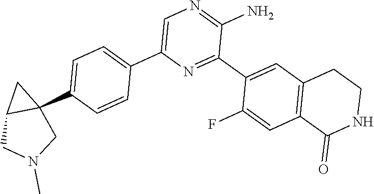 [SMILES: CN1C[C@@H]2C[C@]2(c2ccc(-c3cnc(N)c(-c4cc5c(cc4F)C(=O)NCC5)n3)cc2)C1] |
| I-48 | 6-(3-amino-6-(4-((1S,5R)-3- isopropyl-3- azabicyclo[3.1.0]hexan-1- yl)phenyl)pyrazin-2-yl)-7- fluoro-3,4-dihydroisoquinolin- 1(2H)-one | 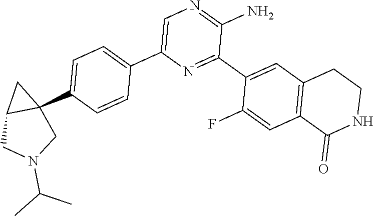 [SMILES: CC(C)N1C[C@@H]2C[C@]2(c2ccc(-c3cnc(N)c(-c4cc5c(cc4F)C(=O)NCC5)n3)cc2)C1] |
| I-49 | 6-(3-amino-6-(4-(4- methylpiperazin-1- yl)phenyl)pyridazin-4-yl)-3,4- dihydroisoquinolin-1(2H)-one | 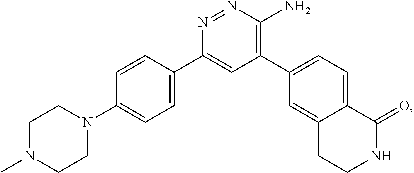 [SMILES: CN1CCN(c2ccc(-c3cc(-c4ccc5c(c4)CCNC5=O)c(N)nn3)cc2)CC1] |
| I-50 | 7-(3-amino-6-(4-(4- methylpiperazin-1- yl)phenyl)pyrazin-2-yl)- 2,3,4,5-tetrahydro-1H- benzo[c]azepin-1-one | 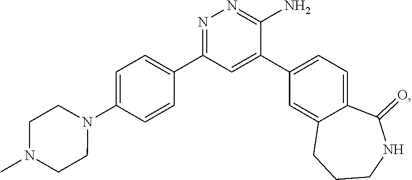 [SMILES: CN1CCN(c2ccc(-c3cc(-c4ccc5c(c4)CCCNC5=O)c(N)nn3)cc2)CC1] |
| I-51 | 6-(3-amino-6-(4-(4- methylpiperazin-1- yl)phenyl)pyrazin-2-yl)-3- methylisoquinolin-1(2H)-one | 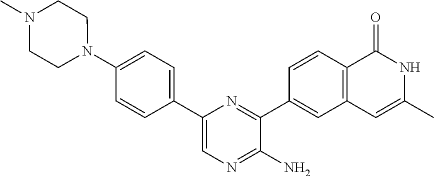 |
| I-52 | 6-(3-amino-6-(4-((1S,5R)-3- cyclopropyl-3- azabicyclo[3.1.0]hexan-1- yl)phenyl)pyrazin-2-yl)-7- fluoro-3,4-dihydroisoquinolin- 1(2H)-one | 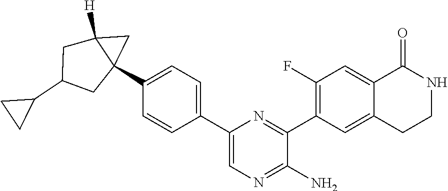 [SMILES: Nc1ncc(-c2ccc([C@@]34CC(C5CC5)C[C@@H]3C4)cc2)nc1-c1cc2c(cc1F)C(=O)NCC2] |
| I-53 | (R)-6-(3-amino-6-(4-(4- isopropyl-2-methylpiperazin- 1-yl)phenyl)pyrazin-2-yl)-3,4- dihydroisoquinolin-1(2H)-one | 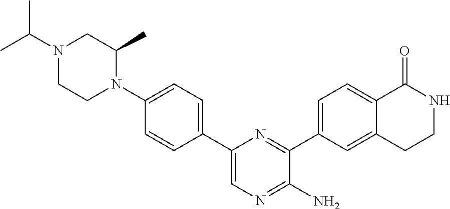 [SMILES: CC(C)N1CCN(c2ccc(-c3cnc(N)c(-c4ccc5c(c4)CCNC5=O)n3)cc2)[C@H](C)C1] |
| I-54 | (R)-6-(3-amino-6-(4-(2,4- dimethylpiperazin-1- yl)phenyl)pyrazin-2-yl)-3,4- dihydroisoquinolin-1(2H)-one | 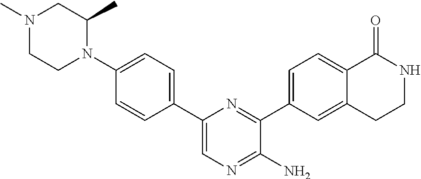 [SMILES: C[C@@H]1CN(C)CCN1c1ccc(-c2cnc(N)c(-c3ccc4c(c3)CCNC4=O)n2)cc1] |
| I-55 | 6-(3-amino-6-(4-((3R,5S)- 3,4,5-trimethylpiperazin-1- yl)phenyl)pyrazin-2-yl)-3,4- dihydroisoquinolin-1(2H)-one | 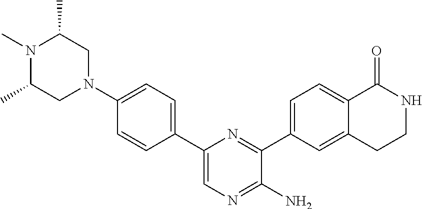 [SMILES: C[C@@H]1CN(c2ccc(-c3cnc(N)c(-c4ccc5c(c4)CCNC5=O)n3)cc2)C[C@H](C)N1C] |
| I-56 | 6-(3-amino-6-(4-(4- ethylpiperazin-1- yl)phenyl)pyrazin-2-yl)-3,4- dihydroisoquinolin-1(2H)-one | 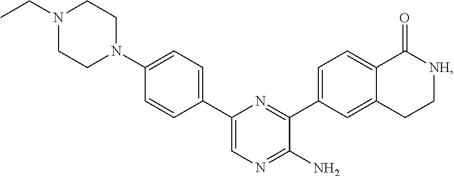 [SMILES: CCN1CCN(c2ccc(-c3cnc(N)c(-c4ccc5c(c4)CCNC5=O)n3)cc2)CC1] |
| I-57 | 7-(3-amino-6-(4-(4- methylpiperazin-1- yl)phenyl)pyrazin-2-yl)-2- methylquinazolin-4(3H)-one | 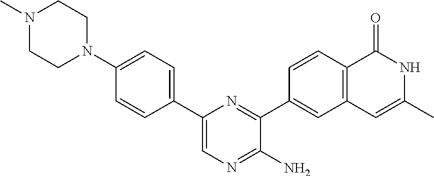 |

or a pharmaceutically acceptable salt, solvate and/or prodrug thereof.

### i) Compounds of Formula (II)

The present application describes a novel class of substituted amino pyridine HPK1 inhibitors.

The application also includes a compound of Formula (II), or a pharmaceutically acceptable salt, solvate and/or prodrug thereof,

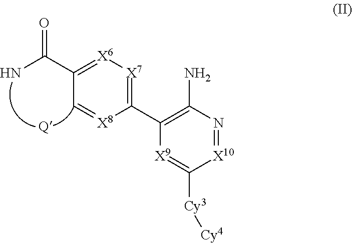

wherein
X⁶ is selected from N and CR¹⁷;
X⁷ and X⁸ are each independently selected from N and CR¹⁸;
X⁹ and X¹⁰ are each independently selected from N and CH, provided at least one of X⁹ and X¹⁰ is N;
Q′ is C₁₋₄alkylene optionally interrupted by a heteromoiety selected from O, S, S(O), SO₂ and NR¹⁹ and/or optionally substituted with one or more of R²⁰ and/or optionally disubstituted on one carbon with R²¹ and R^{21a}, provided that when Q comprises the heteromoiety the heteromoiety is separated from the ring amide NH by other than methylene; or
Q′ is C₂₋₄alkenylene optionally substituted with one or more of R²²; or
Q′ is C═N or N═C optionally substituted with R²²;
R¹⁷ is selected from H, halo, OR²³, NR²⁴R²⁵, C₁₋₆alkyleneNR²⁴R²⁵ and C₁₋₆alkyl;
R¹⁸ is selected from H, halo and C₁₋₆alkyl;
R¹⁹ is selected from H and C₁₋₆alkyl;
each R²⁰ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR²⁶R²⁷ and C₁₋₆alkyleneNR²⁶R²⁷;
R²¹ and R^{21a} are joined to form, together with the carbon atom therebetween, a 3- to 6-membered, saturated or unsaturated ring optionally containing one heteromoiety selected from N, NH, NC₁₋₆alkyl, O, S, S(O), and SO₂ and optionally substituted with one or more of halo and C₁₋₆alkyl;
each R²² is independently selected from halo, C₁₋₆alkyl, C₃₋₆cycloalkyl, C₃-6heterocycloalkyl, C₁₋₆alkyleneC₃₋₆cycloalkyl, C₁₋₆alkyleneC₃₋₆heterocycloalkyl, OH, OC₁₋₆alkyl, NR²⁶R²⁷ and C₁₋₆alkyleneNR²⁶R²⁷;
R²⁴, R²⁵, R²⁶ and R²⁷ are each independently selected from H and C₁₋₆alkyl, or
R²⁴ and R²⁵ or R²⁶ and R²⁷ are joined to form, together with the atom therebetween, a 3- to 7-membered saturated or unsaturated ring, optionally containing one additional heteromoiety selected from N, NH, NC₁₋₆alkyl, O, S, S(O), and SO₂ and optionally substituted with one or more of halo and C₁₋₆alkyl;
Cy³ is C₆₋₁₀aryl or C₅₋₁₀heteroaryl, which substituted with one or two of R²⁸, and optionally further substituted with one to three of R²⁹;
each R²⁸ is independently selected from NR³⁰R³¹, C₁₋₆alkyleneNR³⁰R³¹, C₃₋₇heterocycloalkyl and C₁₋₆alkyleneC₃₋₇heterocycloalkyl, the latter two groups being optionally substituted with one or more of R³²;
each R²⁹ is independently selected from halo, C₁₋₆alkyl, C₃₋₇cycloalkyl, and C₁₋₆alkyleneC₃₋₇cycloalkyl, the latter two groups being optionally substituted with one or more of R³²;
R³⁰ and R³¹ are each independently selected from H and C₁₋₆alkyl;
each R³² is independently selected from halo, C₁₋₆alkyl, CN and NR³³R³⁴;
R³³ and R³⁴ are each independently selected from H and C₁₋₆alkyl;
Cy⁴ is C₃₋₁₄heterocycloalkyl, and Cy⁴ is unsubstituted or substituted with one or more of R³⁵;
each R³⁵ is independently selected from halo, ═O, CN, OH, C₁₋₆alkyl, C₂₋₆alkenyl, C₂₋₆alkynyl, C₃₋₁₀cycloalkyl, C₃₋₁₀heterocycloalkyl, C₁₋₆alkyleneC₃₋₁₀cycloalkyl, C₁₋₆alkyleneC₃₋₁₀heterocycloalkyl, C₁₋₆alkyleneOR³⁶, C₁₋₆alkyleneNR³⁶R³⁷, OC₁₋₆alkyleneOR³⁶, OC₁₋₆alkyleneNR³⁶R³⁷, SR³⁶, C(O)R³⁶ C(O)C₁₋₆alkyleneOR³⁶, C(O)C₁₋₆alkyleneNR³⁶R³⁷, C(O)C₁₋₆alkyleneOC₁₋₆alkyleneNR³⁶R³⁷, C(O)NR³⁶R³⁷, CO₂R³⁶, CO₂C₁₋₆alkyleneOR³⁶, CO₂C₁₋₆alkyleneOC₁₋₆alkyeneNR³⁶R³⁷, NR³⁶R³⁷, NR³⁸SO₂R³⁶, S(O)R³⁶, SO₂R³⁶, SO₂NR³⁶R³⁷ and S(O)(NR³⁸)R³⁶;
R³⁶ is selected from H, C₁₋₆alkyl, C₁₋₆alkyleneOR¹⁴, C₃₋₁₀cycloalkyl, C₃₋₁₀heterocycloalkyl, C₁₋₆alkyleneC₃₋₁₀cycloalkyl and C₁₋₆alkyleneC₃₋₁₀heterocycloalkyl,
R³⁷ is selected from H and C₁₋₆alkyl; or
R³⁶ and R³⁷ are joined to form, together with the nitrogen atom therebetween, a 4- to 6-membered saturated or unsaturated ring, optionally containing one additional heteromoiety selected from N, NR³⁹, O, S, SO and SO₂; and
R³⁸ and R³⁹ are independently selected from H and C₁₋₆alkyl;
wherein all available hydrogen atoms are optionally substituted with a fluorine atom.

In all embodiments below it is to be understood that all available hydrogen atoms are optionally substituted with a fluorine atom. This has not been repeated throughout. Thus in each embodiment where a group is listed that comprises available hydrogen atoms, it is to be understood that all such atoms are optionally replaced with fluorine atoms, for example each recitation of C₁₋₆alkyl is also a recitation of C₁₋₆fluoroalkyl, unless stated otherwise.

In an embodiment, X⁶ is N. In an embodiment, X⁶ is CR¹⁷.

In an embodiment, R¹⁷ is selected from H, F, Cl, OR²³, NR²⁴R²⁵, C₁₋₄alkyleneNR²⁴R²⁵, and C₁₋₄alkyl.

In an embodiment, R¹⁷ is selected from H, F, Cl and C₁₋₄alkyl. In an embodiment, R¹⁷ is selected from H, F, Cl, CH₃, CH₂CH₃, CF₂H, CF₃, CFH₂, CH₂CF₂H and CH₂CF₃. In an embodiment, R¹⁷ is selected from H, F, CF₃, CF₂H, CH₂CF₂H and CH₃. In an embodiment, R¹⁷ is selected from H, F, CF₃, CF₂H and CH₂CF₂H. In an embodiment, R¹⁷ is selected from H, F, CF₃ and CH₃. In an embodiment, R¹⁷ is selected from H and F. In an embodiment, R¹⁷ is H.

In an embodiment, R¹⁷ is OR²³. In an embodiment, R²³ is selected from H, CH₃, CH₂CH₃, CF₃, CFH₂, CF₂H, CH₂CF₂H, and CH₂CF₂H. In an embodiment, R²³ is selected from H and C₁₋₄alkyl. In an embodiment, R²³ is selected from H, CH₃ and CH₂CH₃. In an embodiment, R²³ is selected from H, CH₃, CH₂CH₃, CF₃, CFH₂, CH₂CF₂H, and CH₂CH₂F. In an embodiment, R²³ is CF₂H. Accordingly, in an embodiment, R¹⁷ is selected from OH, OCH₃, OCH₂CH₃, OCF₃, OCFH₂, OCHF₂, OCH₂CF₂H, and OCH₂CF₂H.

In an embodiment, R¹⁷ is selected from NR²⁴R²⁵, and C₁₋₄alkyleneNR²⁴R²⁵. In an embodiment, R¹⁷ is selected from NR²⁴R²⁵ and C₁₋₂alkyleneNR²⁴R²⁵.

In an embodiment, R²⁴ and R²⁵ are each independently selected from H and C₁₋₄alkyl. In an embodiment, R²⁴ and R²⁵ are each independently selected from H, CH₃ and CF₃. In an embodiment, one of R²⁴ and R²⁵ is H and the other is CH₃. In an embodiment, R²⁴ and R²⁵ are both CH₃. In an embodiment, R²⁴ and R²⁵ are both H.

In an embodiment, R²⁴ and R²⁵ are joined to form, together with the atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R²⁴ and R²⁵ are taken together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl ring, and optionally substituted with one or more of halo and C₁₋₆alkyl. In an embodiment, R²⁴ and R²⁵ are joined to form, together with the atom therebetween, a 4- to 6-membered saturated ring, and optionally substituted with one or more of halo and C₁₋₆alkyl. In an embodiment R²⁴ and R²⁵ are joined to form, together with the atom therebetween, aziridinyl, azetidinyl, pyrrolidinyl, or piperidinyl, and optionally substituted with one or more of halo and C₁₋₆alkyl.

In an embodiment, one of X⁷ and X⁸ is N and the other is CR¹⁸. In an embodiment, X⁷ is CR¹⁸ and X⁸ is N. In an embodiment, both X⁷ and X⁸ are independently CR¹⁸.

In an embodiment, each R¹⁸ is independently selected from H, halo and C₁₋₄alkyl. In an embodiment, each R¹⁸ is independently selected from H, F, Cl and C₁₋₄alkyl. In an embodiment, each R¹⁸ is independently selected from H, F, Cl, CH₃, CF₃, CH₂F and CHF₂. In an embodiment, R¹⁸ is selected from CF₂H, CH₃ and CF₃. In an embodiment, each R¹⁸ is independently selected from H, F, Cl, CH₃ and CF₃. In an embodiment, each R¹⁸ is selected from H and F.

In an embodiment, one of X⁷ and X⁸ is N and the other is CH. In an embodiment, X⁷ is CH and X⁸ is N. In an embodiment, X⁷ is CH and X⁸ is CF or CCl. In an embodiment, X⁷ and X⁸ are both CF. In an embodiment, X⁷ is selected from CH, CF, CCl, CCH₃ and CCF₃ and X⁸ is CH. In an embodiment, X⁷ and X⁸ are both CH. In an embodiment, both X⁷ and X⁸ are N.

In an embodiment, X⁶ is CR¹⁷ and X⁷ and X⁸ are both CR¹⁸. In an embodiment, X⁶ is N and an X⁵ and X⁶ are independently CR¹⁸. In an embodiment, X⁶ is N and an X⁷ and X⁸ are both CH. In an embodiment, X⁶ is CR¹⁷ and one of X⁷ and X⁸ is N and the other is CR¹⁸. In an embodiment, X⁶ is CR¹⁷ and one of X⁷ and X⁸ is N and the other is CH.

In an embodiment, one of X⁹ and X¹⁰ is N and the other is CH. In an embodiment, X⁹ is N and X¹⁰ is CH. In embodiment, X⁹ is CH and X¹⁰ is N.

In an embodiment, Q′ is C₁₋₃alkylene optionally interrupted by a heteromoiety selected from O, S, S(O), SO₂, and NR¹⁹ and/or optionally substituted with one or more of R²⁰.

In an embodiment, Q′ is C₁₋₃alkylene optionally interrupted by a heteromoiety selected from O, S, S(O), SO₂, and NR¹⁹. In an embodiment, Q′ is C₁₋₃ alkylene optionally interrupted by a heteromoiety selected from O, SO₂, and NR¹⁹. In an embodiment, Q′ is C₁₋₃alkylene optionally interrupted by a heteromoiety selected from O and NR¹⁹. In an embodiment, Q′ is C₁₋₃alkylene optionally interrupted by a heteromoiety selected from O and SO₂. In an embodiment, Q′ is C₁₋₃alkylene optionally interrupted by O. In an embodiment, Q′ is C₁₋₂alkylene optionally interrupted by SO₂. In an embodiment, Q′ is C₁₋₂alkylene optionally interrupted SO₂. In an embodiment, Q′ is Q is C═C optionally interrupted SO₂.

In an embodiment, R¹⁹ is selected from H and C₁₋₄alkyl. In an embodiment, R¹⁹ is selected from H, CH₃ and CH₂CH₃, In an embodiment, R¹⁹ is selected from, H, CH₃, CH₂CH₃, CF₂H, CF₃, CFH₂, CH₂CF₂H and CH₂CF₃. In an embodiment, R¹⁹ is selected from CF₂H, CH₃ and CF₃. In an embodiment, R¹⁹ is selected from H, CH₃, CH₂CH₃ and CF₃. In an embodiment, R¹⁹ is selected from CH₃ and CF₃. In an embodiment, R¹⁹ is H.

In an embodiment, Q′ is C₁₋₃alkylene and optionally substituted with one to three of R²⁰. In an embodiment, Q′ is CH₂ or CH₂CH₂ and optionally substituted with one or two of R²⁰. In an embodiment, Q′ is C₁alkylene and optionally substituted with one or two of R²⁰. In an embodiment, Q′ is CH₂. In an embodiment, Q′ is CH₂CH₂.

In an embodiment, each R²⁰ is independently selected from ═O, F, Cl, C₁₋₄alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR²⁶R²⁷, and C₁₋₆alkyleneNR²⁶R²⁷. In an embodiment, each R²⁰ is independently selected from ═O, F, Cl, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₄alkyl, NR²⁶R²⁷, and C₁₋₄alkyleneNR²⁶R²⁷. In an embodiment, one of R²⁰ is ═O.

In an embodiment, each R²⁰ is independently selected from F, Cl, OH, C₁₋₄alkyl OC₁₋₄alkyl and NR²⁶R²⁷. In an embodiment, each R²⁰ is independently selected from F, Cl, OH, CH₃, CH₂CH₃, CF₂H, CF₃, CFH₂, CH₂CF₂H, OCH₃, OCH₂CH₃, OCF₃, OCF₂H, OCH(CH₃)₂ and NR²⁶R²⁷. In an embodiment, each R²⁰ is independently selected from F, Cl, OH, CH₃, CF₂H, CF₃, CFH₂, OCH₃, OCF₃, OCF₂H and NR²⁷R²⁸. In an embodiment, one to three of R²⁰ are independently selected from F, Cl, CH₃, CF₂H, CF₃, OCH₃, OCF₃, OCF₂H and NR²⁶R²⁷. In an embodiment, one to three of R²⁰ are independently selected from F, Cl, CH₃, CF₂H, CF₃, OCH₃, OCF₃ and OCF₂H. In an embodiment, one to four of R²⁰ are independently selected from F, CH₃, and OCH₃.

In an embodiment, each R²⁰ is independently selected from F, C and C₁₋₄alkyl. In an embodiment, each R²⁰ is independently selected from F, Cl, CH₃, CH₂CH₃, CF₂H, CF₃, CFH₂, CH₂CF₂H, and CH₂CH₂F. In an embodiment, each R²⁰ is independently selected from F, Cl, CH₃, CF₂H, CF₃ and CH₂CF₂H. In an embodiment, each R²⁰ is independently selected from F, Cl, CH₃, and CF₃. In an embodiment, each R²⁰ is independently selected from F, CH₃, and CF₃. In an embodiment, each R²⁰ is independently selected from F, CH₃, and CF₃. In an embodiment, each R²⁰ is independently selected from F and CH₃. In an embodiment, one or two of R²⁰ is F. In an embodiment, one or more, one to four, one to three, one or two or one of R²⁰ is CH₃.

In an embodiment, one or two R²⁰ are independently selected from C₃₋₆cycloalkyl, C₃₋₆heterocycloalkyl, C₁₋₄alkyleneC₃₋₆cycloalkyl and C₁₋₄alkyleneC₃₋₆heterocycloalkyl. In an embodiment, one of or two of R⁴ are independently selected from C₃₋₆cycloalkyl, C₃₋₆heterocycloalkyl, C₁₋₂alkyleneC₃₋₆cycloalkyl and C₁₋₂alkyleneC₃₋₆heterocycloalkyl. In an embodiment, the cycloalkyl in R²⁰ is selected from cyclopropyl, cyclobutyl, cyclopentyl and cyclohexyl. In an embodiment, the cycloalkyl in R²⁰ is selected from cyclopropyl and cyclobutyl.

In an embodiment, the heterocycloalkyl in R²⁰ is selected from aziridinyl, oxiranyl, thiiranyl, oxaxiridinyl, dioxiranyl, azetidinyl, oxetanyl, thietanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g, piperazinyl), morpholinyl, thiomorpholinyl, dioxanyl and dithianyl, In an embodiment, the heterocycloalkyl in R²⁰ is selected from azetidinyl, oxetanyl, thietanyl, tetrahydrofuranyl, pyrrolidinyl, imidazolidinyl and pyrazolidinyl.

In an embodiment, each R²⁰ is independently selected from OH and OC₁₋₄alkyl In an embodiment, each R²⁰ is independently selected from OH and OC₁₋₄alkyl. In an embodiment, one or two R²⁰ are selected from OH, OCH₃, OCF₃, OCF₂H, OCH₂CH₃ and OCH(CH₃)₂. In an embodiment, one R²⁰ is selected from OH, OCH₃, OCF₃, and OCF₂H.

In an embodiment, one of R²⁰ is NR²⁶R²⁷ or C₁₋₄alkyleneNR²⁶R²⁷. In an embodiment, one of R²⁰ is NR²⁶R²⁷ or C₁₋₂alkyleneNR²⁷R²⁸. In an embodiment, one of R²⁰ is NR²⁶R²⁷. In an embodiment, one R²⁰ is C₁₋₂alkyleneNR²⁶R²⁷. In an embodiment, the R²⁷ and R²⁸ in the NR²⁶R²⁷ or C₁₋₂alkyleneNR²⁶R²⁷ of R²⁰ are both CH₃ or both H. In an embodiment, one R²⁰ is NR²⁷R²⁸ and R²⁷ and R²⁸ in R²⁰ are both H.

In an embodiment, R²⁶ and R²⁷ are independently selected from H and C₁₋₄alkyl. In an embodiment, R²⁶ and R²⁷ are each independently selected from H, CH₃ and CF₃. In an embodiment, one of R²⁶ and R²⁷ is H and the other is CH₃. In an embodiment, R²⁶ and R²⁷ are both CH₃.

In an embodiment, R²⁶ and R²⁷ are joined to form, together with the atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R²⁶ and R²⁷ are taken together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl, optionally substituted with one or more of halo and C₁₋₆alkyl. In an embodiment, R²⁶ and R²⁷ are joined to form, together with the atom therebetween, a 4- to 6-membered heterocyclic ring, optionally substituted with one or more of halo and C₁₋₆alkyl. In an embodiment R²⁶ and R²⁷ are joined to form, together with the atom therebetween, aziridinyl, azetidinyl, pyrrolidinyl or piperidinyl, optionally substituted with one or more of halo and C₁₋₆alkyl.

In an embodiment, Q′ is C₁₋₃alkylene and is unsubstituted. In an embodiment, Q′ is C₁₋₃alkylene and is substituted with one or two of R²⁰. In an embodiment, Q′ is C₁₋₃alkylene and is substituted with one or two of R²⁰, and one R²⁰ is C₁₋₄alkyl. In an embodiment, Q′ is C₁₋₃alkylene and is substituted with one or two of R²⁰, and one or R²⁰ is selected from F, CH₃ and CF₃. In an embodiment, Q′ is C₁₋₃alkylene and is substituted with one or two of R²⁰, and one of R²⁰ is CH₃. In an embodiment, Q′ is C₁₋₃alkylene and is substituted with one to four of R²⁰, and each R²⁰ is independently selected from F, Cl, CH₃, CF₂H, CF₃, OCH₃, OCF₃ and OCF₂H. In an embodiment, Q′ is C₁₋₃alkylene and is substituted with one to four of R²⁰, and each R²⁰ is independently selected from F, CH₃ and OCH₃.

In an embodiment, Q′ is C₁₋₃alkylene and optionally disubstituted on one carbon atom with R²¹ and R^{21a}. In an embodiment, Q′ is C₁alkylene or C₂alkylene, and optionally disubstituted on one carbon atom with R²¹ and R^{21a}. In an embodiment, Q′ is CR²¹R^{21a}.

In an embodiment, R²¹ and R^{21a} are joined to form, together with the carbon atom therebetween, a 3- to 6-membered saturated or unsaturated ring optionally containing one heteromoiety selected from N, NH, NC₁₋₆alkyl, O, S, S(O), and SO₂ optionally substituted with one or more of halo and C₁₋₄alkyl.

In an embodiment, R²¹ and R^{21a} are joined to form, together with the carbon atom therebetween, a 3- to 6-membered cycloalkyl ring, optionally substituted with one or more of halo and C₁₋₄alkyl. In an embodiment, R²¹ and R^{21a} are joined to form, together with the carbon atom therebetween, to form a 3- to 6-membered cycloalkyl selected from cyclopropyl, cyclobutyl, cyclopentyl and cyclohexyl ring optionally substituted with one or more of halo and C₁₋₄alkyl. In an embodiment, R²¹ and R^{21a} are joined to form, together with the carbon atom therebetween, a 3- to 5-membered cycloalkyl ring, optionally substituted with one or more of halo and C₁₋₄alkyl. In an embodiment, R²¹ and R^{21a} are joined to form, together with the carbon atom therebetween, a cyclopropyl, a cyclobutyl or a cyclopentyl ring optionally substituted with one or more of R²¹ and R^{21a} are joined to form, together with the carbon atom therebetween, a cyclopropyl or a cyclobutyl ring, optionally substituted with one or more of halo and C₁₋₄alkyl. In an embodiment, R²¹ and R^{21a} are joined to form, together with the carbon atom therebetween, a cyclopropyl ring, optionally substituted with one or more of halo and C₁₋₄alkyl. In an embodiment, R²¹ and R^{21a} are joined to form, together with the carbon atom therebetween, a cyclopropyl or a cyclobutyl ring. In an embodiment, R²¹ and R^{21a} are joined to form, together with the carbon atom therebetween, a cyclopropyl ring.

In an embodiment, R²¹ and R^{21a} are joined to form, together with the carbon atom therebetween, a 3- to 6-membered heterocycloalkyl ring optionally substituted with one or more of halo and C₁₋₄alkyl. In an embodiment, R²¹ and R^{21a} are joined to form, together with the carbon atom therebetween, a 3- to 6-membered heterocycloalkyl ring selected from aziridinyl, oxiranyl, thiiranyl, oxaxiridinyl, dioxiranyl, azetidinyl, oxetanyl, thietanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g, piperazinyl), morpholinyl, thiomorpholinyl, dioxanyl and dithianyl. In an embodiment, R²¹ and R^{21a} are joined to form, together with the carbon atom therebetween, a 3- to 5-membered heterocycloalkyl ring optionally substituted with one or more of halo and C₁₋₄alkyl. In an embodiment, R²¹ and R^{21a} are joined to form, together with the carbon atom therebetween, a 3- to 5-membered heterocycloalkyl ring selected from oxiranyl, oxetanyl, azetidinyl, thietanyl, pyrrolidinyl, tetrahydrofuranyl, and tetrahydrothiophenyl optionally substituted with one or more of halo and C₁₋₄alkyl. In an embodiment, R²¹ and R^{21a} are joined to form, together with the carbon atom therebetween, oxetanyl, or azetidinyl optionally substituted with one or more of halo and C₁₋₄alkyl. In an embodiment, R²¹ and R^{21a} are joined to form, together with the carbon atom therebetween, oxetanyl. In an embodiment, R²¹ and R^{21a} are joined to form, together with the carbon atom therebetween to form

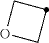

wherein “●” indicates a point of attachment to Q.

In an embodiment, Q′ is C₂₋₄alkenylene optionally substituted with one or two of R²³. In an embodiment, Q′ is C═C optionally substituted with one or two of R²³.

In an embodiment, Q′ is selected from C═N or N═C and is optionally substituted with R²². In an embodiment, Q′ is C═N or N═C.

In an embodiment, each R²² is independently selected from F, Cl, C₁₋₄alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR²⁶R²⁷, and C₁₋₆alkyleneNR²⁶R²⁷. In an embodiment, each R²² is independently selected from F, Cl, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₄alkyl, NR²⁶R²⁷, and C₁₋₄alkyleneNR²⁶R²⁷.

In an embodiment, each R²² is independently selected from F, Cl, OH, C. 4alkyl, OC₁₋₄alkyl, C₁₋₂alkyleneNR²⁶R²⁷ and NR²⁶R²⁷. In an embodiment, each R²² is independently selected from F, Cl, OH, CH₃, CH₂CH₃, CF₂H, CF₃, CFH₂, CH₂CF₂H, OCH₃, OCH₂CH₃, OCF₃, OCF₂H, OCH(CH₃)₂, C₁₋₂alkyleneNR²⁶R²⁷ and NR²⁶R²⁷. In an embodiment, each R²² is independently selected from F, Cl, OH, CH₃, CF₂H, CF₃, CFH₂, OCH₃, OCF₃, OCF₂H, C₁₋₂alkyleneNR²⁶R²⁷ and NR²⁶R²⁷. In an embodiment, one or two of R²² are independently selected from F, Cl, CH₃, CF₂H, CF₃, OCH₃, OCF₃, OCF₂H, C₁₋₂ alkyleneNR²⁶R²⁷ and NR²⁶R²⁷. In an embodiment, one or two of R²² are independently selected from F, Cl, CH₃, CF₂H, CF₃, OCH₃, OCF₃, OCF₂H, C₁₋₂alkyleneNR²⁶R²⁷ and NR²⁶R²⁷.

In an embodiment, each R²² is independently selected from F and C₁₋₄alkyl. In an embodiment, each R²² is independently selected from F, Cl, CH₃, CH₂CH₃, CF₂H, CF₃, CFH₂, CH₂CF₂H, and CH₂CH₂F. In an embodiment, each R²² is independently selected from F, Cl, CH₃, CF₂H, CF₃ and CH₂CF₂H In an embodiment, each R²² is independently selected from F, Cl, CH₃, and CF₃. In an embodiment, each R²² is independently selected from F, CH₃, and CF₃. In an embodiment, each R²² is independently selected from F and CH₃. In an embodiment, one of R²² is F. In an embodiment, one or more, one to four, one to three, one to two, or one of R²² is CH₃. In an embodiment, one of two R²² is CH₃.

In an embodiment, one or two of R²² is independently selected from C₃₋₆cycloalkyl, C₃₋₆heterocycloalkyl, C₁₋₄alkyleneC₃₋₆cycloalkyl and C₁₋₄alkyleneC₃₋₆heterocycloalkyl. In an embodiment, one R²² is selected from C₃₋₆cycloalkyl, C₃₋₆heterocycloalkyl, C₁₋₂alkyleneC₃₋₆cycloalkyl and C₁₋₂alkyleneC₃₋₆heterocycloalkyl. In an embodiment, the cycloalkyl in R²² is selected from cyclopropyl, cyclobutyl, cyclopentyl and cyclohexyl. In an embodiment, the cycloalkyl in R²² is selected from cyclopropyl and cyclobutyl.

In an embodiment, the heterocycloalkyl R²² is selected from aziridinyl, oxiranyl, thiiranyl, oxaxiridinyl, dioxiranyl, azetidinyl, oxetanyl, thietanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g, piperazinyl), morpholinyl, thiomorpholinyl, dioxanyl and dithianyl. In an embodiment, the heterocycloalkyl in R²³ is selected from azetidinyl, oxetanyl, thietanyl, tetrahydrofuranyl, pyrrolidinyl, imidazolidinyl and pyrazolidinyl.

In an embodiment, each R²² is independently selected from OH and OC₁₋₄alkyl In an embodiment, each R²² is independently selected from OH and OC₁₋₄alkyl. In an embodiment, each R²² is independently selected from OH, OCH₃, OCF₃, OCF₂H, OCH₂CH₃ and OCH(CH₃)₂. In an embodiment, one R²² is selected from OH, OCH₃, OCF₃, and OCF₂H.

In an embodiment, one or two of R²² is selected from NR²⁶R²⁷ and C₁₋₄alkyleneNR²⁶R²⁷. In an embodiment, one or two of R²² are C₁₋₄alkyleneNR²⁶R²⁷. In an embodiment, one of R²² is C₁₋₂alkyleneNR²⁶R²⁷. In an embodiment, one of R²² is NR²⁶R²⁷. In an embodiment, R²⁶ and R²⁷ in the NR²⁶R²⁷ or C₁₋₂alkylene NR²⁶R²⁷ of R²² are both CH₃ or both H. In an embodiment, one R²² is NR²⁷R²⁸ or C₁₋₄alkyleneNR²⁶R²⁷ and R²⁶ and R²⁷ in R²² are both H.

In an embodiment, R²⁶ and R²⁷ in R²² are each independently selected from H and C₁₋₄alkyl. In an embodiment, R²⁶ and R²⁷ in R²² are each independently selected from H, CH₃, and CF₃. In an embodiment, one of R²⁶ and R²⁷ in R²² is H and the other is CH₃. In an embodiment, R²⁶ and R²⁷ in R²² are both CH₃.

In an embodiment, R²⁶ and R²⁷ in R²² are joined to form, together with the atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R²⁶ and R²⁷ in R²² are taken together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl, optionally substituted with one or more of halo and C₁₋₆alkyl. In an embodiment, R²⁶ and R²⁷ in R²² are joined to form, together with the atom therebetween, a 4- to 6-membered heterocyclic ring, optionally substituted with one or more of halo and C₁₋₆alkyl In an embodiment R²⁶ and R²⁷ in R²² are joined to form, together with the atom therebetween, aziridinyl, azetidinyl, pyrrolidinyl, or piperidinyl, optionally substituted with one or more of halo and C₁₋₆alkyl.

In an embodiment, Q is C₁₋₃alkylene optionally interrupted by NR¹⁹. In an embodiment, R¹⁹ is selected from H and C₁₋₄alkyl. In an embodiment, R¹⁹ is selected from H, CH₃, CH₂CH₃, CF₂H, CF₃, CFH₂, CH₂CF₂H and CH₂CF₃. In an embodiment, R¹⁹ is selected from H, CH₃, CH₂CH₃ and CH(CH₃)₂.

In an embodiment, Q is C₁alkylene or C₂alkylene, and disubstituted on one carbon atom with R²¹ and R^{21a}, and R²¹ and R^{21a} are joined to form, together with the carbon atom therebetween, a 3- to 5-membered cycloalkyl ring, optionally substituted with one or more of halo and C₁₋₄alkyl. In an embodiment, Q is C₁alkylene or C₂alkylene, and disubstituted on one carbon atom with R²¹ and R^{21a}. In an embodiment, Q is CR²¹R^{21a}. In an embodiment, R²¹ and R^{21a} are joined to form, together with the carbon atom therebetween, a cyclopropyl or a cyclobutyl ring, and optionally substituted with one or more of halo and C₁₋₄alkyl.

In an embodiment, Q is C₂₋₄alkenylene optionally substituted with one or two of R²², and each R²² is independently selected from F, Cl, C₁₋₄alkyl and OC₁₋₄alkyl. In an embodiment, Q is C═C optionally substituted with one or two of R²², and each R²² is independently selected from F, Cl, C₁₋₄alkyl and OC₁₋₄alkyl. In an embodiment, Q is C═C optionally substituted with one or two of R²², and each R²² is independently selected from F, Cl, CH₃ and OCH₃ OCF₃ and OCF₂H. In an embodiment, Q is C═C optionally substituted with one or two of R²², and each R²² is selected from F and C₁₋₄alkyl. In an embodiment, R²² is selected from F and CH₃. In an embodiment, Q is C═C substituted with F. In an embodiment, Q is C═C substituted with Cl. In an embodiment, Q is C═C substituted with OCH₃. In an embodiment, Q is C═C substituted with CH₃. In an embodiment, Q is C═C substituted with F and CH₃.

In an embodiment, Q is selected from C═N and N═C and is optionally substituted with one or two of R²², and each R²² is independently selected from F, Cl, C₁₋₄alkyl, OC₁₋₄alkyl, NR²⁶R²⁷, cyclopropyl and cyclobutyl. In an embodiment, Q is selected from C═N and N═C and is optionally substituted with one or two of R²², and each R²² is independently selected from F, Cl, CH₃, OCH₃, OCF₃ OCF₂H, NR²⁶R²⁷, cyclopropyl and cyclobutyl. In an embodiment, Q is selected from C═N and N═C and is optionally substituted with one or two R²² and each R²² is independently selected from F, Cl, CH₃, OCH₃, OCF₃ and OCF₂H. In an embodiment, Q is selected from C═N and N═C and is optionally substituted with R²², and R²² is selected from F and CH₃. In an embodiment, Q is selected from C═N or N═C and is optionally substituted with R²², and R²² is selected from cyclopropyl and cyclobutyl. In an embodiment, Q is selected from C═N or N═C and is optionally substituted with R²², and R²² is selected from F and C₁₋₄alkyl. In an embodiment, Q is selected from C═N or N═C and is optionally substituted with one or two R²², and one R²² is selected from C₁₋₂alkyleneNR²⁶R²⁷ and NR²⁶R²⁷.

In an embodiment, Q is unsubstituted. In an embodiment, Q is substituted with one or two of R²⁰. In an embodiment, Q is substituted with one or two of R²⁰, and R²⁰ is C₁₋₄alkyl.

In an embodiment, Cy³ is C₆₋₁₀aryl which substituted with one or two of R²⁸, and optionally further substituted with one to three of R²⁹. In an embodiment, Cy³ is phenyl which substituted with one or two of R²⁸, and optionally further substituted with one to three of R²⁹. In an embodiment, Cy³ is phenyl which substituted with one R²⁸, and optionally further substituted with one to three of R²⁹.

In an embodiment, Cy³ is C₅₋₁₀heteroaryl which substituted with one or two of R²⁸, and optionally further substituted with one to three of R²⁹. In an embodiment, the C₅₋₁₀heteroaryl in Cy³ is selected from pyrrolyl, imidazolyl, oxazolyl, pyrazolyl or pyridinyl which substituted with one or two of R²⁸, and optionally further substituted with one to three of R²⁹. In an embodiment, the C₅₋₁₀heteroaryl in Cy³ is pyridine or pyrazolyl which substituted with one or two of R²⁸, and optionally further substituted with one to three of R²⁹.

In an embodiment, Cy³ is substituted with one R²⁸, and optionally further substituted with one to three of R²⁹. In an embodiment, Cy³ is substituted with one R²⁸ and optionally further substituted with one or two of R²⁹.

In an embodiment, each R²⁸ is independently selected from NR³⁰R³¹, C₁₋₄alkyleneNR³⁰R³¹, C₃₋₇heterocycloalkyl and C₁₋₄alkyleneC₃₋₇heterocycloalkyl, the latter two groups being optionally substituted with one or more of R³².

In an embodiment, one R²⁸ is NR³⁰R³¹, and R³⁰ and R³¹ are each independently selected from H and C₁₋₄alkyl. In an embodiment, R²⁸ is NR³⁰R³¹, and R³⁰ and R³¹, and R³⁰ and R³¹ are each independently selected from H, CH₃, CH₂CH₃, CF₂H, CF₃, CFH₂, CH₂CF₂H, CH₂CF₃. In an embodiment, one R²⁸ is NR³⁰R³¹, and R³⁰ and R³¹ are each independently selected from independently selected from H, CH₃, CH₂CH₃, CF₂H, CF₃, CFH₂, CH₂CF₂H and CH₂CF₃. In an embodiment, one R²⁸ is NR³⁰R³¹, and R³⁰ and R³¹ are independently selected from H, CF₃, CH₃ and CH₂CH₃. Accordingly, in an embodiment, one of R²⁸ is selected from NH₂, N(CH₃)₂, NH(CH₃), N(CH₃)(CH₂CH₂), NH(CH₂CH₂) and N(CH₂CH₂). In an embodiment, one R²⁸ is selected from NH₂, N(CH₃)₂ and NH(CH₃).

In an embodiment, one of R²⁸ is C₁₋₄alkyleneNR³⁰R³¹, and R³⁰ and R³¹ are each independently selected from H and C₁₋₄alkyl. In an embodiment, one of R²⁸ is C₁₋₄alkyleneNR³⁰R³¹, and R³⁰ and R³¹ are each independently selected from H, CH₃, CH₂CH₃, CF₂H, CF₃, CFH₂, CH₂CF₂H, CH₂CF₃. In an embodiment, one of R²⁸ is C₁₋₄alkyleneNR³⁰R³¹ and R³⁰ and R³¹ are each independently selected from H, CH₃, CH₂CH₃, CF₂H, CF₃, CFH₂, CH₂CF₂H and CH₂CF₃. In an embodiment, In an embodiment, one of R²⁸ is C₁₋₄alkyleneNR³⁰R³¹ and R³⁰ and R³¹ are each independently selected from H, CF₃, CH₃ and CH₂CH₃. Accordingly, in an embodiment, one of R²⁸ is selected from CH₂N(CH₂CH₃)₂, C(CH₃)₂NH₂, CH₂N(CH₃)₂, CH₂CH₂N(CH₃)₂ and CH₂N(CH₃)₂ and CH₂N(CH₃)₂. In an embodiment, one of R²⁸ is CH₂N(CH₃)₂.

In an embodiment, one of R²⁸ is C₁₋₄alkyleneNR³⁰R³¹ and R³⁰ and R³¹ are each independently selected from H and CH₃. In an embodiment, one of R²⁸ is C₁₋₃alkylene₄alkyleneNR³⁰R³¹ and R³⁰ and R³¹ are each are both H or are both CH₃.

In an embodiment, R³⁰ and R³¹ are each independently selected from H and C₁₋₄alkyl. In an embodiment, R³⁰ and R³¹ are independently selected from H, CH₃, CH₂CH₃, CF₂H, CF₃, CFH₂, CH₂CF₂H and CH₂CF₃. In an embodiment, R³⁰ and R³¹ are independently selected from H, CF₃ and CH₃. In an embodiment, R³⁰ and R³¹ are independently selected from H and CH₃.

In an embodiment, one R²⁸ is selected from C₃₋₇heterocycloalkyl and C₁₋₄alkyleneC₃₋₇heterocycloalkyl, optionally substituted with one to three of R³². In an embodiment, the C₃₋₇heterocycloalkyl in the C₃₋₇heterocycloalkyl and C₁₋₄alkyleneC₃₋₇heterocycloalkyl of R²⁸ is selected from azetidinyl, oxetanyl, tetrahydrofuranyl, tetrahydrothiophenyl, pyrrolidinyl, pyrrolidin-2-onyl, azabicyclohexanyl, azabicycloheptanyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, dioxanyl, azepanyl, diazepanyl, oxepanyl, thiepanyl, azabicyclohexanyl, azabicycloheptanyl, oxabicyclohexanyl, and oxabicycloheptanyl optionally substituted with one to three of R³². In an embodiment, the C₃₋₇heterocycloalkyl in the C₃₋₇heterocycloalkyl and C₁₋₄alkyleneC₃₋₇heterocycloalkyl of R²⁸ is diazepanyl. In an embodiment, one R²⁸ is selected from C₄₋₆ heterocycloalkyl and C₁₋₄alkyleneC₄₋₆heterocycloalkyl, and the C₄₋₆heterocycloalkyl in the C₄₋₆heterocycloalkyl and C₁₋₄alkyleneC₄₋₆heterocycloalkyl of R²⁸ is selected from azetidinyl, oxetanyl, tetrahydrofuranyl, tetrahydrothiophenyl, pyrrolidinyl, pyrrolidin-2-onyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, and dioxanyl, optionally substituted with one to three of R¹⁰. In an embodiment, the C₄₋₆heterocycloalkyl in the C₄₋₆heterocycloalkyl and C₁₋₄alkyleneC₄₋₆heterocycloalkyl of R²⁸ is selected from tetrahydrofuranyl, tetrahydrothiophenyl, pyrrolidinyl, imidazolidinyl, pyrazolidinyl, pyrrolidin-2-onyl, thiazolidinyl and isothiazolidinyl, optionally substituted with one to three of R³². In an embodiment, the C₄₋₆heterocycloalkyl in the C₄₋₆heterocycloalkyl and C₁₋₄alkyleneC₄₋₆ heterocycloalkyl of R²⁸ is selected from pyrrolidinyl, imidazolidinyl, pyrazolidinyl, thiazolidinyl, pyrrolidin-2-onyl, and isothiazolidinyl, optionally substituted with one to three of R¹⁰.

In an embodiment, the C₃₋₇heterocycloalkyl in the C₃₋₇heterocycloalkyl and C₁₋₄alkyleneC₃₋₇heterocycloalkyl of R²⁸ comprises at least one N atom. In an embodiment, the C₃₋₇heterocycloalkyl in the C₃₋₇heterocycloalkyl and C₁₋₄alkyleneC₃₋₇heterocycloalkyl of R²⁸ is selected from azetidinyl, pyrrolidinyl, pyrrolidin-2-onyl, azabicyclohexanyl, azabicycloheptanyl, piperidinyl, piperazinyl and morpholinyl each of which optionally substituted with one or two of R³². In an embodiment, the C₄₋₆heterocycloalkyl in the C₄₋₆heterocycloalkyl and C₁₋₄alkyleneC₄₋₆heterocycloalkyl of R²⁸ is pyrrolidinyl, optionally substituted with one or two of R³².

In an embodiment, one R²⁸ is C₄₋₆heterocycloalkyl, optionally substituted with one or two of R³², and the C₄₋₆heterocycloalkyl is pyrrolidinyl. In an embodiment, one R²⁸ is C₄₋₆heterocycloalkyl, and the C₄₋₆heterocycloalkyl is pyrrolidine selected from

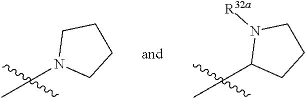

optionally substituted with one or two of R³², wherein R^{32a} is selected from H and R³², and

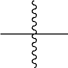

indicates a point of covalent attachment to Cy¹. In an embodiment, the pyrrolidinyl is selected from

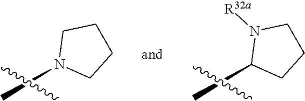

and optionally substituted with one or two of R³², wherein R^{32a} is selected from H and R³², and

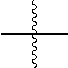

indicates a point of covalent attachment to Cy¹. In an embodiment, R^{32a} is H. In an embodiment, R^{32a} is R³².

In an embodiment, one of R²⁸ is C₁₋₆alkyleneC₃₋₇heterocycloalkyl, optionally substituted with one to four of R³², and the C₃₋₇heterocycloalkyl in the C₁₋₆alkyleneC₃₋₇heterocycloalkyl is selected from azetidinyl, pyrrolidinyl, pyrrolidin-2-onyl, piperidinyl, piperazinyl and morpholinyl. In an embodiment, one of R²⁸ is C₁₋₃alkyleneC₄₋₇heterocycloalkyl, optionally substituted with one to four of R³², and the C₄₋₇heterocycloalkyl in the C₁₋₆alkyleneC₃₋₇heterocycloalkyl is selected from azetidinyl, pyrrolidinyl, piperidinyl, piperazinyl and morpholinyl. Accordingly, in an embodiment, one of R²⁸ is selected from C₁₋₃alkyleneazetidinyl, C₁₋₃alkylenepyrrolidinyl, C₁₋₃alkylenepiperidinyl, C₁₋₃alkylenepiperazinyl and C₁₋₃alkylenemorpholinyl optionally substituted with one to four of R³². In an embodiment, one of R²⁸ is selected from CH₂azetidinyl, CH₂pyrrolidinyl, CH₂piperidinyl, CH₂piperazinyl and CH₂morpholinyl optionally substituted with one to four of R³². In an embodiment, one of R²⁸ is selected from

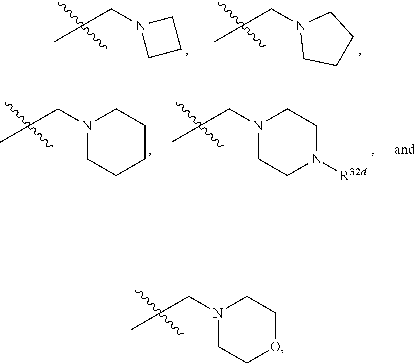

optionally substituted with one or two of R³², wherein R^{32d} is selected from H and R³², and

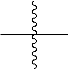

indicates a point of covalent attachment to Cy¹.

In an embodiment, one R²⁸ is C₁₋₆alkyleneC₃₋₇heterocycloalkyl, optionally substituted with one or two of R³², and the C₃₋₇heterocycloalkyl in the C₁₋₆alkyleneC₃₋₇heterocycloalkyl is pyrrolidinyl. In an embodiment, one R²⁸ is C₁₋₄alkylenepyrrolidinyl optionally substituted with one to three of R³². In an embodiment, one R²⁸ is C₁₋₄alkyleneC₄₋₆heterocycloalkyl, and the C₄₋₆heterocycloalkyl in the C₁₋₄alkyleneC₄₋₆ heterocycloalkyl is pyrrolidinyl optionally substituted with one to three of R³².

In an embodiment, one R²⁸ is C₁₋₄alkylenepyrrolidinyl, optionally substituted with one to three of R³², wherein at least one R³² is F. In an embodiment, the C₄₋₆heterocycloalkyl in R²⁸ is

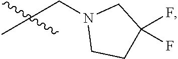 [SMILES: *CN1CCC(F)(F)C1]

wherein

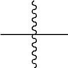

indicates a point of covalent attachment to Cy¹.

In an embodiment, one R²⁸ is pyrrolidinyl which is unsubstituted. In an embodiment, one R²⁸ is pyrrolidinyl selected from and

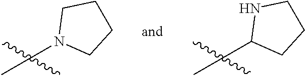 [SMILES: *N1CCCC1 ; *C1CCCN1]

wherein

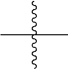

indicates a point of covalent attachment to Cy¹. In an embodiment, one R²⁸ is selected from

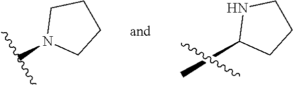

wherein

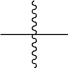

indicates a point of covalent attachment to Cy¹.

In an embodiment, one to three R²⁹ are independently selected from halo, C₁₋₄alkyl, C₃₋₇cycloalkyl, and C₁₋₄alkyleneC₃₋₇cycloalkyl, the latter four groups being optionally substituted with one to three of R³².

In an embodiment, one to three R²⁹ are independently selected from halo and C₁₋₄alkyl. In an embodiment, one to three R²⁹ are independently selected from F, Cl, CH₃, CH₂CH₃, CH(CH₃)₂, CH₂CH₂CH₃, CH₂CH₂CH₂CH₃, CF₂H, CF₃, CFH₂, CH₂CH₂F, CH₂CF₂H, CH₂CF₃ CH₂CH₂F₂H, CH₂CH₂CH₂F₂H and CH(CH₃)₂. In an embodiment, one to three R²⁹ are independently selected from F, Cl, CH₃, CH₂CH₃, CH(CH₃)₂, CF₂H, CF₃, CFH₂, CH₂CH₂F, CH₂CF₂H, CH₂CF₃, CH₂CH₂F₂H and CH₂CH₂CH₂F₂H. In an embodiment, one to three R²⁹ are independently selected from F, Cl, CH₃, CH₂CH₃, CF₂H, CF₃, CFH₂, CH₂CF₂H and CH₂CF₃. In an embodiment, one to three R²⁹ are independently selected from F, Cl, CH₃, CH₂CH₃ and CF₃. In an embodiment, one to three R²⁹ are independently F, CH₃ or CF₃. In an embodiment, one to three R²⁹ are independently CH₃ or CF₃. In an embodiment, one to three of R²⁹ are F. In an embodiment, one to three, one or two or one of R²⁹ is CF₃.

In an embodiment, one to three of R²⁹ are independently selected from C₃₋₇cycloalkyl and C₁₋₄alkyleneC₃₋₇cycloalkyl, optionally substituted with one to three of R³². In an embodiment, one R²⁹ is selected from C₁₋₃alkyleneC₃₋₇cycloalkyl and C₃₋₇cycloalkyl, optionally substituted with one or two of R²⁹. In an embodiment, the C₃₋₇cycloalkyl in the C₁₋₃alkyleneC₃₋₇cycloalkyl and C₃₋₇cycloalkyl of R²⁹ is selected from cyclopropyl, cyclobutyl, cyclopentyl, cyclohexyl, cycloheptyl, bicyclo[2.2.1]hexanyl and bicyclo[2.2.1]heptanyl, optionally substituted with one or two of R³².

In an embodiment, Cy³ is phenyl or pyridinyl which is substituted with one R²⁸ and optionally further substituted with one to three of R²⁹, and R²⁸ is C₁₋₄ alkyleneNR³⁰R³¹. In an embodiment, Cy³ is phenyl or pyridinyl which is substituted with one R²⁸ and optionally further substituted with one to three of R²⁹, and R²⁸ is C₁₋₂alkyleneNR³⁰R³¹. In an embodiment, Cy³ is phenyl or pyridinyl which is substituted with one R²⁸ and optionally further substituted with one to three of R²⁹; and R²⁸ is CH₂NR³OR³¹. In an embodiment, Cy³ is phenyl which is substituted with one R²⁸ and optionally further substituted with one to three of R²⁹ and R²⁸ is C₁₋₄alkyleneNR³⁰R³¹. In an embodiment, Cy³ is phenyl which is substituted with one R²⁸ and optionally further substituted with one to three of R²⁹ and R²⁸ is C₁₋₂alkyleneNR³⁰R³¹. In an embodiment, Cy³ is phenyl which is substituted with one R²⁸, and optionally further substituted with one to three of R²⁹; and R²⁸ is CH₂NR³OR³¹.

In an embodiment, Cy³ is phenyl which is substituted with one R²⁸ and optionally further substituted with one to three of R²⁹, and R²⁸ is C₃₋₇heterocycloalkyl or C₁₋₄alkyleneC₃₋₇heterocycloalkyl optionally substituted with one or more of R³². In an embodiment, Cy³ is phenyl which is substituted with one R²⁸ and optionally further substituted with one to three of R²⁹, and R²⁸ is C₃₋₇heterocycloalkyl or C₁₋₂alkyleneC₃₋₇heterocycloalkyl optionally substituted with one or more of R³².

In an embodiment, each R³² is independently selected from F, Cl, CN, C₁₋₄alkyl and NR³³R³⁴. In an embodiment, each R³² is independently selected from F, C₁₋₄alkyl and NR³³R³⁴. In an embodiment, each R³² is independently selected from F and C₁₋₄alkyl. In an embodiment, each R³² is independently selected from F and CH₃, CH₂CH₃, CF₂H, CF₃, CFH₂, CH₂CF₂H and CH₂CF₃. In an embodiment, at least one of R¹⁰ is F. In an embodiment, one of R³² is NR³³R³⁴.

In an embodiment, R³³ and R³⁴ are independently selected from H and C₁₋₄alkyl. In an embodiment, R³³ and R³⁴ are independently selected from H, CH₃, CH₂CH₃, CF₂H, CF₃, CFH₂, CH₂CF₂H and CH₂CF₃. In an embodiment, R³³ and R³⁴ are independently selected from H, CF₃ and CH₃. In an embodiment, R³³ and R³⁴ are independently selected from H and CH₃.

In an embodiment, Cy⁴ is a monocyclic C₃₋₇heterocycloalkyl which is unsubstituted or substituted with one or more of R³⁵. In an embodiment, Cy⁴ is azetidinyl, oxetanyl, tetrahydrofuranyl, tetrahydrothiophenyl, pyrrolidinyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, dihydropyranyl tetrahydropyranyl, thiomorpholinyl, morpholinyl, dioxanyl, azepanyl, diazepanyl, oxepanyl or thiepanyl, which is substituted with one to three of R¹². In an embodiment, Cy⁴ is diaxepanyl, 5,6-dihydro-1,2,4-triazinyl, 3,4,5,6-tetrahydro-1,2,4-triazinyl, tetrahydrofuranyl, tetrahydropyranyl, pyrrolidinyl, piperidinyl, piperazinyl or morpholinyl, which is substituted with one to three of R³⁵. In an embodiment, Cy⁴ is tetrahydrofuranyl, dihydropyranyl, tetrahydropyranyl, pyrrolidinyl, piperidinyl, piperazinyl or morpholinyl, which is substituted with one to three of R³⁵. In an embodiment, Cy⁴ is pyrrolidinyl, piperidinyl, piperazinyl or morpholinyl, which is substituted with one to three of R³⁵. In an embodiment, Cy⁴ is selected from piperidinyl, piperazinyl, and morpholinyl, and Cy⁴ is unsubstituted or substituted with one or two of R³⁵. In an embodiment, Cy⁴ is piperazinyl and Cy⁴ is unsubstituted or substituted with one or two of R³⁵. In an embodiment, Cy⁴ is selected from tetrahydrofuranyl, dihydropyranyl, morpholinyl, and tetrahydropyranyl, which is substituted with one to three of R³⁵. In an embodiment, R³⁵ is tetrahydropyranyl, which is substituted with one to three of R³⁵.

In an embodiment, Cy⁴ is an unsubstituted monocyclic C₃₋₇heterocycloalkyl.

In an embodiment, Cy⁴ is a bicyclic heterocycle which is unsubstituted or substituted with one or more of R³⁵. In an embodiment, Cy⁴ is a bridged bicyclic heterocycle, fused bicyclic heterocycle or a spirofused bicyclic heterocycle which is substituted with one to three of R³⁵. In an embodiment, Cy⁴ is a fused bicyclic heterocycle which is unsubstituted or substituted with one to three of R³⁵. In an embodiment, Cy⁴ is a C₆-C₁₀ saturated bicyclic ring in which one or two of the ring carbon atoms is replaced with N, NH or NR^{35a}, depending on the valency requirements of the N which is substituted with one to three of R³⁵. In an embodiment, Cy⁴ is bridged azabicyclohexanyl, bridged diazabicycloheptanyl or bridged diazabicyclooctanyl which is substituted with one to three of R³⁵. In an embodiment, Cy⁴ is selected from the following structures:

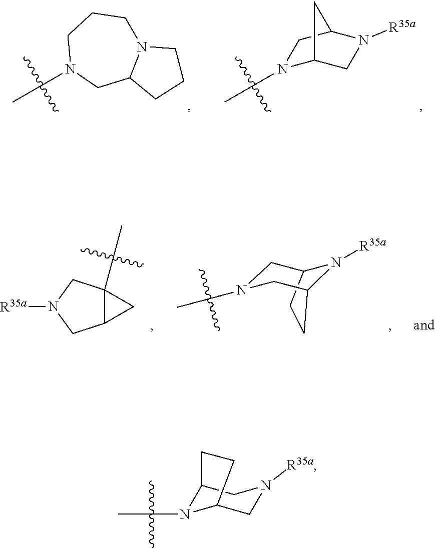

which is substituted with one to three of R³⁵, wherein

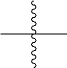

indicates a point of covalent attachment to Cy³, and R^{35a} is selected from with H or R³⁵.

In an embodiment, Cy⁴ is selected from the following structures

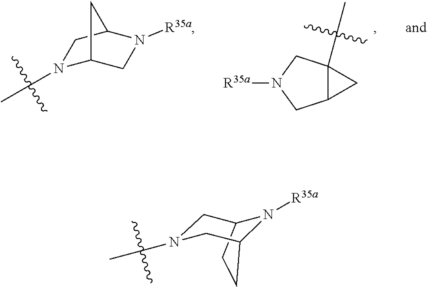

which is substituted with one to three of R³⁵, wherein

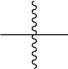

indicates a point of covalent attachment to Cy³, and R^{35a} is selected from with H or R³⁵.

In an embodiment, Cy⁴ is selected from tetrahydrofuropyrrolyl, hexapyrazinooxazinyl, hexahydropyrrolopyrazinyl and hexahydropyrrolodiazepiny and Cy⁴ is unsubstituted or substituted with one or more of R³⁵. In an embodiment, Cy⁴ is selected from

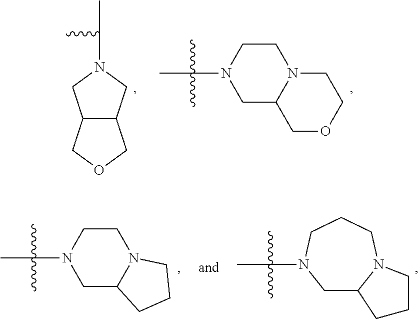

which unsubstituted or substituted with one or more of R³⁵; and
wherein

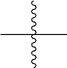

indicates a point of covalent attachment to Cy¹.

In an embodiment, Cy⁴ is an unsubstituted bicyclic heterocycle.

In an embodiment, each R³⁵ is independently selected from halo, ═O, OH, C₁₋₄alkyl, C₃₋₁₀cycloalkyl, C₃₋₁₀heterocycloalkyl, C₁₋₄alkyleneC₃₋₁₀cycloalkyl, C₁₋₄ alkyleneC₃₋₁₀heterocycloalkyl, C₁₋₄alkyleneOR³⁶, C₁₋₄alkyleneNR³⁶R³⁷, OC₁₋₄alkyleneOR³⁶, OC₁₋₄alkyleneNR³⁶R³⁷, C(O)R³⁶, C(O)C₁₋₄alkyleneOR³⁶, C(O)C₁₋₄alkyleneNR³⁶R³⁷, C(O)C₁₋₄alkyleneOC₁₋₄alkyleneNR³⁶R³⁷, C(O)NR³⁶R³⁷, CO₂R³⁶, CO₂C₁₋₄alkyleneOR³⁶, CO₂C₁₋₄alkyleneOC₁₋₄alkyleneNR³⁶R³⁷, NR³⁶R³⁷, NR³⁸SO₂R³⁷, SO₂R³⁶ and SO₂NR³⁶R³⁷.

In an embodiment, R³⁶ is selected from H, C₁₋₄alkyl, C₃₋₆cycloalkyl, C₁₋₄alkyleneC₃₋₆cycloalkyl, C₃₋₆heterocycloalkyl and C₁₋₄alkyleneC₃₋₆heterocycloalkyl In an embodiment, R³⁶ is selected from H, CH₃, CH₂CH₃, CH(CH₃)₂, CF₃, CFH₂, CH₂CF₂H, CH₂CF₃, C₁₋₄alkyleneC₃₋₆cycloalkyl and C₁₋₄alkyleneC₃₋₆heterocycloalkyl.

In an embodiment, R³⁷ is selected from H and C₁₋₄alkyl. In an embodiment, R³⁷ is selected from H, CH₃ and CH₂CH₃, In an embodiment, R³⁷ is selected from H, CH₃, CH₂CH₃, CF₂H, CF₃, CFH₂, CH₂CF₂H and CH₂CF₃. In an embodiment, R³⁷ is selected from CF₂H, CH₃ and CF₃. In an embodiment, R³⁷ is selected from H, CH₃, CH₂CH₃ and CF₃. In an embodiment, R³⁷ is selected from CH₃ and CF₃. In an embodiment, R³⁷ is H.

In an embodiment, R³⁶ and R³⁷ are joined to form, together with the atoms therebetween, a 4- to 6-membered saturated or unsaturated ring, optionally containing one additional heteromoiety selected from N, NR¹⁶, O, S, S(O) and SO₂. In an embodiment, R³⁶ and R³⁷ are joined to form, together the atoms therebetween to form 4- to 6-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl) and morpholinyl. In an embodiment, R³⁶ and R³⁷ are joined to form, together with the atom therebetween, aziridinyl, azetidinyl, pyrrolidinyl, or piperidinyl

In an embodiment, one R³⁵ is CO₂R³⁶. In an embodiment, R³⁵ is selected from H, C₁₋₄alkyl. Therefore, in an embodiment, one R³⁵ is CO₂R³⁶ and R³⁶ is selected from H, C₁₋₄alkyl. Accordingly, in an embodiment, one R³⁵ is CO₂C₁₋₆alkyl. In an embodiment, R¹³ is selected from H, CH₃, CH₂CH₃, CH(CH₃)₂, CF₃, CFH₂, CH₂CF₂H and CH₂CF₃. Accordingly, in an embodiment, one R³⁵ is selected from CO₂CCH₃, CO₂CH₂CH₃, CO₂CF₂H, CO₂CF₃, CO₂CFH₂, CO₂CH₂CF₂H, CO₂CH₂CF₃, CO₂CH₂CH₂F₂H, CO₂CH₂CH₂CH₂F₂H, CO₂CH(CH₃)₂, and CO₂CH₂CH(CH₃)₂, an embodiment, one R³⁵ is CO₂CCH₃.

In an embodiment, one R³⁵ is C(O)R³⁶. In an embodiment, R³⁵ is selected from H, C₁₋₄alkyl. Accordingly, in an embodiment, one R³⁵ is C(O)C₁₋₆alkyl. In an embodiment, R³⁵ is selected from H, CH₃, CH₂CH₃, CH(CH₃)₂, CF₃, CFH₂, CH₂CF₂H and CH₂CF₃. Accordingly In an embodiment, one R³⁵ is selected from COCCH₃, COCH₂CH₃, COCF₂H, COCF₃, COCFH₂, COCH₂CF₂H, COCH₂CF₃, COCH₂CH₂F₂H, COCH₂CH₂CH₂F₂H, COCH(CH₃)₂, and COCH₂CH(CH₃)₂. In an embodiment, one R³⁵ is COCH₃.

In an embodiment, each R³⁵ is independently selected from the substituents listed below:
OH, F, Cl, CF₃, CH₃, CH₂CH₃, CF₂H, CH₂CF₂H, CH₂CH₂CF₂H, CH₂CH₂CH₂F₂H

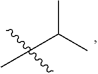 [SMILES: *C(C)C]

OC₁₋₄alkyl, SO₂C₁₋₄alkyl, C₁₋₄alkyleneOH, C₁₋₄alkyleneOCH₃, C₁₋₄alkyleneC₃₋₁₀cycloalkyl
C₁₋₄alkyleneC₃₋₁₀hetereocycloalkyl, C₃₋₁₀cycloalkyl, C₃₋₁₀heterocycloalkyl,

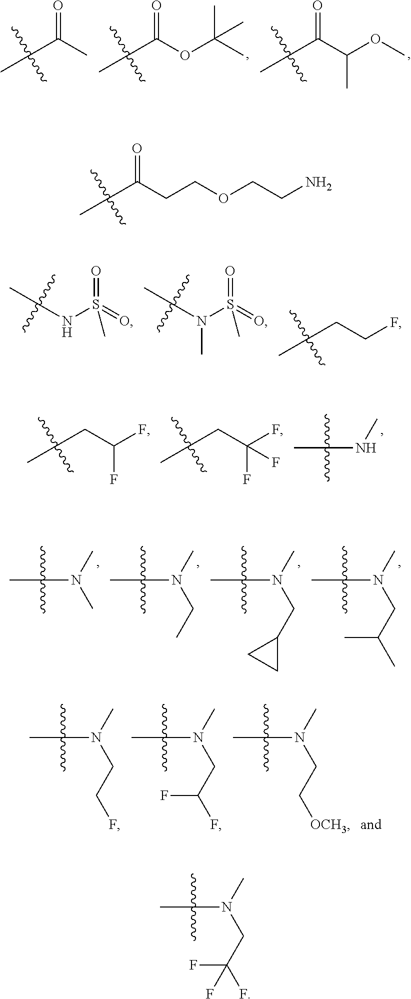

In an embodiment, one or two of R³⁵ are independently selected from C₃₋₆cycloalkyl, C₃₋₆heteroycloalkyl, C₁₋₄alkyleneC₃₋₆cycloalkyl and C₁₋₄alkyleneC₃₋₆heterocycloalkyl. In an embodiment, one R³⁵ is selected from C₃₋₅cycloalkyl, C₃₋₆heteroycloalkyl, C₁₋₄alkyleneC₃₋₅cycloalkyl and C₁₋₄alkyleneC₃₋₅heterocycloalkyl.

In an embodiment, the C₃₋₆cycloalkyl in R³⁵ is selected from cyclopropyl and cyclobutyl.

In an embodiment, one R³⁵ is selected from cyclopropyl, cyclobutyl and cyclopentyl. In an embodiment, one R³⁵ is independently selected from cyclopropyl and cyclobutyl.

In an embodiment, one R³⁵ is C₁₋₄alkyleneC₃₋₅cycloalkyl selected from C₁₋₄alkylenecyclopropyl, C₁₋₄alkylenecyclobutyl. In an embodiment, one R³⁵ is selected from C₁₋₄alkylenecyclopropyl and C₁₋₄alkyleneC₃cyclobutyl In an embodiment, one R³⁵ is C₁₋₃alkylenecyclopropyl. In an embodiment, one R¹² is C₁₋₃alkylenecyclopropyl selected from

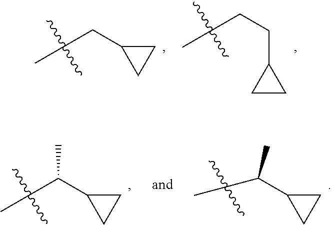

In an embodiment, one R³⁵ is selected from oxetanyl, tetrahydrofuranyl and tetrahydropyranyl. In an embodiment, one R³⁵ is selected from C₁₋₃alkyleneoxetanyl, C₁₋₃alkylenetetrahydrofuranyl and C₁₋₃alkylenetetrahydropyranyl.

In an embodiment, one R³⁵ is selected from CH₃, CH₂CH₃, CF₂H, CF₃, CFH₂, CH₂CF₂H, CH₂CH₂CF₂H, CH₂CH₂CH₂CF₂H and CH₂CF₃. In an embodiment, one R³⁵ is selected from CH₃, CH₂CH₃, CF₂H, CF₃, CFH₂, CH₂CF₂H, and CH₂CF₃. In an embodiment, one R³⁵ is selected from CH₃, CH₂CH₃, CH(CH₃)₂. In an embodiment, one R²¹ is selected from CH₂CH₃ and CH(CH₃)₂. In an embodiment, one R³⁵ is selected from CH₂CH₃ and CH(CH₃)₂. In an embodiment, one R³⁵ is selected CF₂H, CH₂CF₂H, CH₂CH₂CF₂H, and CH₂CH₂CH₂CF₂H. I.

In an embodiment, each R³⁵ is independently selected from the substituents listed below:
OH, F, Cl, CF₃, CH₃, CH₂CH₃, CF₂H, CH₂CF₂H, CH₂CH₂CF₂H, CH₂CH₂CH₂F₂H
OC₁₋₄alkyl, SO₂C₁₋₄alkyl, C₁₋₄alkyleneOH, C₁₋₄alkyleneOCH₃,

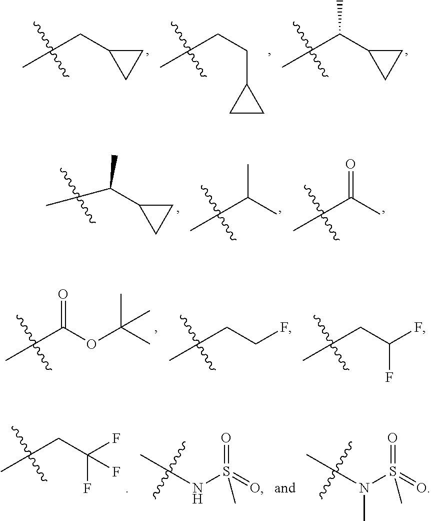

In an embodiment, Cy⁴ is substituted with one to three of R³⁵. In an embodiment, Cy⁴ is substituted with one or two of R³⁵. In an embodiment, Cy⁴ is substituted with one of R³⁵.

In an embodiment, Cy⁴ is pyrrolidinyl, piperidinyl, piperazinyl or morpholinyl, and R³⁵ is CH₃, CH₂CH₃, CF₂H, CF₃, CFH₂, CH₂CF₂H and CH₂CF₃. In an embodiment, Cy⁴ is pyrrolidinyl, piperidinyl, piperazinyl or morpholinyl, and R¹² is CH₂CH₃ or CH(CH₃)₂.

In an embodiment, Cy⁴ is pyrrolidinyl, piperidinyl, piperazinyl or morpholinyl, and R³⁵ is C₁₋₃alkylenecyclopropyl

In an embodiment, R³⁸ and R³⁹ are independently selected from H and C₁₋₄alkyl. In an embodiment, R³⁸ and R³⁹ are independently selected from H, CH₃, CH₂CH₃, and CH(CH₃)₂.

In an embodiment, the compound of Formula (II) is a compound of Formula (II-A), or a pharmaceutically acceptable salt, solvate and/or prodrug thereof,

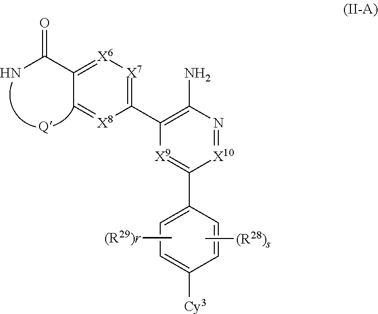

wherein Q′, X⁵, X⁶, X⁷, X⁸, X⁹, R²⁸, R²⁹ and Cy³ are as defined in Formula (II); and
r is an integer selected from 0 to 3; and
s is an integer selected from 1 and 2;
wherein all available hydrogen atoms are optionally substituted with a fluorine atom.

In an embodiment, the compound of Formula (II) is a compound of Formula (II-B), or a pharmaceutically acceptable salt, solvate and/or prodrug thereof,

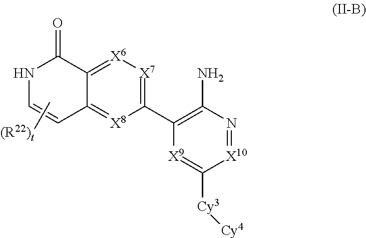

wherein X⁴, X⁵, X⁶, R^{4c}, R²², Cy³ and Cy⁴ are as defined in Formula (II); and
t is an integer selected from 0 to 2,
wherein all available hydrogen atoms are optionally substituted with a fluorine atom.

In an embodiment, the compound of Formula (II) is a compound of Formula (II-C) or (II-D), or a pharmaceutically acceptable salt, solvate and/or prodrug thereof,

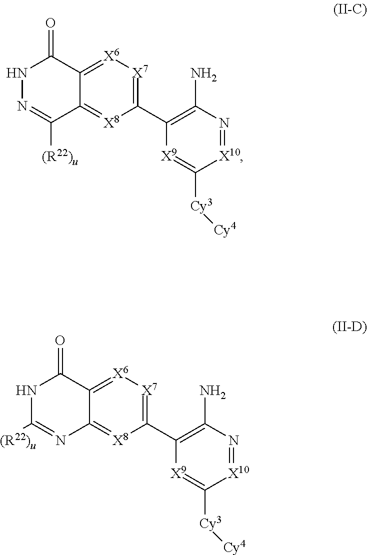

wherein X⁴, X⁵, X⁶, R²², Cy³ and Cy⁴ are as defined in Formula (II); and
u is an integer selected from 0 and 1,
wherein all available hydrogen atoms are optionally substituted with a fluorine atom.

In an embodiment, Cy⁴ is an unsubstituted monocyclic C₃₋₇heterocycloalkyl, and the compound of Formula (II) is a compound of Formula II-(E) or a pharmaceutically acceptable salt, solvate and/or prodrug thereof,

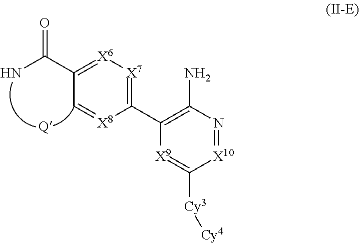

wherein X⁴, X⁵, X⁶, R²² and Cy³ are as defined in Formula (II); and
Cy⁴ is an unsubstituted monocyclic C₃₋₇heterocycloalkyl;
wherein all available hydrogen atoms are optionally substituted with a fluorine atom.

Accordingly, the present application further includes a Formula (II) a compound of Formula II-(E) or a pharmaceutically acceptable salt, solvate and/or prodrug thereof,

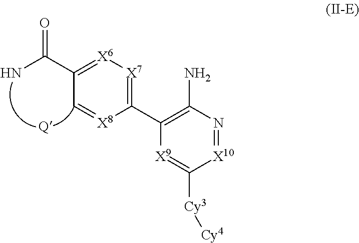

wherein
X⁴ is selected from N and CR¹⁷;
X⁵ and X⁶ are each independently selected from N and CR¹⁸;
Q′ is C₁₋₄alkylene optionally interrupted by a heteromoiety selected from O, S, S(O), SO₂ and NR¹⁹ and/or optionally substituted with one or more of R²⁰ and/or optionally disubstituted on one carbon with R²¹ and R^{21a}, provided that when Q comprises the heteromoiety the heteromoiety is separated from the ring amide NH by other than methylene; or
Q′ is C₂₋₄alkenylene optionally substituted with one or more of R²²; or
Q′ is C═N or N═C optionally substituted with R²²;
R¹⁷ is selected from H, halo, OR²³, NR²⁴R²⁵, C₁₋₆alkyleneNR²⁴R²⁵ and C₁₋₆alkyl;
R¹⁸ is selected from H, halo and C₁₋₆alkyl;
R¹⁹ is selected from H and C₁₋₆alkyl;
each R²⁰ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR²⁶R²⁷ and C₁₋₆alkyleneNR²⁶R²⁷;
R²¹ and R^{21a} are joined to form, together with the carbon atom therebetween, a 3- to 6-membered, saturated or unsaturated ring optionally containing one heteromoiety selected from N, NH, NC₁₋₆alkyl, O, S, S(O), and SO₂ and optionally substituted with one or more of halo and C₁₋₆alkyl;
each R²² is independently selected from halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR²⁶R²⁷ and C₁₋₆alkyleneNR²⁶R²⁷;
R²⁴, R²⁵, R²⁶ and R²⁷ are each independently selected from H and C₁₋₆alkyl, or
R²⁴ and R²⁵ or R²⁶ and R²⁷ are joined to form, together with the atom therebetween, a 3- to 7-membered saturated or unsaturated ring, optionally containing one additional heteromoiety selected from N, NH, NC₁₋₆alkyl, O, S, S(O), and SO₂ and optionally substituted with one or more of halo and C₁₋₆alkyl;
Cy³ is C₆₋₁₀aryl or C₅₋₁₀heteroaryl, which substituted with one or two of R²⁸, and optionally further substituted with one to three of R²⁹;
each R²⁸ is independently selected from NR³⁰R³¹, C₁₋₆alkyleneNR³⁰R³¹, C₃₋₇ heterocycloalkyl and C₁₋₆alkyleneC₃₋₇heterocycloalkyl, the latter two groups being optionally substituted with one or more of R³²;
each R²⁹ is independently selected from halo, C₁₋₆alkyl, C₃₋₇cycloalkyl, and C₁₋₆alkyleneC₃₋₇cycloalkyl, the latter two groups being optionally substituted with one or more of R³²;
R³⁰ and R³¹ are each independently selected from H and C₁₋₆alkyl;
each R³² is independently selected from halo, C₁₋₆alkyl, CN and NR³³R³⁴;
R³³ and R³⁴ are each independently selected from H and C₁₋₆alkyl;
Cy⁴ is an unsubstituted monocyclic C₃₋₇heterocycloalkyl; and
wherein all available hydrogen atoms are optionally substituted with a fluorine atom.

In an embodiment, the compound of Formula I is selected from the compounds listed in Table 1-A:

TABLE 1-A

| Compound | | |
|---|---|---|
| I.D | Compound Name | Structure |
| II-1 | 6-(3-amino-6-(2- ((dimethylamino)methyl)-4- (tetrahydro-2H-pyran-4- yl)phenyl)pyrazin-2-yl)-7- fluoro-3,4-dihydroisoquinolin- 1(2H)-one | 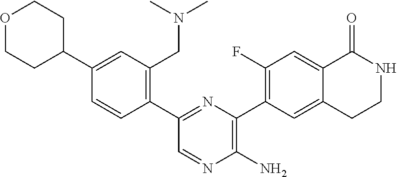 [SMILES: CN(C)Cc1cc(C2CCOCC2)ccc1-c1cnc(N)c(-c2cc3c(cc2F)C(=O)NCC3)n1] |
| II-2 | 6-(3-amino-6-(3- ((dimethylamino)methyl)-4- (tetrahydro-2H-pyran-4- yl)phenyl)pyrazin-2-yl)-7- fluoro-4-methylisoquinolin- 1(2H)-one | 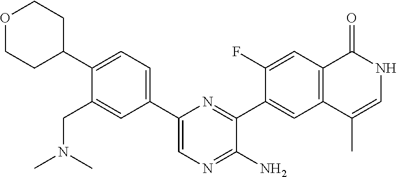 [SMILES: Cc1c[nH]c(=O)c2cc(F)c(-c3nc(-c4ccc(C5CCOCC5)c(CN(C)C)c4)cnc3N)cc12] |
| II-3 | 7-(3-amino-6-(3- ((dimethylamino)methyl)-4- (tetrahydro-2H-pyran-4- yl)phenyl)pyrazin-2-yl)-2- methylquinazolin-4(3H)-one | 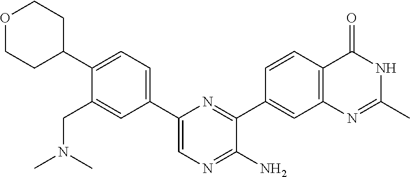 [SMILES: Cc1nc2cc(-c3nc(-c4ccc(C5CCOCC5)c(CN(C)C)c4)cnc3N)ccc2c(=O)[nH]1] |
| II-4 | 6-(3-amino-6-(3- ((dimethylamino)methyl)-4- (tetrahydro-2H-pyran-4- yl)phenyl)pyrazin-2-yl)-8- fluoro-3-methylisoquinolin- 1(2H)-one | 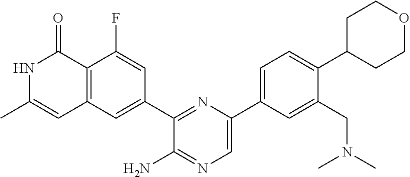 [SMILES: Cc1cc2cc(-c3nc(-c4ccc(C5CCOCC5)c(CN(C)C)c4)cnc3N)cc(F)c2c(=O)[nH]1] |
| II-5 | 6-(3-amino-6-(3- ((dimethylamino)methyl)-4- (tetrahydro-2H-pyran-4- yl)phenyl)pyrazin-2-yl)-4,8- difluoro-3-methylisoquinolin- 1(2H)-one | 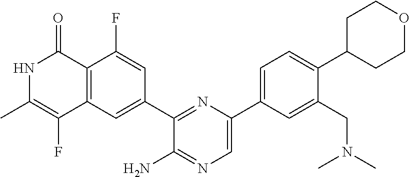 [SMILES: Cc1[nH]c(=O)c2c(F)cc(-c3nc(-c4ccc(C5CCOCC5)c(CN(C)C)c4)cnc3N)cc2c1F] |
| II-6 | 6-(3-amino-6-(3- ((dimethylamino)methyl)-4- (tetrahydro-2H-pyran-4- yl)phenyl)pyrazin-2-yl)-4,7- difluoro-3-methylisoquinolin- 1(2H)-one | 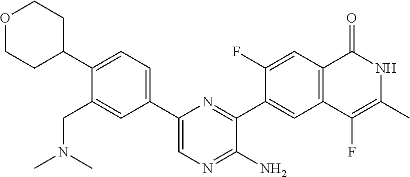 [SMILES: Cc1[nH]c(=O)c2cc(F)c(-c3nc(-c4ccc(C5CCOCC5)c(CN(C)C)c4)cnc3N)cc2c1F] |

or a pharmaceutically acceptable salt, solvate and/or prodrug thereof.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the application include, for example, those made with solvents that are pharmaceutically acceptable. Examples of such solvents include water (resulting solvate is called a hydrate) and ethanol and the like.

Prodrugs of the compounds of the present application may be, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

The compounds of the present application also include compounds having alternate isotopes, including radioactive and non-radioactive isotopes, for any of the atoms. For example, in an embodiment, the compounds of the application include compounds wherein one or more available hydrogen atoms have been substituted with deuterium. in an embodiment, the compounds of the application include compounds wherein one or more available carbon atoms have been substituted with ¹³C.

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

A compound of the application including salts and/or solvates thereof is suitably used on their own but will generally be administered in the form of a composition in which the one or more compounds of the application (the active ingredient) is in association with an acceptable carrier. Depending on the mode of administration, the composition will comprise from about 0.05 wt % to about 99 wt % or about 0.10 wt % to about 70 wt %, of the active ingredient, and from about 1 wt % to about 99.95 wt % or about 30 wt % to about 99.90 wt % of an acceptable carrier, all percentages by weight being based on the total composition.

The compounds of the application may be administered to a subject in a variety of forms depending on the selected route of administration, as will be understood by those skilled in the art. A compound of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery. Conventional procedures and ingredients for the selection and preparation of suitable compositions are described, for example, in Remington's Pharmaceutical Sciences (2000—20th edition) and in The United States Pharmacopeia: The National Formulary (USP 24 NF19) published in 1999.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature (RT) but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, P A, 1980, pp. 1530-1533 for further discussion of suppository dosage forms.

Compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In an embodiment, compounds of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

In some embodiments, depending on the mode of administration, the pharmaceutical composition will comprise from about 0.05 wt % to about 99 wt % or about 0.10 wt % to about 70 wt %, of the active ingredient (one or more compounds of the application), and from about 1 wt % to about 99.95 wt % or about 30 wt % to about 99.90 wt % of one or more pharmaceutically acceptable carriers, all percentages by weight being based on the total composition.

In an embodiment, a compound of the present application is administered with another therapeutic agent simultaneously or sequentially in separate unit dosage forms or together in a single unit dosage form. Accordingly, the present application provides a single unit dosage form comprising one or more compounds of the application (e.g. a compound of Formula (I)), an additional therapeutic agent, and a pharmaceutically acceptable carrier.

To be clear, in the above, the term “a compound” also includes embodiments wherein one or more compounds are referenced.

### III. Methods and Uses of the Application

The compounds of the application have been shown to be capable of inhibiting HPK1 activity. In an embodiment, the HPK1 is human HPK1, see for example, Hu, M. C. et. al.; Genes Dev. 10 (1): 2251-2264, 1996.

Accordingly, the present application includes a method for inhibiting HPK1, in a cell, either in a biological sample or in a patient, comprising administering an effective amount of one or more compounds of the application to the cell. The application also includes a use of one or more compounds of the application for inhibiting HPK1 in a cell as well as a use of one or more compounds of the application for the preparation of a medicament for inhibiting HPK1 in a cell. The application further includes one or more compounds of the application for use in inhibiting HPK1.

As the compounds of the application have been shown to be capable of inhibiting HPK1, the compounds of the application are useful for treating diseases, disorders or conditions by inhibiting HPK1. Therefore the compounds of the present application are useful as medicaments. Accordingly, the present application includes a compound of the application for use as a medicament.

The present application also includes a method of treating a disease, disorder or condition that is treatable by inhibiting HPK1 comprising administering a therapeutically effective amount of one or more compounds of the application to a subject in need thereof.

The present application also includes a use of one or more compounds of the application for treatment of a disease, disorder or condition that is treatable by inhibiting HPK1 as well as a use of one or more compounds of the application for the preparation of a medicament for treatment of a disease, disorder or condition that is treatable by inhibiting HPK1. The application further includes one or more compounds of the application for use in treating a disease, disorder or condition that is treatable by inhibiting HPK1.

In an embodiment, the disease, disorder or condition that is treatable by inhibiting HPK1 is a neoplastic disorder. Accordingly, the present application also includes a method of treating a neoplastic disorder comprising administering a therapeutically effective amount of one or more compounds of the application to a subject in need thereof. The present application also includes a use of one or more compounds of the application for treatment of a neoplastic disorder as well as a use of one or more compounds of the application for the preparation of a medicament for treatment of a neoplastic disorder. The application further includes one or more compounds of the application for use in treating a neoplastic disorder. In an embodiment, the treatment is in an amount effective to ameliorate at least one symptom of the neoplastic disorder, for example, reduced cell proliferation or reduced tumor mass, among others, in a subject in need of such treatment.

Compounds of the application have been demonstrated to inhibit HPK1 and hence cytokine release in immune derived cell (e.g. Jurkat-T cells). Therefore in another embodiment of the present application, the disease, disorder or condition that is treatable by inhibiting HPK1 is cancer. Accordingly, the present application also includes a method of treating cancer comprising administering a therapeutically effective amount of one or more compounds of the application to a subject in need thereof. The present application also includes a use of one or more compounds of the application for treatment of cancer as well as a use of one or more compounds of the application for the preparation of a medicament for treatment of cancer. The application further includes one or more compounds of the application for use in treating cancer. In an embodiment, the compound is administered for the prevention of cancer in a subject such as a mammal having a predisposition for cancer.

In an embodiment, the cancer is selected from hematologic cancers, breast cancers, ovarian cancers, lung cancers, melanomas, colon cancers and glioblastomas.

In an embodiment, the disease, disorder or condition that is treatable by inhibiting HPK1 is a disease, disorder or condition associated with an uncontrolled and/or abnormal cellular activity affected directly or indirectly by inhibiting HPK1. In another embodiment, the uncontrolled and/or abnormal cellular activity that is affected directly or indirectly by inhibiting HPK1 is proliferative activity in a cell. Accordingly, the application also includes a method of inhibiting proliferative activity in a cell, comprising administering an effective amount of one or more compounds of the application to the cell. The present application also includes a use of one or more compounds of the application for inhibition of proliferative activity in a cell as well as a use of one or more compounds of the application for the preparation of a medicament for inhibition of proliferative activity in a cell. The application further includes one or more compounds of the application for use in inhibiting proliferative activity in a cell by boosting immune cell function through HPK1 inhibition.

The present application also includes a method of inhibiting uncontrolled and/or abnormal cellular activities affected directly or indirectly by inhibiting HPK1 in a cell, either in a biological sample or in a subject, comprising administering an effective amount of one or more compounds of the application to the cell. The application also includes a use of one or more compounds of the application for inhibition of uncontrolled and/or abnormal cellular activities affected directly or indirectly by inhibiting HPK1 in a cell as well as a use of one or more compounds of the application for the preparation of a medicament for inhibition of uncontrolled and/or abnormal cellular activities affected directly or indirectly by inhibiting HPK1 in a cell. The application further includes one or more compounds of the application for use in inhibiting uncontrolled and/or abnormal cellular activities affected directly or indirectly by inhibiting HPK1 in a cell.

The present application also includes a method of treating a disease, disorder or condition that is treatable by inhibiting HPK1 comprising administering a therapeutically effective amount of one or more compounds of the application in combination with another known agent useful for treatment of a disease, disorder or condition that is treatable by inhibiting HPK1 to a subject in need thereof. The present application also includes a use of one or more compounds of the application in combination with another known agent useful for treatment of a disease, disorder or condition that is treatable by inhibiting HPK1 for treatment of a disease, disorder or condition that is treatable by inhibiting HPK1, as well as a use of one or more compounds of the application in combination with another known agent useful for treatment of a disease, disorder or condition that is treatable by inhibiting HPK1 for the preparation of a medicament for treatment of a disease, disorder or condition that is treatable by inhibiting HPK1. The application further includes one or more compounds of the application in combination with another known agent useful for treatment of a disease, disorder or condition that is treatable by inhibiting HPK1 for use in treating a disease, disorder or condition that is treatable by inhibiting HPK1. In an embodiment, the disease, disorder or condition treatable by inhibiting HPK1 is cancer.

In a further embodiment, the disease, disorder or condition that is treatable by inhibiting HPK1 is cancer and the one or more compounds of the application are administered in combination with one or more additional cancer treatments. In another embodiment, the additional cancer treatment is selected from radiotherapy, chemotherapy, targeted therapies such as antibody therapies and small molecule tyrosine-kinase inhibitors, immunotherapy, hormonal therapy and anti-angiogenic therapies.

When used in combination with other agents or therapies useful in treating diseases, disorders or conditions that are treatable by inhibiting HPK1, it is an embodiment that the compounds of the application are administered contemporaneously with those agents or therapies. As used herein, “contemporaneous administration” of two substances or therapies to a subject means providing each of the two substances or therapies so that they are both biologically active in the individual at the same time. The exact details of the administration will depend on the pharmacokinetics of the two substances or therapies in the presence of each other, and can include administering the two substances or therapies within a few hours of each other, or even administering one substance or therapy within 24 hours of administration of the other, if the pharmacokinetics are suitable. Design of suitable dosing regimens is routine for one skilled in the art. In particular embodiments, the substances or therapies will be administered substantially simultaneously, i.e., within minutes of each other, or in a single composition in the case of administration of two substances. It is a further embodiment of the present application that a combination of agents or therapies is administered to a subject in a non-contemporaneous fashion.

In an embodiment, the subject is a mammal. In an embodiment, the subject is human.

In the context of treating a disease, disorder or condition treatable by inhibition of HPK1, an effective amount is an amount that, for example, inhibits HPK1, compared to the inhibition without administration of the one or more compounds. Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom. When the disease is cancer, amounts that are effective can cause a reduction in the number, growth rate, size and/or distribution of tumours.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. In an embodiment of the application, compositions are formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Compounds of the application may be administered in a single daily, weekly or monthly dose or the total daily dose may be divided into two, three or four daily doses.

In an embodiment, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

### IV. Methods of Preparing the Compounds of the Application

Compounds of the present application can be prepared by various synthetic processes. The choice of particular structural features and/or substituents may influence the selection of one process over another. The selection of a particular process to prepare a given compound of Formula (I) is within the purview of the person of skill in the art. Some starting materials for preparing compounds of the present application are available from commercial chemical sources. Other starting materials, for example as described below, are readily prepared from available precursors using straightforward transformations that are well known in the art. In the Schemes below showing the preparation of compounds of the application, all variables are as defined in Formula (I), unless otherwise stated.

The compounds of Formula (I) generally can be prepared according to the processes illustrated in the Schemes below. In the structural formulae shown below the variables are as defined in Formula (I) unless otherwise stated. A person skilled in the art would appreciate that many of the reactions depicted in the Schemes below would be sensitive to oxygen and water and would know to perform the reaction under an anhydrous, inert atmosphere if needed. Reaction temperatures and times are presented for illustrative purposes only and may be varied to optimize yield as would be understood by a person skilled in the art.

Accordingly in an embodiment, the compounds of Formula (I) are prepared as shown in Schemes 1-6.

In an embodiment, compounds of Formula (I) are prepared by coupling a boronic acid or boronic ester of compound of Formula Formula (A) with a dihalogenated compound of Formula (B) to form the monohalogenated intermediate compound of Formula (C) as shown in Scheme 1. Intermediate compound of Formula (C) can then be coupled to a boronic acid or boronic ester intermediate compound of Formula (D) to form compounds of Formula (I). In an embodiment Hal¹ and Hal² are different halogens selected to have differing reactivity in the coupling reactions as would be known to those skilled in the art. In an embodiment, Hal¹ and Hal² are Cl and Br respectively. In another embodiment Hal¹ and Hal² are Cl and I respectively. In an embodiment R^{a}, R^{b}, R^{c} and R^{d} are all H. In an embodiment R^{a} and R^{b} together, or R^{c} and R^{d} together form a cycloalkyl ring. The variables Q, X¹, X², X³, X⁴, X⁵, Cy¹ and Cy² are as defined in Formula (I). In an embodiment, both coupling reactions are performed under cross-coupling conditions, such as in the presence of a cross-coupling catalyst and in an inert solvent. In some embodiments the cross-coupling catalyst is a palladium catalyst.

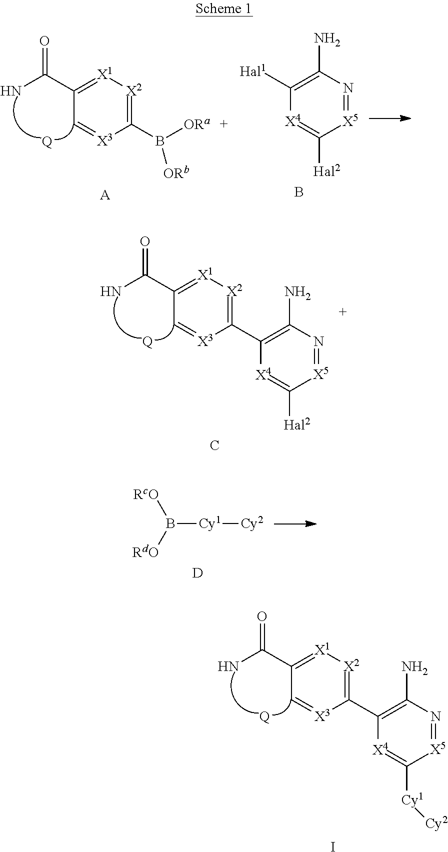

In a further embodiment the compounds of Formula (I) are synthesized as shown in Scheme 2 by first reacting a boronic acid or a boronic ester intermediate compound of Formula (D) with a dihalogenated intermediate compound of Formula (B) using a coupling reaction to form the monohalogenated intermediate compound of Formula (E). Intermediate compound of Formula (E) can then be reacted with a suitable boronic acid or boronic ester intermediate compound of Formula (A) to form a compounds of Formula (I). In an embodiment Hal¹ and Hal² are different halogens selected to have differing reactivity in the coupling reactions as would be known to those skilled in the art. In an embodiment, Hal¹ and Hal² are Cl and Br respectively. In another embodiment Hal¹ and Hal² are Cl and I respectively. In an embodiment R^{a}, R^{b}, R^{c} and R^{d} are all H. In an embodiment R^{a} and R^{b} together, or R^{c} and R^{d} together form a cycloalkyl ring. The variables Q, X¹, X², X³, X⁴, X⁵, Cy¹ and Cy² are as defined in Formula I. In an embodiment, both coupling reactions are performed under cross-coupling conditions, such as in the presence of a cross-coupling catalyst and in an inert solvent. In some embodiments the cross-coupling catalyst is a palladium catalyst

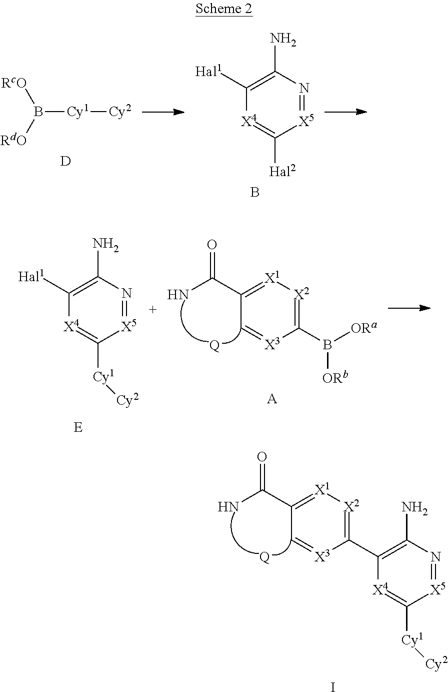

In an embodiment shown in Scheme 3, the compounds of Formula (I) wherein X² or X³ are either N or CR², and wherein X⁴, X⁵, R² are as defined in Formula I, are synthesized in a route in which a boronic acid or a boronic ester intermediate (H) is obtained from a mono-halogenated intermediate compound of Formula (F) wherein Hal³ is halo. The boronic acid or ester compound of Formula (H) is then treated with a compound of Formula (G) wherein Hal⁴ is halo by a coupling reaction to form the tricyclic intermediate compound of Formula (J). Intermediate compound of Formula (J) is then halogenated under standard conditions to form intermediate (C) wherein Hal² is halo, which can be reacted with intermediate compound of Formula (D) to form the compounds of Formula (I). In an embodiment Hal², Hal³ and Hal⁴ are each halogens selected to work in the specific coupling reaction as would be known to those skilled in the art. In an embodiment R^{c}, R^{d}, R^{e} and R^{f} are all H. In an embodiment R^{c} and R^{d} together, or R^{e} and R^{f} together form a cycloalkyl ring. The variables Q, X¹, X², X³, X⁴, X⁵, Cy¹ and Cy² are as defined in Formula I. In an embodiment, the coupling reactions are performed under cross-coupling conditions, such as in the presence of a cross-coupling catalyst and in an inert solvent. In some embodiments the cross-coupling catalyst is a palladium catalyst. In an embodiment the halogenation conditions comprise a halogenation reagent, such as N-bromosuccinamide.

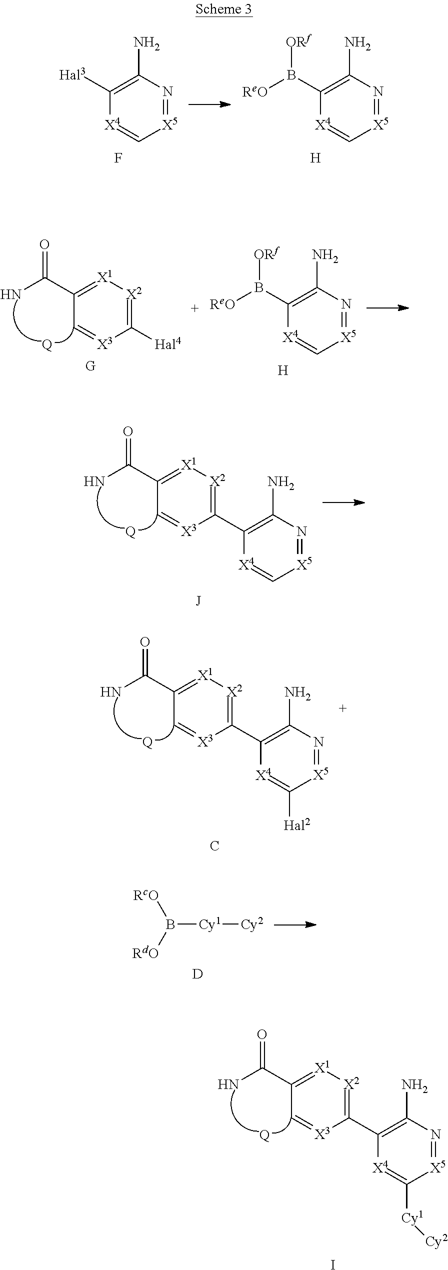

In an embodiment, as shown in Scheme 4, the intermediate compound of Formula (E) in Scheme 2 is synthesized by borylation of intermediate compound of Formula (B) to afford intermediate compound of Formula (K), followed by reaction with halogenated intermediate compound of Formula (L) by a coupling reaction to form the intermediate compound of Formula (E). In an embodiment Hal¹ and Hal² are different halogens selected to have differing reactivity in the coupling reactions as would be known to those skilled in the art. In an embodiment, Hal¹ and Hal² are Cl and Br respectively. In another embodiment Hal¹ and Hal² are Cl and I respectively. In an embodiment, Hal⁵ is any suitable halogen. In an embodiment R^{g} and R^{h} are both H. In an embodiment R^{g} and R^{h} together form a cycloalkyl ring. The variables X⁴, X⁵, Cy¹ and Cy² are as defined in Formula I. In an embodiment, the coupling reaction is performed under cross-coupling conditions, such as in the presence of a cross-coupling catalyst and in an inert solvent. In some embodiments the cross-coupling catalyst is a palladium catalyst. In some embodiments, the borylation comprises a borylation reagent in the presence of a catalyst, such as a palladium catalyst in an inert solvent.

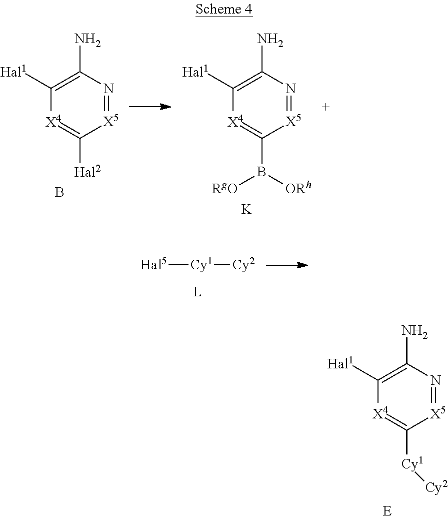

In an embodiment, as shown in Scheme 5, compounds of Formula (I) wherein Cy¹ is phenyl, and R⁷ is

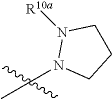

wherein R^{10a} is selected from H and R¹⁰ as defined in Formula (I) are prepared by coupling a boronic acid or boronic ester compound of Formula (M) with a halogenated compound of Formula (N) under suitable conditions, for example, under Suzuki coupling conditions, to form compounds of Formula (I). In an embodiment, R^{10a} is an amino protecting group, for example, tert-butyloxycarbonyl (Boc) which is removed under suitable conditions, for example, with strong acids such as trifluoroacetic acid to form a compound of Formula (I) wherein R^{10a} is H. In an embodiment, Hal⁶ is Br. In an embodiment, Rⁱ and R^{j} are both H. In an embodiment Rⁱ and R^{j} together, form a cycloalkyl ring. The variables Q, X¹, X², X³, Cy¹ and Cy² are as defined in Formula (I). In an embodiment, the compound of Formula I is the S-enantiomer, R⁷ is the S-enantionmer,

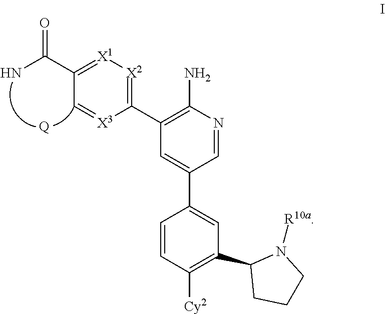

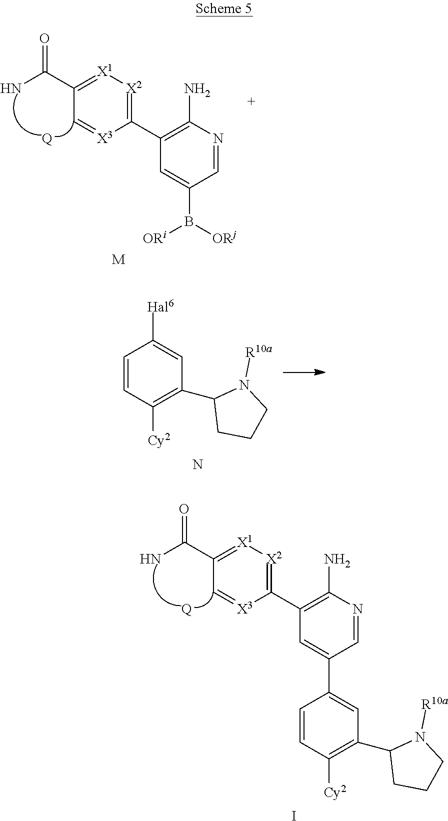

In an embodiment, as shown in Scheme 6, the intermediate compound of Formula (N) in Scheme 5 is synthesized by coupling dihalogenated ester compound of Formula (O) with halogened compound of Formula (P) under suitable coupling conditions such as in the presence of zinc to form halogenated ester compound of Formula (Q). The halogenated ester compound of Formula (Q) is then reduced under suitable reducing conditions such as in the presence of lithium aluminum hydride to form hydroxy compound of Formula (R) which is subsequently oxidized under suitable oxidizing conditions such as in the presence of manganese dioxide (MnO₂) to provide the halogenated aldehyde compound of Formula (S). The compound of Formula (S) is subsequently coupled with tert-butanesulfinamide (compound of Formula (T)) to form the aldimine compound of Formula (U) which is further coupled with a (1,3-dioxan-2-ylethyl) (1,3-dioxan-2-ylethyl)magnesium bromide (compound of Formula (V)) under suitable Grignard reaction conditions to provide intermediate compound of Formula (W) which is cyclized under suitable cyclization conditions such as in the presence of trifluoric acid (TFA) and triethylsilane (EtSiH) to form the compound of Formula (N). In an embodiment, the tert-butanesulfinamide (compound of Formula T) is S-tert-butanesulfinamide and the subsequent compounds of Formula (U), (W) and (N) are S-enantiomers. In an embodiment Hal⁶, Hal⁷ and Hal⁸ are each halogens selected to work in the specific coupling reaction as would be known to those skilled in the art. In an embodiment Hal⁶ and Hal⁷ are Br and I respectively. In an embodiment, Hal⁸ is any suitable halogen. In an embodiment, Hal⁸ is I. The variables Cy¹ and Cy² are as defined in Formula I.

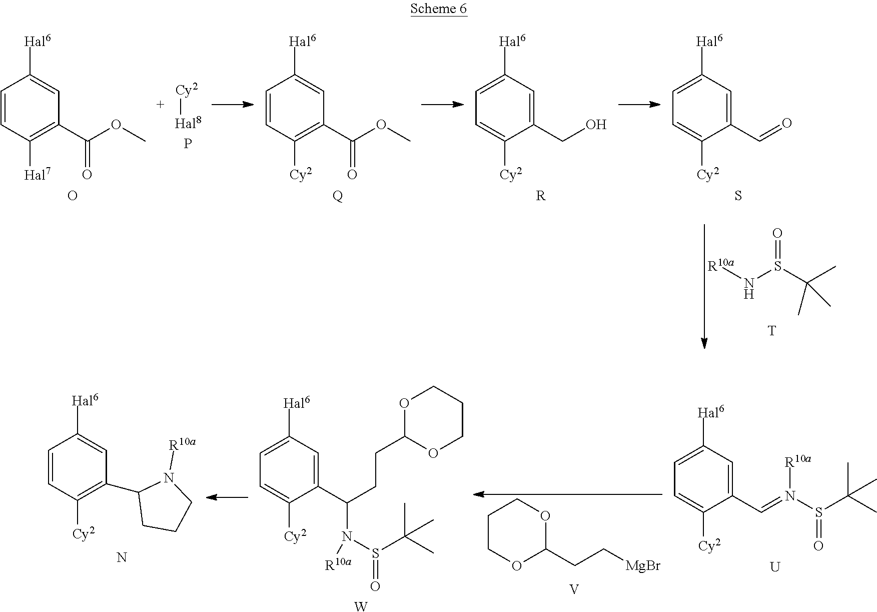

The schemes above are provided for illustration purposes. It will be understood by a person skilled in the art that the use of the most appropriate reagents may vary depend upon of the intermediates (A)-(W), and that the most appropriate route may also be dependent upon the intermediates and the target compound of Formula (I).

Intermediates of Formula (A)-(W) are either commercially available or may be prepared using methods known in the art.

It would be appreciated by the person skilled in the art that the compounds of Formula (II) can be prepared according to the processes and schemes illustrated for the compound of Formula (I) above.

Generally the reactions described above are performed in a suitable inert organic solvent and at temperatures and for times that will optimize the yield of the desired compounds. Examples of suitable inert organic solvents include, but are not limited to, dimethylformamide (DMF), dioxane, methylene chloride, chloroform, tetrahydrofuran (THF), toluene, and the like.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C1-C24) esters, acyloxymethyl esters, carbamates and amino acid esters.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallization, distillation and liquid-liquid or solid-liquid extraction, which will be readily understood by one skilled in the art.

The following non-limiting examples are illustrative of the present application.

### EXAMPLES

### A. Preparation of Exemplary Compounds of the Application

i. Preparation of Exemplary Compounds of Formula I

### Example 1: 6-(3-amino-6-(4-(4-methylpiperazin-1-yl)phenyl)pyrazin-2-yl)-3,4-dihydroisoquinolin-1(2H)-one (I-1)

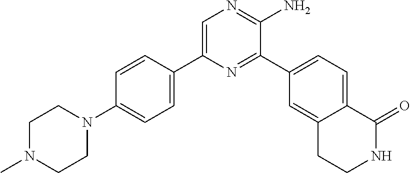 [SMILES: CN1CCN(c2ccc(-c3cnc(N)c(-c4ccc5c(c4)CCNC5=O)n3)cc2)CC1]

### Step 1: 3-chloro-5-(4-(4-methylpiperazin-1-yl)phenyl)pyrazin-2-amine

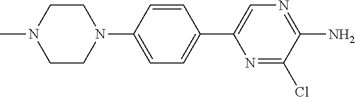 [SMILES: CN1CCN(c2ccc(-c3cnc(N)c(Cl)n3)cc2)CC1]

A vial was charged with 2-amino-5-bromo-3-chloropyrazine (100 mg, 0.480 mmol), Cs₂CO₃ (469 mg, 1.44 mmol), 4-(4-methylpiperazin-1-yl)phenylboronic acid, pinacol ester (145 mg, 0.480 mmol) and [1,1′-bis(diphenylphosphino)ferrocene]dichloropalladium(II) (PdCl₂dppf) (35.1 mg, 0.048 mmol) was suspended in H₂O (2 mL) and (dimethyl ether) DME (4 mL). The reaction was degassed by evacuation-refill with Ar then heated sealed under microwave irradiation at 90° C. for 2 h. The reaction mixture was concentrated under reduced pressure, deposited on Celite® and purified by flash chromatography (25 g SiO₂ InnoFlash® cartridge, using methanol (MeOH) in dichloromethane (CH₂Cl₂) eluting at 9% MeOH) to afford the product as a light yellow solid (162 mg, quant). LCMS: [M+H]+=304.12.

### Step 2: 6-(4,4,5,5-tetramethyl-1,3,2-dioxaborolan-2-yl)-3,4-dihydroisoquinolin-1(2H)-one

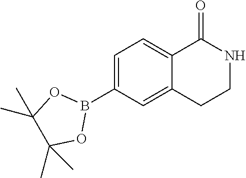 [SMILES: CC1(C)OB(c2ccc3c(c2)CCNC3=O)OC1(C)C]

To a degassed suspension of 6-bromo-3,4-dihydroisoquinolin-1(2H)-one (3 g, 13.3 mmol), 4,4,4′,4′,5,5,5′,5′-octamethyl-2,2′-bi(1,3,2-dioxaborolane) (5.07 g, 20 mmol), and KOAc (3.92 g, 40 mmol) in 1,4-dioxane (30 mL) was added a Pd(dppf)Cl2. CH₂Cl₂ (1.08 g, 1.3 mmol). The reaction mixture was heated to 85° C. for 16 h under argon atmosphere before cooling to RT. The reaction mixture was filtered through a Celite bed, which was washed with ethyl acetate (EtOAc) (100 mL). The combined filtrate was concentrated under reduced pressure to give residue; which was purified by column chromatography (silica gel 100-200 mesh) using an eluent 50-70% EtOAc in pet ether to afford the product (3 g, 82%) as a pale yellow solid. TLC (70% EtOAc:pet ether; R^{f}=0.6).

### Step 3: 6-(3-amino-6-(4-(4-methylpiperazin-1-yl)phenyl)pyrazin-2-yl)-3,4-dihydroisoquinolin-1(2H)-one

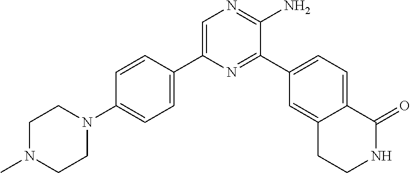 [SMILES: CN1CCN(c2ccc(-c3cnc(N)c(-c4ccc5c(c4)CCNC5=O)n3)cc2)CC1]

A mixture of 3-chloro-5-(4-(4-methylpiperazin-1-yl)phenyl)pyrazin-2-amine (47 mg, 0.147 mmol) and potassium phosphate (K₃PO₄) tribasic reagent grade, >=98% (109 mg, 0.513 mmol), XPhos Pd G2 (11.54 mg, 0.015 mmol) were placed under N₂. 6-(4,4,5,5-Tetramethyl-1,3,2-dioxaborolan-2-yl)-3,4-dihydroisoquinolin-1(2H)-one (42.6 mg, 0.156 mmol) in 1,4-dioxane (1.5 mL), H₂O (2 mL) and CH₃CN (“ACN”) (3 mL) were added. The mixture was degassed with N₂ and then heated in a microwave reactor at 100° C. for 2 h. The reaction mixture was concentrated under reduced pressure, deposited onto Celite® and purified by flash chromatography (12 g SiO₂InnoFlash® cartridge, using MeOH in CH₂Cl₂ eluting with 75% MeOH, pooled fractions:13-23 The fractions were concentrated under reduced pressure, deposited on Celite® and purified by preparative HPLC (30 g Biotage® SNAP KP-C₁₈-HS, MeOH in (H₂O+0.05% TFA)) eluting at 37% MeOH, pooled fractions:26-30 to afford the TFA salt of the title compound as an orange solid (47 mg, 50%). ¹H NMR (500 MHz, CD₃OD) δ 8.37 (br s, 1H), 8.10 (d, J=8.0 Hz, 1H), 7.92 (br d, J=8.7 Hz, 2H), 7.80 (br d, J=8.0 Hz, 1H), 7.75 (s, 1H), 7.12 (br d, J=8.7 Hz, 2H), 3.89-4.00 (m, 2H), 3.61-3.68 (m, 2H), 3.53-3.60 (m, 2H), 3.24-3.30 (m, 2H), 3.06-3.16 (m, 4H), 2.95-3.01 (m, 1H); LCMS: [M+H]⁺=415.48.

### Example 2: 5-(3-amino-6-(4-(4-methylpiperazin-1-yl)phenyl)pyrazin-2-yl)-3,3-dimethylisoindolin-1-one (I-2)

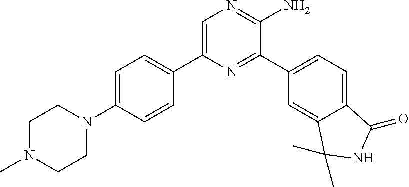 [SMILES: CN1CCN(c2ccc(-c3cnc(N)c(-c4ccc5c(c4)C(C)(C)NC5=O)n3)cc2)CC1]

### Step 1: 3,3-dimethyl-5-(4,4,5,5-tetramethyl-1,3,2-dioxaborolan-2-yl)isoindolin-1-one

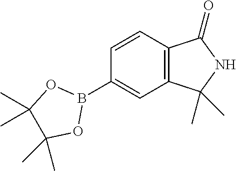

5-Bromo-3,3-dimethyl-2,3-dihydro-1H-isoindol-1-one (50 mg, 0.208 mmol), B₂pin₂ (58.2 mg, 0.229 mmol), KOAc (61.3 mg, 0.625 mmol) and PdCl₂dppf (7.62 mg, 10.41 μmol) were placed under an atmosphere of Ar before anhydrous 1,4-dioxane (7 mL) was added. The mixture was degassed with Ar and then heated in a microwave reactor at 100° C. for 2 h. LCMS: [M+H]⁺=288.10.

### Step 2: 5-(3-amino-6-(4-(4-methylpiperazin-1-yl)phenyl)pyrazin-2-yl)-3,3-dimethylisoindolin-1-one

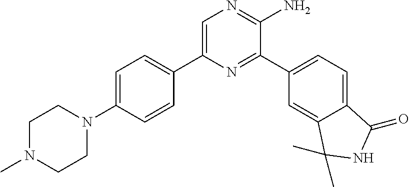 [SMILES: CN1CCN(c2ccc(-c3cnc(N)c(-c4ccc5c(c4)C(C)(C)NC5=O)n3)cc2)CC1]

A procedure similar to Example 1, Step 3 using 3-chloro-5-(4-(4-methylpiperazin-1-yl)phenyl)pyrazin-2-amine (52 mg, 0.164 mmol), K₃PO₄ (105 mg, 0.493 mmol), XPhos Pd G2 (12.93 mg, 0.016 mmol), 3,3-dimethyl-5-(4,4,5,5-tetramethyl-1,3,2-dioxaborolan-2-yl)isoindolin-1-one (61 mg, 2.05 mL in 1,4-dioxane, 0.214 mmol) to afford the title compound (64 mg, 59%) as a pale yellow solid. ¹H NMR (500 MHz, CD₃OD) δ=8.34 (br s, 1H), 7.97 (s, 1H), 7.93-7.88 (m, 4H), 7.11 (br d, J=7.9 Hz, 2H), 3.95 (br d, J=13.2 Hz, 2H), 3.63 (br d, J=11.7 Hz, 2H), 3.29-3.22 (m, 2H), 3.22-3.07 (m, 2H), 2.97 (s, 3H), 1.60 (s, 6H). LCMS: [M+H]⁺=429.33.

### Example 3: 6-(3-amino-6-(4-(4-isopropylpiperazin-1-yl)phenyl)pyrazin-2-yl)-3,4-dihydroisoquinolin-1(2H)-one (I-3)

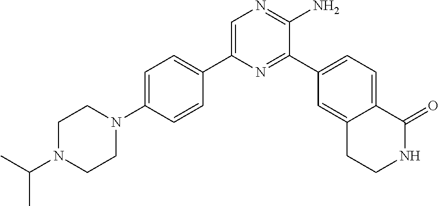 [SMILES: CC(C)N1CCN(c2ccc(-c3cnc(N)c(-c4ccc5c(c4)CCNC5=O)n3)cc2)CC1]

### Step 1: 3-chloro-5-(4-(4-isopropylpiperazin-1-yl)phenyl)pyrazin-2-amine

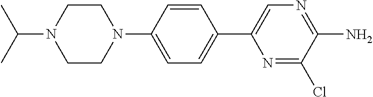 [SMILES: CC(C)N1CCN(c2ccc(-c3cnc(N)c(Cl)n3)cc2)CC1]

The intermediate was prepared by a procedure similar to that described in Example 1, Step 1 using 2-amino-5-bromo-3-chloropyrazine (80 mg, 0.384 mmol), Cs₂CO₃ (375 mg, 1.151 mmol), 4-(4-isopropylpiperazinyl)phenylboronic acid, pinacol ester (127 mg, 0.384 mmol), PdCl₂dppf (28.1 mg, 0.038 mmol), H₂O (2 mL) and DME (4 mL) by heating under microwave irradiation at 90° C. for 2 h. Purification by flash chromatography (12 g SiO₂ InnoFlash® cartridge, using MeOH in CH₂Cl₂ eluting at 7% MeOH) afforded the product as a tan solid (61 mg, 40% based on purity of 84%). LCMS: [M+H]⁺=332.19.

### Step 2: 6-(3-amino-6-(4-(4-isopropylpiperazin-1-yl)phenyl)pyrazin-2-yl)-3,4-dihydroisoquinolin-1(2H)-one

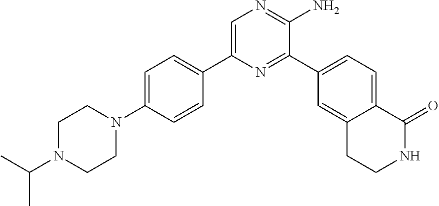 [SMILES: CC(C)N1CCN(c2ccc(-c3cnc(N)c(-c4ccc5c(c4)CCNC5=O)n3)cc2)CC1]

The title compound was prepared by a method similar to Example 1, Step 3 using 3-chloro-5-(4-(4-isopropylpiperazin-1-yl)phenyl)pyrazin-2-amine (61 mg, 0.154 mmol), 3,4-dihydro-1(2H)-isoquinolinone-6-boronic acid pinacol ester (54.8 mg, 0.201 mmol), XPhos Pd G2 (12.15 mg, 0.015 mmol), H₂O (2 mL) and MeCN (3 mL); by heating in a microwave reactor at 100° C. for 3 h. Purified by flash chromatography (25 g SiO₂ InnoFlash® cartridge, using MeOH in CH₂Cl₂ eluting at 4% MeOH) followed by preparative HPLC (30 g Biotage® SNAP KP-C₁₈-HS, MeOH in (H₂O+0.05% TFA) eluting at 35% MeOH) to afford the title compound as a light tan solid (42 mg, 40% based on purity of 98%). ¹H NMR (500 MHz, CD₃OD) δ 8.32 (s, 1H), 8.10 (d, J=7.9 Hz, 1H), 7.91 (br d, J=8.6 Hz, 2H), 7.79 (br d, J=7.9 Hz, 1H), 7.74 (s, 1H), 7.11 (br d, J=8.4 Hz, 2H), 4.06-3.93 (m, 2H), 3.66-3.53 (m, 5H), 3.29-3.25 (m, 2H), 3.16-3.05 (m, 4H), 1.43 (d, J=6.6 Hz, 6H). A 2H peak at ˜ 3.3 ppm (2H) is partially obscured by the solvent. LCMS: [M+H]⁺=443.40.

### Example 4: 6-(3-amino-6-(3-fluoro-4-(4-methylpiperazin-1-yl)phenyl)pyrazin-2-yl)-3,4-dihydroisoquinolin-1(2H)-one (I-4)

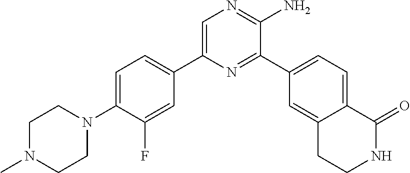 [SMILES: CN1CCN(c2ccc(-c3cnc(N)c(-c4ccc5c(c4)CCNC5=O)n3)cc2F)CC1]

### Step 1: 3-chloro-5-(3-fluoro-4-(4-methylpiperazin-1-yl)phenyl)pyrazin-2-amine

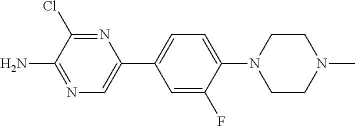 [SMILES: CN1CCN(c2ccc(-c3cnc(N)c(Cl)n3)cc2F)CC1]

The intermediate was prepared by a method similar to Example 1, Step 1 using 2-amino-5-bromo-3-chloropyrazine (100 mg, 0.480 mmol), Cs₂CO₃ (469 mg, 1.439 mmol), 3-fluoro-4-(4-methyl-1-piperazinyl)benzeneboronic acid pinacol ester (154 mg, 0.480 mmol), PdCl₂dppf (35.1 mg, 0.048 mmol), H₂O (2 mL) and DME (4 mL); by heating under microwave irradiation at 90° C. for 3 h. Purification by flash chromatography (25 g SiO₂ InnoFlash® cartridge, using EtOAc in CH₂Cl₂ 0-100% then MeOH in CH₂Cl₂ eluting at 10% MeOH) afforded the product as a yellow solid (91 mg, 59%). LCMS: [M+H]⁺=322.24.

### Step 2: 6-(3-amino-6-(3-fluoro-4-(4-methylpiperazin-1-yl)phenyl)pyrazin-2-yl)-3,4-dihydroisoquinolin-1(2H)-one

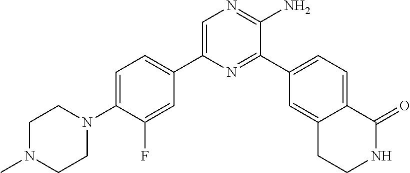 [SMILES: CN1CCN(c2ccc(-c3cnc(N)c(-c4ccc5c(c4)CCNC5=O)n3)cc2F)CC1]

A procedure analogous to that described in Example 1, Step 3 using 3-chloro-5-(3-fluoro-4-(4-methylpiperazin-1-yl)phenyl)pyrazin-2-amine (61 mg, 0.190 mmol), 3,4-dihydro-1(2H)-isoquinolinone-6-boronic acid pinacol ester (57.0 mg, 0.209 mmol), XPhos Pd G2 (14.92 mg, 0.019 mmol) afforded the title compound (17 mg, 14%) as a yellow solid. ¹H NMR (500 MHz, CD₃OD) δ 8.44 (br s, 1H), 8.09 (br d, J=7.9 Hz, 1H), 7.81-7.72 (m, 4H), 7.16 (br t, J=8.6 Hz, 1H), 3.72-3.54 (m, 6H), 3.42-3.33 (m, 2H), 3.20-3.04 (m, 4H), 2.99 (s, 3H); LCMS: [M+H]⁺=433.24.

### Example 5: 6-(3-amino-6-(4-(4-methylpiperazin-1-yl)phenyl)pyrazin-2-yl)-8-fluoro-3,4-dihydroisoquinolin-1(2H)-one (I-5)

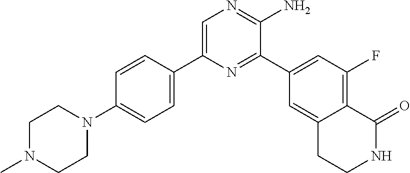 [SMILES: CN1CCN(c2ccc(-c3cnc(N)c(-c4cc(F)c5c(c4)CCNC5=O)n3)cc2)CC1]

### Step 1: 8-fluoro-6-(4,4,5,5-tetramethyl-1,3,2-dioxaborolan-2-yl)-3,4-dihydroisoquinolin-1(2H)-one

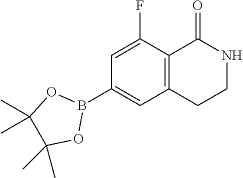 [SMILES: CC1(C)OB(c2cc(F)c3c(c2)CCNC3=O)OC1(C)C]

A sealed, degassed mixture of 6-bromo-8-fluoro-3,4-dihydroisoquinolin-1(2H)-one (125 mg, 0.51 mmol), B₂pin₂ (143 mg, 0.56 mmol), KOAc (151 mg, 1.54 mmol) and PdCl₂dppf (18.74 mg, 0.026 mmol) in anhydrous 1,4-dioxane (6 mL) was heated in a microwave reactor at 100° C. for 2 h. The reaction was worked up using standard methods, and the resulting crude product was used without purification in the following step. LCMS: [M+H]⁺=292.00.

### Step 2: 6-(3-amino-6-(4-(4-methylpiperazin-1-yl)phenyl)pyrazin-2-yl)-8-fluoro-3,4-dihydroisoquinolin-1(2H)-one

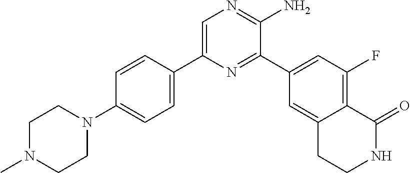 [SMILES: CN1CCN(c2ccc(-c3cnc(N)c(-c4cc(F)c5c(c4)CCNC5=O)n3)cc2)CC1]

The title compound was prepared following a procedure analagous to Example 1, Step 3 using 3-chloro-5-(4-(4-methylpiperazin-1-yl)phenyl)pyrazin-2-amine (62.7 mg, 0.206 mmol) (preparation described in Example 1, Step 1), K₃PO₄ (131 mg, 0.619 mmol), XPhos Pd G2 (16.2 mg, 0.021 mmol), 8-fluoro-6-(4,4,5,5-tetramethyl-1,3,2-dioxaborolan-2-yl)-3,4-dihydroisoquinolin-1(2H)-one (66.1 mg in 1,4-dioxane (2.67 mL), 0.23 mmol; crude from step 1) in H₂O (4 mL) and MeCN (6 mL); by heating sealed in a microwave reactor at 100° C. for 3 h. Purification by flash chromatography (25 g SiO₂ Biotage® cartridge, using MeOH in CH₂Cl₂) followed by preparative HPLC (30 g Biotage® SNAP KP-C₁₈-HS, MeOH in (H₂O+0.05% TFA)) afforded the TFA salt of the title compound (43 mg, 31% based on purity of 98%). ¹H NMR (500 MHz, CD₃OD) δ 8.40 (br s, 1H), 7.91 (m, J=8.8 Hz, 2H), 7.60 (s, 1H), 7.53 (br d, J=12.0 Hz, 1H), 7.11 (m, J=8.9 Hz, 2H), 3.89-4.02 (m, 2H), 3.63 (br d, J=11.7 Hz, 2H), 3.51 (t, J=6.4 Hz, 2H), 3.24-3.29 (m, 2H), 3.02-3.21 (m, 4H), 2.98 (s, 3H); LCMS: [M+H]⁺=433.33.

### Example 6: 6-(3-amino-6-(4-(4-hydroxypiperidin-1-yl)phenyl)pyrazin-2-yl)-3,4-dihydroisoquinolin-1(2H)-one (I-6)

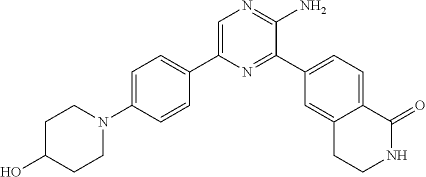 [SMILES: Nc1ncc(-c2ccc(N3CCC(O)CC3)cc2)nc1-c1ccc2c(c1)CCNC2=O]

### Step 1: 1-(4-(4,4,5,5-tetramethyl-1,3,2-dioxaborolan-2-yl)phenyl)piperidin-4-ol

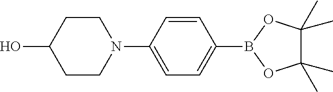 [SMILES: CC1(C)OB(c2ccc(N3CCC(O)CC3)cc2)OC1(C)C]

To 1-(4-bromophenyl)piperidin-4-ol (250 mg, 0.976 mmol), B₂pin₂ (273 mg, 1.074 mmol), KOAc (287 mg, 2.93 mmol) and PdCl₂dppf (35.7 mg, 0.049 mmol) anh 1,4-dioxane (6 mL) was added. The mixture was degassed with N₂ and then heated in an oil bath at 100° C. for 7 h. The resulting product was used as the crude 1,4-dioxane mixture in the following step. LCMS: [M+H]+=304.10.

### Step 2: 1-(4-(5-amino-6-chloropyrazin-2-yl)phenyl)piperidin-4-ol

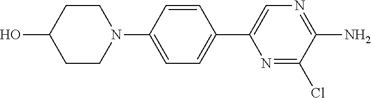 [SMILES: Nc1ncc(-c2ccc(N3CCC(O)CC3)cc2)nc1Cl]

The intermediate was prepared by a procedure similar to Example 1, Step 1 using 2-amino-5-bromo-3-chloropyrazine (76 mg, 0.363 mmol), Cs₂CO₃ (237 mg, 0.726 mmol), 1-(4-(4,4,5,5-tetramethyl-1,3,2-dioxaborolan-2-yl)phenyl)piperidin-4-ol (108 mg, 0.242 mmol) in 1,4-dioxane (3.65 mL), along with PdCl₂dppf (8.9 mg, 0.012 mmol), H₂O (2 mL) and DME (1.25 mL) by heating under microwave irradiation at 90° C. for 1 h. The reaction mixture was concentrated under reduced pressure and purified by flash chromatography (25 g SiO₂ Biotage® cartridge, using MeOH in CH₂Cl₂ eluting at 5% MeOH) to afford the product as a yellow solid (70 mg, 74%, based on the purity of 78%). LCMS: [M+H]⁺=305.23.

### Step 3: 6-(3-amino-6-(4-(4-hydroxypiperidin-1-yl)phenyl)pyrazin-2-yl)-3,4-dihydroisoquinolin-1(2H)-one

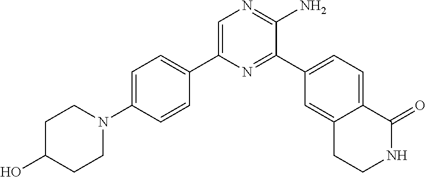 [SMILES: Nc1ncc(-c2ccc(N3CCC(O)CC3)cc2)nc1-c1ccc2c(c1)CCNC2=O]

The title compound was prepared by a procedure analogous to Example 1, Step 3 using 3,4-dihydro-1(2H)-isoquinolinone-6-boronic acid pinacol ester (73.4 mg, 0.269 mmol), K₃PO₄ (114 mg, 0.537 mmol), 1-(4-(5-amino-6-chloropyrazin-2-yl)phenyl)piperidin-4-ol (70 mg, 0.179 mmol), XPhos Pd G2 (14.10 mg, 0.018 mmol), MeCN (9 mL) and H₂O (6 mL) to afford the title compound (6 mg, 6%) as a yellow film. ¹H NMR (500 MHz, CD₃OD) δ 8.54 (s, 1H), 8.20 (m, J=8.7 Hz, 2H), 8.11 (d, J=8.0 Hz, 1H), 7.83 (br d, J=8.1 Hz, 1H), 7.78 (s, 1H), 7.67 (m, J=8.7 Hz, 2H), 4.10 (br s, 1H), 3.88 (br t, J=8.0 Hz, 2H), 3.59 (br t, J=6.6 Hz, 4H), 3.12 (t, J=6.6 Hz, 2H), 2.16-2.31 (m, 2H), 1.98-2.08 (m, 2H); LCMS: [M+H]⁺=416.26.

### Example 7: (R)-6-(3-amino-6-(4-(2-isopropylmorpholino)phenyl)pyrazin-2-yl)-3,4-dihydroisoquinolin-1(2H)-one (I-7)

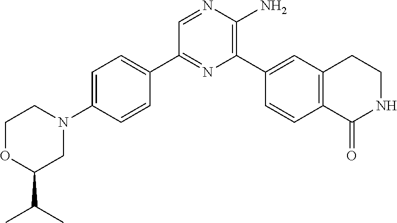 [SMILES: CC(C)[C@@H]1CN(c2ccc(-c3cnc(N)c(-c4ccc5c(c4)CCNC5=O)n3)cc2)CCO1]

### Step 1: (R)-4-(4-chlorophenyl)-2-isopropylmorpholine

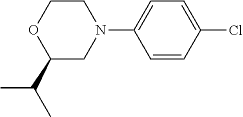 [SMILES: CC(C)[C@@H]1CN(c2ccc(Cl)cc2)CCO1]

A 30 mL vial was charged with 1-chloro-4-iodobenzene (0.500 g, 2.097 mmol), (R)-2-isopropylmorpholine (0.298 g, 2.31 mmol), tris(dibenzylideneacetone)dipalladium (0) (0.058 g, 0.063 mmol), 4,5-bis(diphenylphosphino)-9,9-dimethylxanthene (0.109 g, 0.189 mmol) and Cs₂CO₃ (2.050 g, 6.29 mmol). The vial was sealed with a cap and septum and then the reaction vessel was evacuated and backfilled with nitrogen. Toluene (7 mL) was added and the reaction vessel was evacuated and backfilled with nitrogen an additional time. The reaction was heated at 100° C. for 18 h, then was cooled to RT and partitioned between EtOAc and water. The layers were separated and the aqueous layer was extracted with additional EtOAc (×2). The organic layer was concentrated and the residue was loaded onto Celite and purified by flash chromatography (eluting with 1-15% EtOAc/hexanes) to afford the product (398 mg, 79%). LCMS: [M+H]⁺=240.11.

### Step 2: (R)-2-isopropyl-4-(4-(4,4,5,5-tetramethyl-1,3,2-dioxaborolan-2-yl)phenyl)morpholine

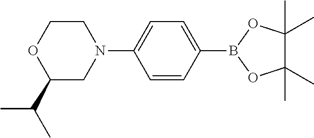 [SMILES: CC(C)[C@@H]1CN(c2ccc(B3OC(C)(C)C(C)(C)O3)cc2)CCO1]

A 30 ml vial was charged with (R)-4-(4-chlorophenyl)-2-isopropylmorpholine (0.397 g, 1.656 mmol), bis(pinacolato)diboron (0.526 g, 2.070 mmol), KOAc (0.325 g, 3.31 mmol) and XPhos Pd G2 (0.098 g, 0.124 mmol). The vial was evacuated and backfilled with nitrogen and 1,4-dioxane (5 mL) was added and the vial was evacuated and backfilled an additional time. The reaction mixture was then heated to 90° C. in an aluminum block for 18 h. The reaction mixture was concentrated onto Celite and purified by flash chromatography (1-15% EtOAc/hexanes) to afford the product (451 mg, 82%). LCMS: [M+H]⁺=332.08.

### Step 3: (R)-3-chloro-5-(4-(2-isopropylmorpholino)phenyl)pyrazin-2-amine

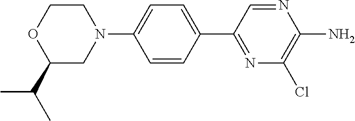 [SMILES: CC(C)[C@@H]1CN(c2ccc(-c3cnc(N)c(Cl)n3)cc2)CCO1]

A 30 mL vial was charged with 2-amino-5-bromo-3-chloropyrazine (0.076 g, 0.362 mmol), (R)-2-isopropyl-4-(4-(4,4,5,5-tetramethyl-1,3,2-dioxaborolan-2-yl)phenyl)morpholine (0.100 g, 0.302 mmol), [1,1′-bis(diphenylphosphino)ferrocene]dichloropalladium(II) (0.022 g, 0.030 mmol) and Cs₂CO₃ (0.246 g, 0.755 mmol). After the vial was sealed with a cap and septum the reaction vessel was evacuated and backfilled with nitrogen. 1,4-dioxane (2 mL) and H2O (1 mL) were added via syringe and the vessel was evacuated and backfilled with nitrogen an additional time. The reaction was heated to 80° C. for 3 h, then stirred at RT overnight and concentrated onto Celite. Purification by flash chromatography (0.5-10% MeOH/CH₂Cl₂+0.5% NH4OH) afforded the product (71 mg, 71%). LCMS: [M+H]⁺=333.21.

### Step 4: (R)-6-(3-amino-6-(4-(2-isopropylmorpholino)phenyl)pyrazin-2-yl)-3,4-dihydroisoquinolin-1(2H)-one

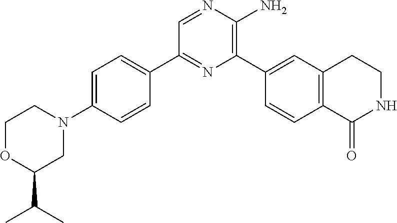 [SMILES: CC(C)[C@@H]1CN(c2ccc(-c3cnc(N)c(-c4ccc5c(c4)CCNC5=O)n3)cc2)CCO1]

In a 30 mL vial with magnetic stir bar was charged (R)-3-chloro-5-(4-(2-isopropylmorpholino)phenyl)pyrazin-2-amine (0.025 g, 0.075 mmol), 3,4-dihydro-1(2H)-isoquinolinone-6-boronic acid pinacol ester (0.025 g, 0.090 mmol, preparation described in Example 1, Step 3) and XPhos Pd G2 (5.91 mg, 7.51 mol). The vial was sealed with a cap and septum and the reaction vessel was evacuated and backfilled with nitrogen. 1,4-Dioxane (1.0 mL) and aqueous K₃PO₄ (0.144 mL, 0.188 mmol) were added and the reaction vessel was evacuated and backfilled with nitrogen an additional time. The reaction mixture was heated at 90° C. for 18 h in an aluminum block. The reaction mixture was concentrated onto Celite and purified by flash chromatography (eluting with 0.5-10% MeOH/CH₂Cl₂+0.5% NH4OH) to afford the title compound (16 mg, 48%). ¹H NMR (500 MHz, DMSO-d₆) δ 8.49 (s, 1H), 7.9-8.0 (m, 2H), 7.86 (d, J=9.0 Hz, 2H), 7.75 (dd, J=8.0, 1.7 Hz, 1H), 7.71 (s, 1H), 7.03 (d, J=9.0 Hz, 2H), 6.24 (s, 2H), 3.97 (dd, J=11.3, 2.3 Hz, 1H), 3.5-3.7 (m, 4H), 3.43 (dt, J=6.5, 2.8 Hz, 3H), 3.25 (ddd, J=10.4, 6.4, 2.1 Hz, 1H), 3.00 (t, J=6.5 Hz, 2H), 2.69 (dt, 11.8, 3.5 Hz, 1H), 1.74 (qd, J=13.4, 6.8 Hz, 1H), 0.96 (dd, J=6.8, 3.1 Hz, 6H); LCMS: [M+H]⁺=444.4.

### Example 8: (S)-6-(3-amino-6-(4-(2-isopropylmorpholino)phenyl)pyrazin-2-yl)-3,4-dihydroisoquinolin-1(2H)-one (I-8)

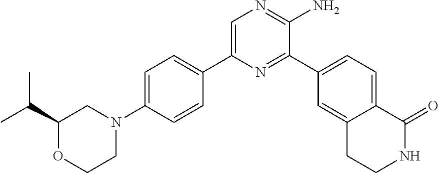 [SMILES: CC(C)[C@H]1CN(c2ccc(-c3cnc(N)c(-c4ccc5c(c4)CCNC5=O)n3)cc2)CCO1]

### Step 1: (S)-4-(4-chlorophenyl)-2-isopropylmorpholine

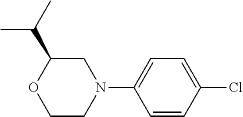 [SMILES: CC(C)[C@H]1CN(c2ccc(Cl)cc2)CCO1]

The procedure followed was analogous to Example 7, Step 1 using 1-chloro-4-iodobenzene (0.500 g, 2.097 mmol), (S)-2-isopropylmorpholine (0.298 g, 2.31 mmol), tris(dibenzylideneacetone)dipalladium (0) (0.058 g, 0.063 mmol), 4,5-bis(diphenylphosphino)-9,9-dimethylxanthene (0.109 g, 0.189 mmol) and Cs₂CO₃ (2.05 g, 6.29 mmol). Workup and purification afforded the product (404 mg, 80%). LCMS: [M+H]⁺=240.03.

### Step 2: (S)-2-isopropyl-4-(4-(4,4,5,5-tetramethyl-1,3,2-dioxaborolan-2-yl)phenyl)morpholine

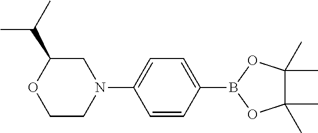 [SMILES: CC(C)[C@H]1CN(c2ccc(B3OC(C)(C)C(C)(C)O3)cc2)CCO1]

A procedure analogous to Example 7, Step 2 using (S)-4-(4-chlorophenyl)-2-isopropylmorpholine (0.404 g, 1.685 mmol), bis(pinacolato)diboron (0.535 g, 2.106 mmol), KOAc (0.331 g, 3.37 mmol) and XPhos Pd G2 (0.099 g, 0.126 mmol) afforded the product (1.43 mmol, 85%). LCMS: [M+H]=332.08.

### Step 3: (S)-3-chloro-5-(4-(2-isopropylmorpholino)phenyl)pyrazin-2-amine

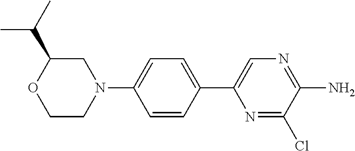 [SMILES: CC(C)[C@H]1CN(c2ccc(-c3cnc(N)c(Cl)n3)cc2)CCO1]

A procedure analogous to Example 7, Step 3 using 2-amino-5-bromo-3-chloropyrazine (0.076 g, 0.362 mmol), (S)-2-isopropyl-4-(4-(4,4,5,5-tetramethyl-1,3,2-dioxaborolan-2-yl)phenyl)morpholine (0.100 g, 0.302 mmol), [1,1′-bis(diphenylphosphino)ferrocene]dichloropalladium(II) (0.022 g, 0.030 mmol) and Cs₂CO₃ (0.246 g, 0.755 mmol) afforded the product (53 mg, 53%). LCMS: [M+H]⁺=333.29.

### Step 4: (S)-6-(3-amino-6-(4-(2-isopropylmorpholino)phenyl)pyrazin-2-yl)-3,4-dihydroisoquinolin-1(2H)-one

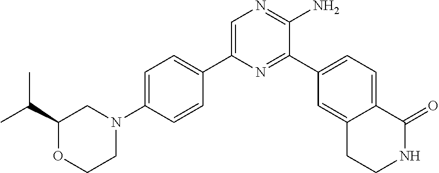 [SMILES: CC(C)[C@H]1CN(c2ccc(-c3cnc(N)c(-c4ccc5c(c4)CCNC5=O)n3)cc2)CCO1]

A procedure analogous to Example 7, Step 4 using (S)-3-chloro-5-(4-(2-isopropylmorpholino)phenyl)pyrazin-2-amine (0.025 g, 0.075 mmol), 3,4-dihydro-1(2H)-isoquinolinone-6-boronic acid pinacol ester (0.025 g, 0.090 mmol), and XPhos Pd G2 (5.91 mg, 7.51 μmol) afforded the title compound (6 mg, 12%). ¹H NMR (500 MHz, CD₃OD) δ 8.22 (d, J=2.3 Hz, 1H), 7.94 (d, J=7.8 Hz, 1H), 7.77 (s, 1H), 7.71 (d, J=2.3 Hz, 1H), 7.69 (d, J=7.8 Hz, 1H), 7.51 (d, J=8.8 Hz, 2H), 7.06 (d, J=8.8 Hz, 2H), 4.56 (s, 2H), 4.03 (dd, J=11.4, 2.0 Hz, 1H), 3.76 (dt, J=11.5, 2.6 Hz, 1H), 3.62 (br d, J=11.7 Hz, 1H), 3.51 (br d, J=11.1 Hz, 1H), 2.78 (dt, J=11.8, 3.3 Hz, 1H), 2.54 (t, J=11.1 Hz, 1H), 1.79 (qd, J=13.6, 6.8 Hz, 1H), 1.03 (dd, J=11.7, 6.8 Hz, 6H); LCMS: [M+H]⁺=429.4.

### Example 9: (R)-N-(1-(4-(5-amino-6-(1-oxo-1,2,3,4-tetrahydroisoquinolin-6-yl)pyrazin-2-yl)phenyl)pyrrolidin-3-yl)-N-methylmethanesulfonamide (I-9)

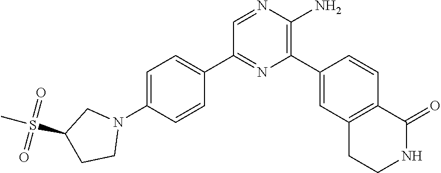 [SMILES: CS(=O)(=O)[C@@H]1CCN(c2ccc(-c3cnc(N)c(-c4ccc5c(c4)CCNC5=O)n3)cc2)C1]

### Step 1: (R)-N-(1-(4-chlorophenyl)pyrrolidin-3-yl)-N-methylmethanesulfonamide

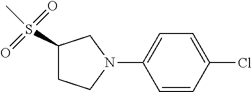 [SMILES: CS(=O)(=O)[C@@H]1CCN(c2ccc(Cl)cc2)C1]

A procedure analogous to Example 7, Step 1 using 1-chloro-4-iodobenzene (0.250 g, 1.05 mmol), tris(dibenzylideneacetone)dipalladium (0) (0.029 g, 0.031 mmol), 4,5-bis(diphenylphosphino)-9,9-dimethylxanthene (0.055 g, 0.094 mmol) and Cs₂CO₃ (1.025 g, 3.15 mmol) afforded the product (149 mg, 49%). LCMS: [M+H]⁺=289.14.

### Step 2: (R)-N-methyl-N-(1-(4-(4,4,5,5-tetramethyl-1,3,2-dioxaborolan-2-yl)phenyl)pyrrolidin-3-yl)methanesulfonamide

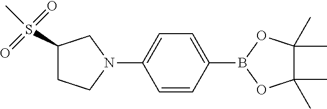

A procedure analogous to Example 7, Step 2 using (R)-N-(1-(4-chlorophenyl)pyrrolidin-3-yl)-N-methylmethanesulfonamide (0.149 g, 0.516 mmol), bis(pinacolato)diboron (0.197 g, 0.774 mmol), KOAc (0.101 g, 1.03 mmol) and XPhos Pd G2 (0.030 g, 0.039 mmol) afforded the product (119 mg, 61%). LCMS: [M+H]⁺=381.28

### Step 3: (R)-N-(1-(4-(5-amino-6-chloropyrazin-2-yl)phenyl)pyrrolidin-3-yl)-N-methylmethanesulfonamide)

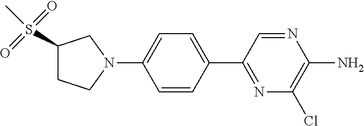 [SMILES: CS(=O)(=O)[C@@H]1CCN(c2ccc(-c3cnc(N)c(Cl)n3)cc2)C1]

A procedure analogous to Example 7, Step 3 using 2-amino-5-bromo-3-chloropyrazine (0.033 g, 0.158 mmol), (R)-N-methyl-N-(1-(4-(4,4,5,5-tetramethyl-1,3,2-dioxaborolan-2-yl)phenyl)pyrrolidin-3-yl)methanesulfonamide (0.050 g, 0.131 mmol), [1,1′-bis(diphenylphosphino)ferrocene]dichloropalladium(II) (9.62 mg, 0.013 mmol) and Cs₂CO₃ (0.107 g, 0.329 mmol) afforded the product (37 mg, 74%). LCMS: [M+H]⁺=382.25

### Step 4: (R)-N-(1-(4-(5-amino-6-(1-oxo-1,2,3,4-tetrahydroisoquinolin-6-yl)pyrazin-2-yl)phenyl)pyrrolidin-3-yl)-N-methylmethanesulfonamide (I-9)

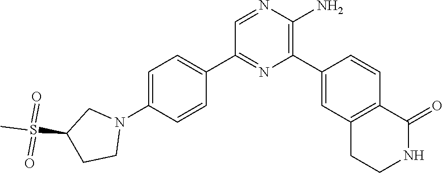 [SMILES: CS(=O)(=O)[C@@H]1CCN(c2ccc(-c3cnc(N)c(-c4ccc5c(c4)CCNC5=O)n3)cc2)C1]

A procedure analogous to Example 7, Step 4 using (R)-N-(1-(4-(5-amino-6-chloropyrazin-2-yl)phenyl)pyrrolidin-3-yl)-N-methylmethanesulfonamide (0.037 g, 0.097 mmol), 3,4-dihydro-1(2H)-isoquinolinone-6-boronic acid pinacol ester (0.032 g, 0.116 mmol) and XPhos Pd G2 (7.62 mg, 9.69 μmol) afforded, after workup and purification (12 mg, 35%) of the title compound. ¹H NMR (500 MHz, DMSO-d₆) δ 8.24 (d, J=2.3 Hz, 1H), 7.9-8.0 (m, 2H), 7.58 (br d, J=2.1 Hz, 1H), 7.5-7.5 (m, 4H), 6.68 (br d, J=8.8 Hz, 2H), 5.72 (br s, 2H), 4.10 (quin, J=7.1 Hz, 1H), 3.62 (br d, J=6.7 Hz, 2H), 3.4-3.5 (m, 5H), 3.06 (s, 3H), 2.96 (br t, J=6.4 Hz, 2H), 2.39 (q, J=7.1 Hz, 2H); LCMS: [M+H]⁺=463.2.

### Example 10: (S)-N-(1-(4-(5-amino-6-(1-oxo-1,2,3,4-tetrahydroisoquinolin-6-yl)pyrazin-2-yl)phenyl)pyrrolidin-3-yl)-N-methylmethanesulfonamide (I-10)

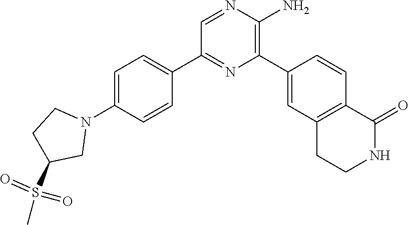 [SMILES: CS(=O)(=O)[C@H]1CCN(c2ccc(-c3cnc(N)c(-c4ccc5c(c4)CCNC5=O)n3)cc2)C1]

### Step 1: (S)-N-(1-(4-chlorophenyl)pyrrolidin-3-yl)-N-methylmethanesulfonamide

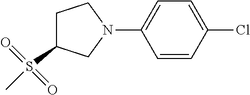 [SMILES: CS(=O)(=O)[C@H]1CCN(c2ccc(Cl)cc2)C1]

A procedure similar to that described in Example 7, Step 1 using 1-chloro-4-iodobenzene (0.250 g, 1.048 mmol), tris(dibenzylideneacetone)dipalladium (0) (0.029 g, 0.031 mmol), 4,5-bis(diphenylphosphino)-9,9-dimethylxanthene (0.055 g, 0.094 mmol) and Cs₂CO₃ (1.025 g, 3.15 mmol) afforded the product (149 mg, 55%). LCMS: [M+H]⁺=288.91.

### Step 2: (S)-N-methyl-N-(1-(4-(4,4,5,5-tetramethyl-1,3,2-dioxaborolan-2-yl)phenyl)pyrrolidin-3-yl)methanesulfonamide

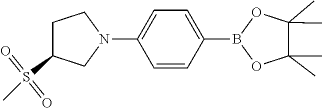

A procedure analogous to that of Example 7, Step 2 using (S)-N-(1-(4-chlorophenyl)pyrrolidin-3-yl)-N-methylmethanesulfonamide (0.166 g, 0.575 mmol), bis(pinacolato)diboron (0.219 g, 0.862 mmol), KOAc (0.113 g, 1.15 mmol) and XPhos Pd G2 (0.034 g, 0.043 mmol) afforded the product (168 mg, 77%). LCMS: [M+H]+=381.05.

### Step 3: S)-N-(1-(4-(5-amino-6-(1-oxo-1,2,3,4-tetrahydroisoquinolin-6-yl)pyrazin-2-yl)phenyl)pyrrolidin-3-yl)-N-methylmethanesulfonamide

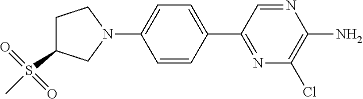 [SMILES: CS(=O)(=O)[C@H]1CCN(c2ccc(-c3cnc(N)c(Cl)n3)cc2)C1]

A procedure analogous to Example 7, Step 3 using 2-amino-5-bromo-3-chloropyrazine (0.057 g, 0.275 mmol), (S)-N-methyl-N-(1-(4-(4,4,5,5-tetramethyl-1,3,2-dioxaborolan-2-yl)phenyl)pyrrolidin-3-yl)methanesulfonamide (0.087 g, 0.229 mmol), [1,1′-bis(diphenylphosphino)ferrocene]dichloropalladium(II) (0.017 g, 0.023 mmol) and Cs₂CO₃ (0.186 g, 0.572 mmol) afforded the product (66 mg, 76%). LCMS: [M+H]⁺=382.25.

### Step 4: S)-N-(1-(4-(5-amino-6-(1-oxo-1,2,3,4-tetrahydroisoquinolin-6-yl)pyrazin-2-yl)phenyl)pyrrolidin-3-yl)-N-methylmethanesulfonamide

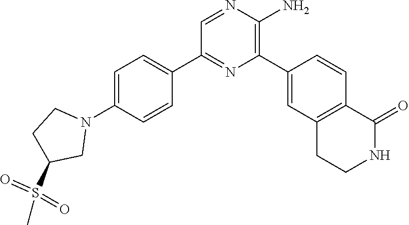 [SMILES: CS(=O)(=O)[C@H]1CCN(c2ccc(-c3cnc(N)c(-c4ccc5c(c4)CCNC5=O)n3)cc2)C1]

A procedure analogous to Example 7, Step 4 using (S)-N-(1-(4-(5-amino-6-chloropyrazin-2-yl)phenyl)pyrrolidin-3-yl)-N-methylmethanesulfonamide (0.066 g, 0.173 mmol), 3,4-dihydro-1(2H)-isoquinolinone-6-boronic acid pinacol ester (0.057 g, 0.207 mmol) and XPhos Pd G2 (0.014 g, 0.017 mmol) afforded the title compound (9 mg, 24%). ¹H NMR (500 MHz, DMSO-d₆) δ 8.25 (d, J=2.3 Hz, 1H), 7.9-8.0 (m, 2H), 7.58 (br d, J=2.2 Hz, 1H), 7.5-7.5 (m, 4H), 6.69 (br d, J=8.7 Hz, 2H), 5.69 (br s, 2H), 4.1-4.1 (m, 1H), 3.63 (br d, J=7.0 Hz, 2H), 3.4-3.5 (m, 6H), 3.07 (s, 3H), 2.97 (br t, J=6.5 Hz, 2H), 2.4-2.4 (m, 3H); LCMS: [M+H]⁺=463.3.

### Example 11: (R)-6-(3-amino-6-(4-(3-methylmorpholino)phenyl)pyrazin-2-yl)-3,4-dihydroisoquinolin-1(2H)-one (I-11)

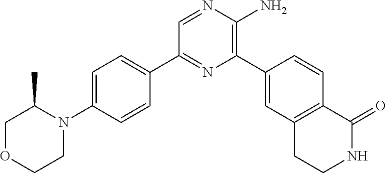 [SMILES: C[C@@H]1COCCN1c1ccc(-c2cnc(N)c(-c3ccc4c(c3)CCNC4=O)n2)cc1]

### Step 2: (R)-4-(4-chlorophenyl)-3-methylmorpholine

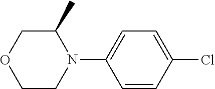

A procedure analogous to Example 7, Step 1 using 1-chloro-4-iodobenzene (0.500 g, 2.097 mmol), tris(dibenzylideneacetone)dipalladium (0) (0.058 g, 0.063 mmol), 4,5-bis(diphenylphosphino)-9,9-dimethylxanthene (0.109 g, 0.189 mmol) and Cs₂CO₃ (2.05 g, 6.29 mmol) afforded the product (150 mg, 34%). LCMS: [M+H]⁺=212.29.

### Step 2: (R)-3-methyl-4-(4-(4,4,5,5-tetramethyl-1,3,2-dioxaborolan-2-yl)phenyl)morpholine

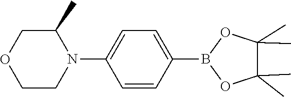 [SMILES: C[C@@H]1COCCN1c1ccc(B2OC(C)(C)C(C)(C)O2)cc1]

Following a procedure analagous to Example 7, Step 2 using (R)-4-(4-chlorophenyl)-3-methylmorpholine (0.120 g, 0.567 mmol), bis(pinacolato)diboron (0.180 g, 0.709 mmol), KOAc (0.111 g, 1.13 mmol) and XPhos Pd G2 (0.033 g, 0.043 mmol) afforded the product (127 mg, 74%). LCMS: [M+H]⁺=304.41.

### Step 3: (R)-3-chloro-5-(4-(3-methylmorpholino)phenyl)pyrazin-2-amine

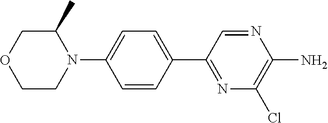 [SMILES: C[C@@H]1COCCN1c1ccc(-c2cnc(N)c(Cl)n2)cc1]

Using a procedure analogous to Example 7, Step 3 with 2-amino-5-bromo-3-chloropyrazine (0.103 g, 0.495 mmol), (R)-3-methyl-4-(4-(4,4,5,5-tetramethyl-1,3,2-dioxaborolan-2-yl)phenyl)morpholine (0.125 g, 0.412 mmol), [1,1′-bis(diphenylphosphino)ferrocene]dichloropalladium(II) (0.030 g, 0.041 mmol) and Cs₂CO₃ (0.336 g, 1.031 mmol) afforded the product (89 mg, 71%). LCMS [M+H]⁺=305.31.

### Step 4: (R)-6-(3-amino-6-(4-(3-methylmorpholino)phenyl)pyrazin-2-yl)-3,4-dihydroisoquinolin-1(2H)-one

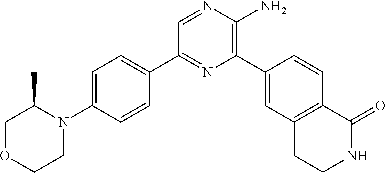 [SMILES: C[C@@H]1COCCN1c1ccc(-c2cnc(N)c(-c3ccc4c(c3)CCNC4=O)n2)cc1]

A procedure analogous to Example 7, Step 4 using (R)-3-chloro-5-(4-(3-methylmorpholino)phenyl)pyrazin-2-amine (0.044 g, 0.144 mmol), 3,4-dihydro-1(2H)-isoquinolinone-6-boronic acid pinacol ester (0.047 g, 0.173 mmol) and XPhos Pd G2 (0.011 g, 0.014 mmol) afforded the title compound (15 mg, 25%)¹H NMR (500 MHz, DMSO-d₆) δ 8.49 (s, 1H), 7.9-8.0 (m, 2H), 7.86 (d, J=8.9 Hz, 2H), 7.75 (dd, J=8.0, 1.7 Hz, 1H), 7.71 (s, 1H), 6.96 (d, J=8.9 Hz, 2H), 6.22 (s, 2H), 3.93 (br d, J=7.1 Hz, 2H), 3.7-3.8 (m, 2H), 3.57 (dt, J=11.3, 3.0 Hz, 1H), 3.43 (dt, J=6.6, 2.6 Hz, 2H), 3.2-3.3 (m, 1H), 3.0-3.1 (m, 3H), 1.02 (d, J=6.5 Hz, 3H); LCMS: [M+H]⁺=416.5.

### Example 12: 6-(3-amino-6-(4-(4-methylpiperazin-1-yl)phenyl)pyrazin-2-yl)-7-fluoro-3,4-dihydroisoquinolin-1(2H)-one (I-12)

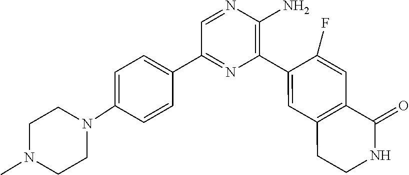 [SMILES: CN1CCN(c2ccc(-c3cnc(N)c(-c4cc5c(cc4F)C(=O)NCC5)n3)cc2)CC1]

### Step 1: 6-bromo-7-fluoro-3,4-dihydroisoquinolin-1(2H)-one

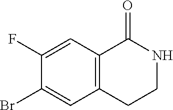 [SMILES: O=C1NCCc2cc(Br)c(F)cc21]

NaN₃ (114 mg, 1.746 mmol, 2 eq) was added to a solution of 5-bromo-6-fluoro-2,3-dihydro-1H-inden-1-one (200 mg, 0.873 mmol, 1 eq) in 0.4 mL of a mixture of methane sulphonic acid and CH₂Cl₂ (1:1) portion wise at 0° C. The resulting mixture was stirred for 8 h at RT. The reaction mixture was cooled to 0° C. in ice bath, neutralized with 5% aq. NaOH and aqueous layer was extracted with EtOAc (2×10 mL). The combined organic layers were washed with H₂O and brine solution, dried over MgSO₄ and filtered. The filtrate was concentrated under vacuum and purified by silica gel flash column chromatography using CH₂Cl₂-MeOH to obtain the product as white solid (150 mg, 71%); LCMS: [M+H]⁺=246.20

### Step 2: 8-fluoro-6-(4,4,5,5-tetramethyl-1,3,2-dioxaborolan-2-yl)-3,4-dihydroiso-quinolin-1(2H)-one

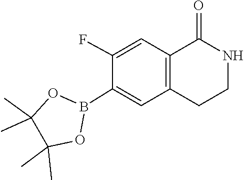 [SMILES: CC1(C)OB(c2cc3c(cc2F)C(=O)NCC3)OC1(C)C]

To 6-bromo-5-fluoro-3,4-dihydroisoquinolin-1(2H)-one (50 mg, 0.205 mmol, 1 eq), bis(pinacolato)diboron (58.2 mg, 0.229 mmol, 1.1 eq), KOAc (61.3 mg, 0.625 mmol, 3 eq) and [1,12-bis(diphenylphosphino)ferrocene]dichloropalladium(II) (7.62 mg, 10.41 μmol, 0.05 eq) in 1,4-dioxane (2.5 mL) was added under Ar. The mixture was heated in a microwave for 2 h at 100° C. The LCMS showed less than 10% of starting material. The reaction mixture was used in the next steps without purification. LCMS: [M+H]⁺=292.0

### Step 3 6-(3-amino-6-(4-(4-methylpiperazin-1-yl)phenyl)pyrazin-2-yl)-7-fluoro-3,4-dihydroisoquinolin-1(2H)-one, Formic acid salt

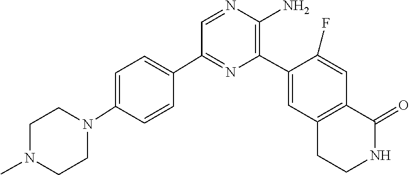 [SMILES: CN1CCN(c2ccc(-c3cnc(N)c(-c4cc5c(cc4F)C(=O)NCC5)n3)cc2)CC1]

XPhos Pd G2 (14.74 mg, 0.019 mmol, 0.1 eq), K₃PO₄ tribasic reagent grade (119 mg, 0.562 mmol, 3 eq), and 3-bromo-5-(4-(4-methylpiperazin-1-yl)phenyl)pyrazin-2-amine (65.2 mg, 0.187 mmol, 1 eq) was dissolved in a mixture of H₂O (4 mL) and ACN (6 mL) in the microwave vial. The reaction was degassed with N₂. 7-Fluoro-6-(4,4,5,5-tetramethyl-1,3,2-dioxaborolan-2-yl)-3,4-dihydroisoquinolin-1(2H)-one (60 mg, 0.206 mmol, 1.1 eq) in dioxane (4 mL) was added and the mixture was degassed again. The vial was sealed and heated overnight at 100° C. The resulted mixture was dried with Celite and purified with reverse phase flash chromatography (water/ACN) to obtain the formic acid salt of the title compound as a dark yellow solid (10 mg, 11%). ¹H NMR (500 MHz, CDCl₃) δ 8.41 (s, 1H), 7.84 (d, J=10.3 Hz, 1H), 7.78 (d, J=7.9 Hz, 2H), 7.47-7.40 (m, 1H), 6.92 (d, J=8.2 Hz, 2H), 5.96-5.89 (m, 1H), 4.58 (s, 2H), 3.55 (dt, J=6.6, 2.8 Hz, 2H), 3.32-3.24 (m, 4H), 3.02-2.97 (m, 2H), 2.73-2.62 (m, 4H), 2.42-2.35 (m, 3H); LCMS: [M+H]⁺=433.41.

### Example 13: (R)-6-(3-amino-6-(2-fluoro-4-(2-isopropylmorpholino)phenyl)pyrazin-2-yl)-3,4-dihydroisoquinolin-1(2H)-one (I-13)

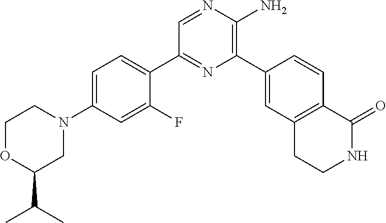 [SMILES: CC(C)[C@@H]1CN(c2ccc(-c3cnc(N)c(-c4ccc5c(c4)CCNC5=O)n3)c(F)c2)CCO1]

### Step 1: (R)-4-(4-chloro-3-fluorophenyl)-2-isopropylmorpholine

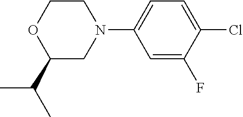 [SMILES: CC(C)[C@@H]1CN(c2ccc(Cl)c(F)c2)CCO1]

A 30 mL vial was charged with 4-bromo-1-chloro-2-fluorobenzene (0.15 g, 0.72 mmol), (R)-2-isopropylmorpholine (0.10 g, 0.79 mmol), tris(dibenzylideneacetone)dipalladium (0) (0.020 g, 0.021 mmol), 4,5-bis(diphenylphosphino)-9,9-dimethylxanthene (0.037 g, 0.064 mmol) and Cs₂CO₃ (0.70 g, 2.1 mmol). The vial was sealed with a cap and septum and then the reaction vial was evacuated and backfilled with nitrogen. Toluene (2.5 mL) was added and the reaction vial was evacuated and backfilled with nitrogen an additional time. The reaction was heated conventionally at 100° C. for 18 h. The reaction mixture was cooled to RT and partitioned between EtOAc and water. The layers were separated and the aqueous layer was extracted with additional EtOAc (×2). The combined organic extracts were dried and concentrated onto celite. Flash chromatography (1-15% EtOAc/hexanes) afforded the product (0.18 g, 99%). LCMS: [M+H]⁺=258.2.

### Step 2: (R)-4-(3-fluoro-4-(4,4,5,5-tetramethyl-1,3,2-dioxaborolan-2-yl)phenyl)-2-isopropylmorpholine

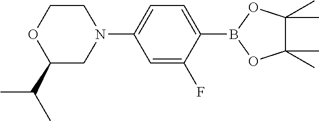 [SMILES: CC(C)[C@@H]1CN(c2ccc(B3OC(C)(C)C(C)(C)O3)c(F)c2)CCO1]

A 30 mL vial was charged with (R)-4-(4-chloro-3-fluorophenyl)-2-isopropylmorpholine (0.67 g, 2.6 mmol), bis(pinacolato)diboron (0.83 g, 3.3 mmol), KOAc (0.51 g, 5.2 mmol) and XPhos Pd G2 (0.15 g, 0.20 mmol). The vial was sealed with a cap and septum and then the reaction vial was evacuated and backfilled with N₂. 1,4-Dioxane (8 mL) was added and the reaction vial was evacuated and backfilled with nitrogen an additional time. The reaction was heated at 90° C. for 18 h. After cooling to RT the reaction mixture was concentrated directly onto Celite and purified by flash chromatography (1-15% EtOAc/hexanes) to afford the product (0.92 g, quantitative yield). LCMS: [M+H]⁺=350.2.

### Step 3: (R)-6-(3-amino-6-(2-fluoro-4-(2-isopropylmorpholino)phenyl)pyrazin-2-yl)-3,4-dihydroisoquinolin-1(2H)-one

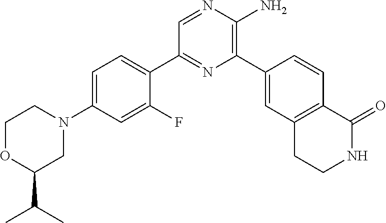 [SMILES: CC(C)[C@@H]1CN(c2ccc(-c3cnc(N)c(-c4ccc5c(c4)CCNC5=O)n3)c(F)c2)CCO1]

A 30 mL vial was charged with (R)-4-(3-fluoro-4-(4,4,5,5-tetramethyl-1,3,2-dioxaborolan-2-yl)phenyl)-2-isopropylmorpholine (0.033 g, 0.094 mmol), 6-(3-amino-6-bromopyrazin-2-yl)-3,4-dihydroisoquinolin-1(2H)-one (0.025 g, 0.078 mmol) and XPhos Pd G2 (0.014 g, 0.018 mmol). The vial was sealed with a cap and septum and the reaction vial was evacuated and backfilled with nitrogen. 1,4-Dioxane (1 mL) and 1.3 M aqueous K₃PO₄ (0.20 mL, 0.26 mmol) were added and the reaction vial was evacuated and backfilled with nitrogen an additional time. The reaction was heated conventionally at 100° C. for 18 h. After cooling to RT the reaction mixture was concentrated directly onto Celite and purified by flash chromatography [0.5-5.0% MeOH/CH₂Cl₂+0.5% NH₄OH] to afford the title compound (19 mg, 53%). ¹H NMR (500 MHz, DMSO-d₆) δ 8.32 (d, J=2.4 Hz, 1H), 7.98 (br s, 1H), 7.95 (d, J=8.1 Hz, 1H), 7.78 (t, J=9.2 Hz, 1H), 7.73 (dd, J=8.0, 1.7 Hz, 1H), 7.69 (s, 1H), 6.8-6.9 (m, 2H), 6.37 (s, 2H), 3.97 (dd, J=10.8, 2.9 Hz, 1H), 3.6-3.7 (m, 4H), 3.42 (dt, J=6.6, 2.8 Hz, 3H), 3.23 (ddd, J=10.4, 6.2, 2.4 Hz, 1H), 2.99 (br t, J=6.5 Hz, 2H), 2.7-2.8 (m, 1H), 1.73 (qd, J=13.4, 6.7 Hz, 1H), 0.96 (d, J=6.7 Hz, 6H); LCMS: [M+H]⁺=462.4.

### Example 14: (S)-6-(3-amino-6-(2-fluoro-4-(2-isopropylmorpholino)phenyl)pyrazin-2-yl)-3,4-dihydroisoquinolin-1(2H)-one (I-14)

### Step 1: 1-(4-bromophenyl)-3-chloropropan-1-one (2)

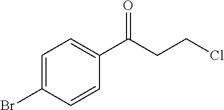 [SMILES: O=C(CCCl)c1ccc(Br)cc1]

To a stirred solution of AlCl₃ (47 g, 352.5 mmol) in CH₂Cl₂ (500 mL), was added bromobenzene (33.3 mL, 320.5 mmol) and 3-chloropropanoyl chloride (31.30 mL, 320.5 mmol) in CH₂Cl₂ (400 mL) drop wise at 0° C. The reaction mixture was stirred at RT for 16 h. The mixture was quenched with ice H₂O (500 mL) and extracted with CH₂Cl₂ (3×300 mL). The combined organic layer was dried over Na₂SO₄ and concentrate under reduced pressure gave crude product (75 g, 95%) as a pale yellow semi solid. TLC: 20% EA in Pet ether; R_{f}=0.4.

### Step 2: 5-Bromo-2,3-dihydro-1H-inden-1-one

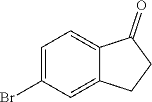 [SMILES: O=C1CCc2cc(Br)ccc21]

To a stirred solution of 1-(4-bromophenyl)-3-chloropropan-1-one (70 g, 71.6 mmol) in H₂SO₄ (700 mL) at RT. The mixture was stirred at 100° C. for 4 h before cooling to RT. The reaction mixture was diluted with ice H₂O and extracted with EtOAc (3×200 mL). The combined organic layer was washed with brine (200 mL), dried over with Na₂SO₄, filtered and concentrated under reduced pressure to afford the crude product (40 g crude) as a brown solid. This material was used in the next step without further purification. TLC: 20% EA in Pet ether; R_{f}=0.5.

### Step 3: 6-bromo-3,4-dihydroisoquinolin-1(2H)-one

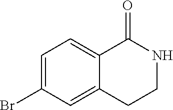 [SMILES: O=C1NCCc2cc(Br)ccc21]

To a stirred solution of 5-bromo-2,3-dihydro-1H-inden-1-one (30 g, 142.8 mmol) in CH₂Cl₂ (240 mL) was added MsOH (120 mL) at 0° C. NaN₃ (32.5 g, 500 mmol) was then added portionwise. The reaction mixture was stirred for 30 min at RT. The reaction mixture was basified with 20% NaOH solution and extracted with CH₂Cl₂ (2×500 mL), the combined organic layers were dried over Na₂SO₄ and concentrated to give crude product which was purified by column chromatography (100-200 silica gel) using 0-7% MeOH in CH₂Cl₂ as an eluent to afford the product (11.5 g, 35%) as a pale yellow solid. LCMS: [M+H]⁺=225.96.

### Step 4: 3,5-dibromopyrazin-2-amine

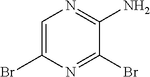

To a stirred solution of pyrazin-2-amine (10 g, 105.2 mmol) in DMSO (100 mL) was added NBS (37.4 g, 210.5 mmol) at RT. The reaction mixture was then stirred at RT for 4 h, diluted with H₂O (200 mL) and extracted with EtOAc (3×200 mL). The combined organic layers were washed with brine solution (200 mL), dried over with Na₂SO₄, filtered and concentrated under reduced pressure to give crude compound which was purified by column chromatography (100-200 mesh, silica gel) using 0-40% EtOAc in pet ether as an eluent to afford the product (12 g, 46%) as a pale orange solid. TLC: 50% EtOAc in pet ether; R_{f}=0.5.

### Step 5: 6-(3-amino-6-bromopyrazin-2-yl)-3,4-dihydroisoquinolin-1(2H)-one

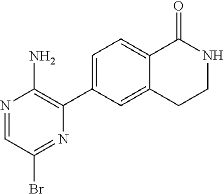 [SMILES: Nc1ncc(Br)nc1-c1ccc2c(c1)CCNC2=O]

To a stirred solution of 6-bromo-3,4-dihydroisoquinolin-1(2H)-one (1.7 g, 7.5 mmol) and 3,5-dibromopyrazin-2-amine (2.8 g, 11.3 mmol) in DMF:H₂O (20:1, 20 mL) was added K₂CO₃ (2 g, 15.1 mmol)) and bubbling with argon for 15 min at RT. Then PdCl₂(PPh₃)₂ (0.053 g, 0.075 mmol) was added and the reaction mixture was heated at 90° C. for 16 h, diluted with H₂O (100 mL) and extracted with EtOAc (3×200 mL). The combined organic layers were washed with brine (200 mL), dried over with Na₂SO₄, filtered and concentrated under reduced pressure to give crude compound. Purification by column chromatography (100-200 mesh silica gel) using 0-10% MeOH in CH₂Cl₂ as an eluent afforded the product (0.7 g, 35.0%) as a brick red solid. ¹H NMR (400 MHz, DMSO-d₆): δ 8.11 (s, 1H), δ 8.01 (brs, 1H), δ 7.96 (d, J=7.6 Hz, 1H), δ 7.63 (d, J=7.6 Hz, 1H), δ 7.60 (s, 1H), δ 6.56 (brs, 2H), 3.42-3.33 (m, 2H), δ 2.97 (t, J=6.4 Hz, 2H); LCMS: [M+H]⁺=319.26

### Step 6: (S)-4-(4-chloro-3-fluorophenyl)-2-isopropylmorpholine

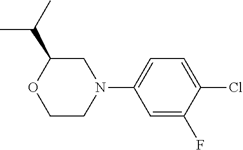 [SMILES: CC(C)[C@H]1CN(c2ccc(Cl)c(F)c2)CCO1]

A procedure analogous to Example 7, Step 1 using (S)-2-Isopropylmorpholine (0.339 g, 2.63 mmol), 4-bromo-1-chloro-2-fluorobenzene (0.500 g, 2.387 mmol), tris(dibenzylideneacetone)dipalladium (0) (0.066 g, 0.072 mmol), 4,5-bis(diphenylphosphino)-9,9-dimethylxanthene (0.124 g, 0.215 mmol) and Cs₂CO₃ (2.33 g, 7.16 mmol) afforded the product (782 mg, quant. yield). LCMS: [M+H]⁺=258.15.

### Step 7: (S)-4-(3-fluoro-4-(4,4,5,5-tetramethyl-1,3,2-dioxaborolan-2-yl)phenyl)-2-isopropylmorpholine

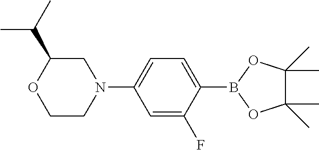 [SMILES: CC(C)[C@H]1CN(c2ccc(B3OC(C)(C)C(C)(C)O3)c(F)c2)CCO1]

A procedure analogous to Example 7, Step 2 using (S)-4-(4-chloro-3-fluorophenyl)-2-isopropylmorpholine (0.782 g, 3.03 mmol), bis(pinacolato)diboron (0.963 g, 3.79 mmol), KOAc (0.596 g, 6.07 mmol) and XPhos Pd G2 (0.179 g, 0.228 mmol) afforded the product (810 mg, 76%). LCMS: [M+H]⁺=350.21.

### Step 8: (S)-6-(3-amino-6-(2-fluoro-4-(2-isopropylmorpholino)phenyl)pyrazin-2-yl)-3,4-dihydroisoquinolin-1(2H)-one

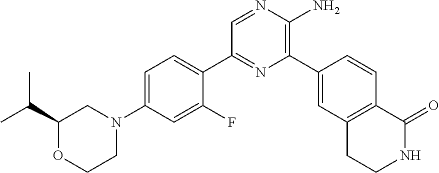 [SMILES: CC(C)[C@H]1CN(c2ccc(-c3cnc(N)c(-c4ccc5c(c4)CCNC5=O)n3)c(F)c2)CCO1]

In a 30 mL vial with magnetic stir bar was charged (S)-4-(3-fluoro-4-(4,4,5,5-tetramethyl-1,3,2-dioxaborolan-2-yl)phenyl)-2-isopropylmorpholine (0.033 g, 0.094 mmol), 6-(3-amino-6-bromopyrazin-2-yl)-3,4-dihydroisoquinolin-1(2H)-one (0.025 g, 0.078 mmol) and XPhos Pd G2 (0.014 g, 0.018 mmol). The vial was sealed with a cap and septum and the reaction vessel was evacuated and backfilled with nitrogen. 1,4-Dioxane (1 mL) and aqueous K₃PO₄ (0.196 mL of a 1.3 M solution, 0.255 mmol) were added and the reaction vessel was evacuated and backfilled with nitrogen an additional time. The reaction mixture was heated at 100° C. for 22 h in an aluminum block, then was concentrated onto Celite and purified by flash chromatography (0.5%-5% MeOH/CH₂Cl₂+0.5% NH₄OH) to afford the title compound (20 mg, 55%). ¹H NMR (500 MHz, DMSO-d₆) δ 8.32 (d, J=2.4 Hz, 1H), 7.98 (br s, 1H), 7.95 (d, J=8.1 Hz, 1H), 7.78 (t, J=9.1 Hz, 1H), 7.73 (dd, J=8.0, 1.3 Hz, 1H), 7.69 (s, 1H), 6.8-6.9 (m, 2H), 6.37 (s, 2H), 3.97 (dd, J=10.5, 3.1 Hz, 1H), 3.6-3.7 (m, 4H), 3.42 (dt, J=6.5, 2.8 Hz, 3H), 3.2-3.3 (m, 1H), 2.99 (t, J=6.5 Hz, 2H), 2.7-2.8 (m, 1H), 1.73 (qd, J=13.4, 6.8 Hz, 1H), 0.96 (d, J=6.7 Hz, 6H); LCMS: [M+H]⁺=462.4.

### Example 15: (R)-6-(3-amino-6-(2,3-difluoro-4-(2-isopropylmorpholino)phenyl)pyrazin-2-yl)-3,4-dihydroisoquinolin-1(2H)-one (I-15)

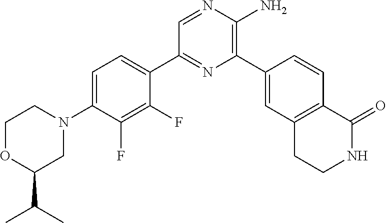 [SMILES: CC(C)[C@@H]1CN(c2ccc(-c3cnc(N)c(-c4ccc5c(c4)CCNC5=O)n3)c(F)c2F)CCO1]

### Step 1: (R)-4-(2,3-difluorophenyl)-2-isopropylmorpholine

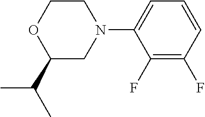 [SMILES: CC(C)[C@@H]1CN(c2cccc(F)c2F)CCO1]

A 30 mL vial was charged with 1-bromo-2,3-difluorobenzene (0.15 g, 0.78 mmol), (R)-2-isopropylmorpholine (0.11 g, 0.86 mmol), tris(dibenzylideneacetone)dipalladium (0) (0.021 g, 0.023 mmol), 4,5-bis(diphenylphosphino)-9,9-dimethylxanthene (0.040 g, 0.070 mmol) and Cs₂CO₃ (0.76 g, 2.3 mmol). The vial was sealed with a cap and septum and then the reaction vial was evacuated and backfilled with N₂. Toluene (2.5 mL) was added and the reaction vial was evacuated and backfilled with nitrogen an additional time. The reaction was heated conventionally at 100° C. for 18 h. The reaction mixture was cooled to RT and partitioned between EtOAc and water. The layers were separated and the aqueous layer was extracted with additional EtOAc (×2). The combined organic extracts were dried and concentrated onto celite. Flash chromatography (1-15% EtOAc/hexanes) afforded the product (0.11 g, 60%). LCMS: [M+H]⁺=241.9.

### Step 2: (R)-4-(4-bromo-2,3-difluorophenyl)-2-isopropylmorpholine

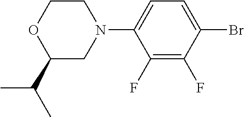 [SMILES: CC(C)[C@@H]1CN(c2ccc(Br)c(F)c2F)CCO1]

N-Bromosuccinimide (0.091 g, 0.51 mmol) was added to a stirring solution of (R)-4-(2,3-difluorophenyl)-2-isopropylmorpholine (0.11 g, 0.46 mmol) in CH₂Cl₂ (2 mL) at 0° C. The reaction was allowed to gradually warm to RT overnight. The reaction mixture was concentrated directly onto Celite and purified by flash chromatography (1-10% EtOAc/hexanes) to afford the product (0.11 g, 77%). LCMS: [M+H]⁺=320.1.

### Step 3: (R)-4-(2,3-difluoro-4-(4,4,5,5-tetramethyl-1,3,2-dioxaborolan-2-yl)phenyl)-2-isopropylmorpholine

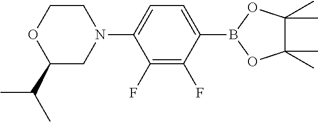 [SMILES: CC(C)[C@@H]1CN(c2ccc(B3OC(C)(C)C(C)(C)O3)c(F)c2F)CCO1]

A 30 mL vial was charged with (R)-4-(4-bromo-2,3-difluorophenyl)-2-isopropylmorpholine (0.44 g, 1.4 mmol), bis(pinacolato)diboron (0.44 g, 1.7 mmol), KOAc (0.27 g, 2.7 mmol) and XPhos Pd G2 (0.08 g, 0.10 mmol). The vial was sealed with a cap and septum and then the reaction vial was evacuated and backfilled with N₂. 1,4-Dioxane (8 mL) was added and the reaction vial was evacuated and backfilled with N₂ an additional time. The reaction was heated conventionally at 90° C. for 18 h. After cooling to RT the reaction mixture was concentrated directly onto Celite and purified by flash chromatography (1-15% EtOAc/hexanes) to afford the product (0.26 g, 51%). LCMS: [M+H]⁺=368.2.

### Step 4: (R)-6-(3-amino-6-(2,3-difluoro-4-(2-isopropylmorpholino)phenyl)pyrazin-2-yl)-3,4-dihydroisoquinolin-1(2H)-one

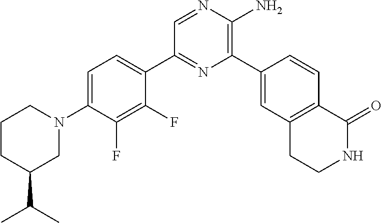 [SMILES: CC(C)[C@H]1CCCN(c2ccc(-c3cnc(N)c(-c4ccc5c(c4)CCNC5=O)n3)c(F)c2F)C1]

A 30 mL vial was charged with (R)-4-(2,3-difluoro-4-(4,4,5,5-tetramethyl-1,3,2-dioxaborolan-2-yl)phenyl)-2-isopropylmorpholine (0.035 g, 0.094 mmol), 6-(3-amino-6-bromopyrazin-2-yl)-3,4-dihydroisoquinolin-1(2H)-one (0.025 g, 0.078 mmol, prepared according to procedure in Example 14, Step 5) and XPhos Pd G2 (0.014 g, 0.018 mmol). The vial was sealed with a cap and septum and the reaction vial was evacuated and backfilled with N₂. 1,4-Dioxane (1 mL) and aqueous K₃PO₄ (0.20 mL of a 1.3 M solution, 0.26 mmol) were added and the reaction vial was evacuated and backfilled with N₂ an additional time. The reaction was heated at 100° C. for 18 h. After cooling to RT the reaction mixture was concentrated directly onto Celite and purified by flash chromatography (0.5-5.0% MeOH/CH₂Cl₂+0.5% NH₄OH) to afford the title compound (240 mg, 64%). ¹H NMR (500 MHz, DMSO-d₆) δ 8.34 (d, J=2.6 Hz, 1H), 7.99 (br s, 1H), 7.95 (d, J=7.9 Hz, 1H), 7.73 (dd, J=8.0 Hz, 1.7, 1H), 7.69 (s, 1H), 7.62 (dt, J=8.6, 1.7 Hz, 1H), 6.98 (t, J=8.0 Hz, 1H), 6.54 (s, 2H), 3.9-4.0 (m, 1H), 3.67 (dt, J=11.4, 2.4 Hz, 1H), 3.42 (dt, J=6.5, 2.8 Hz, 2H), 3.3-3.4 (m, 2H), 3.2-3.3 (m, 1H), 2.99 (t, J=6.5 Hz, 2H), 2.83 (dt, J=11.7, 3.2 Hz, 1H), 2.61 (dd, J=11.4, 10.5 Hz, 1H), 1.71 (qd, J=13.4, 6.7 Hz, 1H), 0.93 (dd, J=16.0, 6.8 Hz, 6H); LCMS: [M+H]⁺=480.4.

### Example 16: (S)-6-(3-amino-6-(2,3-difluoro-4-(2-isopropylmorpholino)phenyl)pyrazin-2-yl)-3,4-dihydroisoquinolin-1(2H)-one (I-16)

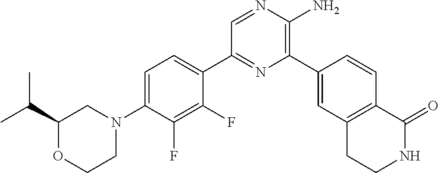 [SMILES: CC(C)[C@H]1CN(c2ccc(-c3cnc(N)c(-c4ccc5c(c4)CCNC5=O)n3)c(F)c2F)CCO1]

### Step 1: (S)-4-(2,3-difluorophenyl)-2-isopropylmorpholine

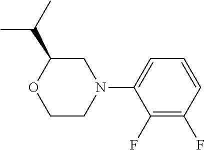 [SMILES: CC(C)[C@H]1CN(c2cccc(F)c2F)CCO1]

A 30 mL vial was charged with (S)-2-isopropylmorpholine (0.331 g, 2.56 mmol), 1-bromo-2,3-difluorobenzene (0.450 g, 2.332 mmol), tris(dibenzylideneacetone)dipalladium (0) (0.064 g, 0.070 mmol), 4,5-bis(diphenylphosphino)-9,9-dimethylxanthene (0.121 g, 0.210 mmol) and Cs₂CO₃ (2.279 g, 7.00 mmol). The vial was sealed with a cap and septum and then the reaction vessel was evacuated and backfilled with nitrogen. Toluene (6 mL) was added and the reaction vessel was evacuated and backfilled with nitrogen an additional time. The reaction was heated at 100° C. for 18 h. The reaction mixture was cooled to RT and partitioned between EtOAc and water. The layers were separated and the aqueous layer was extracted with additional EtOAc (×2). LCMS [01] indicated clean conversion to the desired product. After concentrating the combined extracts to dryness the crude product was passed through a short plug of silica gel eluting with 5% EtOAc/hexanes to afford, after removal of volatiles, the product (628 mg, quantitative yield). LCMS: [M+H]⁺=242.21

### Step 2: (S)-4-(4-bromo-2,3-difluorophenyl)-2-isopropylmorpholine

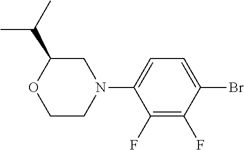 [SMILES: CC(C)[C@H]1CN(c2ccc(Br)c(F)c2F)CCO1]

NBS (0.519 g, 2.91 mmol) was added to a stirring solution of (S)-4-(2,3-difluorophenyl)-2-isopropylmorpholinein in CH₂Cl₂ (6 mL) at RT and the mixture was stirred for 14 h. Standard workup afforded the product (747 mg, 71%). LCMS: [M+H]⁺=320.2.

### Step 2: (S)-4-(2,3-difluoro-4-(4,4,5,5-tetramethyl-1,3,2-dioxaborolan-2-yl)phenyl)-2-isopropylmorpholine

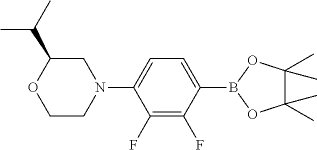 [SMILES: CC(C)[C@H]1CN(c2ccc(B3OC(C)(C)C(C)(C)O3)c(F)c2F)CCO1]

A procedure similar to Example 7, Step 2 using (S)-4-(4-bromo-2,3-difluorophenyl)-2-isopropylmorpholine (0.528 g, 1.65 mmol), bis(pinacolato)diboron (0.523 g, 2.06 mmol), KOAC (0.324 g, 3.30 mmol) and XPhos Pd G2 (0.097 g, 0.124 mmol) afforded the product (143 mg, 24%). LCMS: [M+H]⁺=368.26 Step 4: (S)-6-(3-amino-6-(2,3-difluoro-4-(2-isopropylmorpholino)phenyl)pyrazin-2-yl)-3,4-dihydroisoquinolin-1(2H)-one

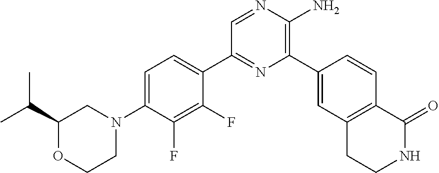 [SMILES: CC(C)[C@H]1CN(c2ccc(-c3cnc(N)c(-c4ccc5c(c4)CCNC5=O)n3)c(F)c2F)CCO1]

A procedure analogous to that used in Example 7, Step 4 using (S)-4-(2,3-difluoro-4-(4,4,5,5-tetramethyl-1,3,2-dioxaborolan-2-yl)phenyl)-2-isopropylmorpholine (0.035 g, 0.094 mmol), 6-(3-amino-6-bromopyrazin-2-yl)-3,4-dihydroisoquinolin-1(2H)-one (0.025 g, 0.078 mmol) and XPhos Pd G2 (0.014 g, 0.018 mmol) afforded the title compound (15 mg, 40%). ¹H NMR (500 MHz, DMSO-d₆) δ 8.34 (d, J=2.3 Hz, 1H), 7.99 (br s, 1H), 7.95 (d, J=7.9 Hz, 1H), 7.72 (d, J=8.1 Hz, 1H), 7.69 (s, 1H), 7.62 (br t, J=8.4 Hz, 1H), 6.98 (br t, J=8.3 Hz, 1H), 6.54 (s, 2H), 3.9-4.0 (m, 1H), 3.67 (dt, J 11.3, 2.2 Hz, 1H), 3.42 (td, J=6.5, 3.2 Hz, 3H), 3.36 (br d, J=12.0 Hz, 2H), 3.27 (br d, J=12.6 Hz, 1H), 2.99 (br t, J=6.5 Hz, 2H), 2.83 (dt, J=11.7, 3.0 Hz, 1H), 2.6-2.6 (m, 1H), 1.71 (qd, J=13.4 Hz, 6.8, 1H), 0.93 (dd, J=15.9, 6.7 Hz, 6H); LCMS: [M+H]⁺=480.4.

### Example 17: (R)-6-(3-amino-6-(2-fluoro-4-(2-methylpiperidin-1-yl)phenyl)pyrazin-2-yl)-3,4-dihydroisoquinolin-1(2H)-one (I-17)

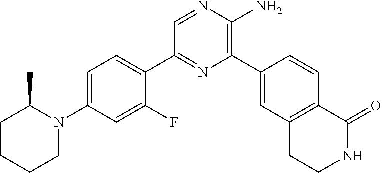 [SMILES: C[C@@H]1CCCCN1c1ccc(-c2cnc(N)c(-c3ccc4c(c3)CCNC4=O)n2)c(F)c1]

### Step 1: (R)-1-(4-chloro-3-fluorophenyl)-2-methylpiperidine

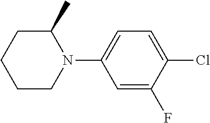

A procedure analogous to Example 7, Step 1 using (R)-2-methylpiperidine (0.284 g, 2.86 mmol), 4-bromo-1-chloro-2-fluorobenzene (0.500 g, 2.387 mmol), tris(dibenzylideneacetone)dipalladium (0) (0.066 g, 0.072 mmol), 4,5-bis(diphenylphosphino)-9,9-dimethylxanthene (0.124 g, 0.215 mmol) and Cs₂CO₃ (2.333 g, 7.16 mmol) afforded the product (140 mg, 26%). LCMS: [M+H]=228.25

### Step 2: (R)-1-(3-fluoro-4-(4,4,5,5-tetramethyl-1,3,2-dioxaborolan-2-yl)phenyl)-2-methylpiperidine

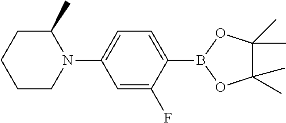 [SMILES: C[C@@H]1CCCCN1c1ccc(B2OC(C)(C)C(C)(C)O2)c(F)c1]

The procedure employed was similar to that described in Example 7, Step 2 using (R)-1-(4-chloro-3-fluorophenyl)-2-methylpiperidine (0.140 g, 0.615 mmol), bis(pinacolato)diboron (0.195 g, 0.769 mmol), KOAc (0.121 g, 1.230 mmol) and XPhos Pd G2 (0.036 g, 0.046 mmol) to afford the product (112 mg, 57%). LCMS: [M+H]⁺=320.43.

### Step 3: (R)-6-(3-amino-6-(2-fluoro-4-(2-methylpiperidin-1-yl)phenyl)pyrazin-2-yl)-3,4-dihydroisoquinolin-1(2H)-one

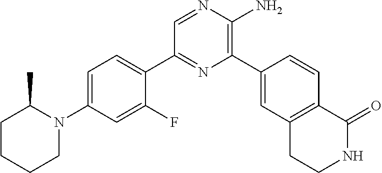 [SMILES: C[C@@H]1CCCCN1c1ccc(-c2cnc(N)c(-c3ccc4c(c3)CCNC4=O)n2)c(F)c1]

A procedure similar to that described in Example 7, Step 4 using (R)-1-(3-fluoro-4-(4,4,5,5-tetramethyl-1,3,2-dioxaborolan-2-yl)phenyl)-2-methylpiperidine (0.036 g, 0.113 mmol), 6-(3-amino-6-bromopyrazin-2-yl)-3,4-dihydroisoquinolin-1(2H)-one (0.030 g, 0.094 mmol) and XPhos Pd G2 (0.011 g, 0.014 mmol) afforded the title compound (24 mg, 59%) as a yellow powder. ¹H NMR (500 MHz, DMSO-d₆) δ 8.30 (d, J=2.3 Hz, 1H), 7.9-8.0 (m, 2H), 7.7-7.8 (m, 2H), 7.69 (s, 1H), 6.82 (dd, J=9.0, 2.4 Hz, 1H), 6.73 (dd, J=15.9, 2.3 Hz, 1H), 6.32 (s, 2H), 4.1-4.2 (m, 1H), 3.4-3.5 (m, 4H), 2.99 (t, J=6.5 Hz, 2H), 2.87 (dt, J=12.4 Hz, 3.1, 1H), 1.7-1.8 (m, 2H), 1.5-1.6 (m, 4H), 1.02 (d, J=6.7 Hz, 3H); LCMS: [M+H]⁺=432.4.

### Example 18: (S)-6-(3-amino-6-(2-fluoro-4-(2-methylpiperidin-1-yl)phenyl)pyrazin-2-yl)-3,4-dihydroisoquinolin-1(2H)-one (I-18)

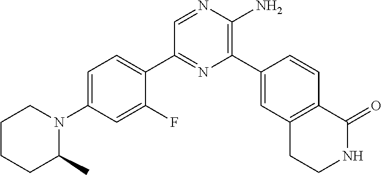 [SMILES: C[C@H]1CCCCN1c1ccc(-c2cnc(N)c(-c3ccc4c(c3)CCNC4=O)n2)c(F)c1]

### Step 1: (S)-1-(3-fluoro-4-(4,4,5,5-tetramethyl-1,3,2-dioxaborolan-2-yl)phenyl)-2-methylpiperidine

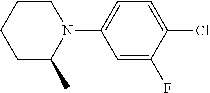

A procedure analogous to that described in Example 7, Step 1 using (S)-(+)-2-methylpiperidine (0.284 g, 2.86 mmol), 4-bromo-1-chloro-2-fluorobenzene (0.500 g, 2.387 mmol), tris(dibenzylideneacetone)dipalladium (0) (0.066 g, 0.072 mmol), 4,5-bis(diphenylphosphino)-9,9-dimethylxanthene (0.124 g, 0.215 mmol) and Cs₂CO₃ (2.33 g, 7.16 mmol) afforded the product (180 mg, 33%) as an oil. LCMS: [M+H]⁺=228.15.

### Step 2: (S)-1-(3-fluoro-4-(4,4,5,5-tetramethyl-1,3,2-dioxaborolan-2-yl)phenyl)-2-methylpiperidine

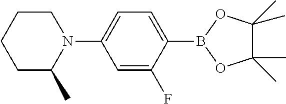 [SMILES: C[C@H]1CCCCN1c1ccc(B2OC(C)(C)C(C)(C)O2)c(F)c1]

The procedure followed was analagous to Example 7, Step 2 using (S)-1-(4-chloro-3-fluorophenyl)-2-methylpiperidine (0.180 g, 0.790 mmol), bis(pinacolato)diboron (0.251 g, 0.988 mmol), KOAc (0.155 g, 1.58 mmol) and XPhos Pd G2 (0.047 g, 0.059 mmol). After workup, (137 mg, 54%) of the product was obtained. LCMS: [M+H]⁺=320.20.

### Step 3: (S)-6-(3-amino-6-(2-fluoro-4-(2-methylpiperidin-1-yl)phenyl)pyrazin-2-yl)-3,4-dihydroisoquinolin-1(2H)-one

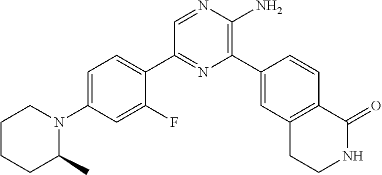 [SMILES: C[C@H]1CCCCN1c1ccc(-c2cnc(N)c(-c3ccc4c(c3)CCNC4=O)n2)c(F)c1]

A procedure similar to that described in Example 7, Step 4 using (S)-1-(3-fluoro-4-(4,4,5,5-tetramethyl-1,3,2-dioxaborolan-2-yl)phenyl)-2-methylpiperidine (0.036 g, 0.113 mmol), 6-(3-amino-6-bromopyrazin-2-yl)-3,4-dihydroisoquinolin-1(2H)-one (0.030 g, 0.094 mmol) and XPhos Pd G2 (0.011 g, 0.014 mmol) afforded the title compound (35 mg, 86%)¹H NMR (500 MHz, DMSO-d₆) δ 8.30 (d, J=2.4 Hz, 1H), 7.9-8.0 (m, 2H), 7.7-7.8 (m, 2H), 7.69 (s, 1H), 6.82 (dd, J=9.0, 2.5 Hz, 1H), 6.73 (dd, J=15.8, 2.4 Hz, 1H), 6.32 (s, 2H), 4.2-4.2 (m, 1H), 3.4-3.5 (m, 4H), 2.99 (t, J=6.5 Hz, 2H), 2.87 (dt, J=12.4, 3.1 Hz, 1H), 1.7-1.8 (m, 2H), 1.5-1.6 (m, 4H), 1.02 (d, J=6.6 Hz, 3H); LCMS: [M+H]⁺=432.5.

### Example 19: 6-(6-(4-(4-acetylpiperazin-1-yl)phenyl)-3-aminopyrazin-2-yl)-3,4-dihydroisoquinolin-1(2H)-one (I-19)

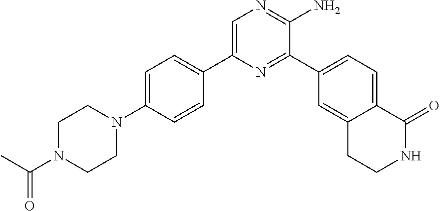 [SMILES: CC(=O)N1CCN(c2ccc(-c3cnc(N)c(-c4ccc5c(c4)CCNC5=O)n3)cc2)CC1]

The title compound was prepared by a method similar to that described in Example 1, Step 3 using 4-(4-acetyl-1-piperazinyl)phenylboronic acid (54.4 mg, 0.219 mmol), PdCl₂dppf (16.05 mg, 0.022 mmol), 6-(3-amino-6-bromopyrazin-2-yl)-3,4-dihydroisoquinolin-1(2H)-one (70 mg, 0.219 mmol) and Cs₂CO₃ (214 mg, 0.658 mmol) to afford the title compound (55 mg, 57%) as a yellow solid. ¹H NMR (500 MHz, DMSO-d₆) b ppm 8.50 (s, 1H), 7.92-8.02 (m, 2H), 7.87 (m, J=8.8 Hz, 2H), 7.73-7.76 (m, 1H), 7.70 (s, 1H), 7.03 (m, J=8.8 Hz, 2H), 6.24 (s, 2H), 3.59 (br s, 4H), 3.39-3.46 (m, 2H), 3.19-3.25 (m, 2H), 3.12-3.19 (m, 2H), 3.00 (br t, J=6.4 Hz, 2H), 2.05 (s, 3H); LCMS: [M+H]⁺=443.57.

### Example 20: 6-(3-amino-6-(4-(3-(dimethylamino)pyrrolidin-1-yl)-2-fluorophenyl)pyrazin-2-yl)-3,4-dihydroisoquinolin-1(2H)-one (I-20)

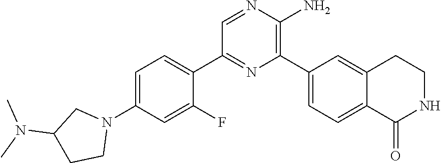 [SMILES: CN(C)C1CCN(c2ccc(-c3cnc(N)c(-c4ccc5c(c4)CCNC5=O)n3)c(F)c2)C1]

### Step 1: 1-(4-chloro-3-fluorophenyl)-N,N-dimethylpyrrolidin-3-amine

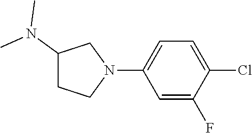 [SMILES: CN(C)C1CCN(c2ccc(Cl)c(F)c2)C1]

A procedure analogous to Example 7, Step 1 using 3-(dimethylamino)pyrrolidine (0.234 mL, 1.43 mmol), 4-bromo-1-chloro-2-fluorobenzene (0.139 mL, 1.19 mmol), tris(dibenzylideneacetone)dipalladium (0) (0.033 g, 0.036 mmol), 4,5-bis(diphenylphosphino)-9,9-dimethylxanthene (0.062 g, 0.107 mmol) and Cs₂CO₃ (1.17 g, 3.58 mmol) afforded the product (286 mg, 99%). LCMS: [M+H]⁺=243.19 Step 2: 1-(3-fluoro-4-(4,4,5,5-tetramethyl-1,3,2-dioxaborolan-2-yl)phenyl)-N,N-dimethylpyrrolidin-3-amine

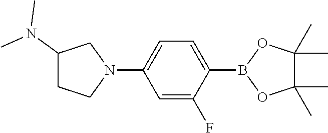

A procedure analogous to that described in Example 7, Step 2 using 1-(4-chloro-3-fluorophenyl)-N,N-dimethylpyrrolidin-3-amine (0.286 g, 1.18 mmol), bis(pinacolato)diboron (0.374 g, 1.47 mmol), KOAc (0.231 g, 2.36 mmol) and XPhos Pd G2 (0.070 g, 0.088 mmol) afforded the product (234 mg, 42%) that was judged to be approximately 70% pure and was used without further purification in the next step. LCMS: [M+H]⁺=335.24.

### Step 3: 6-(3-amino-6-(4-(3-(dimethylamino)pyrrolidin-1-yl)-2-fluorophenyl)pyrazin-2-yl)-3,4-dihydroisoquinolin-1(2H)-one (I-20)

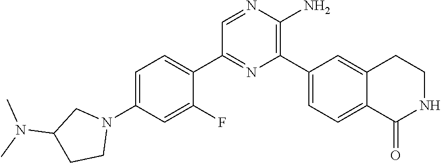 [SMILES: CN(C)C1CCN(c2ccc(-c3cnc(N)c(-c4ccc5c(c4)CCNC5=O)n3)c(F)c2)C1]

A procedure similar to Example 7, Step 4 using 1-(3-fluoro-4-(4,4,5,5-tetramethyl-1,3,2-dioxaborolan-2-yl)phenyl)-N,N-dimethylpyrrolidin-3-amine (0.041 g, 0.122 mmol), 6-(3-amino-6-bromopyrazin-2-yl)-3,4-dihydroisoquinolin-1(2H)-one (0.030 g, 0.094 mmol) and XPhos Pd G2 (7.40 mg, 9.40 μmol) afforded, after workup and purification, the title compound (25 mg, 60%)¹H NMR (500 MHz, DMSO-d₆) δ 8.28 (d, J=2.2 Hz, 1H), 7.9-8.0 (m, 2H), 7.7-7.8 (m, 3H), 6.48 (dd, J=8.8, 1.8 Hz, 1H), 6.4-6.4 (m, 1H), 6.27 (s, 2H), 3.5-3.5 (m, 2H), 3.4-3.4 (m, 4H), 3.2-3.3 (m, 1H), 3.06 (br t, J=8.7 Hz, 1H), 2.99 (br t, J=6.3 Hz, 2H), 2.8-2.8 (m, 1H), 2.1-2.2 (m, 9H), 1.8-1.9 (m, 1H); LCMS: [M+H]⁺=447.8.

### Example 21: 6-(3-amino-6-(4-(3-(dimethylamino)pyrrolidin-1-yl)-2,3-difluorophenyl)pyrazin-2-yl)-3,4-dihydroisoquinolin-1(2H)-one (I-21)

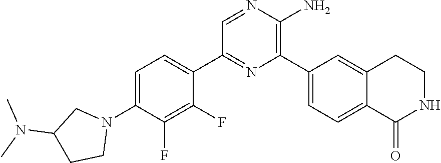 [SMILES: CN(C)C1CCN(c2ccc(-c3cnc(N)c(-c4ccc5c(c4)CCNC5=O)n3)c(F)c2F)C1]

### Step 1: 1-(2,3-difluorophenyl)-N,N-dimethylpyrrolidin-3-amine

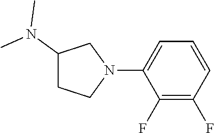 [SMILES: CN(C)C1CCN(c2cccc(F)c2F)C1]

A procedure analogous to that described in Example 7, Step 1 using A using 1-bromo-2,3-difluorobenzene (0.152 mL, 1.30 mmol), 3-(dimethylamino)pyrrolidine (0.254 mL, 1.55 mmol), tris(dibenzylideneacetone)dipalladium (0) (0.036 g, 0.039 mmol), 4,5-bis(diphenylphosphino)-9,9-dimethylxanthene (0.067 g, 0.117 mmol) and Cs₂CO₃ (1.27 g, 3.89 mmol) afforded the product. LCMS: [M+H]+=227.32.

### Step 2: 1-(4-bromo-2,3-difluorophenyl)-N,N-dimethylpyrrolidin-3-amine

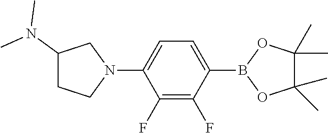

N-Bromosuccinimide (0.288 g, 1.62 mmol) was added to a stirring solution of 1-(2,3-difluorophenyl)-N,N-dimethylpyrrolidin-3-amine in CH₂Cl₂ (8 mL) at RT and the mixture was stirred for 16 h. The mixture was concentrated onto Celite and purified by silica gel chromatography (eluting with 0.5-10% CH₂Cl₂/MeOH+0.5% NH₄OH) to afford the product (261 mg, 66%). LCMS: [M+H]⁺=305.42.

### Step 3: 1-(2,3-difluoro-4-(4,4,5,5-tetramethyl-1,3,2-dioxaborolan-2-yl)phenyl)-N,N-dimethylpyrrolidin-3-amine

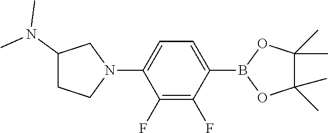

A procedure analogous to that described in Example 7, Step 2 using 1-(4-bromo-2,3-difluorophenyl)-N,N-dimethylpyrrolidin-3-amine (0.261 g, 0.855 mmol), bis(pinacolato)diboron (0.271 g, 1.069 mmol), KOAc (0.168 g, 1.711 mmol) and XPhos Pd G2 (0.050 g, 0.064 mmol) afforded the product (74 mg, 17%) that was judged to be approximately 70% pure and was used without further purification in the next step. LCMS: [M+H]⁺=353.44.

### Step 4: 6-(3-amino-6-(4-(3-(dimethylamino)pyrrolidin-1-yl)-2,3-difluorophenyl)pyrazin-2-yl)-3,4-dihydroisoquinolin-1(2H)-one

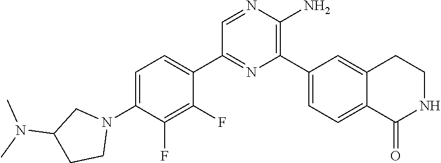 [SMILES: CN(C)C1CCN(c2ccc(-c3cnc(N)c(-c4ccc5c(c4)CCNC5=O)n3)c(F)c2F)C1]

A procedure similar to Example 7, Step 4 using 1-(2,3-difluoro-4-(4,4,5,5-tetramethyl-1,3,2-dioxaborolan-2-yl)phenyl)-N,N-dimethylpyrrolidin-3-amine (0.024 g, 0.069 mmol), 6-(3-amino-6-bromopyrazin-2-yl)-3,4-dihydroisoquinolin-1(2H)-one (0.020 g, 0.063 mmol) and XPhos Pd G2 (4.93 mg, 6.27 μmol) afforded after workup and purification the title compound (11 mg, 38%). ¹H NMR (500 MHz, DMSO-d₆) δ 8.30 (d, J=2.0 Hz, 1H), 7.9-8.0 (m, 2H), 7.7-7.8 (m, 2H), 7.52 (br t, J=8.6 Hz, 1H), 6.63 (br t, J=8.4 Hz, 1H), 6.41 (s, 2H), 3.4-3.6 (m, 10H), 3.25 (br d, 2H, J=8.4 Hz), 2.99 (br t, J=6.2 Hz, 2H), 2.78 (br d, J=4.8 Hz, 2H), 2.21 (br s, 7H), 2.1-2.2 (m, 1H), 1.7-1.8 (m, 1H); LCMS: [M+H]⁺=465.7.

### Example 22: (R)-6-(3-amino-6-(2-fluoro-4-(3-methylmorpholino)phenyl)pyrazin-2-yl)-3,4-dihydroisoquinolin-1(2H)-one (I-22)

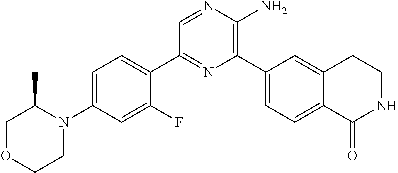 [SMILES: C[C@@H]1COCCN1c1ccc(-c2cnc(N)c(-c3ccc4c(c3)CCNC4=O)n2)c(F)c1]

### Step 1: (R)-4-(4-chloro-3-fluorophenyl)-3-methylmorpholine

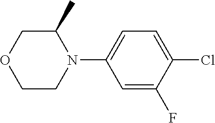

A procedure analagous to Example 7, Step 1 using (R)-3-methylmorpholine (0.21 mL, 1.43 mmol), 4-bromo-1-chloro-2-fluorobenzene (0.139 mL, 1.194 mmol), tris(dibenzylideneacetone)dipalladium (0) (0.033 g, 0.036 mmol), 4,5-bis(diphenylphosphino)-9,9-dimethylxanthene (0.062 g, 0.107 mmol) and Cs₂CO₃ (1.167 g, 3.58 mmol) afforded the product (84 mg, 31%). LCMS: [M+H]⁺=230.11.

### Step 2: (R)-4-(3-fluoro-4-(4,4,5,5-tetramethyl-1,3,2-dioxaborolan-2-yl)phenyl)-3-methylmorpholine

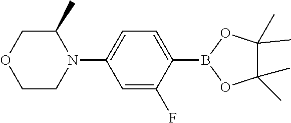 [SMILES: C[C@@H]1COCCN1c1ccc(B2OC(C)(C)C(C)(C)O2)c(F)c1]

A procedure analogous to that described in Example 7, Step 2 using (R)-4-(4-chloro-3-fluorophenyl)-3-methylmorpholine (0.084 g, 0.366 mmol), bis(pinacolato)diboron (0.116 g, 0.457 mmol), KOAc (0.072 g, 0.731 mmol) and XPhos Pd G2 (0.022 g, 0.027 mmol) afforded the product (165 mg, 45%). LCMS: [M+H]⁺=322.23.

### Step 3: (R)-6-(3-amino-6-(2-fluoro-4-(3-methylmorpholino)phenyl)pyrazin-2-yl)-3,4-dihydroisoquinolin-1(2H)-one

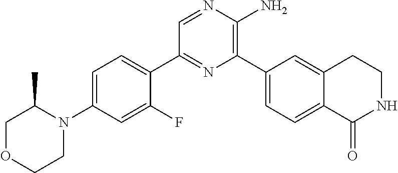 [SMILES: C[C@@H]1COCCN1c1ccc(-c2cnc(N)c(-c3ccc4c(c3)CCNC4=O)n2)c(F)c1]

A procedure similar to Example 7, Step 4 using (R)-4-(3-fluoro-4-(4,4,5,5-tetramethyl-1,3,2-dioxaborolan-2-yl)phenyl)-3-methylmorpholine (0.022 g, 0.069 mmol), 6-(3-amino-6-bromopyrazin-2-yl)-3,4-dihydroisoquinolin-1(2H)-one (0.020 g, 0.063 mmol) and XPhos Pd G2 (4.93 mg, 6.27 μmol) afforded the title compound as a yellow powder (15 mg, 55%). ¹H NMR (500 MHz, DMSO-d₆) δ 8.31 (d, J=2.3 Hz, 1H), 7.9-8.0 (m, 2H), 7.78 (t, J=9.2 Hz, 1H), 7.73 (dd, J=8.0, 1.3 Hz, 1H), 7.69 (s, 1H), 6.82 (dd, J=8.9, 2.2 Hz, 1H), 6.76 (br dd, J=15.5, 2.0 Hz, 1H), 6.35 (s, 2H), 3.9-4.0 (m, 2H), 3.7-3.7 (m, 2H), 3.54 (dt, J=11.5, 2.9 Hz, 1H), 3.4-3.5 (m, 3H), 3.0-3.1 (m, 3H), 1.06 (d, J=6.6 Hz, 3H); LCMS: [M+H]⁺=434.7.

### Example 23: (S)-6-(3-amino-6-(2-fluoro-4-(3-methylmorpholino)phenyl)pyrazin-2-yl)-3,4-dihydroisoquinolin-1(2H)-one (I-23)

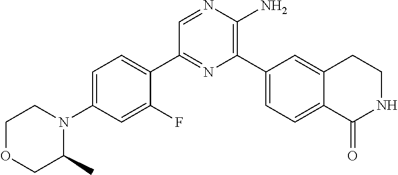 [SMILES: C[C@H]1COCCN1c1ccc(-c2cnc(N)c(-c3ccc4c(c3)CCNC4=O)n2)c(F)c1]

### Step 1: (S)-4-(4-chloro-3-fluorophenyl)-3-methylmorpholine

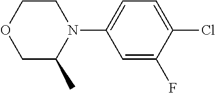

A procedure analagous to Example 7, Step 1 using (S)-3-methylmorpholine (0.21 mL, 1.43 mmol), 4-bromo-1-chloro-2-fluorobenzene (0.139 mL, 1.19 mmol), tris(dibenzylideneacetone)dipalladium (0) (0.033 g, 0.036 mmol), 4,5-bis(diphenylphosphino)-9,9-dimethylxanthene (0.062 g, 0.107 mmol) and Cs₂CO₃ (1.17 g, 3.58 mmol) afforded the product (114 mg, 42%). LCMS: [M+H]⁺=232.29

### Step 2: (S)-4-(3-fluoro-4-(4,4,5,5-tetramethyl-1,3,2-dioxaborolan-2-yl)phenyl)-3-methylmorpholine

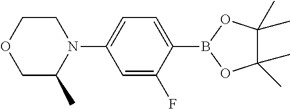 [SMILES: C[C@H]1COCCN1c1ccc(B2OC(C)(C)C(C)(C)O2)c(F)c1]

A procedure analogous to that described in Example 7, Step 2 using (S)-4-(4-chloro-3-fluorophenyl)-3-methylmorpholine (0.114 g, 0.496 mmol), bis(pinacolato)diboron (0.158 g, 0.620 mmol), KOAc (0.097 g, 0.993 mmol) and XPhos Pd G2 (0.029 g, 0.037 mmol) afforded the product (41 mg, 26%). LCMS: [M+H]⁺=322.23.

### Step 3: (S)-6-(3-amino-6-(2-fluoro-4-(3-methylmorpholino)phenyl)pyrazin-2-yl)-3,4-dihydroisoquinolin-1(2H)-one

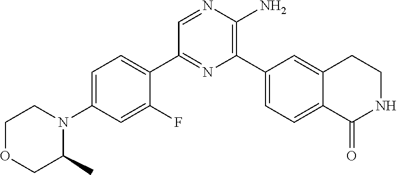 [SMILES: C[C@H]1COCCN1c1ccc(-c2cnc(N)c(-c3ccc4c(c3)CCNC4=O)n2)c(F)c1]

A procedure similar to Example 7, Step 4 using (S)-4-(3-fluoro-4-(4,4,5,5-tetramethyl-1,3,2-dioxaborolan-2-yl)phenyl)-3-methylmorpholine (0.021 g, 0.066 mmol), 6-(3-amino-6-bromopyrazin-2-yl)-3,4-dihydroisoquinolin-1(2H)-one (0.020 g, 0.063 mmol) and XPhos Pd G2 (4.93 mg, 6.27 μmol) afforded the title compound (11 mg, 40%) as a yellow powder. ¹H NMR (500 MHz, DMSO-d₆) δ 8.31 (d, J=2.2 Hz, 1H), 7.9-8.0 (m, 2H), 7.78 (t, J=9.2 Hz, 1H), 7.73 (br d, J=8.1 Hz, 1H), 7.69 (s, 1H), 6.82 (dd, J=8.9, 2.1 Hz, 1H), 6.76 (dd, J=15.5, 2.0 Hz, 1H), 6.35 (s, 2H), 3.9-4.0 (m, 2H), 3.7-3.7 (m, 2H), 3.54 (dt, J=11.5, 2.9 Hz, 1H), 3.4-3.4 (m, 2H), 3.0-3.1 (m, 3H), 1.06 (d, J=6.6 Hz, 3H); LCMS: [M+H]⁺434.7.

### Example 24: 6-(3-amino-6-(4-(3-isopropylpiperidin-1-yl)phenyl)pyrazin-2-yl)-3,4-dihydroisoquinolin-1(2H)-one (I-24)

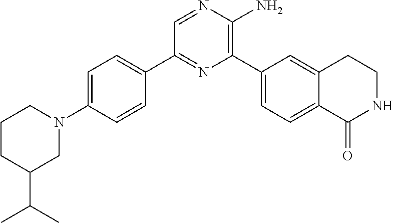 [SMILES: CC(C)C1CCCN(c2ccc(-c3cnc(N)c(-c4ccc5c(c4)CCNC5=O)n3)cc2)C1]

### Step 1: (5-amino-6-(1-oxo-1,2,3,4-tetrahydroisoquinolin-6-yl)pyrazin-2-yl)boronic acid

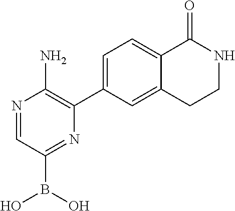 [SMILES: Nc1ncc(B(O)O)nc1-c1ccc2c(c1)CCNC2=O]

To a stirred solution of 6-(3-amino-6-bromopyrazin-2-yl)-3,4-dihydroisoquinolin-1(2H)-one (1 g, 3.1 mmol, preparation described in Example 19) in 1,4-dioxane (20 mL) was added B₂Pin₂ (3.97 g, 15.7 mmol) and KOAc (0.92 g, 98.6 mmol), bubbled with argon for 15 min at RT and treated with Pd(dppf)Cl₂·CH₂Cl₂ (0.25 g, 0.3 mmol). The reaction mixture was heated at 60° C. for 16 h and filtered through a Celite bed, washed with EtOAc and concentrated under reduced pressure to give crude compound which was triturated with 70% EtOAc in hexane to afford the product (0.7 g, 78%) as a black solid. LCMS: [M+H]⁺=285.37.

### Step 2: 6-(3-amino-6-(4-(3-isopropylpiperidin-1-yl)phenyl)pyrazin-2-yl)-3,4-dihydroisoquinolin-1(2H)-one

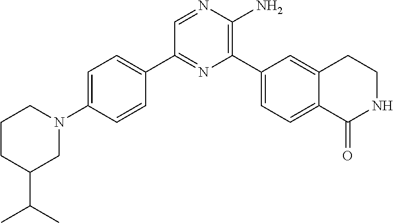 [SMILES: CC(C)C1CCCN(c2ccc(-c3cnc(N)c(-c4ccc5c(c4)CCNC5=O)n3)cc2)C1]

A procedure similar to Example 7, Step 4 using 1-(4-chlorophenyl)-3-isopropylpiperidine (0.023 g, 0.097 mmol), (5-amino-6-(1-oxo-1,2,3,4-tetrahydroisoquinolin-6-yl)pyrazin-2-yl)boronic acid (0.025 g, 0.088 mmol) and XPhos Pd G2 (6.92 mg, 8.80 μmol) afforded the title compound (3 mg, 8%). ¹H NMR (500 MHz, DMSO-d₆) δ 8.47 (s, 1H), 7.9-8.0 (m, 2H), 7.82 (d, J=8.8 Hz, 2H), 7.75 (br d, J=8.1 Hz, 1H), 7.70 (s, 1H), 6.98 (br d, J=8.9 Hz, 2H), 6.20 (s, 2H), 3.72 (br d, J=12.0 Hz, 2H), 3.42 (br dd, J=6.4, 2.4 Hz, 2H), 3.00 (br t, J=6.4 Hz, 2H), 2.6-2.7 (m, 1H), 1.8-1.8 (m, 1H), 1.72 (br d, J=13.2 Hz, 1H), 1.5-1.6 (m, 2H), 1.3-1.4 (m, 1H), 1.13 (dq, J=12.2, 3.7 Hz, 1H), 0.93 (dd, J=15.3, 6.8 Hz, 6H); LCMS: [M+H]⁺=442.6.

### Example 25: 6-(3-amino-6-(2-fluoro-4-(3-isopropylpiperidin-1-yl)phenyl)pyrazin-2-yl)-3,4-dihydroisoquinolin-1(2H)-one (I-25)

### Step 1: 1-(4-chloro-3-fluorophenyl)-3-isopropylpiperidine

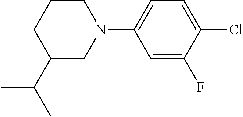 [SMILES: CC(C)C1CCCN(c2ccc(Cl)c(F)c2)C1]

A procedure analogous to Example 7, Step 1 using 3-(propan-2-yl)piperidine (0.182 g, 1.432 mmol), 4-bromo-1-chloro-2-fluorobenzene (0.250 g, 1.19 mmol), tris(dibenzylideneacetone)dipalladium (0) (0.033 g, 0.036 mmol), 4,5-bis(diphenylphosphino)-9,9-dimethylxanthene (0.062 g, 0.107 mmol) and Cs₂CO₃ (1.17 g, 3.58 mmol) afforded the product (214 mg, 70%). LCMS: [M+H]⁺=256.12.

### Step 2: 6-(3-amino-6-(2-fluoro-4-(3-isopropylpiperidin-1-yl)phenyl)pyrazin-2-yl)-3,4-dihydroisoquinolin-1(2H)-one

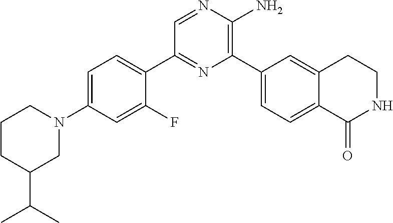 [SMILES: CC(C)C1CCCN(c2ccc(-c3cnc(N)c(-c4ccc5c(c4)CCNC5=O)n3)c(F)c2)C1]

A procedure analogous to Example 7, Step 4 using 1-(4-chloro-3-fluorophenyl)-3-isopropylpiperidine (0.025 g, 0.097 mmol), (5-amino-6-(1-oxo-1,2,3,4-tetrahydroisoquinolin-6-yl)pyrazin-2-yl)boronic acid (0.025 g, 0.088 mmol, from Example 24) and XPhos Pd G2 (6.92 mg, 8.80 μmol) afforded the title compound (7 mg, 17%). ¹H NMR (500 MHz, DMSO-d₆) δ 8.30 (d, J=2.2 Hz, 1H), 7.9-8.0 (m, 2H), 7.7-7.8 (m, 2H), 7.69 (s, 1H), 6.84 (dd, J=8.9, 2.2 Hz, 1H), 6.76 (dd, J=15.6, 2.0 Hz, 1H), 6.33 (s, 2H), 3.76 (br t, J=11.9 Hz, 2H), 3.42 (br dd, J=6.1, 4.0 Hz, 2H), 2.99 (br t, J=6.4 Hz, 2H), 2.71 (dt, J=12.4, 2.5 Hz, 1H), 2.54 (br s, 1H), 1.79 (br d, J=12.2 Hz, 1H), 1.71 (br d, J=13.1 Hz, 1H), 1.4-1.6 (m, 2H), 1.3-1.4 (m, 1H), 1.1-1.2 (m, 1H), 0.92 (dd, J=17.0, 6.7 Hz, 6H); LCMS: [M+H]⁺=460.6.

### Example 26: 6-(3-amino-6-(4-((1S,4S)-5-methyl-2,5-diazabicyclo[2.2.1]heptan-2-yl)phenyl)pyrazin-2-yl)-3,4-dihydroisoquinolin-1(2H)-one (I-26)

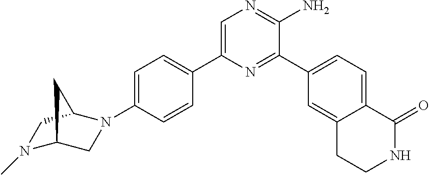 [SMILES: CN1C[C@@H]2C[C@H]1CN2c1ccc(-c2cnc(N)c(-c3ccc4c(c3)CCNC4=O)n2)cc1]

### Step 1: (1S,4S)-2-(4-chlorophenyl)-5-methyl-2,5-diazabicyclo[2.2.1]heptane

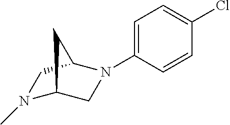

A mixture of (1S,4S)-2-(4-chlorophenyl)-2,5-diazabicyclo[2.2.1]heptane hydrobromide (246 mg, 0.849 mmol) was filtered through a Waters PoraPak CX column (2 g), rinsing with MeOH and eluting with 2 M NH₃ in MeOH. The MeOH/NH₃ fraction was concentrated under reduced pressure and the residue was taken into anh. THF (10 mL) and was treated with formaldehyde solution, 37% wt in water (0.702 mL, 9.34 mmol) at After stirring 3 h at RT, NaBH(OAc)₃ (234 mg, 1.10 mmol) in THF (10 mL) was treated with formaldehyde solution, 37% wt in water (0.702 mL, 9.34 mmol) at RT. After an additional 3 h of stirring at RT, another portion of NaBH(OAc)₃ (234 mg, 1.104 mmol) was added and stirring was continued for 16 h. The mixture was concentrated under reduced pressure, deposited on Biotage® samplet and purified by flash chromatography (25 g SiO₂ Biotage® cartridge, using MeOH in CH₂Cl₂ eluting at 15% MeOH, pooled fractions:15-35 to afford the product as a white solid (199 mg, quantitative yield). LCMS: [M+H]⁺=223.26.

### Step 2: 6-(3-amino-6-(4-((1S,4S)-5-methyl-2,5-diazabicyclo[2.2.1]heptan-2-yl)phenyl)pyrazin-2-yl)-3,4-dihydroisoquinolin-1(2H)-one (I-26)

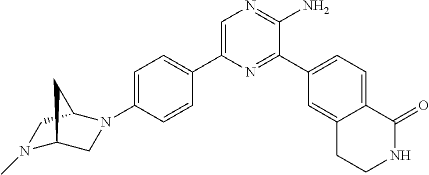 [SMILES: CN1C[C@@H]2C[C@H]1CN2c1ccc(-c2cnc(N)c(-c3ccc4c(c3)CCNC4=O)n2)cc1]

The procedure employed was similar to that described Example 1, Step 3 using XPhos Pd G2 (22.16 mg, 0.028 mmol), (1S,4S)-2-(4-chlorophenyl)-5-methyl-2,5-diazabicyclo[2.2.1]heptane (62.7 mg, 0.282 mmol), 5-amino-6-(1-oxo-1,2,3,4-tetrahydroisoquinolin-6-yl)pyrazin-2-yl)boronic acid (80 mg, 0.282 mmol) (preparation described in Example 24), aq. K₃PO₄ (0.650 mL of a 1.3 M solution, 0.845 mmol), to afford the product (5.0 mg, 4% based on purity of 98%) as a yellow solid. ¹H NMR (500 MHz, CD₃OD) δ ppm 8.25 (s, 1H), 7.98 (d, J=8.1 Hz, 1H), 7.68-7.75 (m, 3H), 7.66 (s, 1H), 6.61 (br d, J=8.8 Hz, 2H), 4.32 (br s, 1H), 3.55 (br s, 1H), 3.47 (br t, J=6.7 Hz, 2H), 3.39-3.43 (m, 1H), 3.27 (br d, J=9.7 Hz, 1H), 2.97-3.04 (m, 2H), 2.72-2.85 (m, 2H), 2.35 (s, 3H), 1.97 (br d, J=9.7 Hz, 1H), 1.82-1.92 (m, 1H); LCMS: [M+H]⁺=427.49

### Example 27: (R)-6-(3-amino-6-(4-(2-isopropylmorpholino)phenyl)pyrazin-2-yl)-7-fluoro-3,4-dihydroisoquinolin-1(2H)-one (I-27)

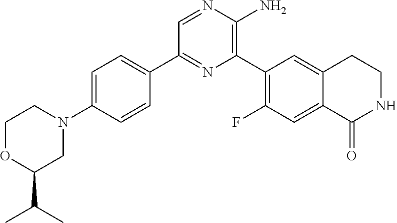 [SMILES: CC(C)[C@@H]1CN(c2ccc(-c3cnc(N)c(-c4cc5c(cc4F)C(=O)NCC5)n3)cc2)CCO1]

### Step 1: 3-(3-bromo-4-fluorophenyl)propanoic acid

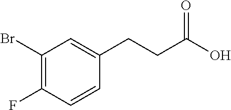 [SMILES: O=C(O)CCc1ccc(F)c(Br)c1]

To a stirred solution of Et₃N (31 mL, 224.6 mmol) was added formic acid (22 mL, 561.6 mmol) portionwise and the mixture was stirred for 15 min at RT. The mixture was then diluted with DMF (150 mL) and 3-bromo-4-fluorobenzaldehyde (38 g, 187.2 mmol) and Meldrum's acid (27 g, 187.2 mmol) were added. The mixture was then heated at 100° C. for 16 h before cooling to RT. The reaction mixture was poured into ice cold water (1.8 l) and conc. HCl (100 mL). The mixture was extracted with CH₂Cl₂ (2×600 mL). The organic layer was washed with 1 N NaOH (2×500 mL). The aqueous layer was acidified with conc. HCl and extracted with EtOAc (2×600 mL). The organic layer was dried over Na₂SO₄ and concentrated under reduced pressure to afford the product (38 g, 46.7% pure by LCMS) as brown liquid. The crude compound taken for next step without further purification. LCMS: [M+H]⁺=247.20

### Step 2: 5-bromo-6-fluoro-2,3-dihydro-1H-inden-1-one

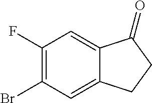 [SMILES: O=C1CCc2cc(Br)c(F)cc21]

To a stirred solution of 3-(3-bromo-4-fluorophenyl)propanoic acid (38 g, 46.7% pure by LCMS) (16 g, 65 mmol) in CH₂Cl₂ (100 mL) was added oxalylchloride (22 mL, 260 mmol) and DMF (1 mL) at RT for 30 min. The solvent was concentrated under reduced pressure to give crude residue. The residue was dissolved in CH₂Cl₂ (200 mL) and was added dropwise to a stirred solution AlCl₃ (35 g, 260 mmol) in CH₂Cl₂ (1 lt). The mixture was stirred for 2 h at RT. The mixture was poured into ice cold water (800 mL) and conc. HCl (50 mL) and extracted with CH₂Cl₂ (2×600 mL). The organic layers were dried over Na₂SO₄ concentrated under reduced pressure. The residue was purified by column chromatography (silica gel, 230-400 mesh) using an eluent 0-10% EtOAc in pet ether to afford the product (14 g, 94%) as an off white solid. LCMS: [M+H]⁺=229.24.

### Step 3: 6-bromo-7-fluoro-3,4-dihydroisoquinolin-1(2H)-one

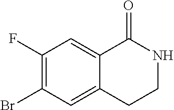 [SMILES: O=C1NCCc2cc(Br)c(F)cc21]

To a stirred solution of 5-bromo-6-fluoro-2,3-dihydro-1H-inden-1-one (14.8 g, 65 mmol) in CH₂Cl₂ (120 mL) and methane sulphonicacid (60 mL) was added NaN₃ (14.7 g, 227.2 mmol) portionwise at 0° C. for 1 h. The mixture was basified with 20% aqueous NaOH solution (300 mL) and extracted with CH₂Cl₂ (2×400 mL). The combined organic layer was dried over Na₂SO₄ and concentrated under reduced pressure to give crude product. The crude compound was purified by column chromatography (silica gel, 230-400 mesh) using an eluent 0-90% EtOAc in Hexane to afford the product (8 g, 51%) as an off white solid. TLC: 80% EtOAc:pet ether; R_{f}=0.3.

### Step 4: 7-fluoro-6-(4,4,5,5-tetramethyl-1,3,2-dioxaborolan-2-yl)-3,4-dihydroisoquinolin-1(2H)-one

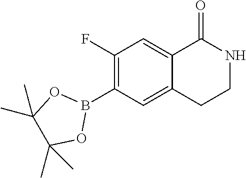 [SMILES: CC1(C)OB(c2cc3c(cc2F)C(=O)NCC3)OC1(C)C]

To a stirred solution of 6-bromo-7-fluoro-3,4-dihydroisoquinolin-1(2H)-one (8 g, 33 mmol) in 1,4-dioxane (100 mL) was added KOAc (9.7 g, 99 mmol) and 6-bromo-7-fluoro-3,4-dihydroisoquinolin-1(2H)-one (12.5 g, 49.3 mmol) at RT then the mixture was de-gassed with argon for 30 min. Pd(dppf)Cl₂·CH₂Cl₂ (0) (2.7 g, 3.3 mmol) was then added and the reaction mixture was heated at 85° C. for 16 h before cooling to RT. The reaction mixture was filtered through Celite bed and washed with EtOAc (700 mL) and the filtrate was concentrated under reduced pressure to give crude compound: which was purified by column chromatography (silica gel, 100-200 mesh) by using an eluent 0-100% EtOAc in Pet ether and 0-5% MeOH in CH₂Cl₂ to afford the product (5.5 g, 57%) as an brown gummy liquid. LCMS: [M+H]⁺=210.32.

### Step 5: 6-(3-amino-6-bromopyrazin-2-yl)-7-fluoro-3,4-dihydroisoquinolin-1(2H)-one

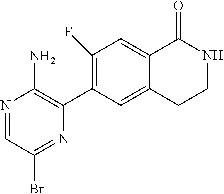 [SMILES: Nc1ncc(Br)nc1-c1cc2c(cc1F)C(=O)NCC2]

To a stirred solution of 7-fluoro-6-(4,4,5,5-tetramethyl-1,3,2-dioxaborolan-2-yl)-3,4-dihydroisoquinolin-1 (2H)-one (7 g, 24 mmol) in DMF:water (70 mL: 7 mL) was added K₂CO₃ (6.6 g, 48 mmol) 3,5-dibromopyrazin-2-amine (7.2 g, 28.8 mmol, preparation described in Example 14, Step 4) at RT then the reaction mixture was de-gassed with argon for 30 min. Trans-dichlorobis (triphenylphosphine) palladium (II) (845 mg, 1.2 mmol) was added and the reaction mixture was heated at 90° C. for 16 h before cooling to RT. The mixture was filtered through Celite bed and washed EtOAc (500 mL) and the filtrate was washed with cold water (200 mL), separated organic layer then dried over Na₂SO₄ and concentrated under reduced pressure to give crude compound: which purified by column chromatography (silica gel, 230-400 mesh) by using an eluent 0-100% EtOAc in Pet ether to afford the product (2.4 g, 40%) as a pale brown solid. LCMS: [M+H]⁺=337.21.

### Step 6: (R)-6-(3-amino-6-(4-(2-isopropylmorpholino)phenyl)pyrazin-2-yl)-7-fluoro-3,4-dihydroisoquinolin-1(2H)-one

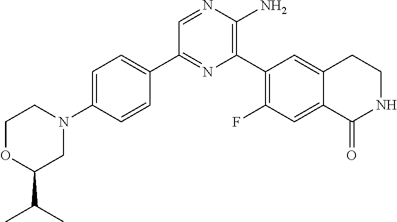 [SMILES: CC(C)[C@@H]1CN(c2ccc(-c3cnc(N)c(-c4cc5c(cc4F)C(=O)NCC5)n3)cc2)CCO1]

A procedure similar to Example 7, Step 4 using (R)-2-isopropyl-4-(4-(4,4,5,5-tetramethyl-1,3,2-dioxaborolan-2-yl)phenyl)morpholine (0.030 g, 0.091 mmol), 6-(3-amino-6-bromopyrazin-2-yl)-7-fluoro-3,4-dihydroisoquinolin-1(2H)-one (0.031 g, 0.091 mmol) and XPhos Pd G2 (7.13 mg, 9.06 μmol) afforded the title compound (19 mg, 46%) as a yellow powder. ¹H NMR (500 MHz, DMSO-d₆) δ 8.53 (s, 1H), 8.14 (br s, 1H), 7.80 (br d, J=8.3 Hz, 2H), 7.64 (br d, J=10.1 Hz, 1H), 7.51 (br d, J=6.7 Hz, 1H), 7.01 (br d, J=8.6 Hz, 2H), 6.18 (s, 2H), 3.97 (br d, J=11.6 Hz, 1H), 3.5-3.7 (m, 4H), 3.42 (br s, 3H), 3.2-3.3 (m, 2H), 2.96 (br t, J=6.0 Hz, 2H), 2.68 (dt, J=11.7, 2.9 Hz, 1H), 2.4-2.5 (m, 1H), 1.73 (qd, J=13.3. 7.5 Hz, 1H), 0.96 (br dd, J=6.4, 2.8 Hz, 6H); LCMS: [M+H]⁺=462.4.

### Example 28: (S)-6-(3-amino-6-(4-(2-isopropylmorpholino)phenyl)pyrazin-2-yl)-7-fluoro-3,4-dihydroisoquinolin-1(2H)-one (I-28)

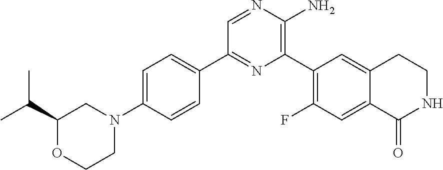 [SMILES: CC(C)[C@H]1CN(c2ccc(-c3cnc(N)c(-c4cc5c(cc4F)C(=O)NCC5)n3)cc2)CCO1]

A procedure similar to Example 7, Step 4 using (S)-2-isopropyl-4-(4-(4,4,5,5-tetramethyl-1,3,2-dioxaborolan-2-yl)phenyl)morpholine (0.030 g, 0.091 mmol), 6-(3-amino-6-bromopyrazin-2-yl)-7-fluoro-3,4-dihydroisoquinolin-1(2H)-one (0.031 g, 0.091 mmol) and XPhos Pd G2 (7.13 mg, 9.06 μmol) afforded the title compound (18 mg, 43%) as a yellow powder. ¹H NMR (500 MHz, DMSO-d₆) δ 8.53 (s, 1H), 8.14 (br s, 1H), 7.80 (br d, J=8.8 Hz, 2H), 7.64 (d, J=10.1 Hz, 1H), 7.51 (br d, J=6.8 Hz, 1H), 7.01 (br d, J=8.8 Hz, 2H), 6.18 (s, 2H), 3.9-4.0 (m, 1H), 3.5-3.6 (m, 5H), 3.42 (br d, J=4.0 Hz, 3H), 3.2-3.3 (m, 3H), 2.96 (br t, J=6.3 Hz, 2H), 2.6-2.7 (m, 1H), 2.4-2.5 (m, 2H), 1.73 (qd, J=13.3, 6.4 Hz, 1H), 0.96 (dd, J=6.6, 2.9 Hz, 6H); LCMS: [M+H]⁺=462.4.

### Example 29: (R)-6-(3-amino-6-(2-fluoro-4-(2-isopropylmorpholino)phenyl)pyrazin-2-yl)-7-fluoro-3,4-dihydroisoquinolin-1(2H)-one (I-29)

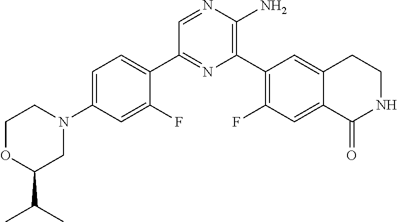 [SMILES: CC(C)[C@@H]1CN(c2ccc(-c3cnc(N)c(-c4cc5c(cc4F)C(=O)NCC5)n3)c(F)c2)CCO1]

A procedure similar to that described in Example 7, Step 4 using (R)-4-(3-fluoro-4-(4,4,5,5-tetramethyl-1,3,2-dioxaborolan-2-yl)phenyl)-2-isopropylmorpholine (0.030 g, 0.086 mmol), 6-(3-amino-6-bromopyrazin-2-yl)-7-fluoro-3,4-dihydroisoquinolin-1(2H)-one (0.029 g, 0.086 mmol) and XPhos Pd G2 (6.76 mg, 8.59 μmol) afforded the title compound (14 mg, 34%) as a yellow powder. ¹H NMR (500 MHz, DMSO-d₆) δ 8.35 (d, J=1.7 Hz, 1H), 8.15 (br s, 1H), 7.70 (br t, J=9.2 Hz, 1H), 7.64 (d, J=10.1 Hz, 1H), 7.52 (br d, J=6.8 Hz, 1H), 6.8-6.9 (m, 2H), 6.32 (s, 2H), 3.9-4.0 (m, 1H), 3.6-3.7 (m, 4H), 3.42 (br s, 3H), 3.2-3.3 (m, 1H), 2.95 (br t, J=6.2 Hz, 2H), 2.7-2.8 (m, 1H), 1.73 (qd, J=13.2, 6.6 Hz, 1H), 0.96 (br d, J=6.7 Hz, 6H); LCMS: [M+H]⁺=480.4.

### Example 30: (S)-6-(3-amino-6-(2-fluoro-4-(2-isopropylmorpholino)phenyl)pyrazin-2-yl)-7-fluoro-3,4-dihydroisoquinolin-1(2H)-one (I-30)

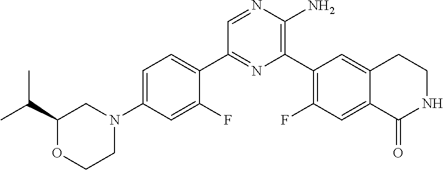 [SMILES: CC(C)[C@H]1CN(c2ccc(-c3cnc(N)c(-c4cc5c(cc4F)C(=O)NCC5)n3)c(F)c2)CCO1]

A procedure similar to that described in Example 7, Step 4 using (S)-4-(3-fluoro-4-(4,4,5,5-tetramethyl-1,3,2-dioxaborolan-2-yl)phenyl)-2-isopropylmorpholine (0.030 g, 0.086 mmol), 6-(3-amino-6-bromopyrazin-2-yl)-7-fluoro-3,4-dihydroisoquinolin-1(2H)-one (preparation described in Example 27, Step 5) (0.029 g, 0.086 mmol) and XPhos Pd G2 (6.76 mg, 8.59 μmol) afforded the title compound (13 mg, 32%) as a yellow powder. ¹H NMR (500 MHz, DMSO-d₆) δ 8.35 (d, J=2.2 Hz, 1H), 8.15 (br s, 1H), 7.70 (br t, J=9.2 Hz, 1H), 7.64 (d, J=10.1 Hz, 1H), 7.52 (d, J=6.7 Hz, 1H), 6.8-6.9 (m, 2H), 6.32 (s, 2H), 3.97 (br dd, J=11.1, 2.6 Hz, 1H), 3.6-3.7 (m, 3H), 3.4-3.5 (m, 3H), 3.2-3.3 (m, 1H), 2.95 (br t, J=6.2 Hz, 2H), 2.71 (dt, J=12.4, 3.4 Hz, 1H), 1.73 (qd, J=13.3, 6.8 Hz, 1H), 0.96 (d, J=6.7 Hz, 6H); LCMS: [M+H]⁺=480.4.

### Example 31: (R)-6-(3-amino-6-(2,3-difluoro-4-(2-isopropylmorpholino)phenyl)pyrazin-2-yl)-7-fluoro-3,4-dihydroisoquinolin-1(2H)-one (I-31)

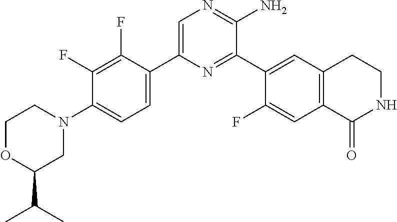 [SMILES: CC(C)[C@@H]1CN(c2ccc(-c3cnc(N)c(-c4cc5c(cc4F)C(=O)NCC5)n3)c(F)c2F)CCO1]

A mixture of (R)-4-(2,3-difluoro-4-(4,4,5,5-tetramethyl-1,3,2-dioxaborolan-2-yl)phenyl)-2-isopropylmorpholine (60 mg, 0.163 mmol) (preparation described in Example 15, Step 3), 6-(3-amino-6-bromopyrazin-2-yl)-7-fluoro-3,4-dihydroisoquinolin-1(2H)-one (preparation descried in Example 27, Step 5) (55.1 mg, 0.163 mmol) and XPhos Pd G2 (12.85 mg, 0.016 mmol) was added to a reaction vessel which was sealed, evacuated and backfilled with N₂. 1,4-Dioxane (2 mL) and K₃PO₄ tribasic (87 mg, 0.408 mmol) in H₂O (0.4 mL) were added and the reaction vessel was evacuated and backfilled with nitrogen an additional time. The reaction mixture was heated at 90° C. overnight, worked up and the product was purified in a manner similar to previous examples to afford the title compound (20 mg, 23%). ¹H NMR (500 MHz, DMSO-d₆) δ 8.4-8.4 (m, 1H), 8.15 (br s, 1H), 7.64 (d, J=10.1 Hz, 1H), 7.5-7.6 (m, 2H), 6.96 (br t, J=8.1 Hz, 1H), 6.50 (s, 2H), 3.94 (br d, J=11.0 Hz, 1H), 3.6-3.7 (m, 1H), 3.4-3.5 (m, 2H), 3.4-3.4 (m, 1H), 3.2-3.3 (m, 2H), 2.95 (br t, J=6.1 Hz, 2H), 2.8-2.9 (m, 1H), 2.6-2.6 (m, 1H), 1.71 (qd, J=13.4, 6.7 Hz, 1H), 0.95 (br d, J=6.7 Hz, 3H), 0.91 (br d, J=6.7 Hz, 3H); LCMS: [M+H]⁺=498.3.

### Example 32: (S)-6-(3-amino-6-(2,3-difluoro-4-(2-isopropylmorpholino)phenyl)pyrazin-2-yl)-7-fluoro-3,4-dihydroisoquinolin-1(2H)-one (I-32)

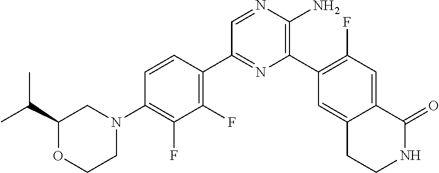 [SMILES: CC(C)[C@H]1CN(c2ccc(-c3cnc(N)c(-c4cc5c(cc4F)C(=O)NCC5)n3)c(F)c2F)CCO1]

(S)-4-(2,3-difluoro-4-(4,4,5,5-tetramethyl-1,3,2-dioxaborolan-2-yl)phenyl)-2-isopropylmorpholine (40 mg, 0.109 mmol, 1 eq), 6-(3-amino-6-bromopyrazin-2-yl)-7-fluoro-3,4-dihydroisoquinolin-1(2H)-one (36.7 mg, 0.109 mmol, 1 eq) and XPhos Pd G2 (8.57 mg, 10.89 μmol, 0.1 eq) were placed in the reaction vial. 1,4-dioxane (2 mL) and K₃PO₄ tribasic (57.8 mg, 0.272 mmol) in H₂O (0.4 mL) were added under a nitrogen atmosphere. The reaction mixture was bubbled with nitrogen and sealed. The reaction mixture was heated at 90° C. overnight, evaporated with Celite and purified by reverse phase flash chromatography (H₂O/ACN) followed by normal phase flash chromatography (CH₂Cl₂/ACN). Fractions with pure compound were collected and dried to obtain the title compound as a yellow solid (10 mg, 18%). ¹H NMR (500 MHz, DMSO-d₆) δ 8.39 (d, J=2.3 Hz, 1H), 8.15 (br s, 1H), 7.64 (d, J=10.3 Hz, 1H), 7.5-7.6 (m, 2H), 6.96 (br t, J=8.1 Hz, 1H), 6.50 (s, 2H), 3.94 (br d, J=10.1 Hz, 1H), 3.6-3.7 (m, 1H), 3.4-3.5 (m, 2H), 3.4-3.4 (m, 1H), 3.2-3.3 (m, 2H), 2.95 (br t, J=6.3 Hz, 2H), 2.82 (dt, J=11.6 Hz, 2.9, 1H), 2.5-2.6 (m, 1H), 1.71 (qd, J=13.4, 6.7 Hz, 1H), 0.95 (br d, J=6.7 Hz, 3H), 0.91 (br d, J=6.8 Hz, 3H); LCMS: [M+H]⁺=498.51.

### Example 33: 6-(3-amino-6-(4-(1-methyl-5,6-dihydro-1,2,4-triazin-4(1H)-yl)phenyl)pyrazin-2-yl)-3,4-dihydroisoquinolin-1(2H)-one (I-33)

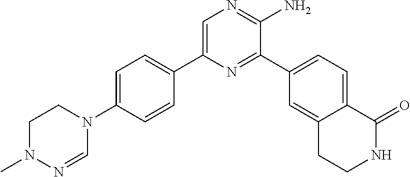 [SMILES: CN1CCN(c2ccc(-c3cnc(N)c(-c4ccc5c(c4)CCNC5=O)n3)cc2)C=N1]

The title compound was obtained following a procedure analogous to that described in Example 1, Step 3 using 5-amino-6-(1-oxo-1,2,3,4-tetrahydroisoquinolin-6-yl)pyrazin-2-yl)boronic acid (224 mg, 0.787 mmol) (preparation described in Example 24, Step 1), Pd(amphos)Cl₂ (13.93 mg, 0.020 mmol), 4-(4-bromophenyl)-1-methyl-1,4,5,6-tetrahydro-1,2,4-triazine (100 mg, 0.393 mmol) (preparation described in Example 31, Step 8) K₃PO₄ (251 mg, 1.180 mmol), H₂O (0.75 mL) to afford the title compound as a yellow solid (16.0 mg, 10% based on purity of 97%). ¹H NMR (500 MHz, DMSO-d₆) δ ppm 8.54 (s, 1H), 7.90-8.03 (m, 4H), 7.75 (br d, J=8.0 Hz, 1H), 7.69 (br s, 1H), 7.42-7.49 (m, 1H), 7.21 (br d, J=8.8 Hz, 2H), 6.31 (s, 2H), 3.76 (br t, J=4.8 Hz, 2H), 3.42-3.46 (m, 2H), 3.00 (br t, J=6.4 Hz, 2H), 2.90-2.97 (m, 2H), 2.65 (s, 3H); LCMS: [M+H]⁺=414.30.

### Example 34: 6-(3-amino-6-(4-((1S,4S)-5-(2,2,2-trifluoroethyl)-2,5-diazabicyclo[2.2.1]heptan-2-yl)phenyl)pyrazin-2-yl)-3,4-dihydroisoquinolin-1(2H)-1-one (I-34)

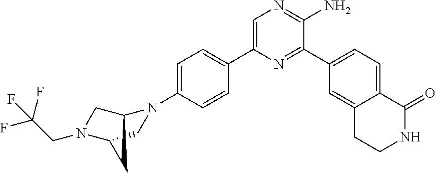 [SMILES: Nc1ncc(-c2ccc(N3C[C@@H]4C[C@H]3CN4CC(F)(F)F)cc2)nc1-c1ccc2c(c1)CCNC2=O]

### Step 1: (1S,4S)-2-(4-chlorophenyl)-5-(2,2,2-trifluoroethyl)-2,5-diazabicyclo[2.2.1]heptane

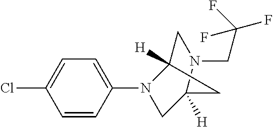 [SMILES: FC(F)(F)CN1C[C@@H]2C[C@H]1CN2c1ccc(Cl)cc1]

(1S,4S)-(−)-2-(4-Chlorophenyl)-2,5-diazabicyclo[2.2.1]heptane hydrobromide (70 mg, 0.242 mmol) was filtered through a Waters PoraPak CX column to generate the corresponding free base. A solution of this free base in THF (15 mL), in a round bottom flask, fitted with a reflux condenser, was heated to 70° C. Phenylsilane (0.060 mL, 0.483 mmol) was added to it, followed by TFA (0.032 mL, 0.423 mmol) at 70° C. After stirring at 70° C. for 3 h, another batch of TFA (1.75 eq) and phenylsilane (2 eq) were added at 70° C. Finally, after an additional 2 h of stirring at 70° C., a third batch of TFA (1.75 eq) and phenylsilane (2 eq) were added and the reaction was continuously stirred overnight at 70° C. The reaction mixture was concentrated to dryness and adsorbed onto Celite. Silica gel chromatography (eluting with 0-8% EtOAc/hexanes) afforded the product as a white solid (86 mg, 100% based on purity of 82%). LCMS: [M+H]⁺=291.32.

### Step 2: 6-(3-amino-6-(4-((1S,4S)-5-(2,2,2-trifluoroethyl)-2,5-diazabicyclo[2.2.1]heptan-2-yl)phenyl)pyrazin-2-yl)-3,4-dihydroisoquinolin-1(2H)-one

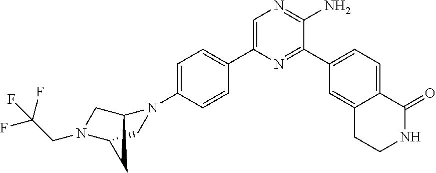 [SMILES: Nc1ncc(-c2ccc(N3C[C@@H]4C[C@H]3CN4CC(F)(F)F)cc2)nc1-c1ccc2c(c1)CCNC2=O]

A microwave vial charged with ((1S,4S)-2-(4-chlorophenyl)-5-(2,2,2-trifluoroethyl)-2,5-diazabicyclo[2.2.1]heptane (43 mg, 0.126 mmol), (5-amino-6-(1-oxo-1,2,3,4-tetrahydroisoquinolin-6-yl)pyrazin-2-yl)boronic acid (preparation described in Example 24, Step 1) (42.9 mg, 0.151 mmol), XPhos Pd G2 (12.86 mg, 0.016 mmol), K₃PO₄ (80 mg, 0.377 mmol), 1-butanol (4 mL) and water (0.8 mL) was flushed with argon. The reaction mixture was heated at 90° C. in a microwave reactor for 1.75 h. The reaction mixture was partitioned between brine and EtOAc. The layers were separated, and the aqueous layer was further extracted with CHCl₃/IPA (4:1) solvent mixture (2×4 mL). The combined extracts were dried and concentrated onto Celite. Silica gel chromatography (eluting with 0-80% EtOAc/hexanes) followed by a second silica gel column (eluting with 0-2% MeOH/CH₂Cl₂) and filtration through a Waters PoraPak CX column afforded the title compound as a pale yellow solid (13.5 mg, 20%)¹H NMR (500 MHz, DMSO-d₆) δ 8.4-8.5 (m, 1H), 7.9-8.0 (m, 2H), 7.8-7.8 (m, 2H), 7.7-7.8 (m, 1H), 7.7-7.7 (m, 1H), 6.6-6.7 (m, 2H), 6.14 (s, 2H), 4.40 (br s, 1H), 3.7-3.7 (m, 1H), 3.4-3.5 (m, 4H), 3.3-3.3 (m, 1H), 3.2-3.3 (m, 1H), 3.0-3.1 (m, 3H), 2.6-2.7 (m, 1H), 1.8-1.9 (m, 2H). LCMS: [M+H]⁺=495.55.

### Example 35: 6-(3-amino-6-(4-(4-(2,2,2-trifluoroethyl)piperazin-1-yl)phenyl)pyrazin-2-yl)-3,4-dihydroisoquinolin-1(2H)-one (I-35)

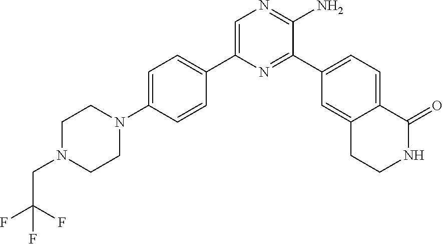 [SMILES: Nc1ncc(-c2ccc(N3CCN(CC(F)(F)F)CC3)cc2)nc1-c1ccc2c(c1)CCNC2=O]

### Step 1: 1-(4-chlorophenyl)-4-(2,2,2-trifluoroethyl)piperazine

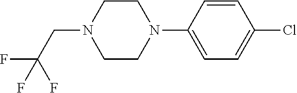 [SMILES: FC(F)(F)CN1CCN(c2ccc(Cl)cc2)CC1]

A slurry of 1-chloro-4-iodobenzene (250 mg, 1.05 mmol), 1-(2,2,2-trifluoroethyl)piperazine (176 mg, 1.05 mmol), 4,5-bis(diphenylphosphino)-9,9-dimethylxanthene (54.6 mg, 0.094 mmol), tris(dibenzylideneacetone)dipalladium (0) (28.8 mg, 0.031 mmol) and potassium tert-butoxide (353 mg, 3.15 mmol) in toluene (5 ml) was flushed with argon. The slurry was heated in an oil bath at 110° C. for 3 h. The reaction was partitioned between EtOAc (10 mL) and water. The layers were separated and the organic layer was further washed twice with water. The organic layer was concentrated onto Celite. Silica gel chromatography (eluting with 0-2% EtOAc/hexanes) afforded the product as a white powder (166 mg, 57%). LCMS: [M+H]⁺=279.47 Step 2: 6-(3-amino-6-(4-(4-(2,2,2-trifluoroethyl)piperazin-1-yl)phenyl)pyrazin-2-yl)-3,4-dihydroisoquinolin-1(2H)-one

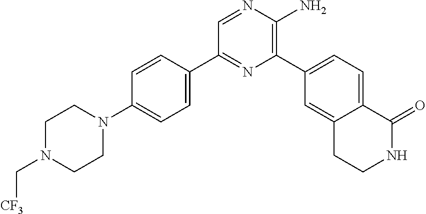 [SMILES: Nc1ncc(-c2ccc(N3CCN(CC(F)(F)F)CC3)cc2)nc1-c1ccc2c(c1)CCNC2=O]

A procedure analogous to that of Example 34, Step 2, using 1-(4-chlorophenyl)-4-(2,2,2-trifluoroethyl)piperazine (25 mg, 0.090 mmol) with (5-amino-6-(1-oxo-1,2,3,4-tetrahydroisoquinolin-6-yl)pyrazin-2-yl)boronic acid (preparation described in Example 24, Step 1) (30.6 mg, 0.108 mmol) at 90° C. in a microwave reactor for 1.75 h. The reaction mixture was partitioned between brine and EtOAc. The layers were separated, and the aqueous layer was further extracted with CH₂Cl₂. The combined extract was dried and concentrated onto Celite. Silica gel chromatography (eluting with EtOAc/Hexanes) and filtration through a Waters PoraPak CX column afforded the title compound as a pale yellow solid (7.5 mg, 16%)¹H NMR (500 MHz, DMSO-d₆) δ 8.55-8.41 (m, 1H), 7.96 (br d, J=8.2 Hz, 2H), 7.85 (br d, J=8.6 Hz, 2H), 7.79-7.73 (m, 1H), 7.72-7.68 (m, 1H), 7.01 (br d, J=8.6 Hz, 2H), 6.31-6.14 (m, 2H), 3.46-3.45 (m, 2H), 3.25-3.19 (m, 6H), 3.02-2.98 (m, 2H), 2.80-2.75 (m, 4H). LCMS: [M+H]⁺=483.46.

### Example 36: 6-(3-amino-6-(4-(1-(2,2,2-trifluoroethyl)piperidin-4-yl)phenyl)pyrazin-2-yl)-3,4-dihydroisoquinolin-1(2H)-one (I-36)

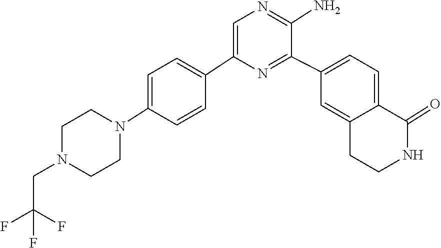 [SMILES: Nc1ncc(-c2ccc(N3CCN(CC(F)(F)F)CC3)cc2)nc1-c1ccc2c(c1)CCNC2=O]

### Step 1: Preparation of 4-(4-bromophenyl)-1-(2,2,2-trifluoroethyl)piperidine

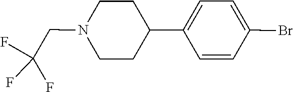 [SMILES: FC(F)(F)CN1CCC(c2ccc(Br)cc2)CC1]

A procedure analogous to that of Example 34, Step 1, using 4-(4-bromophenyl)piperidine (250 mg, 1.04 mmol) in THF (5 mL) at 70° C. in an round bottom flask fitted with a reflux condenser, with phenylsilane (0.257 mL, 2.08 mmol), and TFA (0.140 mL, 1.82 mmol) for 2 h. The reaction mixture was concentrated, the residue was taken up in dichloromethane and washed with saturated aqueous NaHCO₃ solution (×2). The organic solution was then dried and concentrated onto Celite. Silica gel chromatography (eluting with 0-60% EtOAc/Hexanes) afforded the product as a pale yellow oil (301 mg, 79% based on purity of 89%). LCMS: [M+H]⁺=322.41.

### Step 2: 6-(3-amino-6-(4-(1-(2,2,2-trifluoroethyl)piperidin-4-yl)phenyl)pyrazin-2-yl)-3,4-dihydroisoquinolin-1(2H)-one

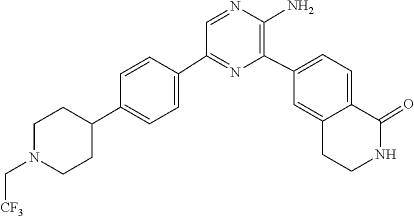 [SMILES: Nc1ncc(-c2ccc(C3CCN(CC(F)(F)F)CC3)cc2)nc1-c1ccc2c(c1)CCNC2=O]

A procedure analogous to that of Example 34, Step 2, using 4-(4-bromophenyl)-1-(2,2,2-trifluoroethyl)piperidine (25 mg, 0.078 mmol, 88.5% purity) with (5-amino-6-(1-oxo-1,2,3,4-tetrahydroisoquinolin-6-yl)pyrazin-2-yl)boronic acid (preparation described in Example 24, Step 1) (26.5 mg, 0.093 mmol) at 90° C. in a microwave reactor for 1.75 h. The reaction mixture was partitioned between brine and EtOAc. The layers were separated, and the aqueous layer was further extracted with CH₂Cl₂ (×2). The combined extract was dried and concentrated onto Celite. Silica gel chromatography (eluting with 0-3.5% MeOH/CH₂Cl₂) afforded the title compound as a pale yellow solid (9 mg, 23%). ¹H NMR (500 MHz, CDCl₃) δ 8.51-8.42 (m, 1H), 8.25-8.19 (m, 1H), 7.94-7.87 (m, 2H), 7.83 (br d, J=7.8 Hz, 1H), 7.71 (s, 1H), 7.36-7.29 (m, 2H), 6.21-6.12 (m, 1H), 4.94-4.73 (m, 2H), 3.67-3.62 (m, 2H), 3.14-3.09 (m, 4H), 3.07-3.01 (m, 2H), 2.56-2.47 (m, 3H), 1.90-1.84 (m, 4H); LCMS: [M+H]⁺=482.46

### Example 37: 6-(3-amino-6-(4-((1R,5S)-3-(2-fluoroethyl)-3-azabicyclo[3.1.0]hexan-1-yl)phenyl)pyrazin-2-yl)-3,4-dihydroisoquinolin-1(2H)-one (I-37)

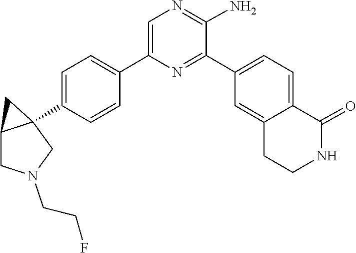 [SMILES: Nc1ncc(-c2ccc([C@@]34C[C@@H]3CN(CCF)C4)cc2)nc1-c1ccc2c(c1)CCNC2=O]

### Step 1: (1R,5S)-1-(4-bromophenyl)-3-(2-fluoroethyl)-3-azabicyclo[3.1.0]hexane

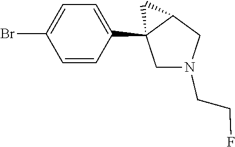 [SMILES: FCCN1C[C@H]2C[C@@]2(c2ccc(Br)cc2)C1]

To a slurry of (1R,5S)-1-(4-bromophenyl)-3-azabicyclo[3.1.0]hexane (100 mg, 0.420 mmol) in DMF (2 mL) at RT, was added 2-fluoroethyl 4-methylbenzenesulfonate (183 mg, 0.840 mmol) and triethylamine (0.585 mL, 4.20 mmol) and the reaction mixture was heated overnight in an oil bath at 100° C. The reaction mixture was partitioned between water (6 mL) and (6 mL) of CHCl₃/IPA (4:1) solvent mixture. The layers were separated and the aqueous layer was further extracted twice with CHCl₃/IPA (4:1) solvent mixture. The combined extracts were dried and concentrated onto Celite. Silica gel chromatography of the residue (eluting with CH₂Cl₂ containing 0-2.5% MeOH and 0-0.25% NH₄OH) afforded the title compound as a brown oil (84 mg, 62% based on purity of 88%). LCMS: [M+H]+=484.36.

### Step 2: 6-(3-amino-6-(4-((1R,5S)-3-(2-fluoroethyl)-3-azabicyclo[3.1.0]hexan-1-yl)phenyl)pyrazin-2-yl)-3,4-dihydroisoquinolin-1(2H)-one

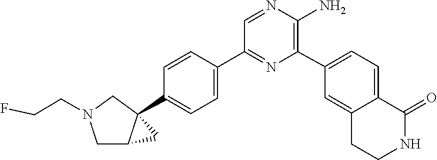 [SMILES: Nc1ncc(-c2ccc([C@@]34C[C@@H]3CN(CCF)C4)cc2)nc1-c1ccc2c(c1)CCNC2=O]

A procedure analogous to that of Example 34, Step 2 using (1R,5S)-1-(4-bromophenyl)-3-(2-fluoroethyl)-3-azabicyclo[3.1.0]hexane (42 mg, 0.129 mmol, 88% purity) and (5-amino-6-(1-oxo-1,2,3,4-tetrahydroisoquinolin-6-yl)pyrazin-2-yl)boronic acid (preparation described in Example 24, Step 1) (47.5 mg, 0.167 mmol) afforded the title compound as a pale yellow solid (10 mg, 23%). ¹H NMR (500 MHz, DMSO-d₆) δ 8.61-8.49 (m, 1H), 8.02-7.95 (m, 2H), 7.93-7.87 (m, 2H), 7.77-7.73 (m, 1H), 7.72-7.69 (m, 1H), 7.25-7.18 (m, 2H), 6.45-6.30 (m, 2H), 4.63-4.56 (m, 1H), 4.53-4.47 (m, 1H), 3.45-3.43 (m, 4H), 3.15-3.10 (m, 1H), 3.03-2.98 (m, 2H), 2.82-2.76 (m, 1H), 2.73-2.66 (m, 1H), 2.56 (br s, 1H), 1.91-1.82 (m, 1H), 1.39-1.34 (m, 1H), 0.82-0.78 (m, 1H); LCMS: [M+H]⁺=444.47.

### Example 38: (R)-6-(3-amino-6-(4-(3-isopropyl-4-methylpiperazin-1-yl)phenyl)pyrazin-2-yl)-3,4-dihydroisoquinolin-1(2H)-one 19399 (I-38)

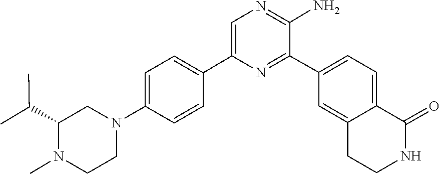 [SMILES: CC(C)[C@@H]1CN(c2ccc(-c3cnc(N)c(-c4ccc5c(c4)CCNC5=O)n3)cc2)CCN1C]

### Step 1: tert-butyl (R)-3-isopropyl-4-methylpiperazine-1-carboxylate

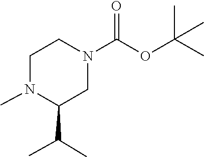 [SMILES: CC(C)[C@@H]1CN(C(=O)OC(C)(C)C)CCN1C]

To a solution of (R)-1-Boc-3-isopropyl-piperazine (1.0 g, 4.4 mmol) in 1:1 MeOH:THF (20 mL) was added an aqueous formaldehyde solution (37%, 0.5 mL, 6.6 mmol) followed by sodium triacetoxyborohydride (1.4 g, 6.6 mmol). The reaction was stirred at RT for 18 h. The volatiles were removed in vacuo and the residue was partitioned between aqueous KOH (1N) and CH₂Cl₂. The layers were separated and the aqueous layer was extracted twice with additional CH₂Cl₂. The combined organic extracts were dried over MgSO₄ and concentrated to dryness to afford the product (1.1 g, quantitative yield). LCMS: [M+H]⁺=243.3.

### Step 2: (R)-2-isopropyl-1-methylpiperazine

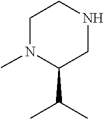

TFA (2 mL, 26 mmol) was added to a solution of tert-butyl (R)-3-isopropyl-4-methylpiperazine-1-carboxylate (0.50 g, 2.06 mmol) in CH₂Cl₂ (10 mL) at RT. The reaction mixture was allowed to stir at RT for 18 h. The volatiles were removed under a stream of compressed air and the residue was dried under reduced pressure to afford the trifluoroacetic acid salt of (R)-2-isopropyl-1-methylpiperazine (1.1 g, quantitative yield). LCMS: [M+H]⁺=143.4.

### Step 3: (R)-4-(4-chlorophenyl)-2-isopropyl-1-methylpiperazine

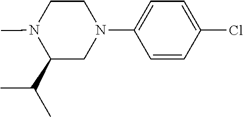 [SMILES: CC(C)[C@@H]1CN(c2ccc(Cl)cc2)CCN1C]

A 30 mL vial was charged with the trifluoroacetic acid salt of (R)-2-isopropyl-1-methylpiperazine (0.50 g, 1.4 mmol), 1-chloro-4-iodobenzene (0.40 g, 1.7 mmol), tris(dibenzylideneacetone)dipalladium (0) (0.037 g, 0.041 mmol), 4,5-bis(diphenylphosphino)-9,9-dimethylxanthene (0.070 g, 0.12 mmol) and Cs₂CO₃ (2.6 g, 8.1 mmol). The vial was sealed with a cap and septum and then the reaction vial was evacuated and backfilled with nitrogen. Toluene (6 mL) was added and the reaction vial was evacuated and backfilled with nitrogen an additional time. The reaction was heated conventionally at 100° C. for 18 h. After cooling to RT the reaction mixture was concentrated directly onto Celite® and purified by flash chromatography (0.5-9.5% CH₂Cl₂/MeOH+0.5% NH₄OH) to afford the product (0.18 g, 51%). LCMS: [M+H]⁺=253.3.

### Step 4: (R)-2-isopropyl-1-methyl-4-(4-(4,4,5,5-tetramethyl-1,3,2-dioxaborolan-2-yl)phenyl)piperazine

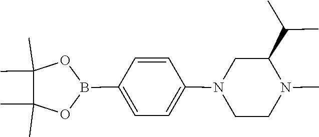

A 30 mL vial was charged with (R)-4-(4-chlorophenyl)-2-isopropyl-1-methylpiperazine (0.18 g, 0.69 mmol), bis(pinacolato)diboron (0.26 g, 1.0 mmol), KOAc (0.14 g, 1.4 mmol) and XPhos® Pd G2 (0.041 g, 0.052 mmol). The vial was sealed with a cap and septum and then the reaction vial was evacuated and backfilled with nitrogen. 1,4-Dioxane (6 mL) was added and the reaction vial was evacuated and backfilled with nitrogen an additional time. The reaction was heated conventionally at 110° C. for 18 h. After cooling to RT the reaction mixture was concentrated directly onto Celite and purified by flash chromatography (0.5-7.5% CH₂Cl₂/MeOH+0.5% NH₄OH) to afford the product (0.20 g, 83%). LCMS: [M+H]⁺=345.4.

### Step 5: (R)-6-(3-amino-6-(4-(3-isopropyl-4-methylpiperazin-1-yl)phenyl)pyrazin-2-yl)-3,4-dihydroisoquinolin-1(2H)-one

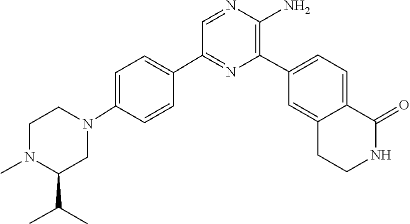 [SMILES: CC(C)[C@@H]1CN(c2ccc(-c3cnc(N)c(-c4ccc5c(c4)CCNC5=O)n3)cc2)CCN1C]

A 30 mL vial was charged with 6-(3-amino-6-bromopyrazin-2-yl)-3,4-dihydroisoquinolin-1(2H)-one (0.055 g, 0.17 mmol), (R)-2-isopropyl-1-methyl-4-(4-(4,4,5,5-tetramethyl-1,3,2-dioxaborolan-2-yl)phenyl)piperazine (0.065 g, 0.19 mmol) and [1,12-bis(diphenylphosphino)ferrocene]dichloropalladium(II) CH₂Cl₂ complex (0.014 g, 0.017 mmol). The vial was sealed with a cap and septum and the reaction vessel was evacuated and backfilled with nitrogen. 1,4-Dioxane (2.0 mL) and 2 M aqueous Na₂CO₃ (0.26 mL, 0.52 mmol) were added and the reaction vessel was evacuated and backfilled with nitrogen an additional time. The reaction mixture was heated at 90° C. for 18 h in an aluminum block. The reaction mixture was concentrated onto Celite® and purified by flash chromatography (0.5-9.5% CH₂Cl₂/MeOH+0.5% NH₄OH). The product containing fractions were concentrated and further purified by reverse phase chromatography (Biotage SNAP C18; 5-60% MeCN/water+0.1% Formic Acid). Isolation of the title compound was achieved by a catch and release procedure using Biotage SCX2 silica gel to afford the title compound (120 mg, 14%) as a beige solid. ¹H NMR (500 MHz; DMSO-d₆) δ 8.48 (s, 1H), 7.9-8.0 (m, 2H), 7.84 (d, J=8.8 Hz; 2H), 7.75 (dd, J=8.0, 1.5 Hz, 1H), 7.71 (s, 1H), 6.99 (d, J=8.9 Hz, 2H), 6.22 (s, 2H), 3.60 (br d, J=10.8 Hz, 1H), 3.52 (br d, J=11.5 Hz, 1H), 3.43 (dt, J=6.5, 2.6 Hz, 2H), 2.99 (t, J=6.5 Hz, 2H), 2.8-2.9 (m, 1H), 2.74 (dt, J=11.8, 2.9 Hz, 1H), 2.31 (dt, J=11.6, 3.2 Hz, 1H), 2.20 (s, 3H), 2.13 (dt, J=6.9, 4.3 Hz, 1H), 1.95 (td, J=10.9, 3.3 Hz, 1H), 1.00 (d, J=7.0 Hz, 3H), 0.88 (d, J=7.0 Hz, 3H); LCMS: [M+H]⁺=457.3.

### Example 39: (R)-6-(3-amino-6-(2-fluoro-4-(3-isopropyl-4-methylpiperazin-1-yl)phenyl)pyrazin-2-yl)-3,4-dihydroisoquinolin-1(2H)-one (I-39)

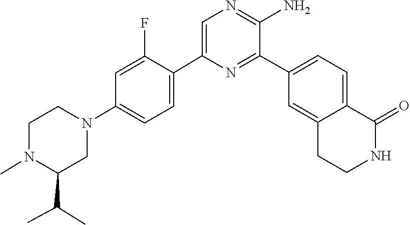 [SMILES: CC(C)[C@@H]1CN(c2ccc(-c3cnc(N)c(-c4ccc5c(c4)CCNC5=O)n3)c(F)c2)CCN1C]

### Step 1: tert-butyl (R)-3-isopropyl-4-methylpiperazine-1-carboxylate

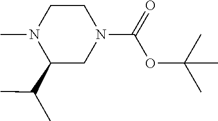 [SMILES: CC(C)[C@@H]1CN(C(=O)OC(C)(C)C)CCN1C]

A procedure analogous to Example 38, Step 1 using (R)-1-Boc-3-isopropyl-piperazine (1.0 g, 4.38 mmol) in MeOH/THF (10 mL each), formaldehyde solution, 37% wt in water (0.489 ml, 6.57 mmol) and NaBH(OAc)₃ (1.39 g, 6.57 mmol) afforded the product (1.06 g, 100%) as a clear oil. LCMS: [M+H]⁺=243.34.

### Step 2: (R)-2-isopropyl-1-methylpiperazine trifluoracetic acid salt

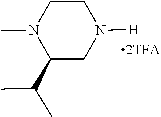

A procedure analogous to Example 38, Step 2 (1.97 ml, 25.8 mmol) using tert-butyl (R)-3-isopropyl-4-methylpiperazine-1-carboxylate (0.50 g, 2.06 mmol) afforded the product (1.07 g, 140%). LCMS: [M+H]⁺=143.35.

### Step 3: (R)-4-(4-chloro-3-fluorophenyl)-2-isopropyl-1-methylpiperazine

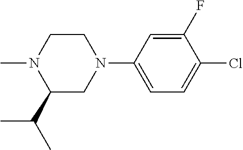 [SMILES: CC(C)[C@@H]1CN(c2ccc(Cl)c(F)c2)CCN1C]

A procedure analogous to Example 38, Step 3 with (R)-2-isopropyl-1-methylpiperazine, 2 TFA (0.500 g, 1.35 mmol) and 4-bromo-1-chloro-2-fluorobenzene (0.196 mL, 1.688 mmol) afforded the product (167 mg, 46%). LCMS: [M+H]⁺=271.09.

### Step 4: (R)-4-(3-fluoro-4-(4,4,5,5-tetramethyl-1,3,2-dioxaborolan-2-yl)phenyl)-2-isopropyl-1-methylpiperazine)

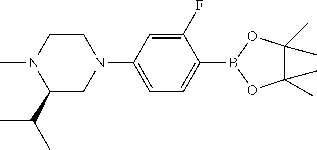

A procedure analogous to Example 38, Step 4 using (R)-4-(4-chloro-3-fluorophenyl)-2-isopropyl-1-methylpiperazine (0.167 g, 0.617 mmol), bis(pinacolato)diboron (0.235 g, 0.925 mmol), K₂CO₃ (0.121 g, 1.233 mmol) and XPhos Pd G2 (36 mg, 0.046 mmol) afforded the product (263 mg, quantitative yield). LCMS: [M+H]⁺=363.30.

### Step 5: (R)-6-(3-amino-6-(2-fluoro-4-(3-isopropyl-4-methylpiperazin-1-yl)phenyl)pyrazin-2-yl)-3,4-dihydroisoquinolin-1(2H)-one (I-39)

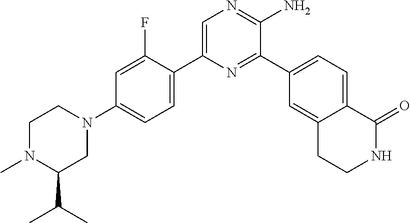 [SMILES: CC(C)[C@@H]1CN(c2ccc(-c3cnc(N)c(-c4ccc5c(c4)CCNC5=O)n3)c(F)c2)CCN1C]

A procedure similar to that used in Example 38, Step 5 using 1,12-bis(diphenylphosphino)ferrocene]dichloropalladium(II), CH₂Cl₂ complex (0.014 g, 0.017 mmol) and (R)-4-(3-fluoro-4-(4,4,5,5-tetramethyl-1,3,2-dioxaborolan-2-yl)phenyl)-2-isopropyl-1-methylpiperazine (0.069 g, 0.190 mmol) afforded the title compound (12 mg, 13%). ¹H NMR (500 MHz, DMSO-d₆) δ 8.31 (d, J=2.4 Hz, 1H), 7.9-8.0 (m, 2H), 7.7-7.8 (m, 2H), 7.69 (s, 1H), 6.87 (dd, J=8.9, 2.4 Hz, 1H), 6.81 (dd, J=15.3, 2.3 Hz, 1H), 6.35 (s, 2H), 3.65 (br d, J=12.1 Hz, 1H), 3.53 (br d, J=11.9 Hz, 1H), 3.42 (dt, J=6.5, 2.7 Hz, 3H), 2.99 (t, J=6.5 Hz, 2H), 2.8-2.9 (m, 1H), 2.78 (dt, J=11.8, 2.9 Hz, 1H), 2.29 (dt, J=11.5, 3.0 Hz, 2H), 2.19 (s, 3H), 2.12 (dt, J=6.9, 4.2 Hz, 1H), 1.92 (td, J=10.7, 3.5 Hz, 1H), 1.00 (d, J=7.0 Hz, 3H), 0.88 (d, J=7.1 Hz, 3H); LCMS: [M+H]⁺=475.3.

### Example 40: (S)-6-(3-amino-6-(4-(3-isopropyl-4-methylpiperazin-1-yl)phenyl)pyrazin-2-yl)-3,4-dihydroisoquinolin-1(2H)-one (I-40)

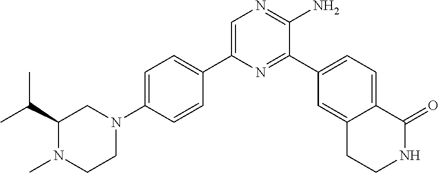 [SMILES: CC(C)[C@H]1CN(c2ccc(-c3cnc(N)c(-c4ccc5c(c4)CCNC5=O)n3)cc2)CCN1C]

### Step 1: tert-butyl (S)-3-isopropyl-4-methylpiperazine-1-carboxylate

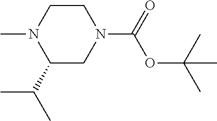 [SMILES: CC(C)[C@H]1CN(C(=O)OC(C)(C)C)CCN1C]

A procedure analogous to Example 38, Step 1 using (S)-1-Boc-3-isopropyl-piperazine (1.0 g, 4.38 mmol), formaldehyde solution, 37% wt in water (0.49 mL, 6.57 mmol) followed by NaBH(OAc)₃ (1.39 g, 6.57 mmol) afforded the product (1.09 g, quantitative yield). LCMS: [M+H]⁺=243.42.

### Step 2: (S)-4-(4-chlorophenyl)-2-isopropyl-1-methylpiperazine

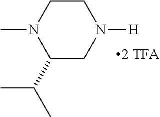

A procedure analogous to Example 38, Step 2 using TFA (1.97 mL, 25.8 mmol), tert-butyl (S)-3-isopropyl-4-methylpiperazine-1-carboxylate (0.50 g, 2.063 mmol) in CH₂Cl₂ (10 mL) at RT afforded the product (1.05 g, quantitative yield). LCMS: [M+H]⁺=143.42.

### Step 3: (S)-4-(4-chlorophenyl)-2-isopropyl-1-methylpiperazine

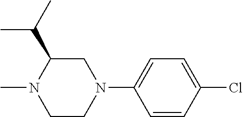 [SMILES: CC(C)[C@H]1CN(c2ccc(Cl)cc2)CCN1C]

A procedure analogous to Example 38, Step 3 using (S)-2-isopropyl-1-methylpiperazine-2TFA (0.500 g, 1.35 mmol), 1-chloro-4-iodobenzene (0.402 g, 1.69 mmol), tris(dibenzylideneacetone)dipalladium (0) (0.037 g, 0.041 mmol), 4,5-bis(diphenylphosphino)-9,9-dimethylxanthene (0.070 g, 0.122 mmol) and Cs₂CO₃ (2.64 g, 8.10 mmol) afforded the product (150 mg, 44%). LCMS: [M+H]⁺=253.27.

### Step 4: (S)-2-isopropyl-1-methyl-4-(4-(4,4,5,5-tetramethyl-1,3,2-dioxaborolan-2-yl)phenyl)piperazine

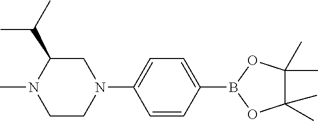

A procedure analogous to Example 38, Step 4 using (S)-4-(4-chlorophenyl)-2-isopropyl-1-methylpiperazine (0.150 g, 0.593 mmol), bis(pinacolato)diboron (0.226 g, 0.890 mmol), KOAc (0.116 g, 1.19 mmol) and XPhos Pd G2 (0.035 g, 0.045 mmol) afforded the product (158 mg, 77%). LCMS: [M+H]⁺=345.32.

### Step 5: (S)-6-(3-amino-6-(4-(3-isopropyl-4-methylpiperazin-1-yl)phenyl)pyrazin-2-yl)-3,4-dihydroisoquinolin-1(2H)-one

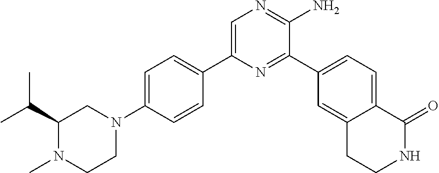 [SMILES: CC(C)[C@H]1CN(c2ccc(-c3cnc(N)c(-c4ccc5c(c4)CCNC5=O)n3)cc2)CCN1C]

A procedure analogous to Example 38, Step 5 using [1,12-bis(diphenylphosphino)ferrocene]dichloropalladium(II), CH₂Cl₂ complex (0.017 g, 0.020 mmol), (S)-2-isopropyl-1-methyl-4-(4-(4,4,5,5-tetramethyl-1,3,2-dioxaborolan-2-yl)phenyl)piperazine (0.077 g, 0.224 mmol) and [1,12-bis(diphenylphosphino)ferrocene]dichloropalladium(II), CH₂Cl₂ complex (0.017 g, 0.020 mmol afforded title compound (13 mg, 13%) as a yellow powder. ¹H NMR (500 MHz, DMSO-d₆) δ 8.48 (s, 1H), 7.9-8.0 (m, 2H), 7.84 (d, J=8.9 Hz, 2H), 7.75 (dd, J=8.0, 1.5 Hz, 1H), 7.71 (s, 1H), 6.99 (d, J=8.9 Hz, 2H), 6.22 (s, 2H), 3.60 (br d, J=11.6 Hz, 1H), 3.52 (br d, J=12.0 Hz, 1H), 3.43 (dt, J=6.5, 2.8 Hz, 2H), 2.99 (t, J=6.5 Hz, 2H), 2.8-2.9 (m, 1H), 2.74 (dt, J=11.8, 2.9 Hz, 1H), 2.31 (dt, J=11.5, 3.1 Hz, 1H), 2.20 (s, 3H), 2.13 (qd, J=11.1, 7.0 Hz, 1H), 1.95 (td, J=10.7, 3.3 Hz, 1H), 1.00 (d, J=7.0 Hz, 3H), 0.88 (d, J=7.0 Hz, 3H); LCMS: [M+H]⁺=457.3.

### Example 41: (S)-6-(3-amino-6-(2-fluoro-4-(3-isopropyl-4-methylpiperazin-1-yl)phenyl)pyrazin-2-yl)-3,4-dihydroisoquinolin-1(2H)-one 19402 (I-40)

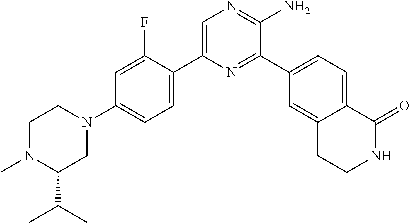 [SMILES: CC(C)[C@H]1CN(c2ccc(-c3cnc(N)c(-c4ccc5c(c4)CCNC5=O)n3)c(F)c2)CCN1C]

### Step 1: (S)-4-(4-chloro-3-fluorophenyl)-2-isopropyl-1-methylpiperazine

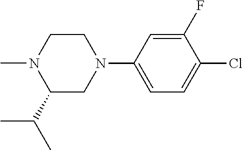 [SMILES: CC(C)[C@H]1CN(c2ccc(Cl)c(F)c2)CCN1C]

A procedure similar to Example 38, Step 3 using (S)-2-isopropyl-1-methylpiperazine-2TFA (preparation described in Example 40, Step 2) (0.500 g, 1.350 mmol), 4-bromo-1-chloro-2-fluorobenzene (0.196 mL, 1.69 mmol), tris(dibenzylideneacetone)dipalladium (0) (0.037 g, 0.041 mmol), 4,5-bis(diphenylphosphino)-9,9-dimethylxanthene (0.070 g, 0.122 mmol) and Cs₂CO₃ (2.64 g, 8.10 mmol) afforded the product (178 mg, 49%). LCMS: [M+H]⁺=271.01.

### Step 2: (S)-4-(3-fluoro-4-(4,4,5,5-tetramethyl-1,3,2-dioxaborolan-2-yl)phenyl)-2-isopropyl-1-methylpiperazine

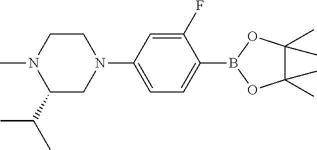

A procedure similar to Example 38, Step 4 using (S)-4-(4-chloro-3-fluorophenyl)-2-isopropyl-1-methylpiperazine (0.178 g, 0.657 mmol), bis(pinacolato)diboron (0.250 g, 0.986 mmol), KOAc (0.129 g, 1.32 mmol) and XPhos Pd G2 (0.039 g, 0.049 mmol) afforded the product (252 mg, quantitative yield). LCMS: [M+H]⁺=363.22.

### Step 3: (S)-6-(3-amino-6-(2-fluoro-4-(3-isopropyl-4-methylpiperazin-1-yl)phenyl)pyrazin-2-yl)-3,4-dihydroisoquinolin-1(2H)-one (I-40)

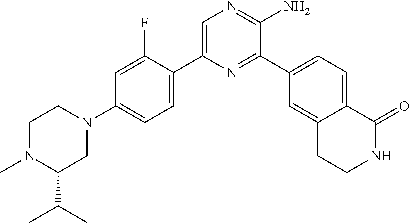 [SMILES: CC(C)[C@H]1CN(c2ccc(-c3cnc(N)c(-c4ccc5c(c4)CCNC5=O)n3)c(F)c2)CCN1C]

A procedure analogous to Example 38, Step 5 using [1,12-bis(diphenylphosphino)ferrocene]dichloropalladium(II), CH₂Cl₂ complex (0.013 g, 0.016 mmol), (S)-4-(3-fluoro-4-(4,4,5,5-tetramethyl-1,3,2-dioxaborolan-2-yl)phenyl)-2-isopropyl-1-methylpiperazine (0.062 g, 0.172 mmol) and [1,12-bis(diphenylphosphino)ferrocene]dichloropalladium(II), CH₂Cl₂ complex (0.013 g, 0.016 mmol) afforded the title compound (10 mg, 13%). ¹H NMR (500 MHz, DMSO-d₆) δ 8.31 (d, J=2.4 Hz, 1H), 7.9-8.0 (m, 2H), 7.7-7.8 (m, 2H), 7.69 (s, 1H), 6.8-6.9 (m, 2H), 6.35 (s, 2H), 3.65 (br dd, J=11.5, 0.9 Hz, 1H), 3.53 (br d, J=12.1 Hz, 1H), 3.42 (dt, J=6.5, 2.8 Hz, 3H), 2.99 (t, J=6.6 Hz, 2H), 2.8-2.9 (m, 1H), 2.78 (dt, J=11.8, 2.9 Hz, 1H), 2.29 (dt, J=11.4, 2.9 Hz, 1H), 2.19 (s, 3H), 2.12 (qd, J=11.0, 6.9 Hz, 1H), 1.92 (td, J=10.8, 3.3 Hz, 1H), 1.00 (d, J=7.0 Hz, 3H), 0.88 (d, J=7.0 Hz, 3H); LCMS: [M+H]⁺=475.3.

### Example 42: (R)-6-(3-amino-6-(4-(2-methylpyrrolidin-1-yl)phenyl)pyrazin-2-yl)-3,4-dihydroisoquinolin-1(2H)-one (I-42)

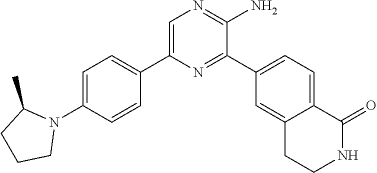 [SMILES: C[C@@H]1CCCN1c1ccc(-c2cnc(N)c(-c3ccc4c(c3)CCNC4=O)n2)cc1]

### Step 1: (R)-1-(4-chlorophenyl)-2-methylpyrrolidine

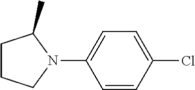

A procedure analogous to Example 38, Step 3 using (R)-2-methylpyrrolidine hydrochloride (0.300 g, 2.47 mmol), 1-chloro-4-iodobenzene (0.735 g, 3.08 mmol), tris(dibenzylideneacetone)dipalladium (0) (0.068 g, 0.074 mmol), 4,5-Bis(diphenylphosphino)-9,9-dimethylxanthene (0.128 g, 0.222 mmol) and Cs₂CO₃ (4.02 g, 12.3 mmol) afforded the product (178 mg, 37%). LCMS: [M+H]⁺=196.05.

### Step 2: (R)-2-methyl-1-(4-(4,4,5,5-tetramethyl-1,3,2-dioxaborolan-2-yl)phenyl)pyrrolidine

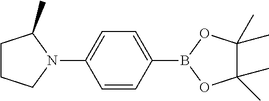 [SMILES: C[C@@H]1CCCN1c1ccc(B2OC(C)(C)C(C)(C)O2)cc1]

A procedure analogous to Example 38, Step 4 using (R)-1-(4-chlorophenyl)-2-methylpyrrolidine (0.178 g, 0.910 mmol), bis(pinacolato)diboron (0.346 g, 1.36 mmol), KOAc (0.179 g, 1.82 mmol) and XPhos Pd G2 (0.054 g, 0.068 mmol) afforded the product (156 mg, 52%). LCMS: [M+H]+=288.09.

### Step 3: (R)-6-(3-amino-6-(4-(2-methylpyrrolidin-1-yl)phenyl)pyrazin-2-yl)-3,4-dihydroisoquinolin-1(2H)-one

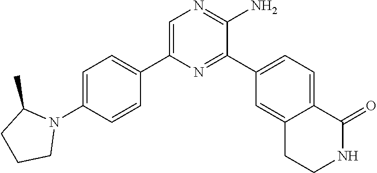 [SMILES: C[C@@H]1CCCN1c1ccc(-c2cnc(N)c(-c3ccc4c(c3)CCNC4=O)n2)cc1]

A procedure analogous to Example 38, Step 5 using 6-(3-amino-6-bromopyrazin-2-yl)-3,4-dihydroisoquinolin-1(2H)-one (0.070 g, 0.219 mmol), (R)-2-methyl-1-(4-(4,4,5,5-tetramethyl-1,3,2-dioxaborolan-2-yl)phenyl)pyrrolidine (0.069 g, 0.241 mmol) and [1,12-bis(diphenylphosphino)ferrocene]dichloropalladium(II), CH₂Cl₂ complex (0.018 g, 0.022 mmol) afforded the title compound (23 mg, 26%) as a yellow powder. ¹H NMR (500 MHz, DMSO-d₆) δ 8.45 (s, 1H), 7.9-8.0 (m, 2H), 7.81 (d, J=8.8 Hz, 2H), 7.76 (d, J=8.1 Hz, 1H), 7.72 (s, 1H), 6.62 (d, J=8.8 Hz, 2H), 6.13 (s, 1H), 3.93 (br t, J=5.9 Hz, 1H), 3.4-3.5 (m, 4H), 3.1-3.2 (m, 1H), 3.00 (br t, J=6.5 Hz, 2H), 2.0-2.1 (m, 2H), 1.69 (br d, J=2.3 Hz, 1H), 1.13 (d, J=6.1 Hz; 3H); LCMS: [M+H]⁺=400.2.

### Example 43: (R)-6-(3-amino-6-(2-fluoro-4-(2-methylpyrrolidin-1-yl)phenyl)pyrazin-2-yl)-3,4-dihydroisoquinolin-1(2H)-one (I-43)

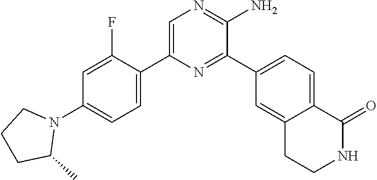 [SMILES: C[C@@H]1CCCN1c1ccc(-c2cnc(N)c(-c3ccc4c(c3)CCNC4=O)n2)c(F)c1]

### Step 1: (R)-1-(4-chloro-3-fluorophenyl)-2-methylpyrrolidine

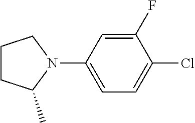

A procedure analogous to Example 38, Step 3 using (R)-2-methylpyrrolidine hydrochloride (0.300 g, 2.467 mmol), 4-bromo-1-chloro-2-fluorobenzene (0.359 mL, 3.08 mmol), tris(dibenzylideneacetone)dipalladium (0) (0.068 g, 0.074 mmol), 4,5-bis(diphenylphosphino)-9,9-dimethylxanthene (0.128 g, 0.222 mmol) and Cs₂CO₃ (4.02 g, 12.33 mmol) afforded the product (268 mg, 51%). LCMS: [M+H]⁺=214.02.

### Step 2: (R)-1-(3-fluoro-4-(4,4,5,5-tetramethyl-1,3,2-dioxaborolan-2-yl)phenyl)-2-methylpyrrolidine

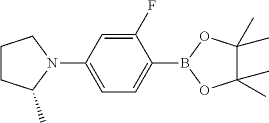 [SMILES: C[C@@H]1CCCN1c1ccc(B2OC(C)(C)C(C)(C)O2)c(F)c1]

A procedure analogous to Example 38, Step 4 using (R)-1-(4-chloro-3-fluorophenyl)-2-methylpyrrolidine (0.268 g, 1.25 mmol), bis(pinacolato)diboron (0.478 g, 1.88 mmol), KOAc (0.246 g, 2.51 mmol) and XPhos Pd G2 (0.074 g, 0.094 mmol) afforded the product (244 mg, 64%). LCMS: [M+H]⁺=306.06.

### Step 3: (R)-6-(3-amino-6-(2-fluoro-4-(2-methylpyrrolidin-1-yl)phenyl)pyrazin-2-yl)-3,4-dihydroisoquinolin-1(2H)-one

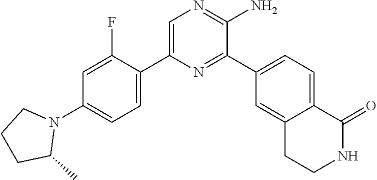 [SMILES: C[C@@H]1CCCN1c1ccc(-c2cnc(N)c(-c3ccc4c(c3)CCNC4=O)n2)c(F)c1]

A procedure analogous to Example 38, Step 5 using 6-(3-amino-6-bromopyrazin-2-yl)-3,4-dihydroisoquinolin-1(2H)-one (0.075 g, 0.235 mmol), (R)-1-(3-fluoro-4-(4,4,5,5-tetramethyl-1,3,2-dioxaborolan-2-yl)phenyl)-2-methylpyrrolidine (0.079 g, 0.258 mmol) and [1,12-bis(diphenylphosphino)ferrocene]dichloropalladium(II), CH₂Cl₂ complex (0.019 g, 0.023 mmol) afforded the title compound (33 mg, 34%) as a yellow powder. ¹H NMR (500 MHz, DMSO-d₆) δ 8.21 (d, J=2.4 Hz, 1H), 7.9-7.9 (m, 2H), 7.6-7.7 (m, 2H), 7.63 (s, 1H), 6.43 (dd, J=8.8. 2.3 Hz, 1H), 6.31 (dd, J=15.2, 2.2 Hz, 1H), 6.20 (s, 2H), 3.85 (quin, J=6.0 Hz, 1H), 3.3-3.4 (m, 3H), 3.0-3.1 (m, 1H), 2.92 (t, J=6.5 Hz, 2H), 1.9-2.0 (m, 3H), 1.62 (br d, J=4.9 Hz, 1H), 1.05 (d, J=6.2 Hz, 3H); LCMS: [M+H]⁺=418.4.

### Example 44: 6-(3-amino-6-(4-(4-isopropylpiperazin-1-yl)phenyl)pyrazin-2-yl)-7-fluoro-3,4-dihydroisoquinolin-1(2H)-one (I-44)

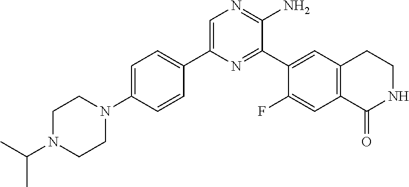 [SMILES: CC(C)N1CCN(c2ccc(-c3cnc(N)c(-c4cc5c(cc4F)C(=O)NCC5)n3)cc2)CC1]

A 30 mL vial was charged with 4-(4-isopropylpiperazinyl)phenylboronic acid, pinacol ester (0.073 g, 0.22 mmol), 6-(3-amino-6-bromopyrazin-2-yl)-7-fluoro-3,4-dihydroisoquinolin-1(2H)-one (0.075 g, 0.22 mmol) and XPhos Pd G2 (0.018 g, 0.022 mmol). The vial was sealed with a cap and septum and then the reaction vial was evacuated and backfilled with nitrogen. 1,4-Dioxane (2.0 mL) and 1.3 M aqueous K₃PO₄ (0.43 mL, 0.56 mmol) were added and the reaction vessel was evacuated and backfilled with nitrogen an additional time. The reaction was heated at 90° C. for 18 h. After cooling to RT the reaction mixture was concentrated directly onto Celite and purified by flash chromatography (0.5-7.5% CH₂Cl₂/MeOH+0.5% NH₄OH). The product containing fractions were concentrated and further purified by reverse phase chromatography (Biotage SNAP C18; 5-60% MeCN/water+0.1% Formic Acid). Isolation of the title compound was achieved by a catch and release procedure using Biotage SCX2 silica gel to afford the title compound (0.55 g, 54%). ¹H NMR (500 MHz, DMSO-d₆) δ 8.52 (s, 1H), 8.14 (br s, 1H), 7.79 (d, J=8.8 Hz, 2H), 7.65 (d, J=10.1 Hz, 1H), 7.52 (d, J=6.8 Hz, 1H), 6.98 (d, J=8.9 Hz, 2H), 6.16 (s, 2H), 3.43 (dt, J=6.5, 2.7 Hz, 3H), 3.1-3.2 (m, 4H), 2.97 (br t, J=6.5 Hz, 2H), 2.7-2.7 (m, 1H), 2.6-2.6 (m, 5H), 1.01 (d, J=6.5 Hz, 6H); LCMS: [M+H]⁺=461.5.

### Example 45: 6-(3-amino-6-(4-((1R,5S)-3-methyl-3-azabicyclo[3.1.0]hexan-1-yl)phenyl)pyrazin-2-yl)-7-fluoro-3,4-dihydroisoquinolin-1(2H)-one (I-45)

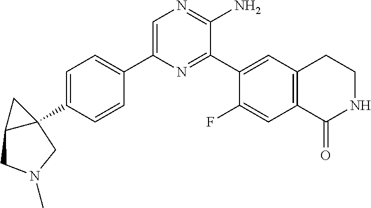 [SMILES: CN1C[C@H]2C[C@@]2(c2ccc(-c3cnc(N)c(-c4cc5c(cc4F)C(=O)NCC5)n3)cc2)C1]

### Step 1: (5-amino-6-(7-fluoro-1-oxo-1,2,3,4-tetrahydroisoquinolin-6-yl)pyrazin-2-yl)boronic acid

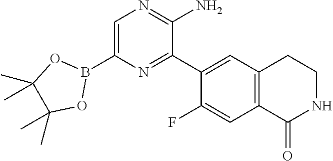 [SMILES: CC1(C)OB(c2cnc(N)c(-c3cc4c(cc3F)C(=O)NCC4)n2)OC1(C)C]

A 30 mL vial was charged with 6-(3-amino-6-bromopyrazin-2-yl)-7-fluoro-3,4-dihydroisoquinolin-1(2H)-one (0.300 g, 0.890 mmol), bis(pinacolato)diboron (0.339 g, 1.34 mmol), [1,12-bis(diphenylphosphino)ferrocene]dichloropalladium(II) (0.065 g, 0.089 mmol) and KOAc (0.218 g, 2.23 mmol). The vial was sealed with a cap and septum and evacuated and back filled with nitrogen gas. 1,4-Dioxane (8 mL) was added and the reaction vial was evacuated and back filled an addition time. The reaction was heated to 90° C. in an aluminum block for 2 h. The mixture was allowed to cool to RT and used as a solution as obtained in the next step. LCMS: [M+H]⁺=303.39 Step 2: (1R,5S)-1-(4-bromophenyl)-3-methyl-3-azabicyclo[3.1.0]hexane

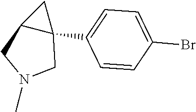

To a solution of (1R,5S)-1-(4-bromophenyl)-3-azabicyclo[3.1.0]hexane (0.500 g, 2.100 mmol) in MeOH/THF (5 mL each) was added formaldehyde solution, 37% wt in water (0.234 mL, 3.15 mmol) followed by NaBH(OAc)₃ (0.668 g, 3.15 mmol). The reaction was stirred at RT for 40 h. The volatiles were removed in vacuo and the residue was partitioned between KOH (1N) and CH₂Cl₂. The layers were separated and the aqueous layer was extracted with additional CH₂Cl₂. The combined extracts were dried over Mg₂SO₄ and concentrated to dryness to afford the product (501 mg, 95%). LCMS: [M+H]⁺=252.21.

### Step 3: 6-(3-amino-6-(4-((1R,5S)-3-methyl-3-azabicyclo[3.1.0]hexan-1-yl)phenyl)pyrazin-2-yl)-7-fluoro-3,4-dihydroisoquinolin-1(2H)-one

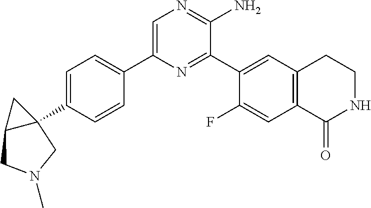 [SMILES: CN1C[C@H]2C[C@@]2(c2ccc(-c3cnc(N)c(-c4cc5c(cc4F)C(=O)NCC5)n3)cc2)C1]

A procedure analogous to Example 38, Step 5 using (1R,5S)-1-(4-bromophenyl)-3-methyl-3-azabicyclo[3.1.0]hexane (0.054 g, 0.215 mmol) and XPhos Pd G2 (0.017 g, 0.022 mmol), (5-amino-6-(7-fluoro-1-oxo-1,2,3,4-tetrahydroisoquinolin-6-yl)pyrazin-2-yl)boronic acid (0.065 g, 0.215 mmol) and aqueous K₃PO₄ (0.414 mL of a 1.3 M solution, 0.538 mmol) afforded the title compound (33 mg, 36%) as a yellow powder. ¹H NMR (500 MHz, DMSO-d₆) δ 8.59 (s, 1H), 8.15 (br s, 1H), 7.85 (d, J=8.4 Hz, 2H), 7.65 (d, J=10.1 Hz, 1H), 7.52 (d, J=6.7 Hz, 1H), 7.18 (d, J=8.4 Hz, 2H), 6.32 (s, 2H), 3.43 (br dd, J=6.1, 4.2 Hz, 5H), 2.9-3.0 (m, 3H), 2.40 (dd, J=8.7, 3.3 Hz, 1H), 2.30 (s, 3H), 1.81 (td, J=7.8 Hz, 3.9, 1H), 1.37 (t, J=4.0 Hz, 1H), 0.77 (dd, J=7.9, 3.8 Hz, 1H); LCMS: [M+H]⁺=430.5.

### Example 46: 6-(3-amino-6-(4-((1R,5S)-3-isopropyl-3-azabicyclo[3.1.0]hexan-1-yl)phenyl)pyrazin-2-yl)-7-fluoro-3,4-dihydroisoquinolin-1(2H)-one (I-46)

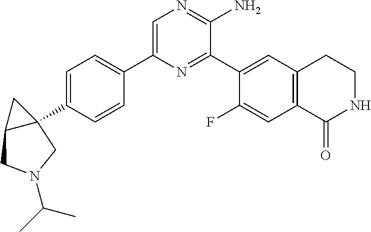 [SMILES: CC(C)N1C[C@H]2C[C@@]2(c2ccc(-c3cnc(N)c(-c4cc5c(cc4F)C(=O)NCC5)n3)cc2)C1]

### Step 1: (1R,5S)-1-(4-bromophenyl)-3-isopropyl-3-azabicyclo[3.1.0]hexane

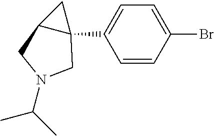 [SMILES: CC(C)N1C[C@H]2C[C@@]2(c2ccc(Br)cc2)C1]

To a solution of (1R,5S)-1-(4-bromophenyl)-3-azabicyclo[3.1.0]hexane (0.500 g, 2.10 mmol) in MeOH/THF (5 mL each) was added acetone (0.231 mL, 3.15 mmol) and acetic acid (0.012 mL, 0.210 mmol) followed by NaBH(OAc)₃ (0.668 g, 3.15 mmol). The reaction was stirred at RT for 40 h. The volatiles were removed in vacuo and the residue was partitioned between KOH (1N) and CH₂Cl₂. The layers were separated and the aqueous layer was extracted with additional CH₂Cl₂. The combined extracts were dried over MgSO₄ and concentrated to dryness to afford the product (587 mg, 100%). LCMS: [M+H]⁺=280.29.

### Step 2: 6-(3-amino-6-(4-((1R,5S)-3-isopropyl-3-azabicyclo[3.1.0]hexan-1-yl)phenyl)pyrazin-2-yl)-7-fluoro-3,4-dihydroisoquinolin-1(2H)-one

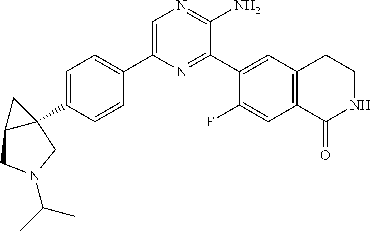 [SMILES: CC(C)N1C[C@H]2C[C@@]2(c2ccc(-c3cnc(N)c(-c4cc5c(cc4F)C(=O)NCC5)n3)cc2)C1]

A procedure similar to Example 38, Step 5 using (1R,5S)-1-(4-bromophenyl)-3-isopropyl-3-azabicyclo[3.1.0]hexane (0.060 g, 0.215 mmol) and XPhos Pd G2 (0.017 g, 0.022 mmol), (5-amino-6-(7-fluoro-1-oxo-1,2,3,4-tetrahydroisoquinolin-6-yl)pyrazin-2-yl)boronic acid (0.065 g, 0.215 mmol, freshly prepared as described in Example 45 and used as a solution in 2.0 mL 1,4-dioxane) afforded the title compound (31 mg, 31%) as a yellow powder. ¹H NMR (500 MHz, DMSO-d₆) δ 8.59 (s, 1H), 8.15 (br s, 1H), 7.84 (d, J=8.3 Hz, 2H), 7.65 (d, J=10.1 Hz, 1H), 7.52 (d, J=6.8 Hz, 1H), 7.20 (d, J=8.4 Hz, 2H), 6.32 (s, 2H), 3.43 (dt, J=6.5, 2.8 Hz, 3H), 3.05 (d, J=8.6 Hz, 1H), 2.97 (br t, J=6.4 Hz, 2H), 2.58 (br d, J=8.3 Hz, 2H), 2.47 (br d, J=5.7 Hz, 2H), 1.82 (td, J=7.7, 3.8 Hz, 1H), 1.32 (t, J=3.8 Hz, 1H), 1.03 (dd, J=14.8, 6.2 Hz, 6H), 0.75 (dd, J=7.9, 3.5 Hz, 1H); LCMS [M+H]⁺=458.6.

### Example 47: 6-(3-amino-6-(4-((1S,5R)-3-methyl-3-azabicyclo[3.1.0]hexan-1-yl)phenyl)pyrazin-2-yl)-7-fluoro-3,4-dihydroisoquinolin-1(2H)-one (I-47)

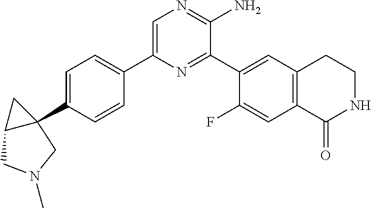 [SMILES: CN1C[C@@H]2C[C@]2(c2ccc(-c3cnc(N)c(-c4cc5c(cc4F)C(=O)NCC5)n3)cc2)C1]

### Step 1: (1S,5R)-1-(4-bromopheny)-3-methyl-3-azabicyclo[3.1.0]hexane

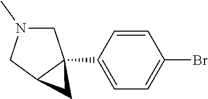

To a solution of (1S,5R)-1-(4-bromophenyl)-3-azabicyclo[3.1.0]hexane (0.50 g, 2.1 mmol) in 1:1 MeOH:THF (10 mL) was added formaldehyde (37% wt, 0.23 mL, 3.2 mmol) followed by NaBH(OAc)₃ (0.67 g, 3.2 mmol). The reaction was stirred at RT for 40 h. The volatiles were removed in vaccuo and the residue was partitioned between 1 M aqueous KOH (15 mL) and CH₂Cl₂ (15 mL). The layers were separated and the aqueous layer was extracted with additional portions of CH₂Cl₂. The combined extracts were dried over MgSO₄ and concentrated to dryness to afford the product (0.73 g, quantitative yield) that was used in the next step without further purification. LCMS: [M+H]⁺=252.3.

### Step 2: 6-(3-amino-6-(4-((1S,5R)-3-methyl-3-azabicyclo[3.1.0]hexan-1-yl)phenyl)pyrazin-2-yl)-7-fluoro-3,4-dihydroisoquinolin-1(2H)-one

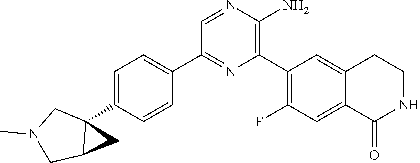 [SMILES: CN1C[C@@H]2C[C@]2(c2ccc(-c3cnc(N)c(-c4cc5c(cc4F)C(=O)NCC5)n3)cc2)C1]

A stock solution of (5-amino-3-fluoro-6-(7-fluoro-1-oxo-1,2,3,4-tetrahydroisoquinolin-6-yl)pyrazin-2-yl)boronic acid was prepared as follows: A 30 mL vial was charged with 6-(3-amino-6-bromopyrazin-2-yl)-7-fluoro-3,4-dihydroisoquinolin-1(2H)-one (0.30 g, 0.89 mmol), bis(pinacolato)diboron (0.34 g, 1.3 mmol), [1,12-bis(diphenylphosphino)ferrocene]dichloropalladium(II) (0.065 g, 0.089 mmol) and KOAc (0.22 g, 2.2 mmol). The vial was sealed with a cap and septum and evacuated and back filled with nitrogen gas. 1,4-Dioxane (8 mL) was added and the reaction vial was evacuated and back filled an addition time. The reaction was heated to 90° C. in an aluminum block for 2 h. After cooling to RT, 2.0 mL of this stock solution was transferred to a sealed 30 mL vial that was charged with (1S,5R)-1-(4-bromophenyl)-3-methyl-3-azabicyclo[3.1.0]hexane (0.054 g, 0.22 mmol) and XPhos Pd G2 (0.017 g, 0.022 mmol). Aqueous K₃PO₄ (0.41 mL of a 1.3 M solution, 0.54 mmol) was added via syringe and the reaction vessel was evacuated and backfilled with nitrogen. The reaction mixture was heated at 90° C. for 18 h in an aluminum block. The reaction mixture was concentrated onto Celite and purified by flash chromatography [0.5-9.5% MeOH/CH₂Cl₂+0.5% NH₄OH]. The product containing fractions were concentrated and further purified by reverse phase chromatography (Biotage SNAP C18; 5-60% MeCN/water+0.1% Formic Acid). Isolation of the title compound was achieved by a catch and release procedure using Biotage SCX2 silica gel to afford the title compound (0.029 g, 31%) as a yellow powder. ¹H NMR (500 MHz, DMSO-d₆) δ 8.59 (s, 1H), 8.15 (br s, 1H), 7.85 (d, J=8.4 Hz, 2H), 7.65 (d, J=10.1 Hz, 1H), 7.52 (d, J=7.0 Hz, 1H), 7.18 (d, J=8.4 Hz, 2H), 6.32 (s, 2H), 3.4-3.5 (m, 4H), 3.26 (d, J=8.3 Hz, 1H), 2.9-3.0 (m, 3H), 2.40 (dd, J=8.6, 3.4 Hz, 1H), 2.30 (s, 3H), 1.81 (td, J=7.9, 3.9 Hz, 1H), 1.37 (t, J=4.0 Hz, 1H), 0.77 (dd, J=7.9, 3.8 Hz, 1H); LCMS: [M+H]⁺=430.5.

### Example 48: 6-(3-amino-6-(4-((1S,5R)-3-isopropyl-3-azabicyclo[3.1.0]hexan-1-yl)phenyl)pyrazin-2-yl)-7-fluoro-3,4-dihydroisoquinolin-1(2H)-one (I-48)

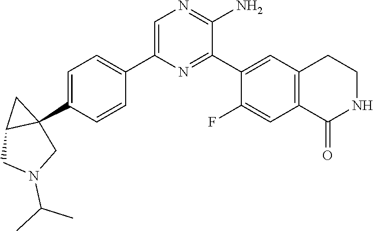 [SMILES: CC(C)N1C[C@@H]2C[C@]2(c2ccc(-c3cnc(N)c(-c4cc5c(cc4F)C(=O)NCC5)n3)cc2)C1]

### Step 1: (1S,5R)-1-(4-bromophenyl)-3-isopropyl-3-azabicyclo[3.1.0]hexane

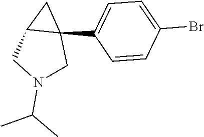 [SMILES: CC(C)N1C[C@@H]2C[C@]2(c2ccc(Br)cc2)C1]

A procedure similar to Example 46, Step 1 using (1S,5R)-1-(4-bromophenyl)-3-azabicyclo[3.1.0]hexane (0.500 g, 2.10 mmol) in MeOH/THF (5 mL each) was added acetone (0.231 mL, 3.15 mmol) and acetic acid (0.012 ml, 0.210 mmol) followed by NaBH(OAc)₃ (0.668 g, 3.15 mmol) afforded the product (665 mg, quantitative yield) as an amber oil. LCMS: [M+H]⁺=280.29.

### Step 2: 6-(3-amino-6-(4-((1S,5R)-3-isopropyl-3-azabicyclo[3.1.0]hexan-1-yl)phenyl)pyrazin-2-yl)-7-fluoro-3,4-dihydroisoquinolin-1(2H)-one (I-48)

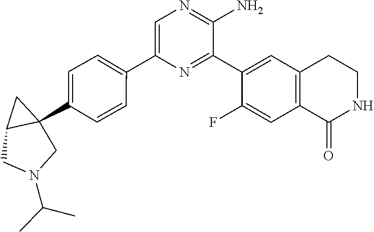 [SMILES: CC(C)N1C[C@@H]2C[C@]2(c2ccc(-c3cnc(N)c(-c4cc5c(cc4F)C(=O)NCC5)n3)cc2)C1]

A procedure similar to Example 38, Step 5 using (1S,5R)-1-(4-bromophenyl)-3-isopropyl-3-azabicyclo[3.1.0]hexane (0.070 g, 0.248 mmol), XPhos Pd G2 (0.020 g, 0.025 mmol) and (5-amino-6-(7-fluoro-1-oxo-1,2,3,4-tetrahydroisoquinolin-6-yl)pyrazin-2-yl)boronic acid (preparation described in Example 45, Step 1) (0.075 g, 0.248 mmol) and aqueous K₃PO₄ (0.477 mL, 0.621 mmol) afforded the title compound (28 mg, 25%) as a yellow powder. ¹H NMR (500 MHz, DMSO-d₆) δ 8.59 (s, 1H), 8.15 (br s, 1H), 7.84 (d, J=8.4 Hz, 2H), 7.65 (d, J=10.1 Hz, 1H), 7.52 (d, J=6.7 Hz, 1H), 7.20 (d, J=8.4 Hz, 2H), 6.32 (s, 2H), 3.43 (td, J=6.5, 3.2 Hz, 3H), 3.05 (d, J=8.6 Hz, 1H), 2.97 (br t, J=6.5 Hz, 2H), 2.58 (br d, J=8.3 Hz, 1H), 2.4-2.5 (m, 2H), 1.82 (td, J=7.8, 3.9 Hz, 1H), 1.32 (t, J=3.9 Hz, 1H), 1.03 (dd, J=14.9, 6.3 Hz, 6H), 0.75 (dd, J=7.8, 3.5 Hz, 1H); LCMS: [M+H]⁺=458.6.

### Example 49: 6-(3-amino-6-(4-(4-methylpiperazin-1-yl)phenyl)pyridazin-4-yl)-3,4-dihydroisoquinolin-1(2H)-one (I-49)

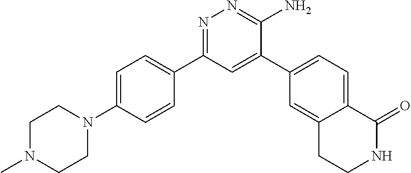 [SMILES: CN1CCN(c2ccc(-c3cc(-c4ccc5c(c4)CCNC5=O)c(N)nn3)cc2)CC1]

### Step 1: 6-(3-amino-6-chloropyridazin-4-yl)-3,4-dihydroisoquinolin-1(2H)-one

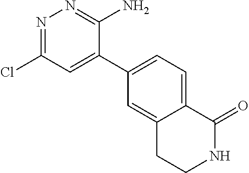 [SMILES: Nc1nnc(Cl)cc1-c1ccc2c(c1)CCNC2=O]

A vial charged with 3,4-dihydro-1(2H)-isoquinolinone-6-boronic acid pinacol ester (92 mg, 0.336 mmol), 3-amino-4-bromo-6-chloropyridazine (70 mg, 0.336 mmol), Cs₂CO₃ (352 mg, 1.08 mmol) and [1,1′-bis(diphenylphosphino)ferrocene]dichloropalladium(II) (26.3 mg, 0.036 mmol) was suspended in water (2 mL). The mixture was degassed with Ar and DME (4 mL) was added. The reaction was degassed again with Ar then heated sealed under microwave irradiation at 90° C. for 2 h. The reaction mixture was concentrated under reduced pressure, deposited on Celite and purified by flash chromatography (12 g SiO₂ InnoFlash® cartridge, using MeOH in CH₂Cl₂ (eluting at 6-12% MeOH) to afford the product as a yellowish solid (65 mg, 43% based on purity of 66%); LCMS: [M+H]⁺=274.93.

### Step 2: 6-(3-amino-6-(4-(4-methylpiperazin-1-yl)phenyl)pyridazin-4-yl)-3,4-dihydroisoquinolin-1(2H)-one

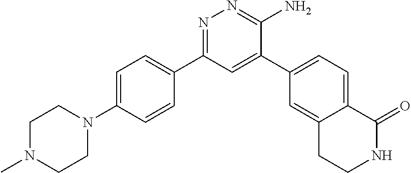 [SMILES: CN1CCN(c2ccc(-c3cc(-c4ccc5c(c4)CCNC5=O)c(N)nn3)cc2)CC1]

4-(4-Methylpiperazin-1-yl)phenylboronic acid pinacol ester (30.7 mg, 0.102 mmol), K₃PO₄ (49.7 mg, 0.234 mmol), and XPhos Pd G2 (6.14 mg, 7.81 μmol) were added to degassed suspension of 6-(3-amino-6-chloropyridazin-4-yl)-3,4-dihydroisoquinolin-1(2H)-one (32.5 mg, 0.078 mmol) in ACN (4 mL) and water (2.6 mL). The mixture was degassed with N₂ and then heated in a microwave reactor at 100° C. for 2 h. The reaction mixture was concentrated under reduced pressure, deposited on Celite and purified by flash chromatography (12 g SiO₂ InnoFlash® cartridge, using MeOH in CH₂Cl₂ eluting at 23% MeOH, and then by preparative HPLC (30 g Biotage® SNAP KP-C₁₈-HS, MeOH in (H₂O+0.05% TFA)) eluting at 25% MeOH). The relevant fractions were concentrated, filtered through a Waters PoraPak™ CX column (0.4 g), rinsing with MeOH and eluting with 2 M NH₃ in MeOH to afford the title compound as a light yellow solid (24.5 mg, 73%). ¹H NMR (500 MHz, CD₃OD) δ 8.13-8.07 (m, 1H), 7.89-7.83 (m, 2H), 7.71-7.66 (m, 1H), 7.62-7.57 (m, 1H), 7.57-7.54 (m, 1H), 7.08 (br d, J=8.6 Hz, 2H), 3.56 (br t, J=6.5 Hz, 2H), 3.12-3.03 (m, 2H), 2.72-2.61 (m, 4H), 2.38 (s, 3H) observed: 19H, required: 26-3H, a signal due to 4H (piperazine) is obscured by CD₃OD solvent peak; LCMS: [M+H]⁺=415.33.

### Example 50: 7-(3-amino-6-(4-(4-methylpiperazin-1-yl)phenyl)pyrazin-2-yl)-2,3,4,5-tetrahydro-1H-benzo[c]azepin-1-one (I-50)

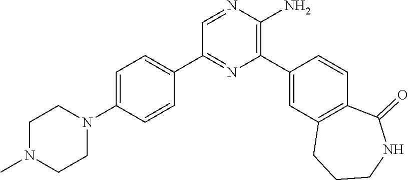 [SMILES: CN1CCN(c2ccc(-c3cnc(N)c(-c4ccc5c(c4)CCCNC5=O)n3)cc2)CC1]

### Step 1: 7-bromo-2,3,4,5-tetrahydro-1H-benzo[c]azepin-1-one

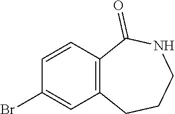 [SMILES: O=C1NCCCc2cc(Br)ccc21]

A stirred solution of 6-bromo-3,4-dihydro-2H-naphthalen-1-one (400 mg, 1.777 mmol, 1 eq) in concentrated HCl (4.2 mL) was cooled to 0° C. and NaN₃ (229 mg, 3.52 mmol, 2 eq) was added. The mixture was allowed to warm to RT and stirred for 12 h. After completion of the reaction, the mixture was poured onto ice and made basic by addition of K₂CO₃. The aqueous mixture was extracted with CH₂Cl₂ two times and the combined organic layers were dried over MgSO₄ and concentrated in vacuo. The crude product obtained was purified by normal phase flash chromatography (gradient: CH₂Cl₂/ACN 0-100%) to afford the product (169 mg, 85%) as white solid. LCMS: [M+H]⁺=240.11.

### Step 2: 7-(4,4,5,5-tetramethyl-1,3,2-dioxaborolan-2-yl)-2,3,4,5-tetrahydro-1H-benzo[c]azepin-1-one

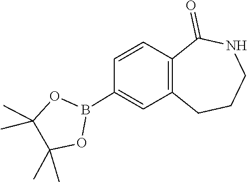 [SMILES: CC1(C)OB(c2ccc3c(c2)CCCNC3=O)OC1(C)C]

To 7-bromo-2,3,4,5-tetrahydro-1H-benzo[c]azepin-1-one (49 mg, 0.208 mmol, 1 eq), bis(pinacolato)diboron (58.2 mg, 0.229 mmol, 1.1 eq) and KOAc (61.3 mg, 0.625 mmol, 3 eq) in 1,4-dioxane (2.5 ml) was added [1,12-bis(diphenylphosphino)ferrocene]dichloropalladium(II) (7.62 mg, 10.41 μmol, 0.05 eq) under Ar. The mixture was heated in a microwave for 2 h at 100° C. LCMS showed full conversion of starting material. The reaction mixture was used in the next step without purification. LCMS: [M+H]⁺=288.16.

### Step 3: 7-(3-amino-6-(4-(4-methylpiperazin-1-yl)phenyl)pyrazin-2-yl)-2,3,4,5-tetrahydro-1H-benzo[c]azepin-1-one

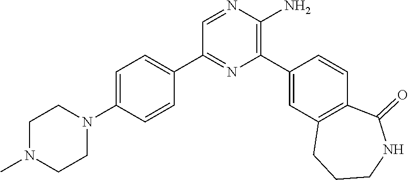 [SMILES: CN1CCN(c2ccc(-c3cnc(N)c(-c4ccc5c(c4)CCCNC5=O)n3)cc2)CC1]

To 7-(4,4,5,5-tetramethyl-1,3,2-dioxaborolan-2-yl)-2,3,4,5-tetrahydro-1H-benzo[c]-azepin-1-one (55 mg, 0.192 mmol, 1 eq) in 1,4-dioxane (2.5 ml) were added 3-bromo-5-(4-(4-methylpiperazin-1-yl)phenyl)pyrazin-2-amine (70 mg, 0.201 mmol, 1.05 eq), K₃PO₄ (111 mg, 0.522 mmol, 2.7 eq) and XPhos Pd G2 (13.7 mg, 0.017 mmol, 0.1 eq) under Ar. The mixture was heated in a microwave overnight at 100° C., evaporated with Celite and purified with RPHLC (ACN/water) to obtain the title compound yellow solid (32 mg, 43%). ¹H NMR (500 MHz, DMSO-d₆) 5=8.47 (s, 1H), 8.11-8.05 (m, 1H), 7.86-7.82 (m, 2H), 7.73 (d, J=7.8 Hz, 1H), 7.66 (s, 1H), 7.63 (d, J=7.8 Hz, 1H), 7.02-6.97 (m, 2H), 6.19 (s, 1H), 6.20 (d, J=5.4 Hz, 1H), 3.21-3.16 (m, 4H), 3.03-2.94 (m, 2H), 2.86-2.80 (m, 2H), 2.48-2.43 (m, 4H), 2.25-2.21 (m, 3H), 1.98-1.91 (m, 2H). LCMS: [M+H]⁺=429.50.

### Example 51: 6-(3-amino-6-(4-(4-methylpiperazin-1-yl)phenyl)pyrazin-2-yl)-3-methylisoquinolin-1(2H)-one (I-51)

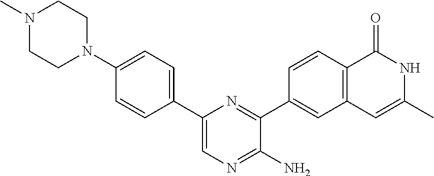

### Step 1: 3-methyl-6-(4,4,5,5-tetramethyl-1,3,2-dioxaborolan-2-yl)isoquinolin-1(2H)-one

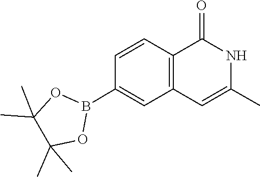

To 6-bromo-3-methyl-2H-isoquinolin-1-one (100 mg, 0.420 mmol), bis(pinacolato)diboron (117 mg, 0.462 mmol), and potassium acetate (124 mg, 1.260 mmol), in a vial was added anhydrous 1,4-dioxane (10.0 ml). The system was flushed with nitrogen then [1,12-bis(diphenylphosphino)ferrocene]dichloropalladium(II) (30.7 mg, 0.042 mmol) was added. The mixture was flushed with nitrogen then heated at 100° C. overnight. The reaction was diluted with acetonitrile, filtered through a pad of celite, concentrated in vacuo and used crude in the next step assuming full conversion (120 mg, 0.341 mmol, 100%) as a mixture of boronate and boronic acid. Boronate LCMS: [M+H]+=285.99, Boronic acid LCMS[M+H]+ 204.17.

### Step 2: 6-(3-amino-6-(4-(4-methylpiperazin-1-yl)phenyl)pyrazin-2-yl)-3-methylisoquinolin-1(2H)-one

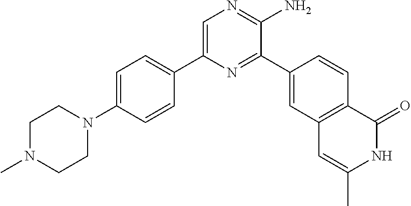 [SMILES: Cc1cc2cc(-c3nc(-c4ccc(N5CCN(C)CC5)cc4)cnc3N)ccc2c(=O)[nH]1]

Prepared from 3-bromo-5-(4-(4-methylpiperazin-1-yl)phenyl)pyrazin-2-amine (50 mg, 0.144 mmol) and 3-methyl-6-(4,4,5,5-tetramethyl-1,3,2-dioxaborolan-2-yl)isoquinolin-1(2H)-one (60.7 mg, 0.172 mmol) to give the title compound (22.8 mg, 37.2% yield) as a bright yellow solid. ¹H NMR (500 MHz, DMSO-d₆) δ ppm 11.29 (s, 1H), 8.51 (s, 1H), 8.22 (d, J=8.31 Hz, 1H), 7.92 (s, 1H), 7.85 (d, J=8.80 Hz, 2H), 7.78 (d, J=8.31 Hz, 1H), 7.00 (d, J=8.93 Hz, 2H), 6.43 (s, 1H), 6.24 (d, J=3.91 Hz, 2H), 3.20 (br. s., 4H), 2.24 (s, 6H); LCMS: [M+H]⁺=427.34.

### Example 52: 6-(3-amino-6-(4-((1S,5R)-3-cyclopropyl-3-azabicyclo[3.1.0]hexan-1-yl)phenyl)pyrazin-2-yl)-7-fluoro-3,4-dihydroisoquinolin-1(2H)-one (I-52)

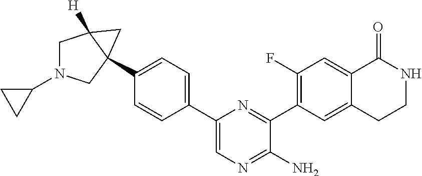 [SMILES: Nc1ncc(-c2ccc([C@]34C[C@H]3CN(C3CC3)C4)cc2)nc1-c1cc2c(cc1F)C(=O)NCC2]

### Step 1: (1S,5R)-1-(4-bromophenyl)-3-cyclopropyl-3-azabicyclo[3.1.0]hexane

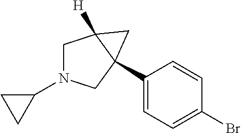 [SMILES: Brc1ccc([C@]23C[C@H]2CN(C2CC2)C3)cc1]

(1S,5R)-1-(4-bromophenyl)-3-azabicyclo[3.1.0]hexane (300 mg, 1.26 mmol) was mixed with (1-Ethoxycyclopropoxy)trimethylsilane (1757 mg, 10.08 mmol) in MeOH (4.5 mL) and AcOH, (1.3 mL), then 1M NaBH₃CN in THF (5.04 mL, 5.04 mmol) was added. The reaction mixture was kept in a sealed vial and stirred under 60-70° C. oil overnight. Additional 1M NaBH₃CN (2 mL, 2 mmol) was added, stirred under 60-70° C. overnight again. The reaction mixture was quenched with sat-NaHCO₃, extracted with DCM. The organic layer was washed with sa-NaHCO₃ and brine, dried over MgSO4, filtered, concentrated by Rotavapor to give the product (350 mg, 100% yield). LCMS: [M+H]⁺=278.38.

### Step 2: (1S,5R)-3-cyclopropyl-1-(4-(4,4,5,5-tetramethyl-1,3,2-dioxaborolan-2-yl)phenyl)-3-azabicyclo[3.1.0]hexane

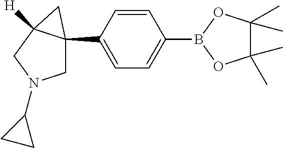 [SMILES: CC1(C)OB(c2ccc([C@]34C[C@H]3CN(C3CC3)C4)cc2)OC1(C)C]

In a similar manner, (1 S,5R)-3-cyclopropyl-1-(4-(4,4,5,5-tetramethyl-1,3,2-dioxaborolan-2-yl)phenyl)-3-azabicyclo[3.1.0]hexane (400 mh, 98%) was prepared from (1S,5R)-1-(4-bromophenyl)-3-cyclopropyl-3-azabicyclo[3.1.0]hexane (350 mg, 1.258 mmol) reacted with bis(pinacolato)diboron (383 mg, 1.31 mmol), [1,12-bis(diphenylphosphino)ferrocene]dichloro-palladium(II) (92 mg, 0.125 mmol) and potassium acetate (370 mg, 3.77 mmol) in 1,4-dioxane (10 mL) under 80° C. oil bath for 2 hour. LCMS: [M+H]⁺=326.21

### Step 3: 6-(3-Amino-6-(4-((1S,5R)-3-cyclopropyl-3-azabicyclo[3.1.0]hexan-1-yl)phenyl)pyrazin-2-yl)-7-fluoro-3,4-dihydroisoquinolin-1(2H)-one

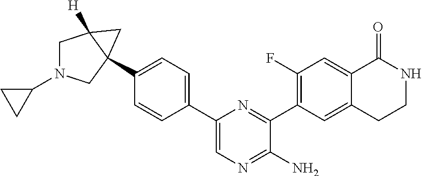 [SMILES: Nc1ncc(-c2ccc([C@]34C[C@H]3CN(C3CC3)C4)cc2)nc1-c1cc2c(cc1F)C(=O)NCC2]

Prepared from 6-(3-amino-6-bromopyrazin-2-yl)-7-fluoro-3,4-dihydroisoquinolin-1(2H)-one (62.2 mg, 0.184 mmol), reacted with (1S,5R)-3-cyclopropyl-1-(4-(4,4,5,5-tetramethyl-1,3,2-dioxaborolan-2-yl)phenyl)-3-azabicyclo[3.1.0]hexane (50 mg, 0.154 mmol), K₂CO₃ (42.5 mg, 0.307 mmol) and tetrakis(triphenylphosphine)palladium(0) (17.8 mg, 0.015 mmol) in 1,4-dioxane (2 mL) and water (0.5 mL) under 110° C. oil bath for 2 hour to give the title compound (13.3 mg, 19%). ¹H NMR (500 MHz, DMSO-d₆) 5=8.59 (s, 1H), 8.15 (br s, 1H), 7.85 (d, J=8.2 Hz, 2H), 7.65 (d, J=10.1 Hz, 1H), 7.52 (br d, J=6.7 Hz, 1H), 7.19 (d, J=8.2 Hz, 2H), 6.33 (s, 2H), 3.48-3.39 (m, 2H), 3.29 (br d, J=8.3 Hz, 1H), 3.03-2.94 (m, 3H), 2.81 (d, J=8.4 Hz, 1H), 2.69 (dd, J=3.2, 8.5 Hz, 1H), 1.83-1.71 (m, 2H), 1.21 (t, J=3.9 Hz, 1H), 0.75 (dd, J=3.7, 7.8 Hz, 1H), 0.40 (br d, J=6.0 Hz, 2H), 0.36-0.25 (m, 2H); LCMS: [M+H]⁺=456.46.

### Example 53: (R)-6-(3-amino-6-(4-(4-isopropyl-2-methylpiperazin-1-yl)phenyl)pyrazin-2-yl)-3,4-dihydroisoquinolin-1(2H)-one (I-53)

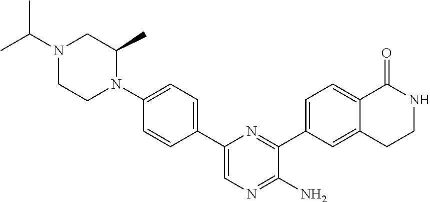 [SMILES: CC(C)N1CCN(c2ccc(-c3cnc(N)c(-c4ccc5c(c4)CCNC5=O)n3)cc2)[C@H](C)C1]

### Step 1: (R)-1-(4-bromophenyl)-4-isopropyl-2-methylpiperazine

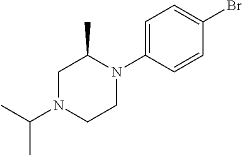 [SMILES: CC(C)N1CCN(c2ccc(Br)cc2)[C@H](C)C1]

### Step 1: tert-butyl (R)-4-isopropyl-2-methylpiperazine-1-carboxylate

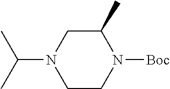 [SMILES: CC(C)N1CCN(C(=O)OC(C)(C)C)[C@H](C)C1]

To a solution of tert-butyl (R)-2-methylpiperazine-1-carboxylate (5 g, 24.96 mmol) in DMF (125 mL), sodium triacetoxyborohydride (10.58 g, 49.92 mmol) and acetone (2.89 g, 49.92 mmol), acetic acid (2.24 g, 37.44 mmol) were added at room temperature and the reaction mass was stirred at 60° C. for 3 h. After completion of the reaction, the mixture was diluted with cold saturated NaHCO₃ solution (150 mL) and extracted with EtOAc (3×60 mL). The aqueous layer was discarded and combined organic layer was washed with saturated citric acid solution (2×60 mL). The aqueous citric acid layer was made basic (pH=9) using NaHCO₃ and extracted with EtOAc (3×60 mL). The combined organic layer was dried over Na₂SO₄ and concentrated under vacuum to afford the title compound (5.4 g, 89% yield) as colourless liquid. LCMS: [M+H]⁺=243.3.

### Step 2: (R)-1-isopropyl-3-methylpiperazine

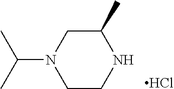

To a stirred solution of tert-butyl (R)-4-isopropyl-2-methylpiperazine-1-carboxylate (5.4 g, 22.28 mmol) in DCM (54 mL) at 0° C. 4N HCl in dioxane (54 mL) was added dropwise and the reaction mass was stirred at RT for 16 h. After completion of the reaction, the mixture was concentrated under vacuum to afford the title compound (5 g, quant. crude yield) as off-white gummy solid. LCMS: [M+H]⁺=143.1.

### Step 3: (R)-1-(4-bromophenyl)-4-isopropyl-2-methylpiperazine

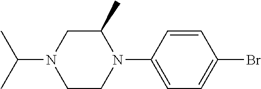 [SMILES: CC(C)N1CCN(c2ccc(Br)cc2)[C@H](C)C1]

To a stirred solution of (R)-1-isopropyl-3-methylpiperazine (2 g, 14.06 mmol) in toluene (30 mL), 1-bromo-4-iodobenzene (4.76 g, 16.87 mmol), palladium acetate (0.16 g, 0.703 mmol), tri-tert-butyl phophonium HBF₄ (0.44 g, 1.406 mmol), potassium tert-butoxide (5.04 g, 49.22 mmol) were added and the mixture was degassed using Ar for 10 min and stirred at 120° C. for 16 h. The mixture was poured into water (80 mL) and extracted with EtOAc (3×40 mL), dried over Na₂SO₄ and concentrated under vacuum to get crude compound. The crude was purified by column chromatography and product was eluted in 1.8% MeOH in DCM to afford the title compound (0.88 g, 21%) as brown. LCMS: [M+H]⁺=299.4.

### Step 2: 6-(3-amino-6-(4-(4-methylpiperazin-1-yl)phenyl)pyrazin-2-yl)-3-methylisoquinolin-1(2H)-one IDW-4019-0240-10

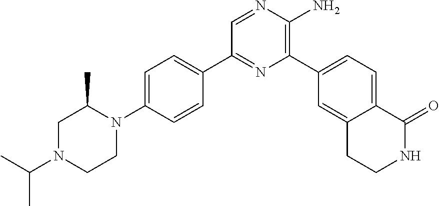 [SMILES: CC(C)N1CCN(c2ccc(-c3cnc(N)c(-c4ccc5c(c4)CCNC5=O)n3)cc2)[C@H](C)C1]

Prepared from 6-(3-amino-6-bromopyrazin-2-yl)-3,4-dihydroisoquinolin-1(2H)-one (96 mg, 0.300 mmol) and (R)-4-isopropyl-2-methyl-1-(4-(4,4,5,5-tetramethyl-1,3,2-dioxaborolan-2-yl)phenyl)piperazine (124 mg, 0.36 mmol) to give the title compound as a yellow solid (0.078 mmol, 25.9% yield). ¹H NMR (500 MHz, DMSO-d₆) δ 8.47 (s, 1H), 7.98 (br s, 1H), 7.95 (d, J=7.9 Hz, 1H), 7.83 (d, J=8.9 Hz, 2H), 7.75 (dd, J=8.0, 1.4 Hz, 1H), 7.70 (s, 1H), 6.93 (d, J=9.0 Hz, 2H), 6.20 (s, 2H), 4.05 (dt, J=6.2, 2.9 Hz, 1H), 3.42 (td, J=6.6, 2.6 Hz, 2H), 3.35 (br d, J=3.1 Hz, 1H), 2.99 (t, J=6.5 Hz, 2H), 2.97-2.90 (m, 1H), 2.83 (br dd, J=10.8, 1.8 Hz, 1H), 2.71-2.64 (m, 2H), 2.45 (dd, J=10.8, 3.2 Hz, 1H), 2.31 (td, J=11.0, 3.2 Hz, 1H), 1.02 (d, J=6.5 Hz, 3H), 1.00 (t, J=6.9 Hz, 6H). LCMS: [M+H]⁺=457.24.

### Example 54: (R)-6-(3-amino-6-(4-(2,4-dimethylpiperazin-1-yl)phenyl)pyrazin-2-yl)-3,4-dihydroisoquinolin-1(2H)-one (I-54)

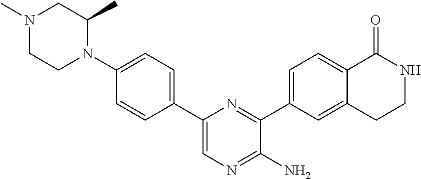 [SMILES: C[C@@H]1CN(C)CCN1c1ccc(-c2cnc(N)c(-c3ccc4c(c3)CCNC4=O)n2)cc1]

### Step 1: tert-butyl (R)-2,4-dimethylpiperazine-1-carboxylate

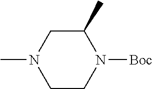

To a solution of tert-butyl (R)-2-methylpiperazine-1-carboxylate (5 g, 24.96 mmol) in DMF (125 mL), sodium triacetoxyborohydride (10.58 g, 49.92 mmol) and 37% formaldehyde (2.25 g, 74.88 mmol), acetic acid (2.24 g, 37.44 mmol) were added at RT and the reaction mass was stirred at 60° C. for 3 h. The mixture was diluted with cold saturated NaHCO₃ solution (150 mL) and extracted with EtOAc (3×60 mL). The aqueous layer was discarded and combined organic layer was washed with saturated citric acid solution (2×60 mL). The aqueous citric acid layer was made basic (pH=9) using NaHCO₃ and extracted with EtOAc (3×60 mL). The combined organic layer was dried over Na₂SO₄ and concentrated under vacuum to afford the product (3.2 g, 59.81% yield) as colourless. LCMS: [M+H]⁺=215.0.

### Step 2: (R)-1,3-dimethylpiperazine

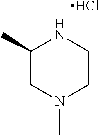

To a stirred solution of tert-butyl (R)-2,4-dimethylpiperazine-1-carboxylate (3.2 g, 14.93 mmol) in DCM (32 mL) at 0° C. 4N HCl in dioxane (32 mL) was added dropwise and the reaction mass was stirred at RT for 16 h. After completion of the reaction, the mixture was concentrated under vacuum to afford the title compound (3 g, quant. crude yield) as off-white gummy solid of (R)-1,3-dimethylpiperazine. ¹H NMR (400 MHz, DMSO-d₆) b 3.75-3.53 (m, 4H), 3.36-3.25 (m, 2H), 3.15 (m, 1H), 2.81 (s, 3H), 1.32 (d, J=6.4 Hz, 3H).

### Step 3: (R)-1-(4-bromophenyl)-2,4-dimethylpiperazine

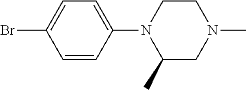

To a stirred solution of (R)-1,3-dimethylpiperazine (0.7 g, 6.12 mmol) in toluene (105 mL), 1-bromo-4-iodobenzene (1.96 g, 7.34 mmol), palladium acetate (0.07 g, 0.306 mmol), tri-tert-butyl phophonium HBF₄ (0.175 g, 0.612 mmol), potassium tert-butoxide (2.38 g, 21.43 mmol) were added and the reaction mass degassed using Ar for 10 min and stirred at 120° C. for 16 h. After completion of reaction, reaction mass was poured into water (30 mL) and extracted with EtOAc (3×15 mL), dried over anhydrous Na₂SO₄ and concentrated under vacuum to get crude compound which was purified by sgc, eluting with 1.6% MeOH in DCM, to afford the product (0.26 g, 15.8% yield) as brown solid. LCMS: [M+H]⁺=271.3.

### Step 4: (R)-2,4-dimethyl-1-(4-(4,4,5,5-tetramethyl-1,3,2-dioxaborolan-2-yl)phenyl)piperazine IDW-4019-0229-10

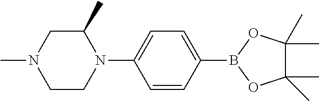

In a microwave vial with magnetic stir bar was placed Bis(pinacolato)diboron (302 mg, 1.189 mmol), (R)-1-(4-bromophenyl)-2,4-dimethylpiperazine (291 mg, 1.081 mmol), [1,12-Bis(diphenylphosphino)ferrocene]dichloropalladium(II) (79 mg, 0.108 mmol), Potassium acetate (318 mg, 3.24 mmol) and 1,4-Dioxane (2 mL). The flask was sealed, then heated to 100° C. on a heating block overnight when LCMS indicated the reaction was complete [01]. The reaction was diluted with acetonitrile, filtered through a pad of Celite, concentrated and then used in the next step assuming full conversion; (R)-2,4-dimethyl-1-(4-(4,4,5,5-tetramethyl-1,3,2-dioxaborolan-2-yl)phenyl)piperazine (1.081 mmol, 100% yield) [10].

### Step 5: (R)-6-(3-amino-6-(4-(2,4-dimethylpiperazin-1-yl)phenyl)pyrazin-2-yl)-3,4-dihydroisoquinolin-1(2H)-one

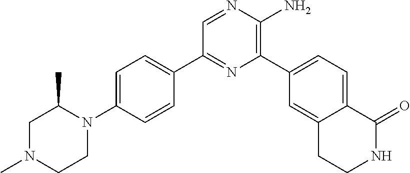 [SMILES: C[C@@H]1CN(C)CCN1c1ccc(-c2cnc(N)c(-c3ccc4c(c3)CCNC4=O)n2)cc1]

Prepared from 6-(3-amino-6-bromopyrazin-2-yl)-3,4-dihydroisoquinolin-1(2H)-one (96 mg, 0.300 mmol) and (R)-2,4-dimethyl-1-(4-(4,4,5,5-tetramethyl-1,3,2-dioxaborolan-2-yl)phenyl)piperazine (114 mg, 0.36 mmol) give the title compound as a yellow solid (0.164 mmol, 54.7% yield). ¹H NMR (500 MHz, DMSO-d₆) δ 8.47 (s, 1H), 7.97 (br s, 1H), 7.95 (d, J=8.1 Hz, 1H), 7.83 (d, J=8.8 Hz, 2H), 7.75 (dd, J=7.9, 1.3 Hz, 1H), 7.70 (s, 1H), 6.94 (d, J=9.0 Hz, 2H), 6.19 (s, 2H), 4.04 (br dd, J=6.2, 3.1 Hz, 1H), 3.42 (td, J=6.5, 2.7 Hz, 2H), 3.38 (br s, 1H), 3.02-2.95 (m, 3H), 2.83-2.77 (m, 1H), 2.64 (br d, J=10.8 Hz, 1H), 2.24 (dd, J=10.9, 3.5 Hz, 1H), 2.19 (s, 3H), 2.04 (td, J=11.2, 3.4 Hz, 1H), 1.04 (d, J=6.5 Hz, 3H). LCMS: [M+1]⁺=429.17.

### Example 55: 6-(3-amino-6-(4-((3R,5S)-3,4,5-trimethylpiperazin-1-yl)phenyl)pyrazin-2-yl)-3,4-dihydroisoquinolin-1(2H)-one (I-55)

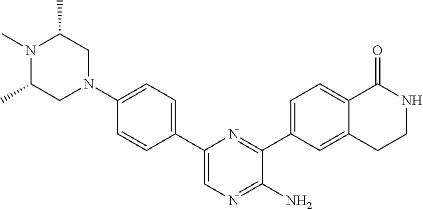 [SMILES: C[C@@H]1CN(c2ccc(-c3cnc(N)c(-c4ccc5c(c4)CCNC5=O)n3)cc2)C[C@H](C)N1C]

### Step 1: (2S,6R)-4-(4-bromophenyl)-1,2,6-trimethylpiperazine

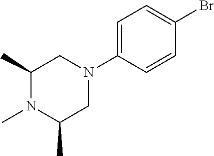 [SMILES: C[C@@H]1CN(c2ccc(Br)cc2)C[C@H](C)N1C]

### Step 2: (2R,6S)-1,2,6-trimethyl-4-(4-(4,4,5,5-tetramethyl-1,3,2-dioxaborolan-2-yl)phenyl)piperazine

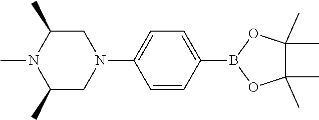

In a microwave vial with magnetic stir bar was placed Bis(pinacolato)diboron (302 mg, 1.189 mmol), (2S,6R)-4-(4-bromophenyl)-1,2,6-trimethylpiperazine (306 mg, 1.080 mmol), [1,12-Bis(diphenylphosphino)ferrocene]dichloropalladium(II) (79 mg, 0.108 mmol), potassium acetate (318 mg, 3.24 mmol) and 1,4-Dioxane (2 mL). The flask was sealed, then heated to 100° C. for 1 h in the microwave (high absorbance) when LCMS indicated incomplete conversion [01]. The reaction was then heated on a heating block at 100° C. overnight when LCMS indicated the reaction was complete. The reaction was diluted with acetonitrile, filtered through a pad of Celite, concentrated and then used in the next step assuming full conversion to give the product (1.081 mmol, 100% yield). LCMS [M+H]⁺=332.1

### Step 3: 6-(3-amino-6-(4-((3S,5R)-3,4,5-trimethylpiperazin-1-yl)phenyl)pyrazin-2-yl)-3,4-dihydroisoquinolin-1(2H)-one

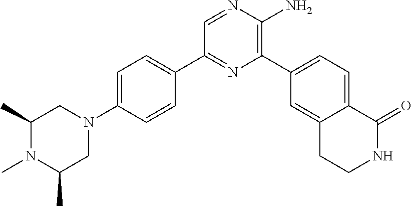 [SMILES: C[C@@H]1CN(c2ccc(-c3cnc(N)c(-c4ccc5c(c4)CCNC5=O)n3)cc2)C[C@H](C)N1C]

Prepared from 6-(3-amino-6-bromopyrazin-2-yl)-3,4-dihydroisoquinolin-1(2H)-one (96 mg, 0.300 mmol) and (2R,6S)-1,2,6-trimethyl-4-(4-(4,4,5,5-tetramethyl-1,3,2-dioxaborolan-2-yl)phenyl)piperazine (119 mg, 0.36 mmol) to give the title compound as a yellow solid (0.126 mmol, 41.9% yield). ¹H NMR (500 MHz, DMSO-d₆) δ 8.48 (s, 1H), 7.98 (br s, 1H), 7.95 (d, J=8.1 Hz, 1H), 7.83 (d, J=8.9 Hz, 2H), 7.74 (dd, J=8.0, 1.5 Hz, 1H), 7.70 (d, J=1.0 Hz, 1H), 6.99 (d, J=9.0 Hz, 2H), 6.21 (s, 2H), 3.61 (br d, J=10.9 Hz, 2H), 3.42 (td, J=6.5, 2.7 Hz, 2H), 2.99 (t, J=6.5 Hz, 2H), 2.42 (t, J=11.4 Hz, 2H), 2.28-2.21 (m, 2H), 2.19 (s, 3H), 1.08 (d, J=6.1 Hz, 6H). LCMS: [M+H]⁺=443.24

### Example 56: 6-(3-amino-6-(4-(4-ethylpiperazin-1-yl)phenyl)pyrazin-2-yl)-3,4-dihydroisoquinolin-1(2H)_-one (I-56)

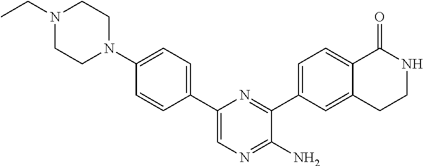 [SMILES: CCN1CCN(c2ccc(-c3cnc(N)c(-c4ccc5c(c4)CCNC5=O)n3)cc2)CC1]

Prepared from 6-(3-amino-6-bromopyrazin-2-yl)-3,4-dihydroisoquinolin-1(2H)-one (101 mg, 0.316 mmol) and (4-(4-ethylpiperizine-1-yl)phenyl)boronic acid pinocol ester (120 mg, 0.380 mmol) to give the title compound as a yellow solid: (0.219 mmol, 69.4% yield). 1H NMR (500 MHz, DMSO-d₆) δ 8.48 (s, 1H), 7.98 (br s, 1H), 7.95 (d, J=7.9 Hz, 1H), 7.84 (d, J=8.9 Hz, 2H), 7.75 (dd, J=8.0, 1.5 Hz, 1H), 7.70 (s, 1H), 6.99 (d, J=8.9 Hz, 2H), 6.22 (s, 2H), 3.42 (td, J=6.6, 2.6 Hz, 2H), 3.21-3.16 (m, 4H), 2.99 (t, J=6.5 Hz, 2H), 2.52 (br s, 4H), 2.38 (q, J=7.1 Hz, 2H), 1.04 (t, J=7.2 Hz, 3H). LCMS: [M+H]⁺=429.32.

### Example 57: 7-(3-amino-6-(4-(4-methylpiperazin-1-yl)phenyl)pyrazin-2-yl)-2-methylquinazolin-4(3H)-one (I-57)

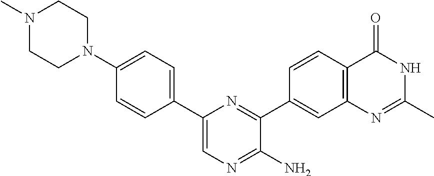

### Step 1: 5-iodopyrazin-2-amine

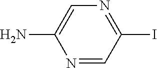

To a stirred solution of 2-aminopyrazine (15 g, 157.9 mmol), in DMF (150 mL) was added NIS (53.23 g, 236.7 mmol) at 0° C. Then, the reaction mixture was slowly warmed to RT stirred for 6 h under argon atmosphere. The reaction mixture was poured into ice water extracted with EtOAc (2×500 mL) washed with water (200 mL), followed by brine (200 mL). The separated organic layer was dried over Na₂SO₄ and concentrated under reduced pressure to give a crude residue, which was purified by column chromatography (silica gel 100-200 mesh) eluting with 10-30% EtOAc in petroleum ether to afford the product (9 g, 26%) as a pale yellow solid. LCMS: [M+H]⁺: =222.09.

### Step 2: 3-bromo-5-iodopyrazin-2-amine

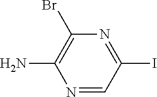

To a stirred solution of 5-iodopyrazin-2-amine (9 g, 40.74 mmol), in DMF (90 mL) was added NBS (5.28 g, 44.8 mmol) at 0° C. The reaction mixture was slowly warmed to RT stirred for 4 h under argon atmosphere, then poured into ice water extracted with EtOAc (2×300 mL), washed with water (200 mL), followed by brine (200 mL). The separated organic layer was dried over Na₂SO₄ and concentrated under reduced pressure to give a crude residue, which was purified by column chromatography (silica gel 100-200 mesh) using an eluent 10-30% EtOAc in petroleum ether to afford the product (5 g, 41%) as a pale-yellow solid. LCMS: [M−H]⁺=300.05.

### Step 3: 1-methyl-4-(4-(4,4,5,5-tetramethyl-1,3,2-dioxaborolan-2-yl)phenyl)piperazine

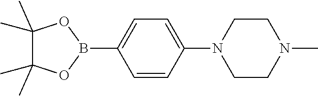

To a solution of 3-bromo-5-iodopyrazin-2-amine (0.25 g, 0.9 mmol), compound 5 (0.27 g, 1.0 mmol) KOAc (0.28 g, 2.9 mmol) in 1,4-dioxane (5 mL) was degassed with argon for 20 min, followed by addition of Pd(dppf)Cl₂ DCM (0.08 g, 0.09 mmol) under argon atmosphere. Then the reaction mixture was heated to 100° C. for 16 h before cooling to RT. The reaction mixture was filtered through a celite bed, which was washed with EtOAc (20 mL). The combined filtrate was concentrated under reduced pressure to afford the product as a black color liquid. LCMS: [M+H]⁺=303.34 Step 4: 3-bromo-5-(4-(4-methylpiperazin-1-yl)phenyl)pyrazin-2-amine

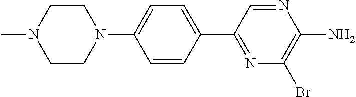 [SMILES: CN1CCN(c2ccc(-c3cnc(N)c(Br)n3)cc2)CC1]

To a degassed suspension of 5-iodopyrazin-2-amine (5 g, 16.7 mmol) and 1-methyl-4-(4-(4,4,5,5-tetramethyl-1,3,2-dioxaborolan-2-yl)phenyl)piperazine (5.56 g, 18.4 mmol) and Na₂CO₃ (3.54 g, 33.4 mmol) in 1,4-dioxane:H₂O (50:10 mL) was added Pd(PPh₃)₄ (0.96 g, 0.8 mmol) at rt−90° C. over 16 h under argon atmosphere before cooling to RT. The reaction mixture was poured into ice water (100 mL) extracted with EtOAc (2×200 mL) washed with water (100 mL), followed by brine (100 mL). The separated organic layer was dried over Na₂SO₄ and concentrated under reduced pressure to give a crude residue, which was purified by column chromatography (silica gel 230-400 mesh) eluting with 0-10% MeOH in DCM to afford the product (2.2 g, 37% yield) as a pale yellow solid. LCMS: [M+H]⁺=348.3.

ii. Preparation of Exemplary Compounds of Formula II

### Example 58: 6-(3-amino-6-(2-((dimethylamino)methyl)-4-(tetrahydro-2H-pyran-4-yl)phenyl)pyrazin-2-yl)-7-fluoro-3,4-dihydroisoquinolin-1(2H)-one (II-1)

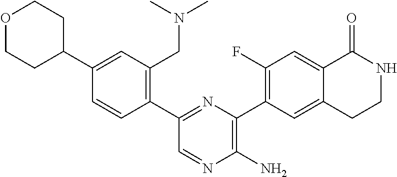 [SMILES: CN(C)Cc1cc(C2CCOCC2)ccc1-c1cnc(N)c(-c2cc3c(cc2F)C(=O)NCC3)n1]

### Step 1: 2-bromo-5-(3,6-dihydro-2H-pyran-4-yl)benzaldehyde

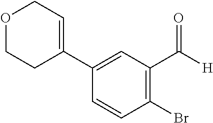 [SMILES: O=Cc1cc(C2=CCOCC2)ccc1Br]

2-(3,6-Dihydro-2H-pyran-4-yl)-4,4,5,5-tetramethyl-1,3,2-dioxaborolane (4.41 g, 21.00 mmol), 2-bromo-5-iodobenzaldehyde (6.218 g, 20.00 mmol), Potassium carbonate (ACS) (5.53 g, 40.0 mmol) and Pd(dppf)₂Cl₂ (0.146 g, 0.200 mmol) were mixed in 1,4-Dioxane (70 ml) and Water (35 ml) under vacuum. The reaction mixture was stirred under 80° C. overnight. The reaction mixture was filtered and the filtrate was concentrated by Rotavapor and the residue was dissolved in EtOAc and concentrated with silica gel. Purification by Biotage column, eluting with 0-20% EtOAc in hexanes gave the product (2.8 g, 52.4% yield). The product was taken into the next step without characterization.

### Step 2: 5-(3,6-dihydro-2H-pyran-4-yl)-2-(4,4,5,5-tetramethyl-1,3,2-dioxaborolan-2-yl)benzaldehyde

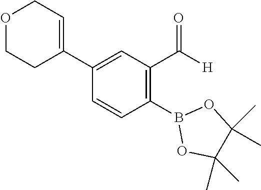 [SMILES: CC1(C)OB(c2ccc(C3=CCOCC3)cc2C=O)OC1(C)C]

2-Bromo-5-(3,6-dihydro-2H-pyran-4-yl)benzaldehyde (2.20 g, 8.24 mmol) was mixed with bis(pinacolato)diboron (2.510 g, 9.88 mmol), [1,1-bis(diphenylphosphino)ferrocene]dichloropalladium(II)·DCM (0.673 g, 0.824 mmol) and potassium acetate (1.617 g, 16.47 mmol) in 1,4-Dioxane (60 ml) under vacuum. The reaction mixture was stirred under 95° C. oil bath overnight. The reaction mixture was filtered, the filtrate was concentrated with silica gel, purified by Biotage (40 g), eluted with 0-10% EtOAc in hexanes. The crude product was dissolved in DCM and concentrated over silica gel again, purified by Biotage Column (25 g), eluted with 0-5% EtOAc in hexanes to give the title compounde (1.2 g, 41.7% yield) as off-white solid. LCMS: [M+H]⁺=215.14.

### Step 3: 1-(5-(3,6-dihydro-2H-pyran-4-yl)-2-(4,4,5,5-tetramethyl-1,3,2-dioxaborolan-2-yl)phenyl)-N,N-dimethylmethanamine, Hydrochloride HCl[B]

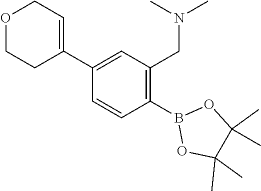 [SMILES: CN(C)Cc1cc(C2=CCOCC2)ccc1B1OC(C)(C)C(C)(C)O1]

Sodium triacetoxyborohydride (424 mg, 2.000 mmol) was added in to a CH₂Cl₂ (10 ml) solution of 5-(3,6-dihydro-2H-pyran-4-yl)-2-(4,4,5,5-tetramethyl-1,3,2-dioxaborolan-2-yl)benzaldehyde (314.2 mg, 1.000 mmol) and dimethylamine, 2.0M solution in THF (2.500 ml, 5.00 mmol) at RT. The reaction mixture was stirred for 30 min then was quenched with sat. aq. NaHCO₃, washed with brine twice, dried over MgSO₄, and filtered. The filtrate was concentrated to give the product (326 mg, 90% yield) as sticky oil (TXX-4019-0087-02). This material was stirred in acetone (7.5 mL). then 4M HCl in dioxane (0.3 mL, 1.2 mmol) was added. The reaction mixture was diluted with ether and the solid was filtered to give 5-(3,6-dihydro-2H-pyran-4-yl)-2-(4,4,5,5-tetramethyl-1,3,2-dioxaborolan-2-yl)benzaldehyde hydrochloride salt 250 mg. LCMS: [M+H]⁺=344.48.

### Step 4: 1-(5-(3,6-dihydro-2H-pyran-4-yl)-2-(4,4,5,5-tetramethyl-1,3,2-dioxaborolan-2-yl)phenyl)-N,N-dimethylmethanamine, Hydrochloride, HCl[B]

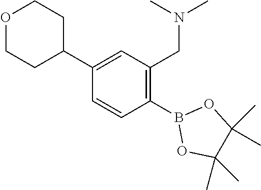 [SMILES: CN(C)Cc1cc(C2CCOCC2)ccc1B1OC(C)(C)C(C)(C)O1]

1-(5-(3,6-Dihydro-2H-pyran-4-yl)-2-(4,4,5,5-tetramethyl-1,3,2-dioxaborolan-2-yl)phenyl)-N,N-dimethylmethanamine, Hydrochloride, HCl (250 mg, 0.658 mmol) was mixed with 10% palladium on carbon wet (28.0 mg, 0.132 mmol) in EtOAc (5 ml) and Methanol (MeOH) (5.00 ml). The reaction mixture was stirred under a hydrogen balloon and stirred for 30 min. The reaction mixture was filtered and the filtrate was concentrated by rotavapor and triturated with ether to give N,N-dimethyl-1-(5-(tetrahydro-2H-pyran-4-yl)-2-(4,4,5,5-tetramethyl-1,3,2-dioxaborolan-2-yl)phenyl)methanamine, Hydrochloride, HCl (245 mg, 97% yield) as off-white solid.

### Step 5: 6-(3-amino-6-(2-((dimethylamino)methyl)-4-(tetrahydro-2H-pyran-4-yl)phenyl)pyrazin-2-yl)-7-fluoro-3,4-dihydroisoquinolin-1(2H)-one (II-1)

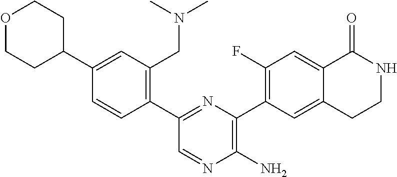 [SMILES: CN(C)Cc1cc(C2CCOCC2)ccc1-c1cnc(N)c(-c2cc3c(cc2F)C(=O)NCC3)n1]

Prepared from 6-(3-amino-6-bromopyrazin-2-yl)-7-fluoro-3,4-dihydroisoquinolin-1(2H)-one (33.6 mg, 0.100 mmol) and N,N-dimethyl-1-(5-(tetrahydro-2H-pyran-4-yl)-2-(4,4,5,5-tetramethyl-1,3,2-dioxaborolan-2-yl)phenyl)methanamine hydrochloride (38.1 mg, 0.100 mmol) to give the title compound (14.7 mg, 30.8% yield). ¹H NMR (500 MHz, METHANOL-d₄) 5=8.24 (s, 1H), 7.80 (d, J=10.1 Hz, 1H), 7.53 (d, J=6.6 Hz, 1H), 7.45 (br d, J=7.7 Hz, 1H), 7.42 (d, J=1.3 Hz, 1H), 7.32 (br d, J=7.6 Hz, 1H), 4.08 (br dd, J=3.0, 10.3 Hz, 2H), 3.76-3.65 (m, 2H), 3.64-3.52 (m, 4H), 3.05 (t, J=6.6 Hz, 2H), 2.94-2.84 (m, 1H), 2.18 (br s, 6H), 1.93-1.76 (m, 4H). [M+H]⁺=476.54.

### Example 59: 6-(3-amino-6-(3-((dimethylamino)methyl)-4-(tetrahydro-2H-pyran-4-yl)phenyl)pyrazin-2-yl)-7-fluoro-4-methylisoquinolin-1(2H)-one (II-2)

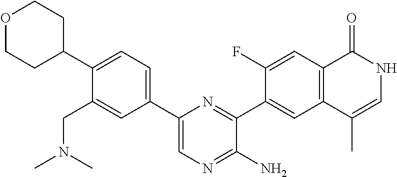 [SMILES: Cc1c[nH]c(=O)c2cc(F)c(-c3nc(-c4ccc(C5CCOCC5)c(CN(C)C)c4)cnc3N)cc12]

### Step 1: Methyl 2-(3,6-dihydro-2H-pyran-4-yl)-5-nitrobenzoate

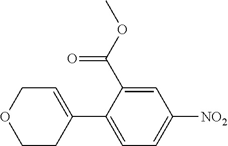 [SMILES: COC(=O)c1cc([N+](=O)[O-])ccc1C1=CCOCC1]

To a degassed solution of methyl 2-iodo-5-nitrobenzoate (4 g, 13.02 mmol), 2-(3,6-dihydro-2H-pyran-4-yl)-4,4,5,5-tetramethyl-1,3,2-dioxaborolane (3.4 g, 13.15 mmol) and K₂CO₃ (4.5 g, 32.56 mmol) in dioxane:water (7.5:2.5, 40 mL), Pd(dppf)Cl₂·DCM (1.06 g, 1.3 mmol) was added and further degassed with N2 for 10 minutes and the reaction mass was stirred at 90° C. for 16 h. After completion of reaction, reaction mass was diluted with water (40 mL) and extracted with EtOAc (3×30 mL). The crude was purified by column chromatography and the product was eluted in 18% EtOAc in hexanes to afford the product (3.1 g, 90%) as off-white solid. ¹H NMR (400 MHz, Chloroform-d) δ 8.74 (d, J=2.4 Hz, 1H), 8.47 (s, 1H), 8.35 (dd, J=8.4, 2.5 Hz, 1H), 7.46 (d, J=8.4 Hz, 1H), 5.7 (s, 1H), 4.35 (q, J=2.7 Hz, 2H), 3.98 (d, J=6.6 Hz, 5H), 2.41 (tq, J=4.9, 2.3 Hz, 2H).

### Step 2: Methyl 5-amino-2-(tetrahydro-2H-pyran-4-yl)benzoate

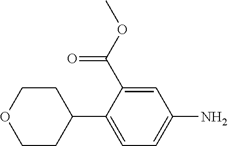 [SMILES: COC(=O)c1cc(N)ccc1C1CCOCC1]

In a 3 neck RBF, 10% Pd/C (50% moist, 2 w/w, 7.6 g) and methyl 2-(3,6-dihydro-2H-pyran-4-yl)-5-nitrobenzoate (3.8 g, 14.43 mmol) were added in EtOAc (40 mL). The reaction mass was stirred at room temperature with continuous purging of hydrogen for 2.5 days. After completion of reaction, reaction mass was filtered through celite bed and the filtrate was concentrated under vacuum to afford the crude product (3.2 g, 94%) as pale yellow gummy solid used as such in next step without further purification. LCMS: [M+H]⁺=236.5.

### Step 3: Methyl 5-bromo-2-(tetrahydro-2H-pyran-4-yl)benzoate

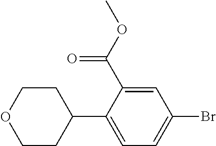 [SMILES: COC(=O)c1cc(Br)ccc1C1CCOCC1]

To a solution of methyl 5-amino-2-(tetrahydro-2H-pyran-4-yl)benzoate (3 g, 12.74 mmol) in bromoform (9.6 mL). To this, tert-butylnitrite (15 mL) was added dropwise at room temperature and the reaction mass was stirred at same temperature for 30 minutes. After completion of reaction, the reaction mass was concentrated to afford crude. The crude was purified by column chromatography and the product was eluted in 10% EtOAc in hexanes to afford the product (1.8 g, 47%) as a yellow solid. ¹H NMR (400 MHz, Chloroform-d) 1.76-1.90 (m, 4H), 3.63 (m, 4H), 3.95 (s, 3H), 4.11 (dt, J=11.1, 2.9 Hz, 1H), 7.32 (d, J=8.4 Hz, 1H), 7.64 (dd, J=8.4, 2.3 Hz, 1H), 7.98 (d, J=2.4 Hz, 1H).

### Step 4: (5-bromo-2-(tetrahydro-2H-pyran-4-yl)phenyl)methanol

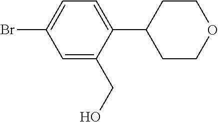 [SMILES: OCc1cc(Br)ccc1C1CCOCC1]

To a cooled solution of methyl 5-bromo-2-(tetrahydro-2H-pyran-4-yl)benzoate (1.8 g, 6.02 mmol) in dry THF (18 mL) at 0° C., LAH (1 M in THF, 6.12 mL, 6.02 mmol) was added dropwise at 0° C. The reaction mass was stirred at RT for 3 h. After completion of reaction, reaction mass was quenched with the dil. HCl (50 mL) and extracted with ethyl acetate (3×50 mL). The combined organic was dried over anhydrous Na₂SO₄, concentrate under vacuum to afford the title compound (1.3 g, 4.79 mmol, 79.68%) as pale-yellow liquid. ¹H NMR (400 MHz, Chloroform-d) 1.67-1.90 (m, 4H), 3.05 (m, 1H), 3.58 (m, 2H), 4.10 (d, J=11.1, 1H), 4.73 (s, 2H), 7.21 (d, J=8.4 Hz, 1H), 7.47 (dd, J=8.4, 2.3 Hz, 1H), 7.55 (d, J=2.4 Hz, 1H).

### Step 5: 5-bromo-2-(tetrahydro-2H-pyran-4-yl)benzaldehyde

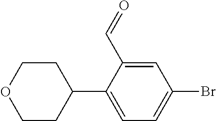 [SMILES: O=Cc1cc(Br)ccc1C1CCOCC1]

To a solution of (5-bromo-2-(tetrahydro-2H-pyran-4-yl)phenyl)methanol (1.3 g, 4.79 mmol) in DCM (26 mL), MnO₂ (3.3 g, 38.35 mmol) was added at room temperature and the reaction mass was stirred at room temperature for 48 h. After completion of reaction, reaction mass was filtered through celite bed and the filtrate was concentrate under vacuum to afford crude product. The crude was purified by column chromatography and the product was eluted in 15% EtOAc in hexanes to afford the product (0.76 g, 58.9%) as light-yellow solid. ¹H NMR (400 MHz, Chloroform-d) δ 1.76-1.84 (m, 2H), 1.86-1.93 (m, J=12.2, 4.3 Hz, 2H), 3.61-3.67 (t, J=11.7 Hz, 2H), 3.89-3.83 (tt, J=3.2, 4.0 Hz, 1H), 4.15-4.12 (dd, J=11.4, 4.6 Hz, 2H), 7.39-7.37 (t, J=8.4 Hz, 1H), 7.77 (d, 1H), 7.98 (s, 1H), 10.27 (s, 1H).

### Step 6: 1-(5-bromo-2-(tetrahydro-2H-pyran-4-yl)phenyl)-N,N-dimethylmethanamine

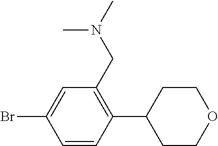 [SMILES: CN(C)Cc1cc(Br)ccc1C1CCOCC1]

To a solution of 5-bromo-2-(tetrahydro-2H-pyran-4-yl)benzaldehyde (400 mg, 1.486 mmol) in dichloromethane (DCM) (10 ml) was added dimethylamine, 2.0M solution in THF (2.229 ml, 4.46 mmol) followed by acetic acid (0.00850 mL, 0.149 mmol). Upon stirring at RT for 10 minutes, sodium triacetoxyborohydride (945 mg, 4.46 mmol) was added portion-wise and the white suspension was stirred at room temperature for 1 hour. The reaction was basified with 1M NaOH (aq) solution. The organic phase was separated, and the aqueous phase was further washed with DCM (2×). The combined organic phases were washed with brine (1×), dried over Na₂SO₄ and concentrated in vacuo to obtain the product (394 mg, 89% yield) as a white solid. The material was carried onto the next step without further purification. LCMS: [M+H]⁺=298.34.

### Step 7: N,N-dimethyl-1-(2-(tetrahydro-2H-pyran-4-yl)-5-(4,4,5,5-tetramethyl-1,3,2-dioxaborolan-2-yl)phenyl)methanamine

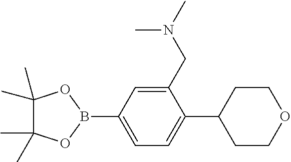 [SMILES: CN(C)Cc1cc(B2OC(C)(C)C(C)(C)O2)ccc1C1CCOCC1]

To a vial charged with 1-(5-bromo-2-(tetrahydro-2H-pyran-4-yl)phenyl)-N,N-dimethylmethanamine (385 mg, 1.291 mmol), bis(pinacolato)diboron (361 mg, 1.420 mmol), and potassium acetate (380 mg, 3.87 mmol), was added anhydrous 1,4-dioxane (5 ml). The system was degassed then [1,12-bis(diphenylphosphino)ferrocene]dichloropalladium(II) (94 mg, 0.129 mmol) was added. The mixture was flushed with nitrogen then heated at 100° C. overnight. The reaction was diluted with acetonitrile, filtered through a pad of celite, concentrated in vacuo and used as-is the next step assuming full conversion to the boronate. LCMS [M+H]⁺=346.55 boronate Step 8: 3-chloro-5-(3-((dimethylamino)methyl)-4-(tetrahydro-2H-pyran-4-yl)phenyl)pyrazin-2-amine

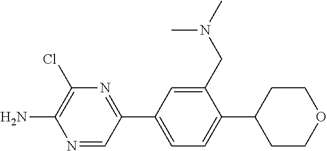 [SMILES: CN(C)Cc1cc(-c2cnc(N)c(Cl)n2)ccc1C1CCOCC1]

A vial was charged with 2-amino-5-bromo-3-chloropyrazine (251 mg, 1.206 mmol), N,N-dimethyl-1-(2-(tetrahydro-2H-pyran-4-yl)-5-(4,4,5,5-tetramethyl-1,3,2-dioxaborolan-2-yl)phenyl)methanamine (347 mg, 1.005 mmol), [1,1′-bis(diphenylphosphino)ferrocene]dichloropalladium(II) (73.5 mg, 0.100 mmol) and cesium carbonate (819 mg, 2.51 mmol). After the vial was sealed the reaction vessel was evacuated and backfilled with nitrogen. 1,4-Dioxane (8 ml) and water (2 ml) were added via syringe and the vessel was evacuated and backfilled with nitrogen an additional time. The reaction was heated to 80° C. overnight. The reaction mixture was concentrated onto celite and purified by flash chromatography eluting with 0-10% MeOH/DCM+1% NH₄OH. The desired fractions were collected, concentrated and dried under vacuum to afford the product (218 mg, 0.628 mmol, 62.5% yield) as a beige solid. LCMS [M+H]⁺=347.42.

### Step 9: (E)-3-(3-bromo-4-fluorophenyl)but-2-enoic acid

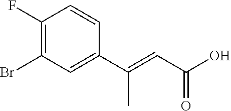

To THF (75 mL) at 0° C. under nitrogen atmosphere, sodium hydride (60%, 1.38 g, 34.56 mmol) was added portion wise. Triethyl phosphonoacetate (6.85 mL, 34.56 mmol) was added dropwise and stirred at 0° C. for 20 min and the resulting mixture was added dropwise to a solution of 1-(3-bromo-4-fluorophenyl)ethan-1-one (5 g, 23.04 mmol) in THF (50 mL) and the resulting mixture was refluxed for 16 h. The mixture was then diluted with water (200 mL) and extracted with DCM (3×150 mL). The organic layer was dried over Na₂SO₄ and concentrated under vacuum to afford a yellow oil. The oil was dissolved in MeOH (25 mL) and NaH (4.6 g, 226.13 mmol) and water (15 mL) were added and the reaction mass was heated at 50° C. for 2 h. The reaction mass was concentrated to evaporate the organics and the resulting aqueous solution was acidified by aqueous 2M HCl and extracted with EtOAc (2×150 mL). The combined organic layers were dried over Na₂SO₄ and concentrated under vacuum to afford the product (3.5 g, 58.6% yield) as white solid. LCMS: [M+H]⁺=258.9.

### Step 10: (E)-3-(3-bromo-4-fluorophenyl)but-2-enoyl chloride

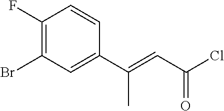

To a cooled solution of (E)-3-(3-bromo-4-fluorophenyl)but-2-enoic acid (3.5 g, 13.58 mmol) in DCM (35 mL) and DMF (0.2 mL) at 0° C., oxalyl chloride (2.05 g, 16.21 mmol) was added dropwise and the reaction mass was allowed to warm to RT and stirred at same temperature for 3 h. After completion the reaction, reaction mass was concentrated and azeotrope with toluene (2×20 mL) and DCM (2×20 mL) to give the crude product (3.5 g) as white solid which was used as such in the next step without analysis and further purification.

### Step 11: (E)-3-(3-bromo-4-fluorophenyl)but-2-enoyl azide

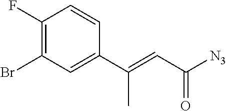 [SMILES: C/C(=C\C(=O)N=[N+]=[N-])c1ccc(F)c(Br)c1]

To a cooled solution of crude of (E)-3-(3-bromo-4-fluorophenyl)but-2-enoyl chloride (3.5 g, 12.61 mmol) in 1,4 dioxane (35 mL) at 0° C., a suspension of sodium azide (1.47 g, 22.70 mmol) in 1:1 mixture of 1,4-dioxane and water (15 mL) was added and the reaction mas was gradually warm to room temperature and stirred at same temperature for a 1.5 h. After completion reaction, reaction mass was diluted with water (30 mL) and extracted with diethyl ether (2×100 mL). The combined organic layers were back washed with saturated aq. NaHCO₃ (3×100 mL) and water (3×100 mL) and dried over Na₂SO₄ and organic layer was directly used for next step.

### Step 12: 6-bromo-7-fluoro-4-methylisoquinolin-1(2H)-one

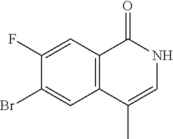

The ether layer of (E)-3-(3-bromo-4-fluorophenyl)but-2-enoyl azide was treated with 1,2 dichlorobenzene (30 mL) and the ether was removed under vacuum to give a solution of (E)-3-(3-bromo-4-fluorophenyl)but-2-enoyl azide in 1,2 dichlorobenzene. The acyl azide solution in 1,2 dichlorobenzene was added dropwise over 30 min to a solution of iodine (0.4 g) in 1,2 dichlorobenzene (30 mL) at 120° C. The mixture was then stirred at 190° C. for 16 h, allowed to cool room temperature and added to hexane (1000 mL). The suspension was stirred for 1 h and the resulting solid was washed with EtOAc (50 mL) and DCM (50 mL) and dried under vacuum to give the product (0.8 g, 25.36%) as pale-yellow solid. LCMS: [M+2]⁺=258.

### Step 13: (7-fluoro-4-methyl-1-oxo-1,2-dihydroisoquinolin-6-yl)boronic acid

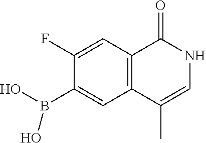 [SMILES: Cc1c[nH]c(=O)c2cc(F)c(B(O)O)cc12]

Prepared from 6-bromo-7-fluoro-4-methylisoquinolin-1(2H)-one (800 mg, 3.12 mmol) to give the boronic acid which was used in the next step without further purification. LCMS: [M+H]⁺=222.29.

### Step 14: 6-(3-amino-6-(3-((dimethylamino)methyl)-4-(tetrahydro-2H-pyran-4-yl)phenyl)pyrazin-2-yl)-7-fluoro-4-methylisoquinolin-1(2H)-one

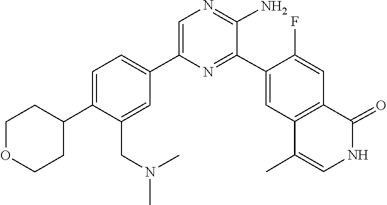 [SMILES: Cc1c[nH]c(=O)c2cc(F)c(-c3nc(-c4ccc(C5CCOCC5)c(CN(C)C)c4)cnc3N)cc12]

Prepared from (7-fluoro-4-methyl-1-oxo-1,2-dihydroisoquinolin-6-yl)boronic acid (26.8 mg, 0.121 mmol) and 3-chloro-5-(3-((dimethylamino)methyl)-4-(tetrahydro-2H-pyran-4-yl)phenyl)pyrazin-2-amine (35 mg, 0.101 mmol) to give the title compound (17.3 mg, 35.2% yield) as a beige solid. ¹H NMR (500 MHz, DMSO-d₆) b ppm 11.27 (br. s., 1H), 8.60 (s, 1H), 8.00 (d, J=10.27 Hz, 1H), 7.79-7.86 (m, 2H), 7.77 (s, 1H), 7.35 (d, J=8.19 Hz, 1H), 7.06 (br. s., 1H), 6.37 (s, 2H), 3.95 (dd, J=10.82, 2.87 Hz, 2H), 3.41-3.46 (m, 4H), 3.18-3.25 (m, 1H), 2.24 (s, 3H), 2.15 (s, 6H), 1.66-1.74 (m, 2H), 1.58-1.65 (m, 2H); LCMS: [M+H]⁺=488.58.

### Example 60: 7-(3-amino-6-(3-((dimethylamino)methyl)-4-(tetrahydro-2H-pyran-4-yl)phenyl)pyrazin-2-yl)-2-methylquinazolin-4(3H)-one (II-3)

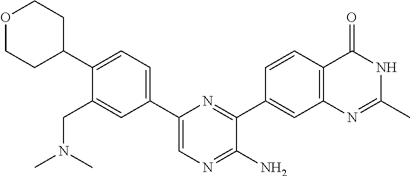 [SMILES: Cc1nc2cc(-c3nc(-c4ccc(C5CCOCC5)c(CN(C)C)c4)cnc3N)ccc2c(=O)[nH]1]

Prepared from (2-methyl-4-oxo-3,4-dihydroquinazolin-7-yl)boronic acid (24.70 mg, 0.121 mmol) and 3-chloro-5-(3-((dimethylamino)methyl)-4-(tetrahydro-2H-pyran-4-yl)phenyl)pyrazin-2-amine (35 mg, 0.101 mmol) to give the title compound (16.2 mg, 34.1% yield) as a yellow solid. ¹H NMR (500 MHz, DMSO-d₆) δ ppm 12.22 (br. s., 1H), 8.57 (s, 1H), 8.19 (d, J=8.19 Hz, 1H), 7.94 (s, 1H), 7.83-7.88 (m, 2H), 7.81 (d, J=1.71 Hz, 1H), 7.37 (d, J=8.19 Hz, 1H), 6.43 (s, 2H), 3.96 (dd, J=10.82, 3.36 Hz, 2H), 3.44-3.48 (m, 4H), 3.19-3.26 (m, 1H), 2.39 (s, 3H), 2.17 (s, 6H), 1.67-1.75 (m, 2H), 1.59-1.65 (m, 2H); LCMS: [M+H]⁺=471.53.

### Example 61: 6-(3-amino-6-(3-((dimethylamino)methyl)-4-(tetrahydro-2H-pyran-4-yl)phenyl)pyrazin-2-yl)-8-fluoro-3-methylisoquinolin-1(2H)-one (II-4)

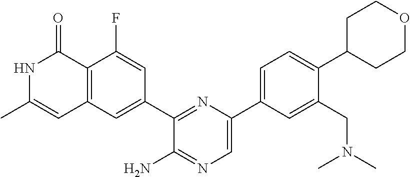 [SMILES: Cc1cc2cc(-c3nc(-c4ccc(C5CCOCC5)c(CN(C)C)c4)cnc3N)cc(F)c2c(=O)[nH]1]

### Step 1: 6-bromo-8-fluoro-3-methylisoquinolin-1(2H)-one

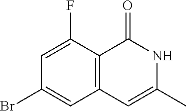

To an RBF was added copper(I) bromide (0.209 g, 1.455 mmol), Cs₂CO₃ (9.48 g, 29.1 mmol), 2,4-dibromo-6-fluorobenzamide (4.32 g, 14.55 mmol), propan-2-one (5.34 mL, 72.7 mmol) and dimethylsulfoxide (DMSO) (150 mL). The reaction was stirred and heated at 80° C. overnight. The reaction mixture was partitioned between brine (200 mL) and DCM (200 mL). The organic layer was separated, and the aqueous layer washed with DCM (2×100 ml). The combined organic layers were washed with brine, dried over anhydrous Na₂SO₄, and concentrated onto celite. The mixture was purified by flash chromatography (Biotage, silica gel) eluting with 0-100% EtOAc/Hexanes. The desired fractions were collected, concentrated and dried under vacuum to afford the product (2.41 g, 64.7% yield) as a pale-yellow solid. ¹H NMR (500 MHz, DMSO-d₆) δ ppm 11.39 (br. s., 1H), 7.62 (s, 1H), 7.38 (d, J=11.00 Hz, 1H), 6.30 (s, 1H), 2.18 (s, 3H); LCMS: [M+H]+=256.08.

### Step 2: (8-fluoro-3-methyl-1-oxo-1,2-dihydroisoquinolin-6-yl)boronic acid

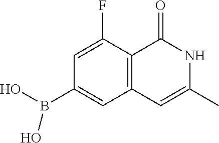 [SMILES: Cc1cc2cc(B(O)O)cc(F)c2c(=O)[nH]1]

Prepared from 6-bromo-8-fluoro-3-methylisoquinolin-1(2H)-one (31 mg, 0.121 mmol) to give (8-fluoro-3-methyl-1-oxo-1,2-dihydroisoquinolin-6-yl)boronic acid assuming full conversion (26.8 mg, 0.121 mmol, 100%) as a brown residue. LCMS: [M+H]⁺=222.35.

### Step 3: 6-(3-amino-6-(3-((dimethylamino)methyl)-4-(tetrahydro-2H-pyran-4-yl)phenyl)pyrazin-2-yl)-8-fluoro-3-methylisoquinolin-1(2H)-one

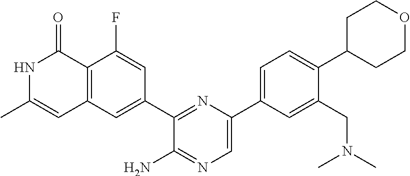 [SMILES: Cc1cc2cc(-c3nc(-c4ccc(C5CCOCC5)c(CN(C)C)c4)cnc3N)cc(F)c2c(=O)[nH]1]

Prepared from (8-fluoro-3-methyl-1-oxo-1,2-dihydroisoquinolin-6-yl)boronic acid (26.8 mg, 0.121 mmol) and 3-chloro-5-(3-((dimethylamino)methyl)-4-(tetrahydro-2H-pyran-4-yl)phenyl)pyrazin-2-amine (35 mg, 0.101 mmol) to give the title compound (13.1 mg, 26.6% yield) as a yellow solid. ¹H NMR (500 MHz, DMSO-d₆) δ ppm 11.31 (s, 1H), 8.57 (s, 1H), 7.86 (dd, J=8.07, 1.71 Hz, 1H), 7.80 (d, J=1.71 Hz, 1H), 7.72 (s, 1H), 7.43 (d, J=12.47 Hz, 1H), 7.37 (d, J=8.19 Hz, 1H), 6.49 (s, 2H), 6.42 (s, 1H), 3.96 (dd, J=10.88, 3.55 Hz, 2H), 3.41-3.49 (m, 4H), 3.18-3.26 (m, 1H), 2.21 (s, 3H), 2.17 (s, 6H), 1.72 (qd, J=12.31, 3.91 Hz, 2H), 1.59-1.66 (m, 2H); LCMS: [M+H]⁺=488.58.

### Example 62: 6-(3-amino-6-(3-((dimethylamino)methyl)-4-(tetrahydro-2H-pyran-4-yl)phenyl)pyrazin-2-yl)-4,8-difluoro-3-methylisoquinolin-1(2H)-one (II-5II)

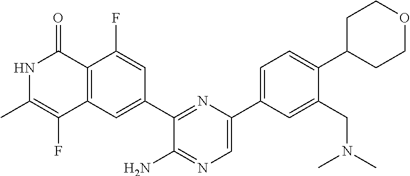 [SMILES: Cc1[nH]c(=O)c2c(F)cc(-c3nc(-c4ccc(C5CCOCC5)c(CN(C)C)c4)cnc3N)cc2c1F]

### Step 1: 6-bromo-4,8-difluoro-3-methylisoquinolin-1(2H)-one

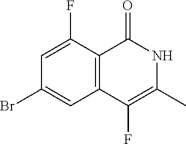

A vial was charged with 6-bromo-8-fluoro-3-methylisoquinolin-1(2H)-one (1.13 g, 4.41 mmol) and Selectfluor™ fluorinating reagent >95% in F+ active (1.641 g, 4.63 mmol). Methanol (10 ml) and acetonitrile (10 ml) were added and the reaction was stirred at room temperature for 5 days. The white suspension was filtered, washed with water, and dried under vacuum to afford the product (1.01 g, 91% yield) as a white solid. LCMS: [M+H]⁺=274.14.

### Step 2: (8-fluoro-3-methyl-1-oxo-1,2-dihydroisoquinolin-6-yl)boronic acid

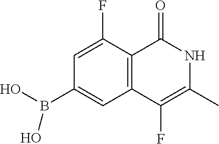 [SMILES: Cc1[nH]c(=O)c2c(F)cc(B(O)O)cc2c1F]

Prepared from 6-bromo-4,8-difluoro-3-methylisoquinolin-1(2H)-one (33 mg, 0.120 mmol) to give (4,8-difluoro-3-methyl-1-oxo-1,2-dihydroisoquinolin-6-yl)boronic acid assuming full conversion (28.8 mg, 0.121 mmol, 100%) as a brown residue. LCMS: [M+H]⁺=240.28.

### Step 3: 6-(3-amino-6-(3-((dimethylamino)methyl)-4-(tetrahydro-2H-pyran-4-yl)phenyl)pyrazin-2-yl)-4,8-difluoro-3-methylisoquinolin-1(2H-one

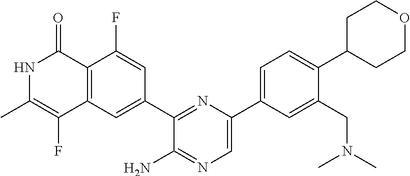 [SMILES: Cc1[nH]c(=O)c2c(F)cc(-c3nc(-c4ccc(C5CCOCC5)c(CN(C)C)c4)cnc3N)cc2c1F]

Prepared from (4,8-difluoro-3-methyl-1-oxo-1,2-dihydroisoquinolin-6-yl)boronic acid (28.9 mg, 0.121 mmol) and 3-chloro-5-(3-((dimethylamino)methyl)-4-(tetrahydro-2H-pyran-4-yl)phenyl)pyrazin-2-amine (35 mg, 0.101 mmol) to give the title compound (12.9 mg, 25.3% yield) as a yellow solid. ¹H NMR (500 MHz, DMSO-d₆) b ppm 11.30 (br. s., 1H), 8.59 (s, 1H), 7.83-7.88 (m, 2H), 7.81 (s, 1H), 7.61 (d, J=12.35 Hz, 1H), 7.38 (d, J=8.19 Hz, 1H), 6.57 (s, 2H), 3.96 (dd, J=10.82, 3.24 Hz, 2H), 3.41-3.48 (m, 4H), 3.19-3.26 (m, 1H), 2.24 (d, J=2.69 Hz, 3H), 2.17 (s, 6H), 1.71 (qd, J=12.21, 3.97 Hz, 2H), 1.59-1.66 (m, 2H); LCMS: [M+H]⁺=506.57.

### Example 63: 6-(3-amino-6-(3-((dimethylamino)methyl)-4-(tetrahydro-2H-pyran-4-yl)phenyl)pyrazin-2-yl)-4,7-difluoro-3-methylisoquinolin-1(2H)-one (II-6)

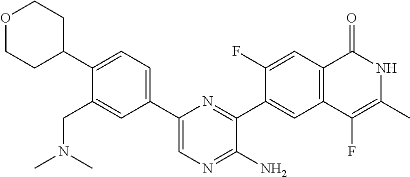 [SMILES: Cc1[nH]c(=O)c2cc(F)c(-c3nc(-c4ccc(C5CCOCC5)c(CN(C)C)c4)cnc3N)cc2c1F]

### Step 1: 6-bromo-7-fluoro-3-methylisoquinolin-1(2H)-one

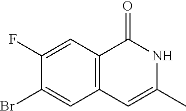

To an RBF was added copper(I) bromide (0.068 g, 0.472 mmol), Cs₂CO₃ (3.07 g, 9.43 mmol), 2,4-dibromo-5-fluorobenzamide (1.40 g, 4.72 mmol), propan-2-one (1.731 ml, 23.58 mmol) and dimethylsulfoxide (20 ml). The reaction was stirred and heated at 80° C. overnight. The reaction mixture was partitioned between brine (200 ml) and DCM (200 ml). The organic layer was separated, and the aqueous layer washed with DCM (2×100 ml). The combined organic layers were washed with brine, dried over anhydrous Na₂SO₄, and concentrated onto celite. The mixture was purified by flash chromatography eluting with 0-10% MeOH/DCM+1% NH₄OH. The desired fractions were collected, concentrated and dried under vacuum to afford the product (489 mg, 40.5% yield) as a beige solid. ¹H NMR (500 MHz, DMSO-d₆) δ ppm 11.50 (br. s., 1H), 8.01 (d, J=5.50 Hz, 1H), 7.88 (d, J=8.56 Hz, 1H), 6.33 (br. s., 1H), 2.20 (br. s., 3H); LCMS: [M+H]⁺=256.21.

### Step 2: 6-bromo-4,7-difluoro-3-methylisoquinolin-1(2H)-one

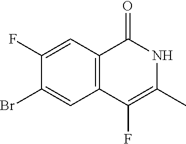

A vial was charged with 6-bromo-7-fluoro-3-methylisoquinolin-1(2H)-one (189 mg, 0.738 mmol) and Selectfluor™ fluorinating reagent >95% in F+ active (275 mg, 0.775 mmol). Methanol (10 ml) and acetonitrile (10 ml) were added and the reaction was stirred at RT. The white suspension was filtered, washed with water, and dried under vacuum to afford the product (158 mg, 78% yield) as a white solid. LCMS: [M+H]⁺=274.27.

### Step 3: (8-fluoro-3-methyl-1-oxo-1,2-dihydroisoquinolin-6-yl)boronic acid

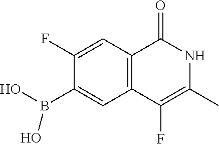 [SMILES: Cc1[nH]c(=O)c2cc(F)c(B(O)O)cc2c1F]

Prepared from 6-bromo-4,7-difluoro-3-methylisoquinolin-1(2H)-one (115 mg, 0.420 mmol) to give the product (100 mg, 0.418 mmol, 100%) as a brown residue. LCMS: [M+H]⁺=240.28.

### Step 4: 6-(3-amino-6-(3-((dimethylamino)methyl)-4-(tetrahydro-2H-pyran-4-yl)phenyl)pyrazin-2-yl)-4,7-difluoro-3-methylisoquinolin-1(2H)-one

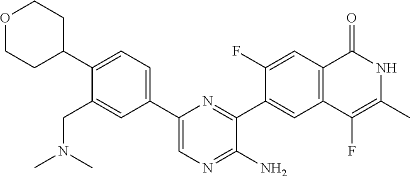 [SMILES: Cc1[nH]c(=O)c2cc(F)c(-c3nc(-c4ccc(C5CCOCC5)c(CN(C)C)c4)cnc3N)cc2c1F]

Prepared from (4,7-difluoro-3-methyl-1-oxo-1,2-dihydroisoquinolin-6-yl)boronic acid (28.9 mg, 0.121 mmol) and 3-chloro-5-(3-((dimethylamino)methyl)-4-(tetrahydro-2H-pyran-4-yl)phenyl)pyrazin-2-amine (35 mg, 0.101 mmol) to give the title compound (5.3 mg, 10.39% yield) as a yellow solid. ¹H NMR (500 MHz, DMSO-d₆) b ppm 11.42 (br. s., 1H), 8.61 (s, 1H), 7.96 (dd, J=9.90, 1.47 Hz, 1H), 7.83 (d, J=6.60 Hz, 1H), 7.80 (dd, J=8.19, 1.71 Hz, 1H), 7.76 (d, J=1.71 Hz, 1H), 7.36 (d, J=8.19 Hz, 1H), 6.41 (s, 2H), 3.95 (dd, J=10.70, 3.36 Hz, 2H), 3.43-3.46 (m, 4H), 3.19-3.25 (m, 1H), 2.25 (d, J=2.93 Hz, 3H), 2.15 (s, 6H), 1.67-1.75 (m, 2H), 1.59-1.64 (m, 2H); LCMS: [M+H]⁺=506.63.

### B. Preparation of Comparator Compounds of the Application

### Example C-1: 6-(3-amino-6-(4-morpholinophenyl)pyrazin-2-yl)-3,4-dihydroisoquinolin-1(2H)-one (Comparator 1)

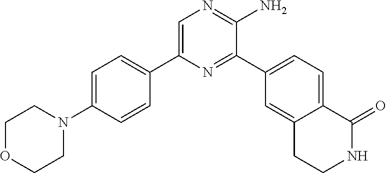 [SMILES: Nc1ncc(-c2ccc(N3CCOCC3)cc2)nc1-c1ccc2c(c1)CCNC2=O]

### Step 1: 3-chloro-5-(4-morpholinophenyl)pyrazin-2-amine

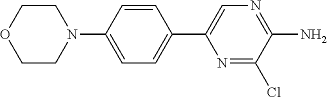 [SMILES: Nc1ncc(-c2ccc(N3CCOCC3)cc2)nc1Cl]

The intermediate was prepared following a procedure similar to that of Example 1, Step 3 using 2-amino-5-bromo-3-chloropyrazine (150 mg, 0.720 mmol), Cs₂CO₃ (703 mg, 2.159 mmol), 4-morpholinophenylboronic acid, pinacol ester (208 mg, 0.720 mmol), PdCl₂dppf (52.7 mg, 0.072 mmol), H₂O (2.5 mL) and DME (5 mL); by heating under microwave irradiation at 90° C. for 2 h. Purification by flash chromatography (12 g SiO₂ InnoFlash® cartridge, using MeOH in CH₂Cl₂ eluting at 25% MeOH) followed by a filtration through a Waters PoraPak™ CX column and another flash chromatography (25 g SiO₂ InnoFlash® cartridge, using MeOH in CH₂Cl₂ eluting at 8% MeOH) afforded the product as a brown solid (95 mg, 24% based on the purity of 52%). LCMS: [M+H]⁺=291.26.

### Step 2: 6-(3-amino-6-(4-morpholinophenyl)pyrazin-2-yl)-3,4-dihydroisoquinolin-1(2H)-one

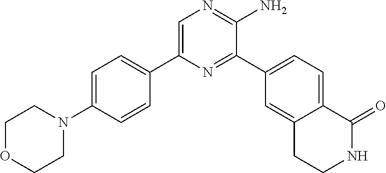 [SMILES: Nc1ncc(-c2ccc(N3CCOCC3)cc2)nc1-c1ccc2c(c1)CCNC2=O]

The title compound was prepared by a procedure similar to Example 1, Step 3 using 3-chloro-5-(4-morpholinophenyl)pyrazin-2-amine (50 mg, 52% purity, 0.089 mmol), K₃PO₄ (95 mg, 0.447 mmol), XPhos Pd G2 (7.04 mg, 8.94 μmol), crude 6-(4,4,5,5-tetramethyl-1,3,2-dioxaborolan-2-yl)-3,4-dihydroisoquinolin-1(2H)-one (42.6 mg, 0.156 mmol) in 1,4-dioxane (1.5 mL), H₂O (2 mL) and ACN (3 mL); by heating in a microwave reactor at 100° C. for 2 h and then again at 100° C. for another 2 h. Purification by flash chromatography (25 g SiO₂ InnoFlash® cartridge, using MeOH in CH₂Cl₂ eluting at 6% MeOH) followed by preparative HPLC (30 g Biotage® SNAP KP-C₁₈-HS, MeOH in (H₂O+0.05% TFA) eluting at 57% MeOH) afforded 6-(3-amino-6-(4-morpholinophenyl)pyrazin-2-yl)-3,4-dihydroisoquinolin-1(2H)-one·TFA as an orange solid (7 mg, 15% based on purity of 97%). ¹H NMR (500 MHz, CD₃OD) δ=8.33 (s, 1H), 8.10 (d, J=7.8 Hz, 1H), 7.94 (br d, J=7.9 Hz, 2H), 7.83-7.77 (m, 1H), 7.75 (s, 1H), 7.17 (br d, J=8.1 Hz, 2H), 3.89 (br s, 4H), 3.57 (br t, J=6.4 Hz, 2H), 3.36-3.31 (m, 4H), 3.10 (br t, J=6.5 Hz, 2H); LCMS: [M+H]⁺=402.39.

### Example C-2: 6-(3-amino-6-(2,3-difluoro-4-morpholinophenyl)pyrazin-2-yl)-3,4-dihydroisoquinolin-1(2H)-one (Comparator 2)

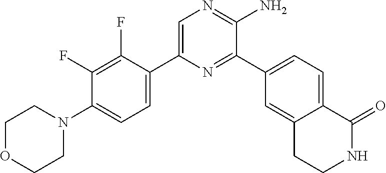 [SMILES: Nc1ncc(-c2ccc(N3CCOCC3)c(F)c2F)nc1-c1ccc2c(c1)CCNC2=O]

The title compound was prepared by a method similar to Example 1, Step 3 using 6-(3-amino-6-bromopyrazin-2-yl)-3,4-dihydroisoquinolin-1(2H)-one (300 mg, 0.940 mmol), Cs₂CO₃ (919 mg, 2.82 mmol), 2,3-difluoro-4-morpholinophenylboronic acid (251 mg, 1.034 mmol), PdCl₂dppf (68.8 mg, 0.094 mmol), H₂O (8 mL) and DME (16 mL); heating with microwave apparatus at 90° C. for 2 h. The crude product was purified by flash chromatography (50 g SiO₂ cartridge, using MeOH in CH₂Cl₂ eluting at 4% MeOH) to afford the title compound as a light yellow solid (210 mg, 51% based on purity of 99%). ¹H NMR (500 MHz, DMSO-d₆) δ 8.35 (d, J=2.6 Hz, 1H), 7.93-8.02 (m, 2H), 7.73 (dd, J=8.0, 1.6 Hz, 1H), 7.69 (s, 1H), 7.63 (td, J=8.6, 1.8 Hz, 1H), 6.95 (t, J=8.0 Hz, 1H), 6.54 (s, 2H), 3.72-3.81 (m, 4H), 3.35-3.48 (m, 2H), 3.07-3.12 (m, 4H), 2.99 (t, J=6.5 Hz, 2H); LCMS: [M+H]⁺=438.38.

### Example C-3: 6-(3-amino-6-(3-fluoro-4-morpholinophenyl)pyrazin-2-yl)-3,4-dihydroisoquinolin-1(2H)-one (Comparator 3)

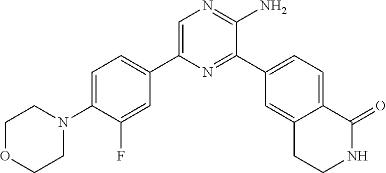 [SMILES: Nc1ncc(-c2ccc(N3CCOCC3)c(F)c2)nc1-c1ccc2c(c1)CCNC2=O]

### Step 1: 3-chloro-5-(3-fluoro-4-morpholinophenyl)pyrazin-2-amine

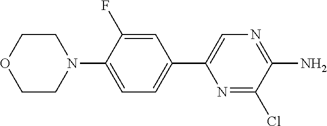 [SMILES: Nc1ncc(-c2ccc(N3CCOCC3)c(F)c2)nc1Cl]

The intermediate was prepared following a procedure similar to Example 1, Step 3 using 2-amino-5-bromo-3-chloropyrazine (200 mg, 0.959 mmol), Cs₂CO₃ (938 mg, 2.88 mmol), 3-fluoro-4-morpholinophenylboronic acid (216 mg, 0.959 mmol), PdCl₂dppf (70.2 mg, 0.096 mmol), H₂O (2.6 mL), DME (5 mL); by heating under microwave irradiation at 90° C. for 3 h. Purification by flash chromatography (25 g SiO₂ InnoFlash® cartridge, using EtOAc in CH₂Cl₂ eluting at 24% Et₂O) afforded the product as a pale yellow solid (166 mg, 53% yield based on purity of 95%). LCMS: [M+H]⁺=308.94.

### Step 2: 6-(3-amino-6-(3-fluoro-4-morpholinophenyl)pyrazin-2-yl)-3,4-dihydroisoquinolin-1(2H)-one

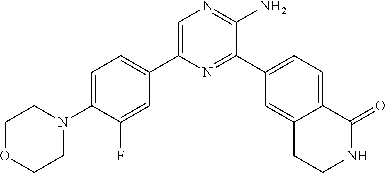 [SMILES: Nc1ncc(-c2ccc(N3CCOCC3)c(F)c2)nc1-c1ccc2c(c1)CCNC2=O]

The title compound was prepared following a procedure similar to Example 1, Step 3 using 3-chloro-5-(3-fluoro-4-morpholinophenyl)pyrazin-2-amine (61 mg, 0.188 mmol), 3,4-dihydro-1(2H)-isoquinolinone-6-boronic acid pinacol ester (56.4 mg, 0.206 mmol), XPhos Pd G2 (14.77 mg, 0.019 mmol), H₂O (2 mL) and ACN (3 mL) by heating in a microwave reactor at 100° C. for 3 h. Purification by flash chromatography (25 g SiO₂ InnoFlash® cartridge, using MeOH in CH₂Cl₂ eluting at 4% MeOH), followed by preparative HPLC (30 g Biotage® SNAP KP-C₁₈-HS, MeOH in (H₂O+0.05% TFA) eluting at 80% MeOH) afforded the TFA salt of the title compound as a light tan solid (24 mg, 24% based on purity of 99%). ¹H NMR (500 MHz, CD₃OD) δ 8.43-8.36 (m, 1H), 8.10 (d, J=8.1 Hz, 1H), 7.81 (br d, J=7.9 Hz, 1H), 7.77-7.68 (m, 3H), 7.11 (br t, J=8.7 Hz, 1H), 3.89-3.83 (m, 4H), 3.61-3.54 (m, 2H), 3.20-3.05 (m, 6H); LCMS: [M+H]⁺=420.22.

### Example C-4: 6-(3-amino-6-(2-fluoro-4-morpholinophenyl)pyrazin-2-yl)-3,4-dihydroisoquinolin-1(2H)-one (Comparator 4)

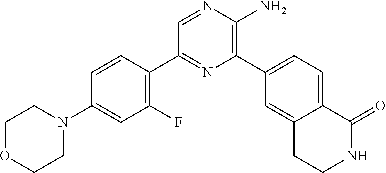 [SMILES: Nc1ncc(-c2ccc(N3CCOCC3)cc2F)nc1-c1ccc2c(c1)CCNC2=O]

### Step 1: 4-(3-fluoro-4-(4,4,5,5-tetramethyl-1,3,2-dioxaborolan-2-yl)phenyl)morpholine

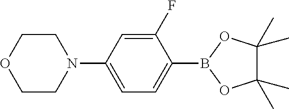 [SMILES: CC1(C)OB(c2ccc(N3CCOCC3)cc2F)OC1(C)C]

In a 5 ml microwave vial, 4-(4-bromo-3-fluorophenyl)morpholine (100 mg, 0.384 mmol), [1,12-bis(diphenylphosphino)ferrocene]dichloropalladium(II) CH₂Cl₂ (31.4 mg, 0.038 mmol), bis(pinacolato)diboron (391 mg, 1.538 mmol) and K₂CO₃ (151 mg, 1.538 mmol) were dissolved in 1,4-dioxane (4 mL) to give a white suspension. The suspension was stirred for 5 min, degassed, purged with N₂, and microwaved for 240 min at 110° C. The red suspension was centrifuged and the supernatant was transferred to another 5 mL vial. The solid was suspended in 1 mL of 1,4-dioxane and the vial was centrifuged, the supernatants were collected and combined and were used immediately in the next reaction without further purification. LCMS: [M+H]⁺=308.39.

### Step 2: 6-(3-amino-6-(2-fluoro-4-morpholinophenyl)pyrazin-2-yl)-3,4-dihydroisoquinolin-1(2H)-one, HCOOH

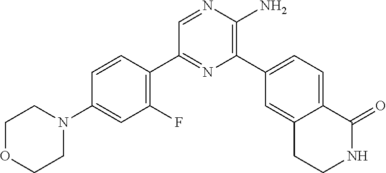 [SMILES: Nc1ncc(-c2ccc(N3CCOCC3)cc2F)nc1-c1ccc2c(c1)CCNC2=O]

A 5 ml microwave vial was charged with 6-(3-amino-6-bromopyrazin-2-yl)-3,4-dihydroisoquinolin-1(2H)-one (0.070 g, 0.219 mmol), 4-isopropylsulfonylphenylboronic acid pinacol ester (0.055 g, 0.178 mmol), [1,1′-bis(diphenylphosphino)ferrocene]dichloropalladium(II) (0.016 g, 0.022 mmol) and Cs₂CO₃ (0.214 g, 0.658 mmol) suspended in 1,4-dioxane/H₂O (0.6 mL) (solvent from the previous reaction) to give a red suspension. The suspension was stirred for 5 min, degassed, purged with N₂, and microwaved for 60 min at 90° C. The volatiles were evaporated and the product was dissolved in H₂O and filtered through a cotton plug. Purification was performed via reverse phase column C-18, (95%-0%, H₂O with 0.1% formic acid:MeCN with 0.1% formic acid) to afford, after lyophilization, the title compound as a white solid (5.8 mg, 18.5%). ¹H NMR (500 MHz, CD₃OD) δ 8.25 (d, J=2.2 Hz, 1H), 7.98 (d, J=8.0 Hz, 1H), 7.76 (t, J=9.1 Hz, 1H), 7.70 (dd, J=8.0, 1.6 Hz, 1H), 7.65 (s, 1H), 6.77 (dd, J=8.8, 2.4 Hz, 1H), 6.67 (dd, J=15.1, 2.6 Hz, 1H), 3.75-3.73 (m, 4H), 3.47 (t, J=6.7 Hz, 2H), 3.14-3.12 (m, 5H), 2.99 (t, J=6.8 Hz, 2H).

### C. Biology

### Biological Assays

### (a) HPK1 Human STE Kinase Enzymatic Radiometric Assay

A stock solution of 10 mM of test compound is prepared in DMSO. The compound plate was prepared by 3-fold and 9-point serial dilutions. Recombinant (1-346) HPK1 (h) is incubated with 8 mM MOPS pH 7.0, 0.2 mM EDTA, 0.33 mg/mL myelin basic protein, 10 mM MgAcetate and [gamma-33P]-ATP (specific activity and concentration as required). The reaction is initiated by the addition of the Mg/ATP mix. After incubation for 40 minutes at RT, the reaction is stopped by the addition of phosphoric acid to a concentration of 0.5%. 10 uL of the reaction is then spotted onto a P30 filtermat and washed four times for 4 minutes in 0.425% phosphoric acid and once in methanol prior to drying and scintillation counting. For more details of kinase assay protocols, see: Gao, Y. et. al.; Biochem J. 451 (2): 313-328, 2013.

Compounds of the application showed activity as inhibitors of HPK1 having IC₅₀'s in the following ranges: A: 0.1-10 nM; B: 11-100 nM; C: 101-1000 nM; D: >1000 nM. Specific ranges for exemplary compounds of Formula (I) are shown in Table 2.

### (b) Lck Human STE Kinase Enzymatic Radiometric Assay

Lck (h) is incubated with 50 mM Tris pH 7.5, 0.1 mM EGTA, 0.1 mM Na3VO4, 250 uM KVEKIGEGTYGVVYK (Cdc2 peptide), 10 mM MgAcetate and [gamma-33P]-ATP (specific activity and concentration as required). The reaction is initiated by the addition of the Mg/ATP mix. After incubation for 40 minutes at room temperature, the reaction is stopped by the addition of phosphoric acid to a concentration of 0.5%. 10 uL of the reaction is then spotted onto a P30 filtermat and washed four times for 4 minutes in 0.425% phosphoric acid and once in methanol prior to drying and scintillation counting. For more details on the kinase assay procedures see: Gao, Y. et. al.; Biochem J., 451 (2): 313-328, 2013. Generally, and advantageously, compounds of the application showed significantly less inhibition of Lck compared with inhibition of HPK1 (see Table 1 where IC₅₀'s are reported the following ranges: A: 0.1-10 nM; B: 11-100 nM; C: 101-1000 nM; D: >1000 nM for the compounds of Formula (I)).

In some embodiments the presence of substituents on monocyclic Cy² rings or when Cy² is a bicyclic ring (unsubstituted or substituted) provides compounds with improved selectivity for inhibition of HPK1 vs Lck (see, for example, Table 3 and Table 4).

### (c) Human Jurkat T Lymphocyte Anti-Proliferation Assay

Assay principle: Jurkat cells are incubated with various concentrations of test compounds for 72 h, and cell proliferation/cytotoxicity is measured via detection of ATP production.

Literature: See Cree, I A et. al.; Toxicol In Vitro., 11 (5): 553-556, 1997 for additional information of ATP detection.

Assay Procedure: Jurkat cells (cultured in α-MEM media with 10% FBS) are seeded at 2000 cells/well (50 μL) in a 384-well well black culture plate (Perkin Elmer). Test compounds (in DMSO) are added to cells using the HP digital dispenser, and incubated at 37° C., 5% CO₂ for 72 hours. ATP production is measured by adding 40 μL/well of ATPLite-1-Step reagent (Perkin Elmer), incubating for 5 min at RT with shaking followed by detection of luminescent signal using an Envision plate reader (Perkin Elmer). Data is normalized to untreated cells, and plotted using XLFit. IC₅₀ values are calculated using a 4 parameter dose-response equation by fitting the curve of % inhibition versus Log of compound concentration.

Results: In an embodiment, exemplary compounds of the application had IC₅₀'s in the range of 0.1 to >10 10 μM in this assay. In an embodiment, exemplary compounds of the application had IC₅₀'s in the range of 1.0 to >10 uM in this assay.

### (d) Human Jurkat T Lymphocyte IL-2 Release Assay

Assay principle: Stimulation of TCR signaling via anti-CD3/CD28 antibody treatment in Jurkat T cells will lead to IL-2 secretion into the culture media, detected by a luminescent energy transfer bead immunoassay (IL-2 AlphaLISA kit).

Literature: See Cauchon, E. et. al.; Analytical Biochemistry., 388 (1): 134-139, 2009 for additional information on AlphaLISA detection.

Assay procedure: Jurkat cells (cultured in α-MEM media with 10% FBS) are seeded at 0.2×10⁶ cells/well (100 μL) in a 96-well round bottom culture plate (Greiner). Test compounds (in DMSO) are added to cells using an HP digital dispenser, and incubated for 15 mins at RT. Cells are stimulated with 15 μL/mL (v/v) of soluble CD3/CD28 antibodies in α-MEM media (Stem Cell Technologies; 50 μL/well, 150 μL final assay volume), and incubated at 37° C., 5% CO₂ for 4 hours. Cells are centrifuged at 1500 rpm for 5 mins at RT, and 5 μL of culture media is transferred to a 96 well % area white plate (Perkin Elmer). IL-2 is detected by adding 20 μL/well of a mixture of both Acceptor beads and Biotin anti-IL-2 antibody (1:200 dilution of each), and incubated for 1 hour at RT with shaking. 25 μL/well of Donor beads (1:63 dilution) are then added and incubated in the dark for 30 mins at RT with shaking, followed by detection of luminescent signal using an Envision plate reader (Perkin Elmer). Data is normalized to untreated/stimulated cells, and plotted using XLFit. EC₁₅₀ values are calculated using a 4 parameter dose-response equation by fitting the curve of % stimulation versus Log of compound concentration.

Results: In an embodiment, exemplary compounds of the application had EC₁₅₀'s in the range of 0.01-5 uM in this assay. In an embodiment, exemplary compounds of the application had EC₁₅₀'s in the range of 0.01-2.2 μM in this assay.

(e) p-SLP76 S376 Phosphorylation Inhibition Assay

Assay principle: Stimulation of TCR signaling via anti-CD3/CD28 antibody treatment in Jurkat T cells will lead to phosphorylation of HPK1 at Serine 376, detected by a luminescent energy transfer bead immunoassay (p-SPL76 AlphaLISA kit).

Literature: See Cauchon, E. et. al.; Analytical Biochemistry., 388 (1): 134-139, 2009 for additional information on AlphaLISA detection.

Assay procedure: Jurkat cells (cultured in α-MEM media with 10% FBS) are collected and centrifuged, and seeded in a 96-well % area white plate (Perkin Elmer) at 0.1×10⁶ cells/well in HBSS (25 μL). Test compounds (in DMSO) are added to cells using an HP digital dispenser, and incubated for 15 mins at RT. Cells are stimulated with 15 μl/mL (v/v) of soluble CD3/CD28 antibodies in HBSS (Stem Cell Technologies; 25 μL, 50 μL final assay volume), and incubated at 37° C., 5% CO₂ for 1 hour. Cells are lysed with 10 μL/well of lysis buffer followed by incubation at RT for 10 mins with shaking. p-SLP76 S376 is detected by adding 15 μl/well of Acceptor beads (1:50 dilution), and incubated for 1 hour at RT with shaking. 15 μL/well of Donor beads (1:50 dilution) are then added and incubated at RT in the dark for 1 hour with shaking, followed by detection of luminescent signal using an Envision plate reader (Perkin Elmer). Data is normalized to untreated/stimulated cells, and plotted using Graph Pad Prism. IC₅₀ values are calculated using a 4 parameter dose-response equation by fitting the curve of % inhibition versus Log of compound concentration.

### f) Glucose Kinase (Glk) Assay

GLK(h) (MAP4K3(h)) is incubated with 8 mM MOPS pH 7.0, 0.2 mM EDTA, 250 μM RLGRDKYKTLRQIRQ, 10 mM Magnesium acetate and [9-33P-ATP] (specific activity and concentration as required). The reaction is initiated by the addition of the Mg/ATP mix. After incubation for 40 minutes at room temperature, the reaction is stopped by the addition of phosphoric acid to a concentration of 0.5%. 10 μl of the reaction is then spotted onto a P30 filtermat and washed four times for 4 minutes in 0.425% phosphoric acid and once in methanol prior to drying and scintillation counting. For more details on the kinase assay procedures see: Gao, Y. et. al.; Biochem J., 451 (2): 313-328, 2013.

Results: In an embodiment, exemplary compounds of the application had IC₅₀'s in the range of greater than 10× greater than the value for HPK1 in this assay. In an embodiment, exemplary compounds of the application had IC₅₀'s in the range of at least 30× greater than HPK1 in this assay.

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 2

| | | | HPK1 | Lck |
|---|---|---|---|---|
| | | | IC₅₀ | IC₅₀, |
| Compound | | | nM | nM |
| I.D | IUPAC Chemical Name | Structure | Range* | Range* |
| I-1 | 6-(3-amino-6-(4-(4- methylpiperazin-1- yl)phenyl)pyrazin-2-yl)-3,4- dihydroisoquinolin-1(2H)- one | 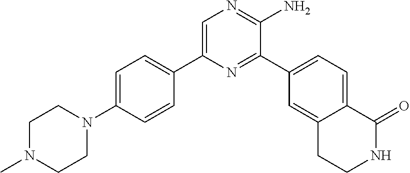 [SMILES: CN1CCN(c2ccc(-c3cnc(N)c(-c4ccc5c(c4)CCNC5=O)n3)cc2)CC1] | A | B |
| I-2 | 5-(3-amino-6-(4-(4- methylpiperazin-1- yl)phenyl)pyrazin-2-yl)-3,3- dimethylisoindolin-1-one | 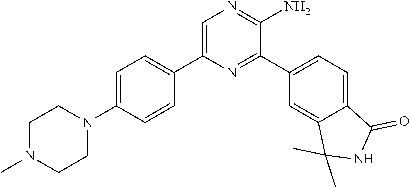 [SMILES: CN1CCN(c2ccc(-c3cnc(N)c(-c4ccc5c(c4)C(C)(C)NC5=O)n3)cc2)CC1] | A | B |
| I-3 | 6-(3-amino-6-(4-(4- isopropylpiperazin-1- yl)phenyl)pyrazin-2-yl)-3,4- dihydroisoquinolin-1(2H)- one | 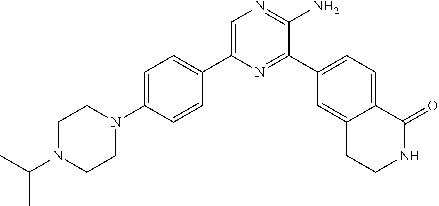 [SMILES: CC(C)N1CCN(c2ccc(-c3cnc(N)c(-c4ccc5c(c4)CCNC5=O)n3)cc2)CC1] | A | B |
| I-4 | 6-(3-amino-6-(3-fluoro-4- (4-methylpiperazin-1- yl)phenyl)pyrazin-2-yl)-3,4- dihydroisoquinolin-1(2H)- one | 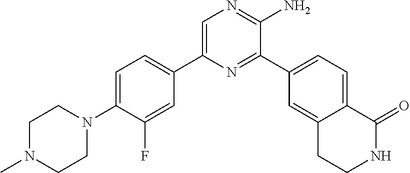 [SMILES: CN1CCN(c2ccc(-c3cnc(N)c(-c4ccc5c(c4)CCNC5=O)n3)cc2F)CC1] | A | B |
| I-5 | 6-(3-amino-6-(4-(4- methylpiperazin-1- yl)phenyl)pyrazin-2-yl)-8- fluoro-3,4- dihydroisoquinolin-1(2H)- one | 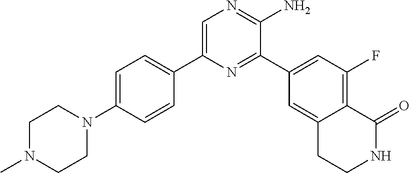 [SMILES: CN1CCN(c2ccc(-c3cnc(N)c(-c4cc(F)c5c(c4)CCNC5=O)n3)cc2)CC1] | A | B |
| I-6 | 6-(3-amino-6-(4-(4- hydroxypiperidin-1- yl)phenyl)pyrazin-2-yl)-3,4- dihydroisoquinolin-1(2H)- one | 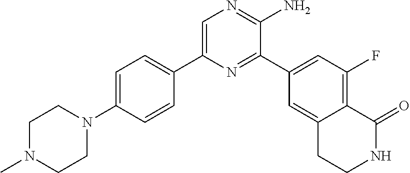 [SMILES: CN1CCN(c2ccc(-c3cnc(N)c(-c4cc(F)c5c(c4)CCNC5=O)n3)cc2)CC1] | A | C |
| I-7 | (R)-6-(3-amino-6-(4-(2- isopropylmorpholino)phenyl) pyrazin-2-yl)-3,4- dihydroisoquinolin-1(2H)- one | 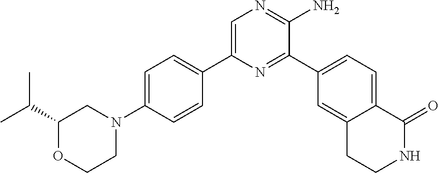 [SMILES: CC(C)[C@@H]1CN(c2ccc(-c3cnc(N)c(-c4ccc5c(c4)CCNC5=O)n3)cc2)CCO1] | A | C |
| I-8 | (S)-6-(3-amino-6-(4-(2- isopropylmorpholino)phenyl) pyrazin-2-yl)-3,4- dihydroisoquinolin-1(2H)- one | 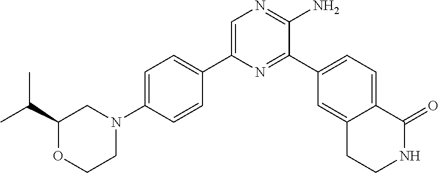 [SMILES: CC(C)[C@H]1CN(c2ccc(-c3cnc(N)c(-c4ccc5c(c4)CCNC5=O)n3)cc2)CCO1] | A | C |
| I-9 | (R)-N-(1-(4-(5-amino-6-(1- oxo-1,2,3,4- tetrahydroisoquinolin-6- yl)pyrazin-2- yl)phenyl)pyrrolidin-3-yl)- N- methylmethanesulfonamide | 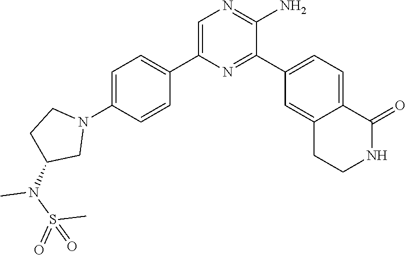 [SMILES: CN([C@@H]1CCN(c2ccc(-c3cnc(N)c(-c4ccc5c(c4)CCNC5=O)n3)cc2)C1)S(C)(=O)=O] | A | C |
| I-10 | (S)-N-(1-(4-(5-amino-6-(1- oxo-1,2,3,4- tetrahydroisoquinolin-6- yl)pyrazin-2- yl)phenyl)pyrrolidin-3-yl)-N- methylmethanesulfonamide | 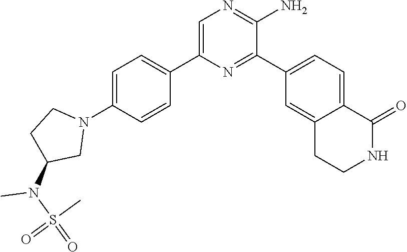 [SMILES: CN([C@H]1CCN(c2ccc(-c3cnc(N)c(-c4ccc5c(c4)CCNC5=O)n3)cc2)C1)S(C)(=O)=O] | A | C |
| I-11 | (R)-6-(3-amino-6-(4-(3- methylmorpholino)phenyl) pyrazin-2-yl)-3,4- dihydroisoquinolin-1(2H)- one | 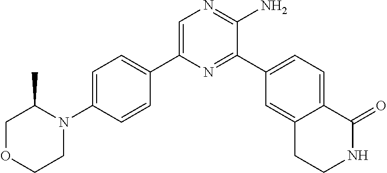 [SMILES: C[C@@H]1COCCN1c1ccc(-c2cnc(N)c(-c3ccc4c(c3)CCNC4=O)n2)cc1] | A | C |
| I-12 | 6-(3-amino-6-(4-(4- methylpiperazin-1- yl)phenyl)pyrazin-2-yl)-7- fluoro-3,4- dihydroisoquinolin-1(2H)- one | 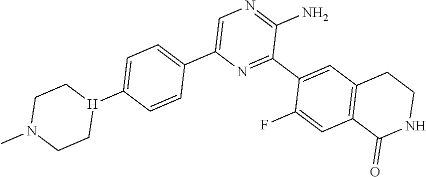 | A | C |
| I-13 | (R)-6-(3-amino-6-(2-fluoro- 4-(2- isopropylmorpholino)phenyl) pyrazin-2-yl)-3,4- dihydroisoquinolin-1(2H)- one | 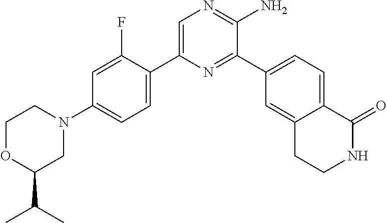 [SMILES: CC(C)[C@@H]1CN(c2ccc(-c3cnc(N)c(-c4ccc5c(c4)CCNC5=O)n3)c(F)c2)CCO1] | A | D |
| I-14 | (S)-6-(3-amino-6-(2-fluoro- 4-(2- isopropylmorpholino)phenyl) pyrazin-2-yl)-3,4- dihydroisoquinolin-1(2H)- one | 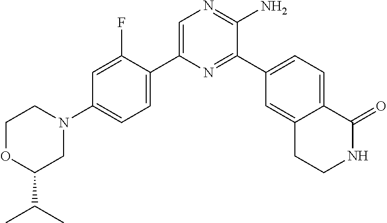 [SMILES: CC(C)[C@H]1CN(c2ccc(-c3cnc(N)c(-c4ccc5c(c4)CCNC5=O)n3)c(F)c2)CCO1] | A | D |
| I-15 | (R)-6-(3-amino-6-(2,3- difluoro-4-(2- isopropylmorpholino)phenyl) pyrazin-2-yl)-3,4- dihydroisoquinolin-1(2H)- one | 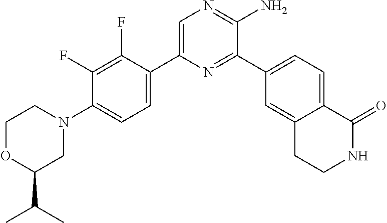 [SMILES: CC(C)[C@@H]1CN(c2ccc(-c3cnc(N)c(-c4ccc5c(c4)CCNC5=O)n3)c(F)c2F)CCO1] | A | D |
| I-16 | (S)-6-(3-amino-6-(2,3- difluoro-4-(2- isopropylmorpholino)phenyl) pyrazin-2-yl)-3,4- dihydroisoquinolin-1(2H)- one | 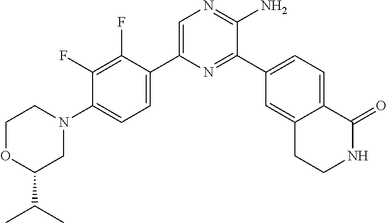 [SMILES: CC(C)[C@H]1CN(c2ccc(-c3cnc(N)c(-c4ccc5c(c4)CCNC5=O)n3)c(F)c2F)CCO1] | A | D |
| I-17 | (R)-6-(3-amino-6-(2-fluoro- 4-(2-methylpiperidin-1- yl)phenyl)pyrazin-2-yl)-3,4- dihydroisoquinolin-1(2H)- one | 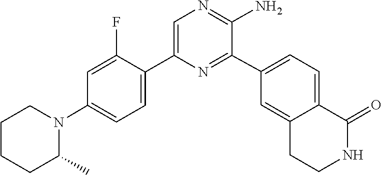 [SMILES: C[C@@H]1CCCCN1c1ccc(-c2cnc(N)c(-c3ccc4c(c3)CCNC4=O)n2)c(F)c1] | A | D |
| I-18 | (S)-6-(3-amino-6-(2-fluoro- 4-(2-methylpiperidin-1- yl)phenyl)pyrazin-2-yl)-3,4- dihydroisoquinolin-1(2H)- one | 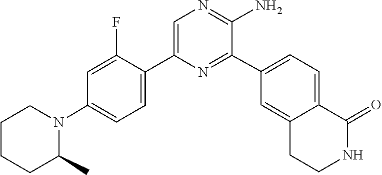 [SMILES: C[C@H]1CCCCN1c1ccc(-c2cnc(N)c(-c3ccc4c(c3)CCNC4=O)n2)c(F)c1] | B | D |
| I-19 | 6-(6-(4-(4-acetylpiperazin- 1-yl)phenyl)-3- aminopyrazin-2-yl)-3,4- dihydroisoquinolin-1(2H)- one | 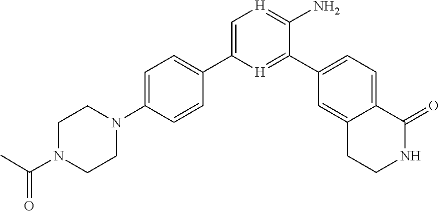 | A | C |
| I-20 | 6-(3-amino-6-(4-(3- (dimethylamino)pyrrolidin- 1-yl)-2- fluorophenyl)pyrazin-2-yl)- 3,4-dihydroisoquinolin- 1(2H)-one | 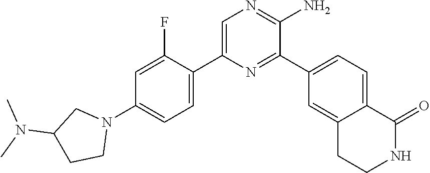 [SMILES: CN(C)C1CCN(c2ccc(-c3cnc(N)c(-c4ccc5c(c4)CCNC5=O)n3)c(F)c2)C1] | A | C |
| I-21 | 6-(3-amino-6-(4-(3- (dimethylamino)pyrrolidin- 1-yl)-2,3- difluorophenyl)pyrazin-2- yl)-3,4-dihydroisoquinolin- 1(2H)-one | 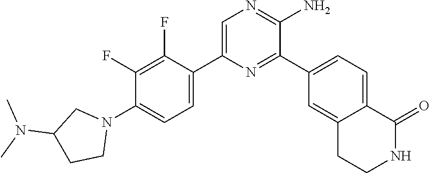 [SMILES: CN(C)C1CCN(c2ccc(-c3cnc(N)c(-c4ccc5c(c4)CCNC5=O)n3)c(F)c2F)C1] | A | C |
| I-22 | (R)-6-(3-amino-6-(2-fluoro- 4-(3- methylmorpholino)phenyl) pyrazin-2-yl)-3,4- dihydroisoquinolin-1(2H)- one | 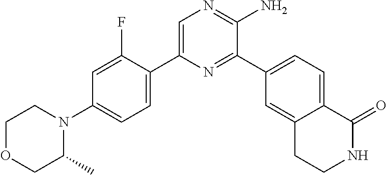 [SMILES: C[C@@H]1COCCN1c1ccc(-c2cnc(N)c(-c3ccc4c(c3)CCNC4=O)n2)c(F)c1] | B | D |
| I-23 | (S)-6-(3-amino-6-(2-fluoro- 4-(3- methylmorpholino)phenyl) pyrazin-2-yl)-3,4- dihydroisoquinolin-1(2H)- one | 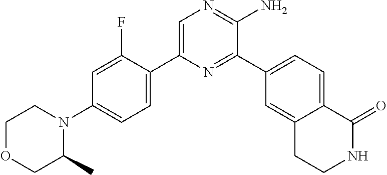 [SMILES: C[C@H]1COCCN1c1ccc(-c2cnc(N)c(-c3ccc4c(c3)CCNC4=O)n2)c(F)c1] | A | C |
| I-24 | 6-(3-amino-6-(4-(3- isopropylpiperidin-1- yl)phenyl)pyrazin-2-yl)-3,4- dihydroisoquinolin-1(2H)- one | 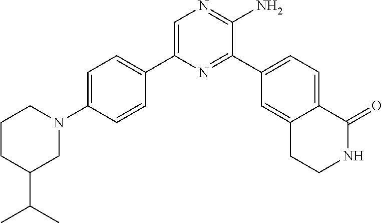 [SMILES: CC(C)C1CCCN(c2ccc(-c3cnc(N)c(-c4ccc5c(c4)CCNC5=O)n3)cc2)C1] | A | D |
| I-25 | 6-(3-amino-6-(2-fluoro-4- (3-isopropylpiperidin-1- yl)phenyl)pyrazin-2-yl)-3,4- dihydroisoquinolin-1(2H)- one | 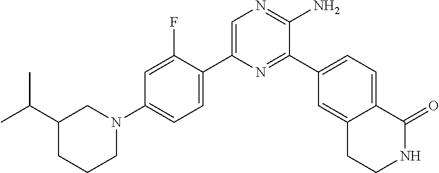 [SMILES: CC(C)C1CCCN(c2ccc(-c3cnc(N)c(-c4ccc5c(c4)CCNC5=O)n3)c(F)c2)C1] | B | D |
| I-26 | 6-(3-amino-6-(4-((1S,4S)- 5-methyl-2,5- diazabicyclo[2.2.1]heptan- 2-yl)phenyl)pyrazin-2-yl)- 3,4-dihydroisoquinolin- 1(2H)-one | 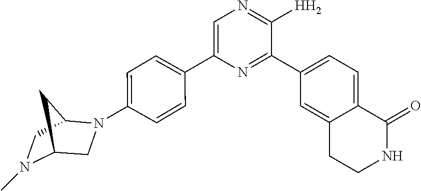 | A | C |
| I-27 | (R)-6-(3-amino-6-(4-(2- isopropylmorpholino)phenyl) pyrazin-2-yl)-7-fluoro-3,4- dihydroisoquinolin-1(2H)- one | 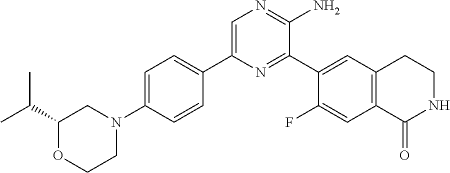 [SMILES: CC(C)[C@@H]1CN(c2ccc(-c3cnc(N)c(-c4cc5c(cc4F)C(=O)NCC5)n3)cc2)CCO1] | A | B |
| I-28 | (S)-6-(3-amino-6-(4-(2- isopropylmorpholino)phenyl) pyrazin-2-yl)-7-fluoro-3,4- dihydroisoquinolin-1(2H)- one | 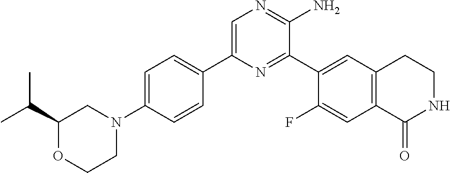 [SMILES: CC(C)[C@H]1CN(c2ccc(-c3cnc(N)c(-c4cc5c(cc4F)C(=O)NCC5)n3)cc2)CCO1] | A | C |
| I-29 | (R)-6-(3-amino-6-(2-fluoro- 4-(2- isopropylmorpholino)phenyl) pyrazin-2-yl)-7-fluoro-3,4- dihydroisoquinolin-1(2H)- one | 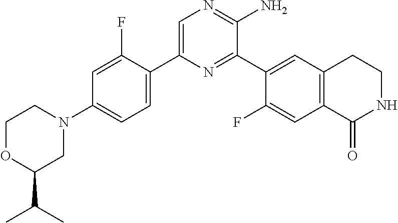 [SMILES: CC(C)[C@@H]1CN(c2ccc(-c3cnc(N)c(-c4cc5c(cc4F)C(=O)NCC5)n3)c(F)c2)CCO1] | A | D |
| I-30 | (S)-6-(3-amino-6-(2-fluoro- 4-(2- isopropylmorpholino)phenyl) pyrazin-2-yl)-7-fluoro-3,4- dihydroisoquinolin-1(2H)- one | 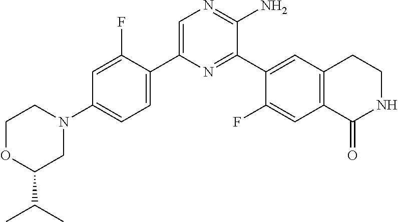 [SMILES: CC(C)[C@H]1CN(c2ccc(-c3cnc(N)c(-c4cc5c(cc4F)C(=O)NCC5)n3)c(F)c2)CCO1] | A | D |
| I-31 | (R)-6-(3-amino-6-(2,3- difluoro-4-(2- isopropylmorpholino)phenyl) pyrazin-2-yl)-7-fluoro-3,4- dihydroisoquinolin-1(2H)- one | 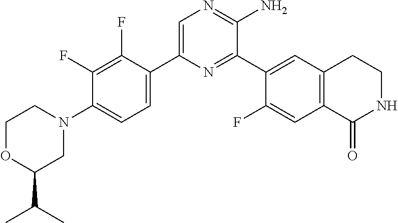 [SMILES: CC(C)[C@@H]1CN(c2ccc(-c3cnc(N)c(-c4cc5c(cc4F)C(=O)NCC5)n3)c(F)c2F)CCO1] | A | D |
| I-32 | (S)-6-(3-amino-6-(2,3- difluoro-4-(2- isopropylmorpholino)phenyl) pyrazin-2-yl)-7-fluoro-3,4- dihydroisoquinolin-1(2H)- one | 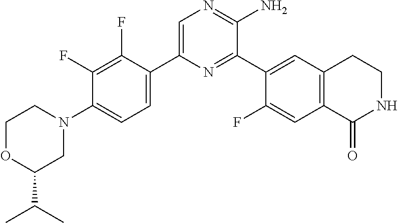 [SMILES: CC(C)[C@H]1CN(c2ccc(-c3cnc(N)c(-c4cc5c(cc4F)C(=O)NCC5)n3)c(F)c2F)CCO1] | A | D |
| I-33 | 6-(3-amino-6-(4-(1-methyl- 5,6-dihydro-1,2,4-triazin- 4(1H)-yl)phenyl)pyrazin-2- yl)-3,4-dihydroisoquinolin- 1(2H)-one | 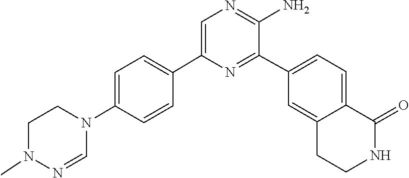 [SMILES: CN1CCN(c2ccc(-c3cnc(N)c(-c4ccc5c(c4)CCNC5=O)n3)cc2)C=N1] | A | C |
| I-34 | 6-(3-amino-6-(4-((1S,4S)- 5-(2,2,2-trifluoroethyl)-2,5- diazabicyclo[2.2.1]heptan- 2-yl)phenyl)pyrazin-2-yl)- 3,4-dihydroisoquinolin- 1(2H)-one | 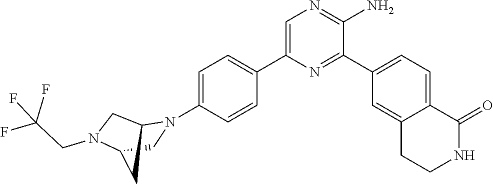 [SMILES: Nc1ncc(-c2ccc(N3C[C@@H]4C[C@H]3CN4CC(F)(F)F)cc2)nc1-c1ccc2c(c1)CCNC2=O] | A | C |
| I-35 | 6-(3-amino-6-(4-(4-(2,2,2- trifluoroethyl)piperazin-1- yl)phenyl)pyrazin-2-yl)-3,4- dihydroisoquinolin-1(2H)- one | 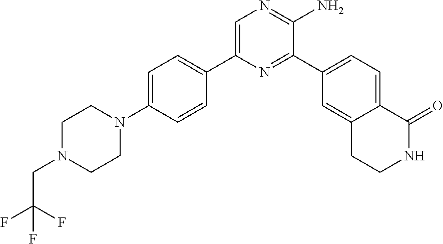 [SMILES: Nc1ncc(-c2ccc(N3CCN(CC(F)(F)F)CC3)cc2)nc1-c1ccc2c(c1)CCNC2=O] | A | C |
| I-36 | 6-(3-amino-6-(4-(1-(2,2,2- trifluoroethyl)piperidin-4- yl)phenyl)pyrazin-2-yl)-3,4- dihydroisoquinolin-1(2H)- one | 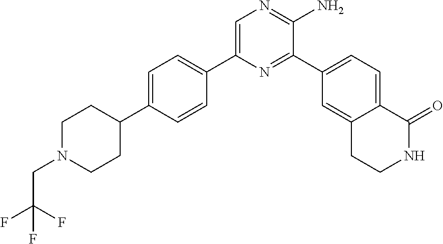 [SMILES: Nc1ncc(-c2ccc(C3CCN(CC(F)(F)F)CC3)cc2)nc1-c1ccc2c(c1)CCNC2=O] | A | C |
| I-37 | 6-(3-amino-6-(4-((1R,5S)- 3-(2-fluoroethyl)-3- azabicyclo[3.1.0]hexan-1- yl)phenyl)pyrazin-2-yl)-3,4- dihydroisoquinolin-1(2H)- one | 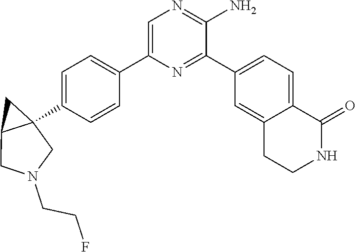 [SMILES: Nc1ncc(-c2ccc([C@@]34C[C@@H]3CN(CCF)C4)cc2)nc1-c1ccc2c(c1)CCNC2=O] | A | B |
| I-38 | (R)-6-(3-amino-6-(4-(3- isopropyl-4- methylpiperazin-1- yl)phenyl)pyrazin-2-yl)-3,4- dihydroisoquinolin-1(2H)- one | 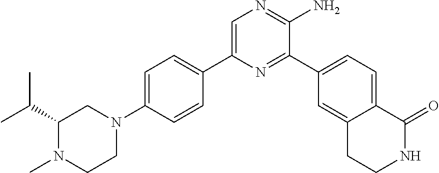 [SMILES: CC(C)[C@@H]1CN(c2ccc(-c3cnc(N)c(-c4ccc5c(c4)CCNC5=O)n3)cc2)CCN1C] | A | B |
| I-39 | (R)-6-(3-amino-6-(2-fluoro- 4-(3-isopropyl-4- methylpiperazin-1- yl)phenyl)pyrazin-2-yl)-3,4- dihydroisoquinolin-1(2H)- one | 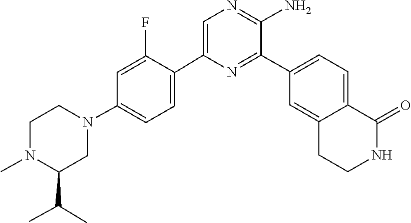 [SMILES: CC(C)[C@@H]1CN(c2ccc(-c3cnc(N)c(-c4ccc5c(c4)CCNC5=O)n3)c(F)c2)CCN1C] | A | C |
| I-40 | (S)-6-(3-amino-6-(4-(3- isopropyl-4- methylpiperazin-1- yl)phenyl)pyrazin-2-yl)-3,4- dihydroisoquinolin-1(2H)- one | 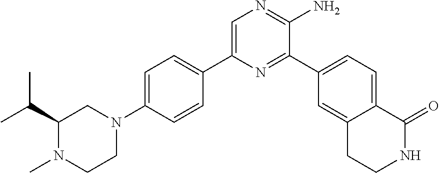 [SMILES: CC(C)[C@H]1CN(c2ccc(-c3cnc(N)c(-c4ccc5c(c4)CCNC5=O)n3)cc2)CCN1C] | A | B |
| I-41 | (S)-6-(3-amino-6-(2-fluoro- 4-(3-isopropyl-4- methylpiperazin-1- yl)phenyl)pyrazin-2-yl)-3,4- dihydroisoquinolin-1(2H)- one | 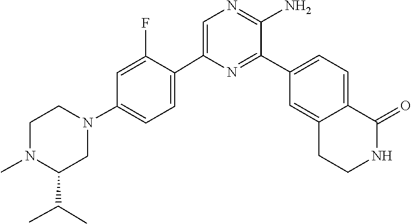 [SMILES: CC(C)[C@H]1CN(c2ccc(-c3cnc(N)c(-c4ccc5c(c4)CCNC5=O)n3)c(F)c2)CCN1C] | A | D |
| I-42 | (R)-6-(3-amino-6-(4-(2- methylpyrrolidin-1- yl)phenyl)pyrazin-2-yl)-3,4- dihydroisoquinolin-1(2H)- one | 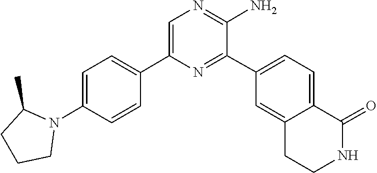 [SMILES: C[C@@H]1CCCN1c1ccc(-c2cnc(N)c(-c3ccc4c(c3)CCNC4=O)n2)cc1] | A | C |
| I-43 | (R)-6-(3-amino-6-(2-fluoro- 4-(2-methylpyrrolidin-1- yl)phenyl)pyrazin-2-yl)-3,4- dihydroisoquinolin-1(2H)- one | 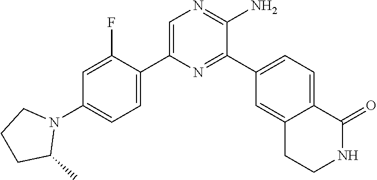 [SMILES: C[C@@H]1CCCN1c1ccc(-c2cnc(N)c(-c3ccc4c(c3)CCNC4=O)n2)c(F)c1] | B | D |
| I-44 | 6-(3-amino-6-(4-(4- isopropylpiperazin-1- yl)phenyl)pyrazin-2-yl)-7- fluoro-3,4- dihydroisoquinolin-1(2H)- one | 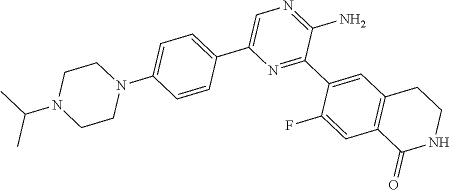 [SMILES: CC(C)N1CCN(c2ccc(-c3cnc(N)c(-c4cc5c(cc4F)C(=O)NCC5)n3)cc2)CC1] | A | C |
| I-45 | 6-(3-amino-6-(4-((1R,5S)- 3-methyl-3- azabicyclo[3.1.0]hexan-1- yl)phenyl)pyrazin-2-yl)-7- fluoro-3,4- dihydroisoquinolin-1(2H)- one | 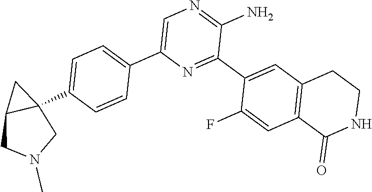 [SMILES: CN1C[C@H]2C[C@@]2(c2ccc(-c3cnc(N)c(-c4cc5c(cc4F)C(=O)NCC5)n3)cc2)C1] | A | C |
| I-46 | 6-(3-amino-6-(4-((1R,5S)- 3-isopropyl-3- azabicyclo[3.1.0]hexan-1- yl)phenyl)pyrazin-2-yl)-7- fluoro-3,4- dihydroisoquinolin-1(2H)- one | 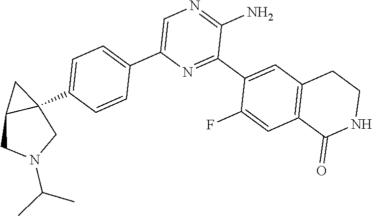 [SMILES: CC(C)N1C[C@H]2C[C@@]2(c2ccc(-c3cnc(N)c(-c4cc5c(cc4F)C(=O)NCC5)n3)cc2)C1] | A | C |
| I-47 | 6-(3-amino-6-(4-((1S,5R)- 3-methyl-3- azabicyclo[3.1.0]hexan-1- yl)phenyl)pyrazin-2-yl)-7- fluoro-3,4- dihydroisoquinolin-1(2H)- one | 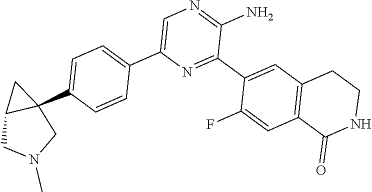 [SMILES: CN1C[C@@H]2C[C@]2(c2ccc(-c3cnc(N)c(-c4cc5c(cc4F)C(=O)NCC5)n3)cc2)C1] | A | C |
| I-48 | 6-(3-amino-6-(4-((1S,5R)- 3-isopropyl-3- azabicyclo[3.1.0]hexan-1- yl)phenyl)pyrazin-2-yl)-7- fluoro-3,4- dihydroisoquinolin-1(2H)- one | 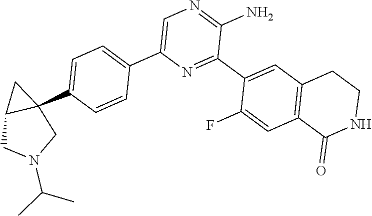 [SMILES: CC(C)N1C[C@@H]2C[C@]2(c2ccc(-c3cnc(N)c(-c4cc5c(cc4F)C(=O)NCC5)n3)cc2)C1] | A | C |
| I-49 | 6-(3-amino-6-(4-(4- methylpiperazin-1- yl)phenyl)pyridazin-4-yl)- 3,4-dihydroisoquinolin- 1(2H)-one | 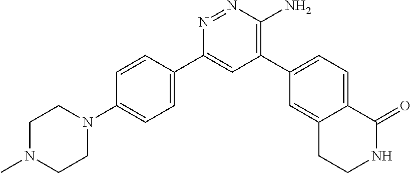 [SMILES: CN1CCN(c2ccc(-c3cc(-c4ccc5c(c4)CCNC5=O)c(N)nn3)cc2)CC1] | D | |
| I-50 | 7-(3-amino-6-(4-(4- methylpiperazin-1- yl)phenyl)pyrazin-2-yl)- 2,3,4,5-tetrahydro-1H- benzo[c]azepin-1-one | 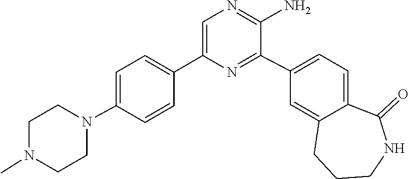 [SMILES: CN1CCN(c2ccc(-c3cnc(N)c(-c4ccc5c(c4)CCCNC5=O)n3)cc2)CC1] | A | B |
| I-51 | 6-(3-amino-6-(4-(4- methylpiperazin-1- yl)phenyl)pyrazin-2-yl)-3- methylisoquinolin-1(2H)- one | 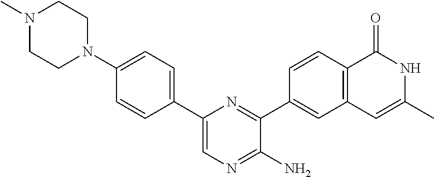 | A | |
| I-52 | 6-(3-amino-6-(4-((1S,5R)- 3-cyclopropyl-3- azabicyclo[3.1.0]hexan-1- yl)phenyl)pyrazin-2-yl)-7- fluoro-3,4- dihydroisoquinolin-1(2H)- one | 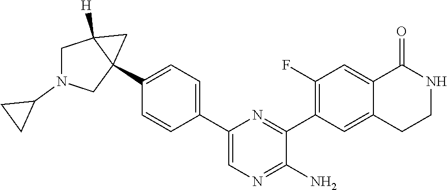 [SMILES: Nc1ncc(-c2ccc([C@]34C[C@H]3CN(C3CC3)C4)cc2)nc1-c1cc2c(cc1F)C(=O)NCC2] | A | B |
| I-53 | (R)-6-(3-amino-6-(4-(4- isopropyl-2- methylpiperazin-1- yl)phenyl)pyrazin-2-yl)-3,4- dihydroisoquinolin-1(2H)- one | 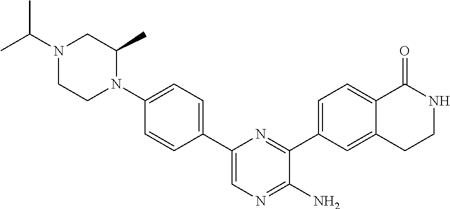 [SMILES: CC(C)N1CCN(c2ccc(-c3cnc(N)c(-c4ccc5c(c4)CCNC5=O)n3)cc2)[C@H](C)C1] | A | |
| I-54 | (R)-6-(3-amino-6-(4-(2,4- dimethylpiperazin-1- yl)phenyl)pyrazin-2-yl)-3,4- dihydroisoquinolin-1(2H)- one | 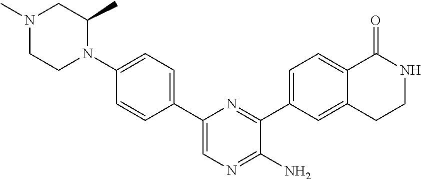 [SMILES: C[C@@H]1CN(C)CCN1c1ccc(-c2cnc(N)c(-c3ccc4c(c3)CCNC4=O)n2)cc1] | A | |
| I-55 | 6-(3-amino-6-(4-((3R,5S)- 3,4,5-trimethylpiperazin-1- yl)phenyl)pyrazin-2-yl)-3,4- dihydroisoquinolin-1(2H)- one | 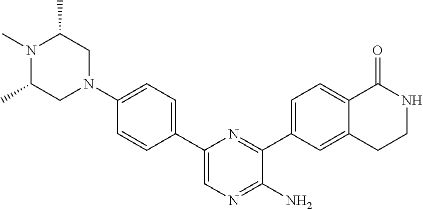 [SMILES: C[C@@H]1CN(c2ccc(-c3cnc(N)c(-c4ccc5c(c4)CCNC5=O)n3)cc2)C[C@H](C)N1C] | A | |
| I-56 | 6-(3-amino-6-(4-(4- ethylpiperazin-1- yl)phenyl)pyrazin-2-yl)-3,4- dihydroisoquinolin-1(2H)- one | 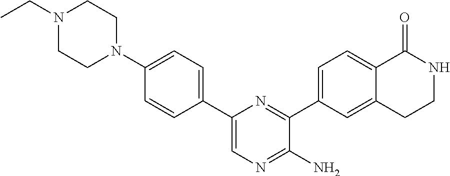 [SMILES: CCN1CCN(c2ccc(-c3cnc(N)c(-c4ccc5c(c4)CCNC5=O)n3)cc2)CC1] | A | |
| I-57 | 7-(3-amino-6-(4-(4- methylpiperazin-1- yl)phenyl)pyrazin-2-yl)-2- methylquinazolin-4(3H)- one | 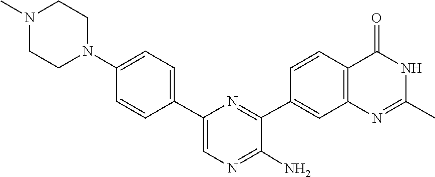 | B | |
| II-1 | 6-(3-amino-6-(2- ((dimethylamino)methyl)-4- (tetrahydro-2H-pyran-4- yl)phenyl)pyrazin-2-yl)-7- fluoro-3,4- dihydroisoquinolin-1(2H)- one | 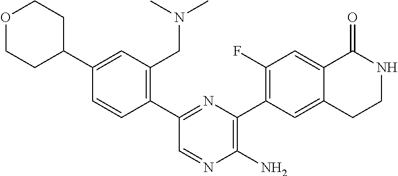 [SMILES: CN(C)Cc1cc(C2CCOCC2)ccc1-c1cnc(N)c(-c2cc3c(cc2F)C(=O)NCC3)n1] | B | |
| II-2 | 6-(3-amino-6-(3- ((dimethylamino)methyl)-4- (tetrahydro-2H-pyran-4- yl)phenyl)pyrazin-2-yl)-7- fluoro-4-methylisoquinolin- 1(2H)-one | 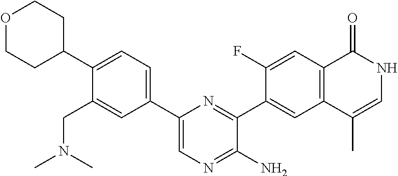 [SMILES: Cc1c[nH]c(=O)c2cc(F)c(-c3nc(-c4ccc(C5CCOCC5)c(CN(C)C)c4)cnc3N)cc12] | A | |
| II-3 | 7-(3-amino-6-(3- ((dimethylamino)methyl)-4- (tetrahydro-2H-pyran-4- yl)phenyl)pyrazin-2-yl)-2- methylquinazolin-4(3H)- one | 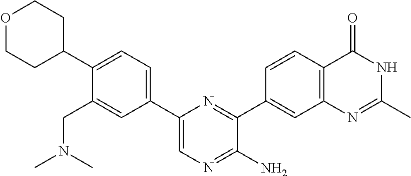 [SMILES: Cc1nc2cc(-c3nc(-c4ccc(C5CCOCC5)c(CN(C)C)c4)cnc3N)ccc2c(=O)[nH]1] | A | D |
| II-4 | 6-(3-amino-6-(3- ((dimethylamino)methyl)-4- (tetrahydro-2H-pyran-4- yl)phenyl)pyrazin-2-yl)-8- fluoro-3-methylisoquinolin- 1(2H)-one | 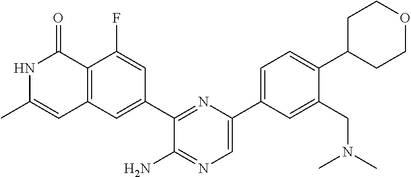 [SMILES: Cc1cc2cc(-c3nc(-c4ccc(C5CCOCC5)c(CN(C)C)c4)cnc3N)cc(F)c2c(=O)[nH]1] | A | |
| II-5 | 6-(3-amino-6-(3- ((dimethylamino)methyl)-4- (tetrahydro-2H-pyran-4- yl)phenyl)pyrazin-2-yl)-4,8- difluoro-3- methylisoquinolin-1(2H)- one | 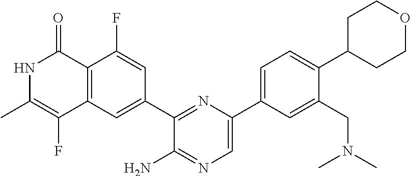 [SMILES: Cc1[nH]c(=O)c2c(F)cc(-c3nc(-c4ccc(C5CCOCC5)c(CN(C)C)c4)cnc3N)cc2c1F] | A | |
| II-6 | 6-(3-amino-6-(3- ((dimethylamino)methyl)-4- (tetrahydro-2H-pyran-4- yl)phenyl)pyrazin-2-yl)-4,7- difluoro-3- methylisoquinolin-1(2H)- one | 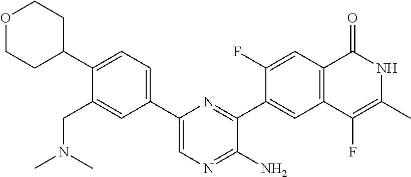 [SMILES: Cc1[nH]c(=O)c2cc(F)c(-c3nc(-c4ccc(C5CCOCC5)c(CN(C)C)c4)cnc3N)cc2c1F] | A | D |

* A: 0.1-5 nm; B: 6-10 nm; C: 11-20 nm; D: 21-200 nm; and E: >200 nm

TABLE 3

| Comparative | | |
|---|---|---|
| Compound | IUPAC Chemical Name | Structure |
| Comparator 1 | 6-(3-amino-6-(4- morpholinophenyl)pyrazin-2-yl)- 3,4-dihydroisoquinolin-1(2H)-one | 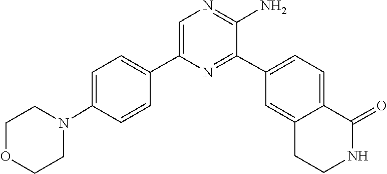 [SMILES: Nc1ncc(-c2ccc(N3CCOCC3)cc2)nc1-c1ccc2c(c1)CCNC2=O] |
| Comparator 2 | 6-(3-amino-6-(2,3-difluoro-4- morpholinophenyl)pyrazin-2-yl)- 3,4-dihydroisoquinolin-1(2H)-one | 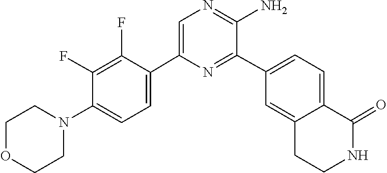 [SMILES: Nc1ncc(-c2ccc(N3CCOCC3)c(F)c2F)nc1-c1ccc2c(c1)CCNC2=O] |
| Comparator 3 | 6-(3-amino-6-(3-fluoro-4- morpholinophenyl)pyrazin-2-yl)- 3,4-dihydroisoquinolin-1(2H)-one | 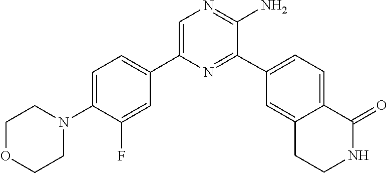 [SMILES: Nc1ncc(-c2ccc(N3CCOCC3)c(F)c2)nc1-c1ccc2c(c1)CCNC2=O] |
| Comparator 4 | 6-(3-amino-6-(2-fluoro-4- morpholinophenyl)pyrazin-2-yl)- 3,4-dihydroisoquinolin-1(2H)-one | 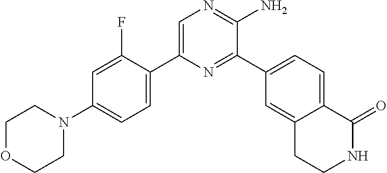 [SMILES: Nc1ncc(-c2ccc(N3CCOCC3)cc2F)nc1-c1ccc2c(c1)CCNC2=O] |

TABLE 4

| Comparative Compound | Selectivity | Compound of Formula (I) | Selectivity |
|---|---|---|---|
| 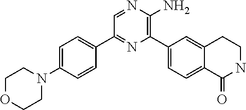 Comparator 1 | 31 | 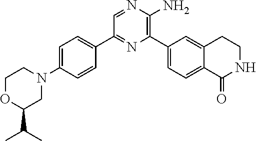 [SMILES: CC(C)[C@@H]1CN(c2ccc(-c3cnc(N)c(-c4ccc5c(c4)CCNC5=O)n3)cc2)CCO1] | 170 |
| | | 1-8 | |
| 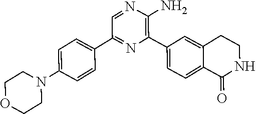 [SMILES: Nc1ncc(-c2ccc(N3CCOCC3)cc2)nc1-c1ccc2c(c1)CCNC2=O] Comparator 1 | 31 | 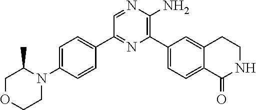 [SMILES: C[C@@H]1COCCN1c1ccc(-c2cnc(N)c(-c3ccc4c(c3)CCNC4=O)n2)cc1] 1-11 | 170 |
| 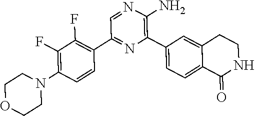 [SMILES: Nc1ncc(-c2ccc(N3CCOCC3)c(F)c2F)nc1-c1ccc2c(c1)CCNC2=O] Comparator 2 | 930 | 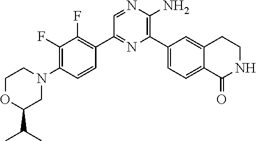 [SMILES: CC(C)[C@@H]1CN(c2ccc(-c3cnc(N)c(-c4ccc5c(c4)CCNC5=O)n3)c(F)c2F)CCO1] | 7200 |
| | | 1-31 | |
| 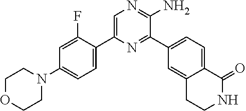 [SMILES: Nc1ncc(-c2ccc(N3CCOCC3)cc2F)nc1-c1ccc2c(c1)CCNC2=O] Comparator 4 | 405 | 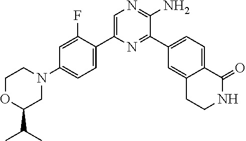 [SMILES: CC(C)[C@@H]1CN(c2ccc(-c3cnc(N)c(-c4ccc5c(c4)CCNC5=O)n3)c(F)c2)CCO1] | 2740 |
| | | 1-13 | |
| 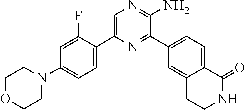 [SMILES: Nc1ncc(-c2ccc(N3CCOCC3)cc2F)nc1-c1ccc2c(c1)CCNC2=O] Comparator 4 | 405 | 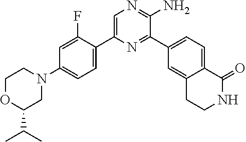 [SMILES: CC(C)[C@H]1CN(c2ccc(-c3cnc(N)c(-c4ccc5c(c4)CCNC5=O)n3)c(F)c2)CCO1] | 1066 |
| | | 1-14 | |

1. A compound of Formula (I), or a pharmaceutically acceptable salt, solvate and/or prodrug thereof, 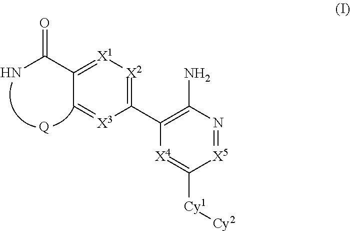
wherein:
X¹ is selected from N and CR¹;
X² and X³ are each independently selected from N and CR²;
X⁴ and X⁵ are each independently selected from N and CH, provided at least one of X⁴ and X⁵ is N;
Q is C₁₋₄alkylene optionally interrupted by a heteromoiety selected from O, S, S(O), SO₂ and NR³ and/or optionally substituted with one or more of R⁴ and/or optionally disubstituted on one carbon with R^{4a} and R^{4b}, provided that when Q comprises the heteromoiety the heteromoiety is separated from the ring amide NH by other than methylene; or
Q is C₂₋₄alkenylene optionally substituted with one or more of R^{4c}; or
Q is C═N or N═C optionally substituted with R^{4c};
R¹ is selected from H, halo, OR^{3a}, NR^{5a}R^{6a}, C₁₋₆alkyleneNR^{5a}R^{6a} and C₁₋₆alkyl;
R² is selected from H, halo and C₁₋₆alkyl;
R³ is selected from H and C₁₋₆alkyl;
each R⁴ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR⁵R⁶ and C₁₋₆alkyleneNR⁵R⁶;
R^{4a} and R^{4b} are joined to form, together with the atom therebetween, a 3- to 6-membered, saturated or unsaturated ring optionally containing one additional heteromoiety selected from N, NH, NC₁₋₆alkyl, O, S, S(O), and SO₂ and optionally substituted with one or more of halo and C₁₋₆alkyl;
each R^{4c} is independently selected from halo, C₁₋₆alkyl, C₃₋₆cycloalkyl, C₃₋₆heterocycloalkyl, C₁₋₆alkyleneC₃₋₆cycloalkyl and C₁₋₆alkyleneC₃₋₆heterocycloalkyl, OH, OC₁-6alkyl, NR⁵R⁶, and C₁₋₆alkyleneNR⁵R⁶;
R⁵, R^{5a}, R⁶ and R^{6a} are each independently selected from H and C₁₋₆alkyl, or
R⁵ and R⁶ or R^{5a} and R^{6a} are joined to form, together with the nitrogen atom therebetween, a 3- to 7-membered, saturated or unsaturated ring optionally containing one additional heteromoiety selected from N, NH, NC₁₋₆alkyl, O, S, S(O), and SO₂ and optionally substituted with one or more of halo and C₁₋₆alkyl;
Cy¹ is C₆₋₁₀aryl or C₅₋₁₀heteroaryl, which is unsubstituted or substituted with one or more of R⁷;
each R⁷ is independently selected from halo, ═O, C₁₋₆alkyl, NR⁸R⁹, and C₁₋₆alkyleneNR⁸R⁹, C₃₋₇cycloalkyl, C₃₋₇heterocycloalkyl, C₁₋₆alkyleneC₃₋₇cycloalkyl and C₁₋₆alkyleneC₃₋₇heterocycloalkyl, the latter four groups being optionally substituted with one or more of R¹⁰;
R⁸ and R⁹ are each independently selected from H and C₁₋₆alkyl;
each R¹⁰ is independently selected from halo, C₁₋₆alkyl, CN and NR¹¹R^{11a};
R¹¹ and R^{11a} are each independently selected from H and C₁₋₆alkyl;
Cy² is a monocyclic C₃₋₇heterocycloalkyl which is substituted with one or more of R¹² or a bicyclic C₆₋₁₂heterocycloalkyl which is unsubstituted or substituted with one or more of R¹²;
each R¹² is independently selected from halo, CN, ═O, OH, C₁₋₆alkyl, C₂₋₆alkenyl, C₂₋₆alkynyl, C₃₋₁₀cycloalkyl, C₃₋₁₀heterocycloalkyl, C₁₋₆alkyleneC₃₋₁₀cycloalkyl, C₁₋₆alkyleneC₃₋₁₀heterocycloalkyl, C₁₋₆alkyleneOR¹³, C₁₋₆alkyleneNR¹³R¹⁴, OC₁₋₆alkyleneOR¹³, OC₁₋₆alkyleneNR¹³R¹⁴, SR¹³, C(O)R¹³, C(O)C₁₋₆alkyleneOR¹³, C(O)C₁₋₆alkyleneNR¹³R¹⁴, C(O)C₁₋₆alkyleneOC₁-6alkyleneNR¹³R¹⁴, C(O)NR¹³R¹⁴, CO₂R¹³, CO₂C₁₋₆alkyleneOR¹³, CO₂C₁₋₆alkyleneOC₁₋₆alkyleneNR¹³R¹⁴, NR¹³R¹⁴, NR¹⁵SO₂R¹³, S(O)R¹³, SO₂R¹³, SO₂NR¹³R¹⁴ and S(O)(NR¹⁵)R¹³;
R¹³ is selected from H, C₁₋₆alkyl, C₁₋₆alkyleneOR¹⁴, C₃₋₁₀cycloalkyl, C₃₋₁₀heterocycloalkyl, C₁₋₆alkyleneC₃₋₁₀cycloalkyl and C₁₋₆alkyleneC₃₋₁₀heterocycloalkyl, R¹⁴ is selected from H and C₁₋₆alkyl; or
R¹³ and R¹⁴ are joined to form, together with the nitrogen atom therebetween, a 4- to 6-membered saturated or unsaturated ring, optionally containing one additional heteromoiety selected from N, NR¹⁶, O, S, S(O) and SO₂; and
R¹⁵ and R¹⁶ are selected from H and C₁₋₆alkyl;
wherein all available hydrogen atoms are optionally substituted with a fluorine atom, provided Cy² is not 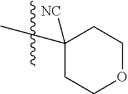 [SMILES: *C1(C#N)CCOCC1]
when Cy¹ is unsubstituted phenyl wherein 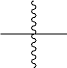
represents a point of covalent attachment to Cy¹.

2. The compound of claim 1, wherein X¹ is N, or wherein X¹ is CR¹ and R¹ is selected from OR^{3a}, NR^{5a}R^{6a}, C₁₋₄alkyleneNR^{5a}R^{6a} and C₁₋₄alkyl.

3. (canceled)

4. (canceled)

5. The compound of claim 1, wherein Q is C₁₋₃alkylene optionally substituted with one to three of R⁴, and each R⁴ is independently selected from ═O, F, Cl, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁-4alkyl, NR⁵R⁶, and C₁₋₄alkyleneNR⁵R⁶; or
Q is C₂₋₄alkenylene optionally substituted with one or two of R^{4c}, and each R^{4c} is independently selected from F, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁-6alkyl, NR⁵R⁶, and C₁₋₄alkyleneNR⁵R⁶; or
Q is selected from C═N and N═C and is optionally substituted with R^{4c}, and R^{4c} is selected from F, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₆alkyl, NR⁵R⁶, and C₁₋₄alkyleneNR⁵R⁶; or
Q is C₁₋₃alkylene optionally disubstituted on one carbon atom with R^{4a} and R^{4b}, and R^{4a} and R^{4b} are joined to form, together with the carbon atom therebetween, a 3- to 6-membered saturated or unsaturated ring optionally containing one heteromoiety selected from N, NH, NC₁₋₆alkyl, O, S, S(O), and SO₂ and optionally substituted with one or more of halo and C₁₋₄alkyl; and
wherein all available hydrogen atoms are optionally substituted with a fluorine atom.

6.-21. (canceled)

22. The compound of claim 1, wherein one of X² and X³ is N and the other is CR²; or
both X² and X³ are independently CR²;
each R² is independently selected from H, F, Cl and C₁₋₄alkyl, and
one of X⁴ and X⁵ is N and the other is CH;
wherein all available hydrogen atoms are optionally substituted with a fluorine atom.

23.-28. (canceled)

29. The compound of claim 1, wherein Cy¹ is phenyl which is unsubstituted or substituted with one or more of R⁷, or
Cy¹ is C₅₋₁₀heteroaryl which is unsubstituted or substituted with one or more of R⁷;
wherein each R⁷ is independently selected from halo, C₁₋₄alkyl, NR⁸R⁹, C₁₋₄alkyleneNR⁸R⁹, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹⁰—;
each R¹⁰ is independently selected from F, Cl, CN, C₁₋₄alkyl and NR¹¹R^{11a}, and R¹¹ and R^{11a} are each independently selected from H and C₁₋₄alkyl,
wherein all available hydrogen atoms are optionally substituted with a fluorine atom.

30.-50. (canceled)

51. The compound of claim 1, wherein Cy² is a monocyclic C₃₋₇heterocycloalkyl which is substituted with one to three of R¹², or
Cy² is a bicyclic heterocycle which is substituted with one to three of R¹²,
each R¹² is independently selected from halo, ═O, OH, C₁₋₄alkyl, C₃₋₁₀cycloalkyl, C₃₋₁₀heterocycloalkyl, C₁₋₄alkyleneC₃₋₁₀cycloalkyl, C₁₋₄alkyleneC₃₋₁₀heterocycloalkyl, C₁₋₄alkyleneOR¹³, C₁₋₄alkyleneNR¹³R¹⁴, OC₁₋₄alkyleneOR¹³, OC₁-4alkyleneNR¹³R¹⁴, C(O)R¹³, C(O)C₁₋₄alkyleneOR¹³, C(O)C₁₋₄alkyleneNR¹³R¹⁴, C(O)C₁₋₄alkyleneOC₁₋₄alkyleneNR¹³R¹⁴, C(O)NR¹³R¹⁴, CO₂R¹³, CO₂C₁₋₄alkyleneOR¹³, CO₂C₁₋₄alkyleneOC₁₋₄alkyleneNR¹³R¹⁴, NR¹³R¹⁴, NR¹⁵SO₂R¹³, SO₂R¹³ and SO₂NR¹³R¹⁴,
R¹³ is selected from H, C₁₋₄alkyl, C₃₋₆cycloalkyl, C₁₋₄alkyleneC₃₋₆cycloalkyl, C₃₋₆heterocycloalkyl and C₁₋₄alkyleneC₃₋₆heterocycloalkyl; or
R¹³ and R¹⁴ are joined to form, together with the nitrogen atom therebetween, a 5- or 6-membered saturated or unsaturated ring, optionally containing one additional heteromoiety selected from N, NR¹⁶, O, S, S(O) and SO₂; and
R¹⁵ and R¹⁶ are independently selected from H and C₁₋₄alkyl;
wherein all available hydrogen atoms are optionally substituted with a fluorine atom.

52.-70. (canceled)

71. The compound of claim 1, wherein the compound of Formula (I) is selected from the compounds listed in Table 1, or a pharmaceutically acceptable salt, solvate and/or prodrug thereof.

72. A compound of Formula (II), or a pharmaceutically acceptable salt, solvate and/or prodrug thereof, 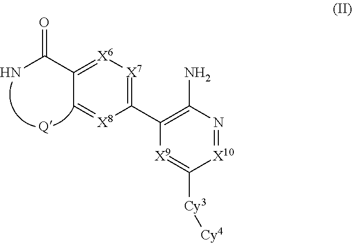
wherein
X⁶ is selected from N and CR¹⁷;
X⁷ and X⁸ are each independently selected from N and CR¹⁸;
X⁹ and X¹⁰ are each independently selected from N and CH, provided at least one of X⁹ and X¹⁰ is N;
Q′ is C₁₋₄alkylene optionally interrupted by a heteromoiety selected from O, S, S(O), SO₂ and NR¹⁹ and/or optionally substituted with one or more of R²⁰ and/or optionally disubstituted on one carbon with R²¹ and R^{21a}, provided that when Q comprises the heteromoiety the heteromoiety is separated from the ring amide NH by other than methylene; or
Q′ is C₂₋₄alkenylene optionally substituted with one or more of R²²; or
Q′ is C═N or N═C optionally substituted with R²²;
R¹⁷ is selected from H, halo, OR²³, NR²⁴R²¹, C₁₋₆alkyleneNR²⁴R²¹ and C₁₋₆alkyl;
R¹⁸ is selected from H, halo and C₁₋₆alkyl;
R¹⁹ is selected from H and C₁₋₆alkyl;
each R²⁰ is independently selected from ═O, halo, C₁₋₆alkyl, C₃₋₆cycloalkyl, C₃-6heterocycloalkyl, C₁₋₆alkyleneC₃₋₆cycloalkyl, C₁₋₆alkyleneC₃₋₆heterocycloalkyl, OH, OC₁₋₆alkyl, NR²⁶R²⁷ and C₁₋₆alkyleneNR²⁶R²⁷;
R²¹ and R^{21a} are joined to form, together with the carbon atom therebetween, a 3- to 6-membered, saturated or unsaturated ring optionally containing one heteromoiety selected from N, NH, NC₁-6alkyl, O, S, S(O), and SO₂ and optionally substituted with one or more of halo and C₁₋₆alkyl;
each R²² is independently selected from halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR²⁶R²⁷ and C₁₋₆alkyleneNR²⁶R²⁷;
R²⁴, R²⁵, R²⁶ and R²⁷ are each independently selected from H and C₁₋₆alkyl, or
R²⁴ and R²⁵ or R²⁶ and R²⁷ are joined to form, together with the atom therebetween, a 3- to 7-membered saturated or unsaturated ring, optionally containing one additional heteromoiety selected from N, NH, NC₁₋₆alkyl, O, S, S(O), and SO₂ and optionally substituted with one or more of halo and C₁₋₆alkyl;
Cy³ is C₆₋₁₀aryl or C₅₋₁₀heteroaryl, which substituted with one or two of R²⁸, and optionally further substituted with one to three of R²⁹;
each R²⁸ is independently selected from NR³⁰R³¹, C₁₋₆alkyleneNR³⁰R³¹, C₃₋₇heterocycloalkyl and C₁₋₆alkyleneC₃₋₇heterocycloalkyl, the latter two groups being optionally substituted with one or more of R³²;
each R²⁹ is independently selected from halo, C₁₋₆alkyl, C₃₋₇cycloalkyl, and C₁₋₆alkyleneC₃₋₇cycloalkyl, the latter two groups being optionally substituted with one or more of R³²;
R³⁰ and R³¹ are each independently selected from H and C₁₋₆alkyl;
each R³² is independently selected from halo, C₁₋₆alkyl, CN and NR³³R³⁴;
R³³ and R³⁴ are each independently selected from H and C₁₋₆alkyl;
Cy⁴ is C₃₋₁₄heterocycloalkyl, and Cy⁴ is unsubstituted or substituted with one or more of R³⁵;
each R³⁵ is independently selected from halo, ═O, CN, OH, C₁₋₆alkyl, C₂₋₆alkenyl, C₂₋₆alkynyl, C₃₋₁₀cycloalkyl, C₃₋₁₀heterocycloalkyl, C₁₋₆alkyleneC₃₋₁₀cycloalkyl, C₁₋₆alkyleneC₃₋₁₀heterocycloalkyl, C₁₋₆alkyleneOR³⁶, C₁₋₆alkyleneNR³⁶R³⁷, OC₁₋₆alkyleneOR³⁶, OC₁₋₆alkyleneNR³⁶R³⁷, SR³⁶, C(O)R³⁶ C(O)C₁₋₆alkyleneOR³⁶, C(O)C₁₋₆alkyleneNR³⁶R³⁷, C(O)C₁₋₆alkyleneOC₁-6alkyleneNR³⁶R³⁷, C(O)NR³⁶R³⁷, CO₂R³⁶, CO₂C₁₋₆alkyleneOR³⁶, CO₂C₁₋₆alkyleneOC₁-6alkyleneNR³⁶R³⁷, NR³⁶R³⁷, NR³⁸SO₂R³⁶, S(O)R³⁷, SO₂R³⁷, SO₂NR³⁶R³⁷ and S(O)(NR³⁸)R³⁶;
R³⁶ is selected from H, C₁₋₆alkyl, C₁₋₆alkyleneOR¹⁴, C₃₋₁₀cycloalkyl, C₃₋₁₀heterocycloalkyl, C₁₋₆alkyleneC₃₋₁₀cycloalkyl and C₁₋₆alkyleneC₃₋₁₀heterocycloalkyl,
R³⁷ is selected from H and C₁₋₆alkyl; or
R³⁶ and R³⁷ are joined to form, together with the nitrogen atom therebetween, a 4- to 6-membered saturated or unsaturated ring, optionally containing one additional heteromoiety selected from N, NR³⁹, O, S, SO and SO₂; and
R³⁸ and R³⁹ are independently selected from H and C₁₋₆alkyl;
wherein all available hydrogen atoms are optionally substituted with a fluorine atom.

73. The compound of claim 72, wherein X⁶ is N, or wherein X⁶ is CR¹⁷ and R¹⁷ is selected from H, F, Cl, OR²³, NR²⁴R²⁵, C₁₋₄alkyleneNR²⁴R²⁵ and C₁₋₄alkyl,
wherein R²⁴ and R²⁵ are each independently selected from H and C₁₋₄alkyl, or
wherein R²⁴ and R²⁵ are joined to form, together with the atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl, and
wherein all available hydrogen atoms are optionally substituted with a fluorine atom.

74.-80. (canceled)

81. The compound of claim 72, wherein one of X⁷ and X⁸ is N and the other is CR¹⁸, or both X⁷ and X⁸ are independently CR¹⁸; and each R¹⁸ is independently selected from H, halo and C₁₋₄alkyl, and
wherein X⁹ is N and X¹⁰ is CH;
wherein all available hydrogen atoms are optionally substituted with a fluorine atom.

82.-85. (canceled)

86. The compound of claim 72, wherein Q′ is C₁₋₃alkylene optionally interrupted by a heteromoiety selected from O, SO₂, and NR¹⁹ and R¹⁹ is selected from H and C₁₋₄alkyl; or
Q′ is C₁₋₃alkylene and optionally substituted with one to three of R²⁰, each R²⁰ is independently selected from F, Cl, OH, C₁₋₄alkyl, OC₁-4alkyl and NR²⁶R²⁷; or
Q′ is C₁₋₃alkylene and optionally disubstituted on one carbon atom with R²¹ and R^{21a} wherein R²¹ and R^{21a} are joined to form, together with the carbon atom therebetween, a 3- to 5-membered cycloalkyl ring, optionally substituted with one or more of halo and C₁₋₄alkyl; or
Q′ is C₂₋₄alkenylene optionally substituted with one or two of R²³;
Q′ is selected from C═N or N═C and is optionally substituted with R²²; wherein each R²² is independently selected from F, Cl, C₁₋₄alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR²⁶R²⁷, and C₁₋₆alkyleneNR²⁶R²⁷;
R²⁶ and R²⁷ are independently selected from H and C₁₋₄alkyl, or
R²⁶ and R²⁷ are joined to form, together with the nitrogen atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl; and
wherein available hydrogen atoms are optionally substituted with a fluorine atom.

87.-108. (canceled)

109. The compound of claim 72, wherein Cy³ is phenyl which is substituted with one or two of R²⁸, and optionally further substituted with one to three of R²⁹; or
wherein Cy³ is C₅₋₁₀heteroaryl which substituted with one or two of R²⁸, and optionally further substituted with one to three of R²⁹; and
wherein one of R²⁸ is C₁₋₄alkyleneNR³⁰R³¹, and R³⁰ and R³¹ are each independently selected from H and C₁₋₄alkyl; or
one of R²⁸ is selected from C₃₋₇heterocycloalkyl and C₁₋₄alkyleneC₃₋₇heterocycloalkyl, optionally substituted with one to three of R³².

110.-120. (canceled)

121. The compound of claim 72, wherein Cy⁴ is a monocyclic C₃₋₇heterocycloalkyl which is unsubstituted or substituted with one or more of R³⁵; or
Cy⁴ is a bridged bicyclic heterocycle, fused bicyclic heterocycle or a spirofused bicyclic heterocycle which is substituted with one to three of R³⁵;
each R³⁵ is independently selected from halo, ═O, OH, C₁₋₄alkyl, C₃₋₁₀cycloalkyl, C₃₋₁₀heterocycloalkyl, C₁₋₄alkyleneC₃₋₁₀cycloalkyl, C₁₋₄alkyleneC₃₋₁₀heterocycloalkyl, C₁₋₄alkyleneOR³⁶, C₁₋₄alkyleneNR³⁶R³⁷, OC₁₋₄alkyleneOR³⁶, OC₁-4alkyleneNR³⁶R³⁷, C(O)R³⁶, C(O)C₁₋₄alkyleneOR³⁶, C(O)C₁₋₄alkyleneNR³⁶R³⁷, C(O)C₁₋₄alkyleneOC₁₋₄alkyleneNR³⁶R³⁷, C(O)NR³⁶R³⁷, CO₂R³⁶, CO₂C₁₋₄alkyleneOR³⁶, CO₂C₁₋₄alkyleneOC₁₋₄alkyleneNR³⁶R³⁷, NR³⁶R³⁷, NR³⁸SO₂R³⁷, SO₂R³⁶ and SO₂NR³⁶R³⁷, R³⁶ is selected from H, C₁₋₄alkyl, C₃₋₆cycloalkyl, C₁₋₄alkyleneC₃₋₆cycloalkyl, C₃₋₆heterocycloalkyl and C₁₋₄alkyleneC₃₋₆heterocycloalkyl and R³⁷ is selected from H and C₁₋₄alkyl, or
R³⁶ and R³⁷ are joined to form, together with the nitrogen atom therebetween, a 4- to 6-membered saturated or unsaturated ring, optionally containing one additional heteromoiety selected from N, NR¹⁶, O, S, S(O) and SO₂,
wherein all available hydrogen atoms are optionally substituted with a fluorine atom.

122.-128. (canceled)

129. The compound of claim 72, wherein the compound of Formula (II) is selected from the compounds listed in Table 1-A:

| Compound | | |
|---|---|---|
| I.D | Compound Name | Structure |
| II-1 | 6-(3-amino-6-(2- ((dimethylamino)methyl)-4- (tetrahydro-2H-pyran-4- yl)phenyl)pyrazin-2-yl)-7-fluoro- 3,4-dihydroisoquinolin-1(2H)-one | 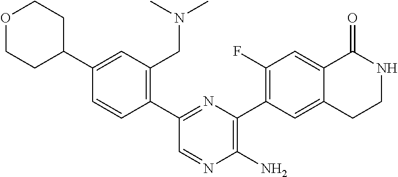 [SMILES: CN(C)Cc1cc(C2CCOCC2)ccc1-c1cnc(N)c(-c2cc3c(cc2F)C(=O)NCC3)n1] |
| II-2 | 6-(3-amino-6-(3- ((dimethylamino)methyl)-4- (tetrahydro-2H-pyran-4- yl)phenyl)pyrazin-2-yl)-7-fluoro- 4-methylisoquinolin-1(2H)-one | 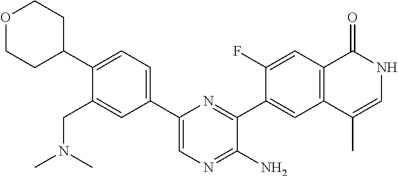 [SMILES: Cc1c[nH]c(=O)c2cc(F)c(-c3nc(-c4ccc(C5CCOCC5)c(CN(C)C)c4)cnc3N)cc12] |
| II-3 | 7-(3-amino-6-(3- ((dimethylamino)methyl)-4- (tetrahydro-2H-pyran-4- yl)phenyl)pyrazin-2-yl)-2- methylquinazolin-4(3H)-one | 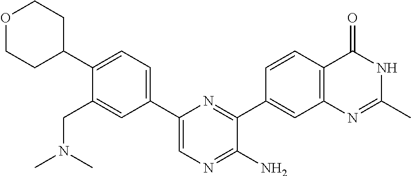 [SMILES: Cc1nc2cc(-c3nc(-c4ccc(C5CCOCC5)c(CN(C)C)c4)cnc3N)ccc2c(=O)[nH]1] |
| II-4 | 6-(3-amino-6-(3- ((dimethylamino)methyl)-4- (tetrahydro-2H-pyran-4- yl)phenyl)pyrazin-2-yl)-8-fluoro- 3-methylisoquinolin-1(2H)-one | 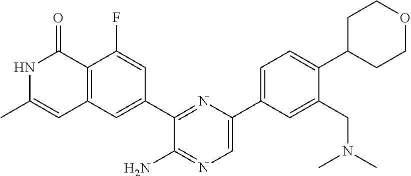 [SMILES: Cc1cc2cc(-c3nc(-c4ccc(C5CCOCC5)c(CN(C)C)c4)cnc3N)cc(F)c2c(=O)[nH]1] |
| II-5 | 6-(3-amino-6-(3- ((dimethylamino)methyl)-4- (tetrahydro-2H-pyran-4- yl)phenyl)pyrazin-2-yl)-4,8- difluoro-3-methylisoquinolin- 1(2H)-one | 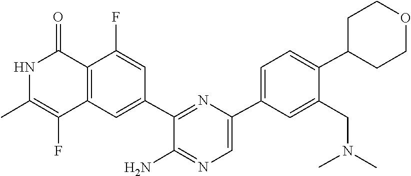 [SMILES: Cc1[nH]c(=O)c2c(F)cc(-c3nc(-c4ccc(C5CCOCC5)c(CN(C)C)c4)cnc3N)cc2c1F] |
| II-6 | 6-(3-amino-6-(3- ((dimethylamino)methyl)-4- (tetrahydro-2H-pyran-4- yl)phenyl)pyrazin-2-yl)-4,7- difluoro-3-methylisoquinolin- 1(2H)-one | 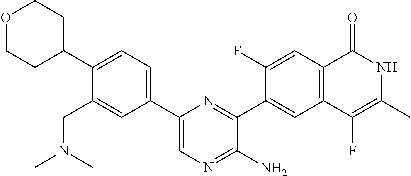 [SMILES: Cc1[nH]c(=O)c2cc(F)c(-c3nc(-c4ccc(C5CCOCC5)c(CN(C)C)c4)cnc3N)cc2c1F] |

or a pharmaceutically acceptable salt, solvate and/or prodrug thereof.

130. A pharmaceutical composition comprising one or more compounds of claim 1, or a pharmaceutically acceptable salt, solvate and/or prodrug thereof, and a pharmaceutically acceptable carrier and/or diluent.

131-134. (canceled)

135. A method of treating cancer comprising administering a therapeutically effective amount of one or more compounds of claim 1, or a pharmaceutically acceptable salt, solvate and/or prodrug thereof, to a subject in need thereof.

136. (canceled)

137. A method of inhibiting proliferative activity in a cell, comprising administering an effective amount of one or more compounds of claim 1, or a pharmaceutically acceptable salt, solvate and/or prodrug thereof, to the cell.

138. (canceled)

139. (canceled)

140. A pharmaceutical composition comprising one or more compounds of claim 72, or a pharmaceutically acceptable salt, solvate and/or prodrug thereof, and a pharmaceutically acceptable carrier and/or diluent.

141. A method of treating cancer comprising administering a therapeutically effective amount of one or more compounds of claim 72, or a pharmaceutically acceptable salt, solvate and/or prodrug thereof, to a subject in need thereof.

142. A method of inhibiting proliferative activity in a cell, comprising administering an effective amount of one or more compounds of claim 72, or a pharmaceutically acceptable salt, solvate and/or prodrug thereof, to the cell.
